(19) United States
(12) Patent Application Publication
(10) Pub. No.: US 2025/0002934 A1
(43) Pub. Date: Jan. 2, 2025

(54) METHODS AND COMPOSITIONS RELATING TO MAINTAINER LINES FOR MALE-STERILITY

(71) Applicant: ELSOMS DEVELOPMENTS LIMITED, Spalding, Lincolnshire (GB)

(72) Inventors: Anthony Gordon KEELING, Spalding (GB); Matthew MILNER, Cambridge (GB)

(73) Assignee: ELSOMS DEVELOPMENTS LIMITED, Spalding, Lincolnshire (GB)

(21) Appl. No.: 18/291,728

(22) PCT Filed: Jul. 26, 2022
(86) PCT No.: PCT/US2022/074126

§ 371 Date: Jan. 24, 2024

Related U.S. Application Data
(60) Provisional application No. 63/321,392, filed on Mar. 18, 2022.
(60) Provisional application No. 63/279,275, filed on Nov. 15, 2021.
(60) Provisional application No. 63/232,735, filed on Aug. 13, 2021.
(60) Provisional application No. 63/225,686, filed on Jul. 26, 2021.

(51) Int. Cl. C12N 15/82; A01H 1/00; A01H 1/02

(52) U.S. Cl. CPC C12N 15/8289; A01H 1/023; A01H 1/107; C12N 15/8212

(57) ABSTRACT

The methods and compositions described herein relate to maintainer lines (e.g, male-fertile lines) for fertilizing male-sterile plants and, due to their pollen not containing any expressed male-fertility gene, permitting the production or propagation of plants with a maintained male-sterile phenotype.

### CROSS-REFERENCE TO RELATED APPLICATIONS

This application claims benefit under 35 U.S.C. § 119(e) of U.S. Provisional Application Nos. 63/225,686 filed Jul. 26, 2021, 63/232,735 filed Aug. 13, 2021, 63/279,275 filed Nov. 15, 2021, and 63/321,392 filed Mar. 18, 2022, the contents of which are incorporated herein by reference in their entireties.

### SEQUENCE LISTING

The instant application contains a Sequence Listing which has been submitted in XML format via EFS-Web and is hereby incorporated by reference in its entirety. Said XML copy, created on Jul. 25, 2022, is named 077524-098450WOPT_SL.xml and is 1,000,749 bytes in size.

### TECHNICAL FIELD

The technology described herein relates to engineered plants, e.g., maintainer lines for male-sterile plants.

### BACKGROUND

Male-sterile lines, particularly recessive male-steriles which can be pollinated by wild-type pollen which restores fertility to the progeny, are of significant value in plant breeding operations, allowing certainty in the production of hybrids and avoiding costly manual procedures. However, a male-sterile line obviously cannot propagate itself. Instead, the male-sterile line is propogated via the use of a maintainer line whose pollen carries the same male-sterile alleles as the cognate male-sterile plant. The genetics of maintainer lines vary, but the general concept is that the line is arranged in such a way that the pollen produced can cross with a cognate male-sterile plant to produce a next generation of male-sterile plants without transferring male-fertilty. The maintainer line is further arranged such that at least a proportion of self-pollination propogates the same maintainer line genotype of the parent plant.

However, maintainer lines for recessive male-sterility lines have traditionally necessitated heavily transgenic and/or GMO approaches. Typical approaches that are incorporated into maintainer lines include expression cassettes or transgenes to “rescue” the male-sterility or transgenic cassettes designed to induce death or ineffectiveness of pollen or ovules of the undesired genotypes. In view of current worldwide agricultural regulatory approaches, such maintainer lines can be difficult and expensive to bring to bear and, in some regions/jurisdictions, unacceptable to the market.

### SUMMARY

Described herein is an approach to engineering a maintainer line (e.g., a wheat maintainer line) that minimizes or eliminates transgenic sequence use. As described herein, this maintainer lines requires the introduction or introgression of only two genes, both of which are Triticum genes from the same or a cross-transferable species. In some embodiments, the maintainer lines described herein relate to the introduction or introgression of only two constructs. In some embodiments, the maintainer lines described herein relate to the introduction or introgression of only three genes. This advance in maintainer line technology provides plants which are cheaper to produce and have broader applicability, e.g., by avoiding or minimizing the transgenic material that is utilized. Furthermore such advances in inserting native genes in their endogenous genomic form (cis-genesis or intra-genesis; with “cis-genesis” as used herein referring to both cis-genesis and intragenesis inclusively) and at defined, more beneficial loci, mean that knockouts of the genes concerned in endogenous loci are now feasible.

In one aspect of any of the embodiments, described herein is a maintainer line comprising the modifications engineered by the following process, or a maintainer line made by the following process. In one aspect of any of the embodiments, described herein is the following process for preparing or providing a maintainer line.
1. A pre-meiosis male-fertility gene is designed which is based on an endogenous gene (e.g., MF) but is subtly changed or different from the wild-type DNA sequence to have a DNA sequence which, at a gene-editing point, has a few bp that are different to the endogenous version [so denoted MF′] but which are ‘synonyms’ of the endogenous original and so translate to the identical amino-acids and protein. As explained below, the use of MF′ and PV′ genes can permit selection for the insertions and any other traits in a fully-fertile form before native MF or PV genes are knocked out. As used herein, “pre-meiosis”, used in reference to a gene, encompasses the time prior to the conclusion of meiosis while the relevant cells are still diploid. Genes can exert an effect while the cell is still sporophyte/diploid (with expression of both relevant alleles taking place (including during meiosis)), rather than when the cell is a gametophyte/haploid (e.g., when each allele is the only allele present to be expressed towards the end of meiosis and post-meiosis).
2. A same-species endogenous endosperm-expressed seed colour gene (e.g., denoted BA for blue aleurone) is provided in a construct with the above MF′ in tight genetic linkage (e.g., immediately adjoining it) so that progeny with the above pre-meiosis (e.g., pre-conclusion of meiosis) male-fertility genotype can be colour-selected and there is no risk of the two genes being delinked by crossing over between them with resultant wrong sorting and contamination.
3. The above pair of genes is targeted to be inserted into one chromosome at a selected locus in the plant genome. The selected locus can be, e.g, the endogenous MF or a pollen vital gene (denoted PV) locus in the plant genome, or a site which is at a different locus on the same or a separate chromosome from the MF or PV gene's endogenous locus in the plant genome (e.g., to facilitate selecting the inserted cassettes separately from the MF and PV genes which may be on the same chromosome (such as is the case with Mfw2 and Ms1). Where the plant genome is polyploid, the insertion can be made into the genome which most highly expresses the gene(s) at the selected locus.
4. A post-meiosis male-fertility gene is designed which is based on an endogenous gene but, as in step 1 above, it is subtly changed to have a DNA sequence which, at a gene-editing point, has a few bp that are different to the endogenous version [so denoted PV′] but which are ‘synonyms’ of the endogenous original and so translate to the identical amino-acids and protein.
5. The PV′ gene immediately above is targeted to be inserted into the same locus as described in step 3 above, but on the second chromosome of that genome, so that, after selection, it becomes an alternative allele to MF:BA at its homolgous locus.
6. After the concurrent insertion of the MF′:BA and PV′ constructs, plants are selected which have both inserts together in the same genome like a heterozygous pair of alleles at that locus: MF′:BA/PV′. (After natural selfing of these plants, they will have seed/progeny plants 50% of which will have repeats of such heterozygous genotype, 25% which will be homozygous MF′:BA, and 25% which will be homozygous PV′.)
7. Alternatively each of MF:BA′ and PV′ are inserted into separate wild-type plants/embryos at the same targeted locus; then, having established with PCR checks etc., that they are stably inserted the two plant types are crossed. The progeny can be screened to select a plant/plants which have heterozygous MF′:BA/PV′, or the F2 progeny can be subjected to step 8 and selection/screening performed after the knockout.
8. Knockout of the endogenous MF and PV genes is then performed. The knockout guides will not recognise the newly inserted versions of MF and PV which comprise the changed DNA sequence at a gene-editing point (e.g., MF′ and PV′). TO plants can then be found which have a complete knockout of the endogenous MF and PV genes, leaving just the new inserts (e.g., MF′ and PV′) unaffected to be expressed and active.
9. Successful knockout plants now endogenous pv/pv and mf/mf in any genome and homozygous PV′/PV′ or, at the same locus, PV′/null or null/null embryos/plants are immediately the new male-sterile. See, e.g., FIG. 23.
10. Successful knockout plants from the heterozygous MF′:BA/PV′ embryos/plants—now endogenous mf/mf and pv/pv in any genome—are immediately the fertile new maintainer for the above male-sterile (but with pollen which can only contain mf knocked out pre-meiosis male-fertility, e.g., it contains inserted gene PV′ but not MF′:BA), so that, crucially, male-fertility is not transferred to the male-sterile with its knocked out, mf genes. By genetically stopping the spread of MF genes to the male-sterile from the fertile maintainer using cis-genesis and endogenous gene knockouts, the current technology avoids the need, after the event, to eliminate, e.g., by colour sorting on a large scale, the fertility genes which have been spread into the male-sterile. In this way, the current technology avoids substantial waste of resources and product and provides improvements and advantages over previously known technologies.

In one aspect of any of the embodiments, described herein is a male-fertile maintainer plant for a male-sterile polyploid plant comprising:
a first genome comprising:
   on a first chromosome of a pair of homologous chromosomes, at a single target locus, at least one functional ectopic allele of a MF gene and at least one functional ectopic allele of a seed color gene (e.g., seed coat and/or seed endosperm gene) (or at least one functional ectopic allele of each member of a set of seed color genes);
   on a second chromosome of the pair of homologous chromosomes, at the target locus corresponding to the target locus of the first chromosome of the pair of homologous chromosomes, at least one functional ectopic allele of a PV gene;
   loss-of-function alleles of the endogenous MF genes at the native MF gene loci and loss-of-function alleles of the endogenous PV genes at the native PV gene loci.
      In one aspect of any of the embodiments, described herein is a method of preparing a male-fertile maintainer plant for a male-sterile polyploid plant, the method comprising engineering a plant to comprise:
in a first genome:
   on a first chromosome of a pair of homologous chromosomes, at a single target locus, at least one functional ectopic allele of a MF gene and at least one functional ectopic allele of a seed color gene (e.g., seed coat and/or seed endosperm gene) (or at least one functional ectopic allele of each member of a set of seed color genes);
   on a second chromosome of the pair of homologous chromosomes, at the target locus corresponding to the target locus of the first chromosome of the pair of homologous chromosomes, at least one functional ectopic allele of a PV gene; and
   loss-of-function alleles of the endogenous MF genes at the native MF gene loci and loss-of-function alleles of the endogenous PV genes at the native PV gene loci.
      In some embodiments of any of the aspects, the plant further comprises at least one further genome, each of the further genomes comprising loss-of-function alleles of the endogenous MF genes at the native MF gene loci and loss-of-function alleles of the PV gene at the native PV gene loci. In some embodiments of any of the aspects, the plant further comprises at least one further genome, and the method further comprises engineering loss-of-function alleles of the endogenous MF genes at the native MF gene loci and loss-of-function alleles of the PV gene at the native PV gene loci in each of the at least one further genomes.

In some embodiments of any of the aspects, the target locus is the native MF gene locus. In some embodiments of any of the aspects, the target locus is the native PV gene locus. In some embodiments of any of the aspects, the target locus is not the native MF gene locus or the native PV gene locus. In some embodiments of any of the aspects, the ectopic allele of the MF gene and/or the ectopic allele of the PV gene is a nuclease-null allele. In some embodiments of any of the aspects, the ectopic allele of the MF gene and/or the ectopic allele of the PV gene is a CRISPR-null allele.

In one aspect of any of the embodiments, described herein is a male-fertile maintainer plant for a male-sterile polyploid plant comprising:
a first genome comprising:
   on a first chromosome of a pair of homologous chromosomes, at least one functional allele of a MF gene at the MF gene locus and at least one allele of a seed color gene (e.g., seed coat and/or seed endosperm gene) (or at least one functional ectopic allele of each member of a set of seed color genes);
   on a second chromosome of the pair of homologous chromosomes, a loss-of-function allele of the MF gene at the MF gene locus and at least one ectopic functional allele of a PV gene;
   and loss-of-function alleles of the PV gene at the native PV gene loci; and
at least one further genome, each of the further genomes comprising loss-of-function alleles of the MF gene at the native MF gene loci and loss-of-function alleles of the PV gene at the native PV gene loci.
   In one aspect of any of the embodiments, described herein is a method of preparing a male-fertile maintainer plant for a male-sterile polyploid plant, the method comprising, simultaneously or sequentially: inserting, on a first chromosome of a pair of homologous chromosomes in a first genome, at a single target locus, a construct comprising at least one functional ectopic allele of a MF gene and at least one functional ectopic allele of a seed color gene (e.g., seed coat and/or seed endosperm gene) (or at least one functional ectopic allele of each member of a set of seed color genes), optionally wherein the inserting comprises nuclease cleavage of the target locus (e.g., zinc-finger nuclease or CRISPR nuclease cleavage) and recombination or end-joining of the construct; inserting, on a second chromosome of the pair of homologous chromosomes in the first genome, at the target locus corresponding to the target locus of the first chromosome of the pair of homologous chromosomes, a construct comprising at least one functional ectopic allele of a PV gene, optionally wherein the inserting comprises nuclease cleavage of the target locus (e.g., zinc-finger nuclease or CRISPR nuclease cleavage) and/or recombination or end-joining of the construct; and mutating the endogenous MF genes at the native MF gene loci and the endogenous PV genes at the native PV gene loci to create loss-of-function alleles, optionally wherein the loss-of-function alleles are caused by contacting the genome with a site-specific guided nuclease (e.g., CRISPR) and one or more guide RNA sequences or multi-guide constructs.

In one aspect, described herein is a method of preparing a male-fertile maintainer plant for a male-sterile polyploid plant, the method comprising:

inserting, on a first chromosome of a pair of homologous chromosomes in a first genome, at a single target locus, a cassette comprising in 5′ to 3′ or 3′ to 5′ order:
a first recognition site for a first recombinase;
at least one functional ectopic nuclease null allele of a MF gene and at least one functional ectopic allele of a seed color gene (e.g., seed coat and/or seed endosperm gene) (or at least one functional ectopic allele of each member of a set of seed color genes) in either relative order;
a first recognition site for a second recombinase;
a selection gene;
a second recognition site for the first recombinase;
at least one functional ectopic nuclease-null allele of a PV gene;
a second recognition site for the second recombinase;
   thereby providing a full-cassette insertion plant;
   contacting a first progeny of the full-cassette insertion plant, or a cell thereof, with the first recombinase,
   thereby excising:
one recognition site for the first recombinase, the at least one functional ectopic nuclease null allele of a MF gene and at least one functional ectopic allele of a seed color gene (e.g., seed coat and/or seed endosperm gene) (or at least one functional ectopic allele of each member of a set of seed color genes), the first recognition site for the second recombinase, and the selection gene from the genome of the first progeny and
   thereby providing an excised first progeny comprising:
one recognition site for the first recombinase, the at least one functional ectopic nuclease-null allele of a PV gene, and the second recognition site for the second recombinase portions of the construct;
   contacting a second progeny of the full-cassette insertion plant, or a cell thereof, with the second recombinase, thereby excising:
one recognition site for the second recombinase, the selection gene, the second recognition site for the first recombinase and at least one functional ectopic nuclease-null allele of a PV gene, and
   thereby providing an excised second progeny comprising:
one recognition site for the second recombinase, the first recognition site for the first recombinase, and the at least one functional ectopic nuclease null allele of a MF gene and at least one functional ectopic allele of a seed color gene (e.g., seed coat and/or seed endosperm gene) (or at least one functional ectopic allele of each member of a set of seed color genes) portions of the construct;
   crossing the excised first progeny provided in step ii) and the excised second progeny provided in step iii), thereby providing a third progeny comprising, in a first genome,
on a first chromosome of a pair of homologous chromosomes, at a single target locus, the at least one functional ectopic nuclease-null allele of a MF gene and the at least one functional ectopic allele of a seed color gene (e.g., seed coat and/or seed endosperm gene) (or at least one functional ectopic allele of each member of a set of seed color genes);
on a second chromosome of the pair of homologous chromosomes, at the target locus corresponding to the target locus of the first chromosome of the pair of homologous chromosomes, the at least one functional ectopic nuclease-null allele of a PV gene; and
   mutating the the endogenous MF genes at the native MF gene loci and the endogenous PV genes at the native PV gene loci to create loss-of-function alleles, optionally wherein the loss-of-function alleles are caused by contacting the genome with a site-specific guided nuclease (e.g., CRISPR) and one or more guide RNA sequences or multi-guide constructs, thereby providing the male-fertile maintainer plant. In some embodiments of any of the aspects, one of first recombinase and second recombinase is Cre and the other recombinase is Flp. In some embodiments of any of the aspects, the construct is a T-DNA construct. In some embodiments of any of the aspects, one or more of the steps further comprise selection of the provided plants or cells, optionally wherein the selection is PCR selection.

In one aspect, described herein is a method of preparing a male-fertile maintainer plant for a male-sterile polyploid plant, the method comprising:
i) contacting a cell comprising a PV locus in a first chromosome and a second chromosome of a pair of homologous chromosomes in a first genome, with:
   1) a site-specific guided nuclease (e.g., CRISPR);
   2) one or more guide RNA sequences or multi-guide constructs specific to one or more sequences at the PV locus; and
   3) a targeting insertion cassette comprising in 5′ to 3′ or 3′ to 5′ order: a first recognition site for a first recombinase and a second recognition site for the first recombinase;
   thereby providing a targeting insertion plant;
ii) contacting the targeting insertion plant, or first progeny of the targeting insertion plant, or a cell thereof with the first recombinase and a cassette comprising in 5′ to 3′ or 3′ to 5′ order:
   1) a first recombination site for the first recombinase;
   2) at least one functional ectopic nuclease null allele of a MF gene and at least one functional ectopic allele of a seed color gene (e.g., seed coat and/or seed endosperm gene) (or at least one functional ectopic allele of each member of a set of seed color genes) in either relative order; and
   3) a second recombination site for the first recombinase;
thereby providing a cassette insertion plant;
iii) selecting a cassette insertion plant comprising a cassette insertion at one allele of the PV locus, or crossing a cassette insertion plant comprising a cassette insertion at both alleles of the PV locus with a plant with a functional PV allele at the PV locus,
thereby providing a cassette insertion plant with a cassette insertion at one PV allele in the first genome and a functional PV allele at the second PV allele in the first genome,
iv) contacting the cassette insertion plant selected in iii), or a first progeny or cell thereof, with:
   1) a site-specific guided nuclease (e.g., CRISPR);
   2) one or more guide RNA sequences or multi-guide constructs flanking the insertion sites, thereby excising the inserted recombination sites;
   3) one or more guide RNA sequences or multi-guide constructs specific to the endogenous MF genes and/or flanking the endogenous MF genes, thereby mutating the endogenous MF genes at the native MF gene loci to create loss-of-function alleles;
      thereby providing the male-fertile maintainer plant. In some embodiments of any of the aspects, the contacting of step i) comprises biolistic delivery or integration. In some embodiments of any of the aspects, the contacting of step ii) comprises transforming the plant, progeny, or cell thereof with one or more T-DNAs comprising the recombinase and cassette. In some embodiments of any of the aspects, the method further comprises a step v) of segregating remaining T-DNA out of the plant or plant cells. In some embodiments of any of the aspects, the PV gene is endogenously expressed only from the first genome. In some embodiments of any of the aspects, the PV gene is PV1. In some embodiments of any of the aspects, the PV gene is PV3. In some embodiments of any of the aspects, the one or more sequences at the PV locus is one or more of SEQ ID NOs: 253-255 and 266 or the reverse complement thereof. In some embodiments of any of the aspects, the PV gene is endogenously expressed from the first genome and at least one further genome and in step iv) the plant, first progeny, or cell thereof is further contacted with one or more guide RNA sequences or multi-guide constructs specific to the endogenous PV genes and/or flanking the endogenous PV genes, thereby mutating the endogenous PV genes at the native PV gene loci to create loss-of-function alleles.

In some embodiments of any of the aspects, the at least one functional allele of a MF gene is the endogenous wild-type functional allele of the MF gene. In some embodiments of any of the aspects, the at least one functional allele of a MF gene is an ectopic copy of the MF gene. In some embodiments of any of the aspects, the at least one functional allele of a MF gene and the at least one allele of a seed color gene (e.g., seed coat and/or seed endosperm gene) (or at least one functional ectopic allele of each member of a set of seed color genes) are part of single construct. In some embodiments of any of the aspects, an ectopic allele or ectopic copy of a gene is a nuclease-null or CRISPR-null allele. In some embodiments of any of the aspects, the MF gene displays the same type of activity and shares at least 80%, at least 85%, at least 90%, at least 95%, or greater sequence identity with one or more of the genes of Table 1. In some embodiments of any of the aspects, the MF gene is selected from Table 1. In some embodiments of any of the aspects, the MF gene displays the same type of activity and shares at least 80%, at least 85%, at least 90%, at least 95%, or greater sequence identity with Mfw2. In some embodiments of any of the aspects, the MF gene is Mfw2. In some embodiments of any of the aspects, the MF gene displays the same type of activity and shares at least 80%, at least 85%, at least 90%, at least 95%, or greater sequence identity with Ms1. In some embodiments of any of the aspects, the MF gene is Ms1. In some embodiments of any of the aspects, the PV gene displays the same type of activity and shares at least 80%, at least 85%, at least 90%, at least 95%, or greater sequence identity with one or more of the genes of Table 2. In some embodiments of any of the aspects, the PV gene is selected from Table 2. In some embodiments of any of the aspects, the PV gene displays the same type of activity and shares at least 80%, at least 85%, at least 90%, at least 95%, or greater sequence identity with PV1, PV2, or PV3. In some embodiments of any of the aspects, the PV gene is PV1, PV2, or PV3. In some embodiments of any of the aspects, the MF gene displays the same type of activity and shares at least 80%, at least 85%, at least 90%, at least 95%, or greater sequence identity with Mfw2 and the PV gene displays the same type of activity and shares at least 80%, at least 85%, at least 90%, at least 95%, or greater sequence identity with one of PV1, PV2, or PV3.

In some embodiments of any of the aspects, the MF gene displays the same type of activity and shares at least 80%, at least 85%, at least 90%, at least 95%, or greater sequence identity with Mfw2 and the PV gene displays the same type of activity and shares at least 80%, at least 85%, at least 90%, at least 95%, or greater sequence identity with PV1. In some embodiments of any of the aspects, the MF gene displays the same type of activity and shares at least 80%, at least 85%, at least 90%, at least 95%, or greater sequence identity with Mfw2 and the PV gene displays the same type of activity and shares at least 80%, at least 85%, at least 90%, at least 95%, or greater sequence identity with PV3.

In some embodiments of any of the aspects, the MF gene is Mfw2 and the PV gene is one of PV1, PV2, or PV3. In some embodiments of any of the aspects, the MF gene is Mfw2 and the PV gene is PV1. In some embodiments of any of the aspects, the MF gene is Mfw2 and the PV gene is PV3.

In some embodiments of any of the aspects, the at least one allele of a seed color gene (e.g., seed coat and/or seed endosperm gene) (or at least one functional ectopic allele of each member of a set of seed color genes) is exogenous. In some embodiments of any of the aspects, the at least one allele of a seed color gene (e.g., seed coat and/or seed endosperm gene) is blue aleurone (BA). In some embodiments of any of the aspects, the at least one allele of a seed color gene (e.g., seed coat and/or seed endosperm gene) (or at least one functional ectopic allele of each member of a set of seed color genes) comprises sequences obtained from a species within the same genus as the plant. In some embodiments of any of the aspects, the the at least one allele of a seed color gene (e.g., seed coat and/or seed endosperm gene) is at least two copies and/or individual alleles of the seed color gene (e.g., seed coat and/or seed endosperm gene). In some embodiments of any of the aspects, the the at least one allele of a seed color gene (e.g., seed coat and/or seed endosperm gene) is at least three copies and/or individual alleles of the seed color gene (e.g., seed coat and/or seed endosperm gene). In some embodiments of any of the aspects, the the at least one allele of a seed color gene (e.g., seed coat and/or seed endosperm gene) is at least four copies and/or individual alleles of the seed color gene (e.g., seed coat and/or seed endosperm gene).

In some embodiments of any of the aspects, the at least one allele of a seed color gene (e.g., seed coat and/or seed endosperm gene) (or at least one functional ectopic allele of each member of a set of seed color genes) is located within 10 cM of the MF gene loci. In some embodiments of any of the aspects, the at least one allele of a seed color gene (e.g., seed coat and/or seed endosperm gene) (or at least one functional ectopic allele of each member of a set of seed color genes) is located within 1 cM of the MF gene loci. In some embodiments of any of the aspects, the at least one ectopic functional allele of a PV gene is located within 10 cM of the MF gene loci. In some embodiments of any of the aspects, the at least one ectopic functional allele of a PV gene is located within 1 cM of the MF gene loci.

In some embodiments of any of the aspects, the only exogenous sequence in the genomes is the at least one allele of a seed color gene (e.g., seed coat and/or seed endosperm gene) (or at least one functional ectopic allele of each member of a set of seed color genes). In some embodiments of any of the aspects, the only ectopic sequence in the genomes is the at least one ectopic functional allele of a PV gene.

In some embodiments of any of the aspects, the plant is tetraploid and the second genome comprises loss-of-function alleles of the MF gene at the native MF gene loci and loss-of-function alleles of the PV gene at the native PV gene loci. In some embodiments of any of the aspects, the plant is hexaploid and the second and third genomes both comprise loss-of-function alleles of the MF gene at the native MF gene loci and loss-of-function alleles of the PV gene at the native PV gene loci.

In some embodiments of any of the aspects, a loss-of-function allele comprises an engineered knock-out modification. In some embodiments of any of the aspects, a loss-of-function allele comprises an engineered excision of at least part of a coding or regulatory sequence. In some embodiments of any of the aspects, the loss-of-function allele is engineered using a site-specific guided nuclease. In some embodiments of any of the aspects, the site-specific guided nuclease is a form of CRISPR-Cas (such as CRISPR-Cas9).

In some embodiments of any of the aspects, the plant is wheat, triticale, canola/oilseed rape, indian mustard, barley, rice, oat, or rye. In some embodiments of any of the aspects, the plant is wheat.

In some embodiments of any of the aspects, the at least one allele of a seed color gene (e.g., seed coat and/or seed endosperm gene) (or at least one functional ectopic allele of each member of a set of seed color genes) comprises a sequence from T. aestivum, T durum, T. monococcum or another Triticum aestivum-crossable species.

In some embodiments of any of the aspects, the plant is hexaploid wheat or tetraploid wheat, Triticum aestivum, or Triticum durum.

In some embodiments of any of the aspects, the at least one functional ectopic allele of a MF gene and at least one functional ectopic allele of a seed color gene (e.g., seed coat and/or seed endosperm gene) (or at least one functional ectopic allele of each member of a set of seed color genes) comprises the sequence of SEQ ID NO: 173 (or the coding sequence portion thereof) or a sequence with at least 80%, 85%, 90%, or 95% sequence identity thereto; and/or wherein the at least one functional ectopic allele of a PV gene comprises the sequence of SEQ ID NO: 172 (or the coding sequence portion thereof) or 258 or a sequence encoding SEQ ID NO: 258 or a sequence with at least 80%, 85%, 90%, or 95% sequence identity thereto. In some embodiments of any of the aspects, the guide RNA sequences and/or multi-guide constructs comprise one or more of SEQ ID NOs: 22-29, 131-154, 156, 210-213, 217, 235-237, 253-255 and 266-267.

In one aspect of any of the embodiments, described herein is a method of providing a male sterile plant seed, the method comprising selecting, from seed produced by selfing a maintainer plant as described herein, seed not displaying a phenotype provided by the seed endosperm gene. In one aspect of any of the embodiments, described herein is a method of providing male sterile plant seed, the method comprising selfing a maintainer plant described herein, whereby the resulting seed not displaying a phenotype provided by the seed endosperm gene is the male sterile plant seed.

In one aspect of any of the embodiments, described herein is a method of providing a F1 hybrid seed for crop production, the method comprising collecting the seed produced by a male-sterile plant pollinated by a male-fertile plant, wherein the male-sterile plant is:
a) a plant grown from male sterile plant seed obtained by a method described herein; and/or b) comprises:
   i) loss-of-function alleles of an endogenous MF gene at each of the native MF gene loci;
   ii) loss-of-function alleles of an endogenous PV gene at each of the native PV gene loci; and
   iii) two ectopic alleles of the PV gene at a target locus.
      In one aspect of any of the embodiments, described herein is a method of providing a F1 hybrid seed for crop production, the method comprising crossing a male-sterile plant with a male-fertile plant, wherein the male-sterile plant is:
a) a plant grown from male sterile plant seed obtained by a method described herein; and/or
b) comprises:
   i) loss-of-function alleles of an endogenous MF gene at each of the native MF gene loci;
   ii) loss-of-function alleles of an endogenous PV gene at each of the native PV gene loci; and
   iii) two ectopic alleles of the PV gene at a target locus.
      In one aspect of any of the embodiments, described herein is a method of providing a F1 hybrid seed for crop production, the method comprising planting a male-sterile plant within pollination range of a male-fertile plant, wherein the male-sterile plant is:
a) a plant grown from male sterile plant seed obtained by a method described herein; and/or
b) comprises:
   i) loss-of-function alleles of an endogenous MF gene at each of the native MF gene loci;
   ii) loss-of-function alleles of an endogenous PV gene at each of the native PV gene loci; and
   iii) two ectopic alleles of the PV gene at a target locus; and

whereby the male-fertile plant pollinates the male-sterile plant and F1 hybrid seed is produced. In some embodiments of any of the aspects, the pollination range is 200 metres. In one aspect of any of the embodiments, described herein the male-sterile plant and male fertile plant are different lines.

In one aspect of any of the embodiments, described herein is a method of producing a plant crop, the method comprising:
a) planting and/or harvesting a plant or portion thereof, wherein the plant:
   i) is plant grown from F1 hybrid seed obtained by a method described herein; and/or
   ii) comprises:
      1) in each genome of the plant, at a native MF gene locus, one functional endogenous allele of the endogenous MF gene and one loss-of-function allele of the endogenous MF gene;
      2) in each genome of the plant, at a native PV gene locus, one functional endogenous allele of the endogenous PV gene and one loss-of-function allele of the endogenous PV gene;
      3) one ectopic allele of the PV gene at a target locus.

### BRIEF DESCRIPTION OF THE DRAWINGS

FIG. 1 depicts a first step in producing an exemplary maintainer line.

FIG. 2 depicts how, in an exemplary embodiment, the maintainer line and initial male sterile lines are created in parallel.

FIGS. 3-4 depict how an exemplary maintainer line is propagated.

FIGS. 5-6 depict how, in an exemplary embodiment, a maintainer line is used to maintain the cognate male-sterile plants.

FIG. 7 depicts how, in an exemplary embodiment, the male-sterile plants are used for F1 seed production.

FIGS. 8-10 depict a method transferring the genetic elements of the described maintainer of male-sterility line into a second genotype by ‘conventional’ crossing and selection. Such methods can be utilized to move the genetic elements into elite lines or germplasm. Accordingly, the figures depict crossing an elite wildtype (wt) line onto a maintainer of male-sterility plant as described herein and selecting out new maintainer and male-sterile lines. In FIG. 8, seed harvested from the cross (ex maintainer) will be a 50% mix of the two depicted genotypes. This is colour-sorted, separating the 50% with darker-coloured (BA) grains (and MFW male-fertility), bottom right, from the non-coloured plants (no BA), bottom left. These two populations are planted, allowed to self-fertizile, and in the ensuing generation, individuals which are mfw/mf vx2 and mfw:PV/mfw:PV and pv/pv×3 (left, providing male-sterile individuals) and mfw/mfw×2 and MFW:BA/MFW:BA and PV/PV×3 are selected by PCR analysis (FIG. 9). These individuals are also selected for having an overall phenotype which is closest to the WT elite parent. The two selected individual plants or populations are then crossed. The plants from this cross are grown (FIG. 10, top left) and, from their progeny, PCR analysis is used to select those plants with a mfw/mfw×2+mfw:PV/MFW:BA+pv/pv×3 genotype (no wt PV allele) and maximum WT elite line genotype (FIG. 10, top center). These plants are allowed to self-fertilize. Harvested seed will be a 50% mix of the two genotypes indicated at the bottom of FIG. 10. This seed is colour-sorted, selecting the 50% with darker-coloured (BA) grains and so MFW male-fertility, (FIG. 10, bottom right), to become the new maintainer line and separately, the non-coloured seeds, (FIG. 10, bottom left), which become the new male-sterile line. The seed/plants can be subject to standard selection in recurrent pollinator/maintainer for a further five generations to achieve introgression in the elite line.

FIG. 11 depicts a schematic of the maintainer-line background (e.g., starting genetic material) genetics.

FIG. 12 depicts a schematic of the first stage of making the maintainer of male-sterility line.

FIG. 13 depicts a schematic of the maintainer and male-sterile lines.

FIG. 14 depicts the pollen and ovule production of the maintainer line.

FIG. 15 depicts the production of F1 seed by the maintainer line.

FIG. 16 depicts the maintenance of the male-sterile plant.

FIG. 17 depicts the use of the male-sterile plant to produce hybrid F1 seed.

FIGS. 18-20 depict the creation of new maintainers and male-steriles by crossing with an elite line.

FIG. 21 depicts using a herbicide tolerance gene to select the maintainer cassettes.

FIG. 22 depicts a method for creating the TO plants necessary for maintainer line production.

FIG. 23 depicts a method for creating maintainer and male-sterile lines together.

FIG. 24 depicts the maintenance of the maintainer line.

FIGS. 25-26 depict the creation of new maintainers and male-steriles by crossing with an elite line.

FIGS. 27-28 depict the maintenance of the maintainer line.

FIG. 29 depicts F1 hybrid crop seed production.

FIG. 30 depicts the creation of new maintainers and male-steriles by crossing with an elite line.

FIGS. 31A-31B depict diagrams of exemplary MF′:BA construct, utilizing Ms1′ as the MF′ gene and BA1 as the BA gene. In some embodiments, the coding sequence of BA1 is used (FIG. 31A), providing a shorter construct than required for the full length genomic BA sequence (FIG. 31B). FIG. 31C depicts a digram of an illustrative embodiment of a gene cassette for the initial stage of production of the maintainer. The maintainer can be produced by transforming a wild-type elite line with a T-DNA cassette containing the genomic sequence of Mfw2′ followed by BA1 or BA2. This will be followed by a selection gene for example nptII finally followed by the genomic sequence of PV1′ (Mfw2′ and PV1′ are the subtly different/adapted versions of Mfw2 and PV1 as described elsewhere herein). Four sequences can also be included for future modifications to separate the genes: two Cre-lox cut sequences flanking the Mfw2′, BA and Nptll selection gene and two flippase cut sequences flanking nptII, PV1′ as shown in the figure. The Mfw2′:BA and PV1′ parts can then be made into separate alleles at the same locus as follows. One or more plants/embryos retransformed with Cre-lox to drop out everything between the cre-lox sites to leave PV1′ or, similarly, a different one or more plant/embryos retransformed with flippase to leave Mfw2′:BA. The heterozygous combination of the maintainer system's two alleles can than be produced by crossing successful products of these two retransformations. The final stage to produce the maintainer is to knockout the endogenous Mfw2 and PV1 genes. This approach is described in more detail in Example 9. FIG. 31D depicts a version of the gene cassette utilizing PV3′ instead of PV1′.

FIGS. 32A and 32B depict the genotype of plants described in Example 9. FIGS. 32A-32B illustrate the genotypes by referring to MFW, MFW, PV, and PV′, while Example 9 utilizes the exemplary Mfw2 and PV1 genes. Example 9 is an exemplary embodiment and is not limiting on the technology described herein or as illustrated in FIGS. 32A-32B.

FIG. 33 depicts a schemative of the insertion of the Mfw2′:BA allele at the PV1-B locus.

FIG. 34 depicts the sequence of the gRNA locations illustrated in FIG. 33. FIG. 34 depicts SEQ ID NO: 234 gRNA, which includes sequence base pair No's 927-979, inclusive, of SEQ ID NO 188. The figure depicts the three gRNA sequences as SEQ ID NOs: 235-237.

FIGS. 35A-35C depict an overview of a procedure for producing both new maintainer and male-sterile plants together, utilizing only crossing and selection. The process proceeds from FIG. 35A to FIG. 35B to FIG. 35C as indicated by the provided arrows. See, e.g, Example 10.

### DETAILED DESCRIPTION

Described herein are plants, plant cells, and methods that relate to a F1 hybrid wheat system that can be readily incorporated into an established breeding programme. Specifically, the system comprises a male-sterile line and a cognate maintainer line. The maintainer line can 1) pollinate the male-sterile without transferring male-fertility and 2) self-pollinate without losing its necessary genetic traits. By pollinating the male-sterile without transferring male-fertility, this keeps the ‘purity’ of the male-sterile's recessive male-sterility. As a result, the male-sterile line can, in the final seed production field, be pollinated by any ‘wild-type’ elite breeding line. The system therefore provides tools for low cost-of-sale F1 seed, e.g., for sale to farmers.

In some embodiments, the methods and compositions described herein relate to polyploidal maintainer plants in which a first genome is engineered to provide a locus which controls male fertility. Specifically, on one chromosome of a homologous pair, the locus comprises a dominant male-fertile allele(s) of a male fertility (MF) gene that cosegregates with at least one allele of a seed color gene (e.g., seed coat and/or seed endosperm gene) (or at least one functional ectopic allele of each member of a set of seed color genes). On the second chromosome of the homologous pair, the locus comprises a dominant viable-pollen allele(s) of a pollen-viability (PV) gene. All other copies of MF or PV alleles are recessive loss-of-function alleles of those MF and PV genes, e.g., the second and/or further genomes comprise only recessive loss-of-function alleles of those MF and PV genes. MF genes function largely pre-meiosis and therefore, the presence of the dominant MF allele(s) in the maintainer line's pre-meiosis reproductive cells will provide reproductive functionality for the MF gene's activity, so the MF allele(s) carried by an individual pollen grain post-meiosis is not determinative of its viability. As used herein, “pre-meiosis”, used in reference to a MF gene, encompasses the time prior to the conclusion of meiosis. MF genes can exert an effect while the cell still sporophyte/diploid (with expression of both relevant alleles taking place (including during meiosis)), rather than when the cell is a gametophyte/haploid (e.g., when each allele is the only allele present to be expressed post-meiosis). Stated another way, the MF allele(s) on the first chromosome is sufficient to confer male fertility on the plant, while the absence of a functional copy(ies) results in a male-sterile plant. However, the PV gene (as described below) is post-meiosis in function, so each pollen grain carrying only pv alleles will be non-viable whatever its MF gene status. That is, at least one copy of the PV gene in a pollen grain is sufficient to support pollen development, while the absence of a functional PV allele in a pollen grain will prevent development of the pollen. Thus, male-fertility and pollen production are controlled by the genotype of the first genome.

Accordingly, in one aspect of any of the embodiments, provided herein is a male-fertile maintainer plant or cell (e.g., a maintainer plant for a male-sterile plant), the maintainer comprising: 1) a first genome comprising: a) on a first chromosome of a pair of homologous chromosomes, a functional allele(s) of a MF gene at a first locus and at least one allele of a seed color gene (e.g., seed coat and/or seed endosperm gene) (or at least one functional ectopic allele of each member of a set of seed color genes); b) on a second chromosome of the pair of homologous chromosomes, a loss-of-function allele of the MF gene at the native MF locus and at least one ectopic functional allele of a PV gene; c) loss-of-function alleles of the PV gene at the native PV gene loci; and 2) at least one further genome, each of the further genomes comprising loss-of-function alleles of the MF gene at the native MF gene loci and loss-of-function alleles of the PV gene at the native PV gene loci. In some embodiments of any of the aspects, the first locus is the native MF gene locus. In some embodiments of any of the aspects, the at least one ectopic functional allele of a PV gene is located at the native MF gene locus. In some embodiments of any of the aspects, the at least one allele of a seed color gene (e.g., seed coat and/or seed endosperm gene) (or at least one functional ectopic allele of each member of a set of seed color genes) is located at the native MF gene locus. In some embodiments of any of the aspects, the at least one allele of a seed color gene (e.g., seed coat and/or seed endosperm gene) (or at least one functional ectopic allele of each member of a set of seed color genes) is ectopic. In some embodiments of any of the aspects, the at least one allele of a seed color gene (e.g., seed coat and/or seed endosperm gene) (or at least one functional ectopic allele of each member of a set of seed color genes) is exogenous.

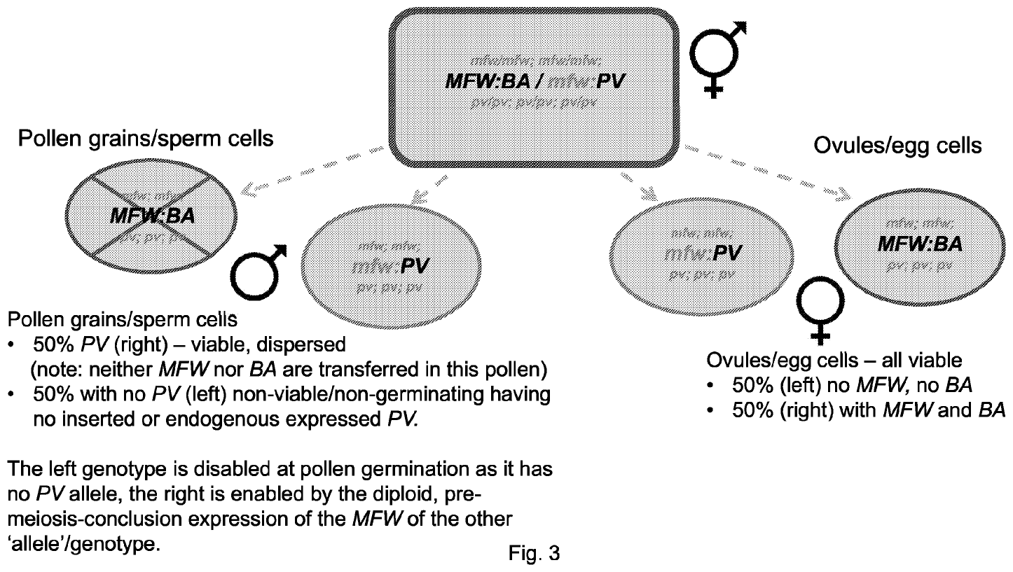

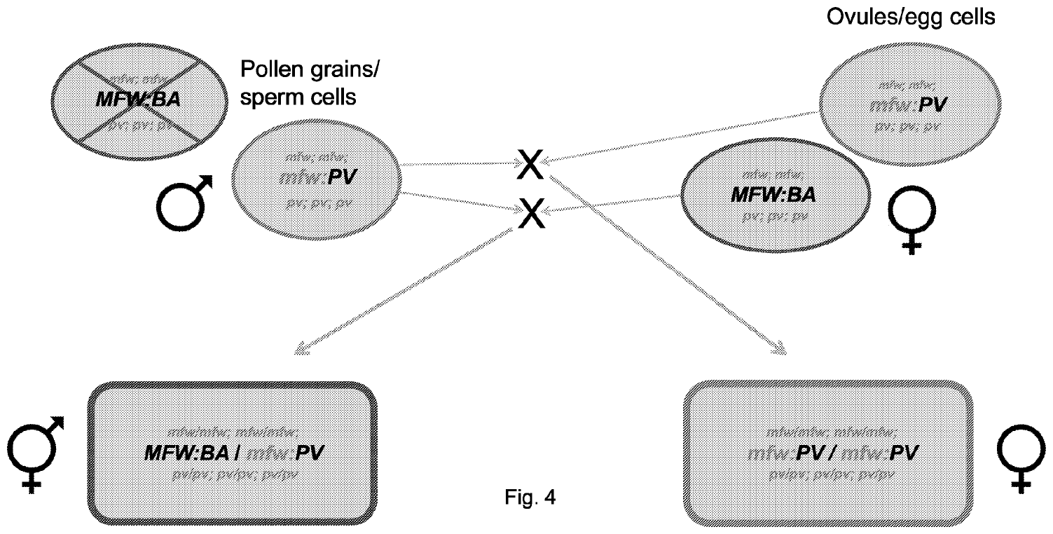

For illustrative purposes, FIG. 3 provides a schematic of the modifications described herein and FIGS. 3-4 depict how the maintainer line's genetics function during propagation in an exemplary embodiment. Specifically, the maintainer plant will produce viable pollen grains which comprise the second chromosome of the first genome and never the first chromosome of the first genome as the latter will comprise pollen-grains without a functional PV gene and will not be viable. When this maintainer plant self-fertilizes, the seeds can be sorted by seed endosperm color to obtain progeny with the genotype of the parent, allowing a heterozygous maintainer line to be propagated and provide a new generation of heterozygous maintainer plants at low costs of labor and time.

It is noted that the methods and compositions described herein provide surprising advantages over existing approaches based on cytoplasmic male-sterility. A major problem with cytoplasmic male-sterility is that one needs to breed the final ‘male’ pollinator-line, used to produce the F1 seed, to comprise a ‘restorer’ gene(s) to overcome the male-sterility of the ‘female line’ so that the customer's commercial crop grown from the F1 seed has full fertility. In the systems described herein, the male-sterility is recessive so any wild-type cultivar (e.g., any wild-type elite breeding line) will act as a restorer. This means that production of hybrid seed can be conducted normally by crossing the male-sterile line with a different cultivar of choice without the use of a particular restorer line. This permits production of hybrid F1 seed at lower costs than current F1 cereal plant breeding technologies.

Furthermore, the methods and compositions described herein permit these advantages without the male-sterile or F1 seed being transgenic, for example, as explained in more detail in Example 1. For example, by using genes from the same species and/or genus, which could have been introduced by traditional crossing, the instant plants and systems are considered cis-genesis genome editing, which is already accepted as non-regulated/non-GM in the US and is likely to be regulated lightly in the EU. This provides for more widespread use and available markets as compared to transgenic plants (with transgenes from non-crossable species).

It is noted that the MF, PV, and seed color gene (e.g., seed coat and/or seed endosperm gene) (or at least one functional ectopic allele of each member of a set of seed color genes) alleles/loci may be in any 5′ to 3′ order and any recitation of the genes provided herein is not meant to limit the embodiments to a particular 5′ to 3′ order. Within the term “plants” in this specification is included seeds and seedlings.

Different alleles described herein are referred to as either functional or loss-of-function alleles. As used herein, “functional” refers to a portion and/or variant of a polypeptide or gene that retains at least a detectable level of the activity of the native polypeptide or gene from which it is derived. Methods of detecting, e.g. activity and/or functionality are known in the art for various types of polypeptides. In some embodiments of any of the aspects, a functional allele can be an allele comprising, consisting of, or consisting essentially of a wild-type allele of a gene, e.g., one of the sequences provided herein. In some embodiments of any of the aspects, a functional allele can be an allele comprising, consisting of, or consisting essentially of a sequence with at least 80%, at least 85%, at least 90%, at least 95%, or greater sequence identity with a wild-type allele of a gene, e.g., one of the sequences provided herein. In some embodiments of any of the aspects, a functional allele can be an unengineered or unmodified allele, e.g., it is the wild-type allele. In some embodiments of any of the aspects, an ectopic functional allele can be a copy of a wild-type allele inserted or introduced into a different location in the genome, e.g., the ectopic functional allele does not comprise any sequence exogenous to the plant/cell. In some embodiments of any of the aspects, a functional allele comprises a coding sequence encoding a protein sequence. In some embodiments of any of the aspects, a functional allele comprises a cDNA encoding a protein sequence. In some embodiments of any of the aspects, a functional allele comprises a cDNA corresponding to a coding sequence and/or mRNA. In some embodiments of any of the aspects, a functional allele comprises a genomic sequence encoding a protein sequence. In some embodiments of any of the aspects, a functional allele comprises a genomic sequence. In some embodiments of any of the aspects, a functional allele comprises a coding sequence encoding a protein sequence described herein. In some embodiments of any of the aspects, a functional allele comprises a cDNA encoding a protein sequence described herein. In some embodiments of any of the aspects, a functional allele comprises a cDNA corresponding to a coding sequence and/or mRNA described herein. In some embodiments of any of the aspects, a functional allele comprises a genomic sequence encoding a protein sequence described herein. In some embodiments of any of the aspects, a functional allele comprises a genomic sequence described herein.

In some embodiments of any of the aspects, a construct or chromosome comprising at least one functional ectopic allele of a gene (e.g. a MF, PV, or seed color gene) comprises one functional ectopic allele of the gene. In some embodiments of any of the aspects, a construct or chromosome comprising at least one functional ectopic allele of a gene (e.g. a MF, PV, or seed color gene) comprises two functional ectopic alleles of the gene. In some embodiments of any of the aspects, a construct or chromosome comprising at least one functional ectopic allele of a gene (e.g. a MF, PV, or seed color gene) comprises three functional ectopic alleles of the gene.

In some embodiments of any of the aspects, the plant is a polyploid and a construct or chromosome comprising at least one functional ectopic allele of a gene (e.g. a MF, PV, or seed color gene) comprises one functional ectopic allele of the gene, wherein the one functional ectopic allele comprises one of the multiple homeologues of the gene. In some embodiments of any of the aspects, the plant is a polyploid and a construct or chromosome comprising at least one functional ectopic allele of a gene (e.g. a MF, PV, or seed color gene) comprises two functional ectopic alleles of the gene, wherein the two functional ectopic alleles comprise two of the multiple homeologues of the gene. In some embodiments of any of the aspects, the plant is a polyploid and a construct or chromosome comprising at least one functional ectopic allele of a gene (e.g. a MF, PV, or seed color gene) comprises three functional ectopic alleles of the gene, wherein the three functional ectopic alleles comprise three of the multiple homeologues of the gene.

In some embodiments of any of the aspects, the plant is a hexaploid and a construct or chromosome comprising at least one functional ectopic allele of a gene (e.g. a MF, PV, or seed color gene) comprises one functional ectopic allele of the gene, wherein the one functional ectopic allele comprises one of the three homeologues of the gene. In some embodiments of any of the aspects, the plant is a hexaploid and a construct or chromosome comprising at least one functional ectopic allele of a gene (e.g. a MF, PV, or seed color gene) comprises two functional ectopic alleles of the gene, wherein the two functional ectopic alleles comprise two of the three homeologues of the gene. In some embodiments of any of the aspects, the plant is a hexaploid and a construct or chromosome comprising at least one functional ectopic allele of a gene (e.g. a MF, PV, or seed color gene) comprises three functional ectopic alleles of the gene, wherein the three functional ectopic alleles comprise all three homeologues of the gene.

The term “wild type” refers to the naturally-occurring polynucleotide sequence encoding a protein, or a portion thereof, or protein sequence, or portion thereof, respectively, as it normally exists in vivo. It may also refer to the original plant genotype which was used for any transformation, gene-editing or gene-repression experiments herein, e.g., the genotype as it existed prior to any of the engineering steps described herein. Exemplary wild-type and functional alleles of MF and PV genes are provided herein, or can be a naturally-occurring MF or PV allele in a fertile plant.

As used herein “loss-of-function” refers to partial or complete reduction of the expression or activity of a protein encoded by an endogenous DNA sequence in a cell such that the protein can no longer accomplish its function. In some embodiments of any of the aspects, a loss-of-function allele comprises an engineered modification. A “modification” in a nucleic acid sequence refers to any detectable change in the genetic material, e.g., a change or alteration relative to a reference sequence, e.g, the wild-type sequence. Modifications can be insertions, deletions, replacements, indels, SNPs, mutations, substitutions, or the like. A modification is usually a change of one or more deoxyribonucleotides, the modification being obtained by, for example, adding, deleting, inverting, or substituting nucleotides.

In some embodiments of any of the aspects, a loss-of-function allele comprises, consists of, or consists essentially of an engineered excision of at least part of a coding or regulatory sequence. In some embodiments of any of the aspects, a loss-of-function allele comprises, consists of, or consists essentially of an engineered excision of an allele's promoter. In some embodiments of any of the aspects, a loss-of-function allele comprises, consists of, or consists essentially of an engineered excision of at least 5%, at least 10%, at least 20%, at least 30% or more of an allele's coding sequence. In some embodiments of any of the aspects, a loss-of-function allele comprises, consists of, or consists essentially of an engineered excision of at least 90%, at least 95%, or 100% of an allele's coding sequence. In some embodiments of any of the aspects, a loss-of-function allele comprises, consists of, or consists essentially of an engineered missense or nonsense mutation within the first 10% of the coding sequence of an allele.

In some embodiments of any of the aspects, a loss-of-function allele comprises, consists of, or consists essentially of an engineered knock-out modification. As used herein, “knock-out” refers to partial or complete reduction of the expression of a protein encoded by an endogenous DNA sequence in a cell such that the protein can no longer accomplish its function. In some embodiments, the “knock-out” can be produced by targeted deletion of the whole or part of a gene encoding a protein. In some embodiments, the deletion may prevent or reduce the expression of the functional protein in a cell in which it is normally expressed. A knock-out plant can be a transgenic plant, or can be created without transgenic methods, e.g. without the introduction of exogenous DNA to the genome.

In some embodiments of any of the aspects, a knock-out modification comprises a deletion of the whole or part of a gene encoding a protein in a cell. In some embodiments of any of the aspects, a knock-out modification comprises deletion of the entire coding sequence of the relevant gene. In some embodiments of any of the aspects, a knock-out allele does not comprise any of the coding sequence of the relevant gene. In some embodiments of any of the aspects, a knock-out modification comprises deletion of a part of the coding sequence of the relevant gene, e.g, 10%, 20%, 30%, 40%, 50%, 60%, 70%, 80%, 90% or more of the coding sequence of the relevant gene. In some embodiments of any of the aspects, a knock-out modification comprises a non-sense mutation of the relevant gene, e.g, in the first 10%, first 20%, first 30%, first 40%, first 50%, first 60%, or first 70% of the coding sequence of the relevant gene. In some embodiments of any of the aspects, a knock-out modification comprises a missense mutation of the relevant gene, e.g, in the first 10%, first 20%, first 30%, first 40%, first 50%, first 60%, or first 70% of the coding sequence of the relevant gene. In some embodiments of any of the aspects, a knock-out modification comprises the introduction of a stop codon in the relevant gene, e.g, in the first 10%, first 20%, first 30%, first 40%, first 50%, first 60%, or first 70% of the coding sequence of the relevant gene. In some embodiments of any of the aspects, a knock-out modification comprises deletion of the promoter of the relevant gene, e.g, deletion of at least 40%, at least 50%, at least 60%, at least 70%, at least 80%, at least 90% or more of the promoter of the relevant gene.

In some embodiments of any of the aspects, a loss-of-function allele comprises, consists of, or consists essentially of methylation and/or hypermethylation of the coding and/or regulatory sequence of a the relevant gene. For example, methods of introducing heritable CG-specific methylation that provides loss-of-function alleles is known in the art and can be produced using artificial zinc finger protein targeting of the CG-specific methyltransferase M.SssI, or using CRISPR technology. Further discussion of such methods can be found in the art, e.g., Liu et al. Nature Communications 2021 12:3130 and Ghoshal et al. PNAS 2021 118:e2125016118; each of which is incorporated by reference herein in its entirety. In some embodiments of any of the aspects, a loss-of-function allele comprises methylation in the allele's promoter. In some embodiments of any of the aspects, a loss-of-function allele comprises methylation of at least one cytosine in the allele's promoter. In some embodiments of any of the aspects, a loss-of-function allele comprises methylation of at least two cytosines in the allele's promoter. In some embodiments of any of the aspects, a loss-of-function allele comprises methylation of at least three cytosines in the allele's promoter. In some embodiments of any of the aspects, a loss-of-function allele comprises methylation of at least ten cytosines in the allele's promoter. In some embodiments of any of the aspects, a loss-of-function allele comprises methylation of at least twenty cytosines in the allele's promoter. In some embodiments of any of the aspects, the methylation results in an alteration of the expression of the gene relative to expression in the absence of the methylation.

As used herein, a “MF” or “malefertility” gene is a gene which, when its expression is inhibited, decreases male-fertility and which functions pre-meiosis. MF genes can be specific for male-fertility, rather than female-fertility. In some embodiments of any of the aspects, a MF gene, when fully deactivated (i.e., all copies are deactivated) in a plant, is sufficient to render the plant male-sterile, e.g., one or more copies of the MF gene is strictly necessary for male-fertility. In some embodiments of any of the aspects, the MF gene is a gene which has been identified to produce a male-sterile phenotype when a plant was modified to comprise loss-of-function alleles for that gene. In some embodiments of any of the aspects, the MF gene is pre-meiotic, e.g., it functions before meiosis or before the conclusion of meiosis (e.g., the diploid phases of meiosis). “Mfv” is used at times herein interchangeably with “MF” and may refer to wheat MF genes, e.g., where the wheat genome is used as an illustrative embodiment. Where “Mfw” is used, one of skill in the art will understand that those embodiments are equally applicable in other plant species using suitable MF genes for that species.

MF genes for various species have been described in the art, and exemplary, but non-limiting, MF genes include those described in International Patent Application PCT/US2017/043009 (referred to therein as Mpew or Mfw genes), International Patent Application PCT/US2019/019139; each of which is incorporated by reference herein in its entirety. In some embodiments of any of the aspects, the MF gene is a gene which displays the same type of activity, and/or shares at least 80%, at least 85%, at least 90%, at least 95%, or greater sequence identity with a MF gene of any of the foregoing references. In some embodiments of any of the aspects, a MF gene can be the gene from a species, cultivar, or variety which has the highest degree of homology and/or sequence identity of the genes in that species', cultivar's or variety's genome with a gene selected from one of the foregoing references.

In some embodiments of any of the aspects, the MF gene is a dominant male-fertility gene. That is, one functional allele of the MF gene is sufficient to provide male fertility. In some embodiments of any of the aspects, the dominant MF gene is Mfw2.

A non-limiting list of exemplary pre-meiosis MF genes is provided in Table 1. In some embodiments of any of the aspects, the MF gene is a gene selected from Table 1. In some embodiments of any of the aspects, the MF gene has at least 80%, at least 85%, at least 90%, at least 95%, at least 98%, or greater sequence identity with a MF gene of Table 1. In some embodiments of any of the aspects, the MF gene is a gene which displays the same type of activity, and has at least 80%, at least 85%, at least 90%, at least 95%, at least 98%, or greater sequence identity with a MF gene of Table 1. In some embodiments of any of the aspects, the MF gene has at least 95% sequence identity with a MF gene of Table 1. In some embodiments of any of the aspects, the MF gene is a gene which displays the same type of activity, and has at least 95% sequence identity with a MF gene of Table 1. In some embodiments of any of the aspects, the MF gene is a gene of Table 1. In some embodiments of any of the aspects, a MF gene can be the gene from a species, cultivar, or variety which has the highest degree of homology and/or sequence identity of the genes in that species', cultivar's or variety's genome with a gene selected from Table 1.

In some embodiments of any of the aspects, a functional allele of a MF gene shares at least 80% sequence identity with at least one sequence of Table 1. In some embodiments of any of the aspects, a functional allele of a MF gene displays the same type of activity and shares at least 80% sequence identity with at least one sequence of Table 1. In some embodiments of any of the aspects, a functional allele of a MF gene shares at least 80%, at least 85%, at least 90%, at least 95%, at least 98%, or greater sequence identity with at least one sequence of Table 1. In some embodiments of any of the aspects, a functional allele of a MF gene displays the same type of activity and shares at least 80%, at least 85%, at least 90%, at least 95%, at least 98%, or greater sequence identity with at least one sequence of Table 1. In some embodiments of any of the aspects, a functional allele of a MF gene shares at least 95% sequence identity with at least one sequence of Table 1. In some embodiments of any of the aspects a functional allele of a MF gene displays the same type of activity and shares at least 95% sequence identity with at least one sequence of Table 1. In some embodiments of any of the aspects, the functional allele of a MF gene is a sequence of Table 1.

TABLE 1
Exemplary MF genes

| | | | TGAC v1 homoeologues* - | |
|---|---|---|---|---|
| | | | the copies on the other | |
| Assigned | | | subgenomes of wheat and | |
| Mfw | | TGAC v1 gene | their associated gene | RefSeq v1.1 |
| name | Blast hit | model* | models | Sequences |
| Mfw2-A | callose | TRIAE_CS42_7AS_{—} | TRIAE_CS42_7BS_TGACV1_{—} | TraesCS7A02G146200 |
| | synthase 5 | TGACv1_569258_{—} | 593715_AA1953990; | |
| | | AA1811650 | TRIAE_CS42_7DS_TGACv1_{—} | |
| | | | 622598_AA2042310 | |
| Mfw2-B | callose | TRIAE_CS42_7BS_{—} | TRIAE_CS42_7AS_TGACV1_{—} | TraesCS7B02G048700 |
| | synthase 5 | TGACv1_593715_{—} | 569258_AA1811650; | |
| | | AA1953990 | TRIAE_CS42_7DS_TGACv1_{—} | |
| | | | 622598_AA2042310 | |
| ″ | callose | TRIAE_CS42_7BS_{—} | TRIAE_CS42_7AS_TGACV1_{—} | |
| | synthase 5 | TGACv1_593715_{—} | 569258_AA1811650; | |
| | | AA1953990 | TRIAE_CS42_7DS_TGACv1_{—} | |
| | | | 622598_AA2042310 | |
| ″ | callose | TRIAE_CS42_7BS_{—} | TRIAE_CS42_7AS_TGACV1_{—} | |
| | synthase 5 | TGACv1_593715_{—} | 569258_AA1811650; | |
| | | AA1953990 | TRIAE_CS42_7DS_TGACv1_{—} | |
| | | | 622598_AA2042310 | |
| ″ | callose | TRIAE_CS42_7BS_{—} | TRIAE_CS42_7AS_TGACV1_{—} | |
| | synthase 5 | TGACv1_593715_{—} | 569258_AA1811650; | |
| | | AA1953990 | TRIAE_CS42_7DS_TGACv1_{—} | |
| | | | 622598_AA2042310 | |
| Mfw2-D | callose | TRIAE_CS42_7DS_{—} | TRIAE_CS42_7BS_TGACv1_{—} | TraesCS7D02G147700 |
| | synthase 5 | TGACv1_622598_{—} | 593715_AA1953990; | |
| | | AA2042310 | TRIAE_CS42_7AS_TGACV1_{—} | |
| | | | 569258_AA1811650 | |
| Mfw3-A | Aborted | TRIAE_CS42_6AS_{—} | TRIAE_CS42_6BS_TGACv1_{—} | TraesCS6A02G268800 |
| | microspore | TGACv1_486918_{—} | 514404_AA1659330; | |
| | 1 like | AA1566480 | TRIAE_CS42_U_TGACv1_{—} | |
| | | | 643846_AA2135420 | |
| Mfw3-B | Aborted | TRIAE_CS42_6BS_{—} | TRIAE_CS42_6AS_TGACv1_{—} | TraesCS6B02G295900 |
| | microspore | TGACv1_514404_{—} | 486918_AA1566480; | |
| | 1 like | AA1659330 | TRIAE_CS42_U_TGACv1_{—} | |
| | | | 643846_AA2135420 | |
| Mfw3-D | Aborted | TRIAE_CS42_U_{—} | TRIAE_CS42_6AS_TGACv1_{—} | TraesCS6D02G246100 |
| | microspore | TGACv1_643846_{—} | 486918_AA1566480; | |
| | 1 like | AA2135420 | TRIAE_CS42_6BS_TGACv1_{—} | |
| | | | 514404_AA1659330 | |
| Mfw9-B | member of | TRIAE_CS42_2DS_{—} | TRIAE_CS42_2AS_TGACv1_{—} | TraesCS2B02G055800; |
| | the sweet | TGACv1_177708_{—} | 113352_AA0354890; | TraesCS2B02G055900: |
| | family | AA0582810 | TRIAE_CS42_2BS_TGACv1_{—} | TraesCS2B02G056100 |
| | | | 149844_AA0497680 | |
| Mfw10-A | member of | TRIAE_CS42_7AS_{—} | TRIAE_CS42_7BS_TGACv1_{—} | TraesCS7A02G261100 |
| | the sweet | TGACv1_570345_{—} | 591914_AA1925470 | |
| | family | AA1834200 | | |
| Mfw11-B | Similar to | TRIAE_CS42_U_{—} | no strong hit | TraesCS5B02G288600 |
| | OsSweet7e | TGACv1_640821_{—} | | |
| | | AA2075730 | | |
| Mfw12-D | Sweet4 | TRIAE_CS42_1DL_{—} | TRIAE_CS42_1AL_TGACv1_{—} | TraesCS1D02G365200 |
| | | TGACv1_065128_{—} | 002319_AA0040790; | |
| | | AA0236610 | TRIAE_CS42_1BL_TGACv1_{—} | |
| | | | 030610_AA0095680 | |
| Ms8 | | | | See Wu et al. Plant |
| | | | | Biotechnology Journal |
| | | | | 14: 1046-1054 (2015) |
| Ms32 | | | | See Wu et al. Plant |
| | | | | Biotechnology Journal |
| | | | | 14: 1046-1054 (2015) |
| Ocl14 | | | | See Wu et al. Plant |
| | | | | Biotechnology Journal |
| | | | | 14: 1046-1054 (2015) |
| Mac1 | | | | See Wu et al. Plant |
| | | | | Biotechnology Journal |
| | | | | 14: 1046-1054 (2015) |
| Ms22 | | | | See Wu et al. Plant |
| | | | | Biotechnology Journal |
| | | | | 14: 1046-1054 (2015) |
| Ms23 | | | | See Wu et al. Plant |
| | | | | Biotechnology Journal |
| | | | | 14: 1046-1054 (2015) |
| Mfw5-A | bHLH91 | TRIAE_CS42_2AL_{—} | TRIAE_CS42_2BL_TGACV1_{—} | TraesCS2A02G442700 |
| | | TGACv1_094707_{—} | 129925_AA0399500; | |
| | | AA0301850 | TRIAE_CS42_2DL_TGACv1_{—} | |
| | | | 158620_AA0523420 | |
| Mfw5-B | bHLH91 | TRIAE_CS42_2BL_{—} | TRIAE_CS42_2AL_TGACv1_{—} | TraesCS2B02G463800 |
| | | TGACv1_129925_{—} | 094707_AA0301850; | |
| | | AA0399500 | TRIAE_CS42_2DL_TGACv1_{—} | |
| | | | 158620_AA0523420 | |
| Mfw5-D | bHLH91 | TRIAE_CS42_2DL_{—} | TRIAE_CS42_2AL_TGACv1_{—} | TraesCS2D02G441800 |
| | | TGACv1_158620_{—} | 094707_AA0301850; | |
| | | AA0523420 | TRIAE_CS42_2BL_TGACv1_{—} | |
| | | | 129925_AA0399500 | |
| Mfw6-A | GAMYB | TRIAE_CS42_6AS_{—} | TRIAE_CS42_6DS_TGACv1_{—} | TraesCS6A02G137800 |
| | (AtMYB101) | TGACv1_485682_{—} | 543879_AA1744870 | |
| | | AA1550030 | | |
| ″ | GAMYB | TRIAE_CS42_6AS_{—} | TRIAE_CS42_6DS_TGACv1_{—} | |
| | (AtMYB101) | TGACv1_485682_{—} | 543879_AA1744870 | |
| | | AA1550030 | | |
| Mfw6-D | GAMYB | TRIAE_CS42_6DS_{—} | TRIAE_CS42_6AS_TGACV1_{—} | TraesCS6D02G126900 |
| | (AtMYB101) | TGACv1_543879_{—} | 485682_AA1550030 | |
| | | AA1744870 | | |
| ″ | GAMYB | TRIAE_CS42_6DS_{—} | TRIAE_CS42_6AS_TGACV1_{—} | |
| | (AtMYB101) | TGACv1_543879_{—} | 485682_AA1550030 | |
| | | AA1744870 | | |
| Mfw7-B | Hothead | TRIAE_CS42_4BL_{—} | TRIAE_CS42_4DL_TGACv1_{—} | TraesCS5A02G522000 |
| | | TGACv1_320326_{—} | 343496_AA1135340; | |
| | | AA1035360 | TRIAE_CS42_5AL_TGACv1_{—} | |
| | | | 375593_AA1224180 | |
| ″ | ″ | TRIAE_CS42_4BL_{—} | TRIAE_CS42_4DL_TGACv1_{—} | |
| | | TGACv1_320326_{—} | 343496_AA1135340; | |
| | | AA1035360 | TRIAE_CS42_5AL_TGACv1_{—} | |
| | | | 375593_AA1224180 | |
| Mfw7-D | Hothead | TRIAE_CS42_4DL_{—} | TRIAE_CS42_4BL_TGACv1_{—} | TraesCS4B02G353200 |
| | | TGACv1_343496_{—} | 320326_AA1035360; | |
| | | AA1135340 | TRIAE_CS42_5AL_TGACv1_{—} | |
| | | | 375593_AA1224180 | |
| Mfw8-D | Hothead | TRIAE_CS42_6DL_{—} | TRIAE_CS42_6AL_TGACv1_{—} | TraesCS6D02G238700 |
| | | TGACv1_527115_{—} | 470984_AA1500160; | |
| | | AA1698830 | TRIAE_CS42_6BL_TGACv1_{—} | |
| | | | 500863_AA1610910 | |
| Mfw13-D | Hothead | TRIAE_CS42_1DL_{—} | TRIAE_CS42_1AL_TGACv1_{—} | TraesCS1D02G189200 |
| | | TGACv1_063432_{—} | 001690_AA0034080; | |
| | | AA0227210 | TRIAE_CS42_1BL_TGACV1_{—} | |
| | | | 032570_AA0131570 | |
| Ms1 | | | | SEQ ID NOs: 183-191, |
| | | | | 214-216 |
| | | | | See also Tucker et al. |
| | | | | Nature Communications |
| | | | | 2017 8: 869; which is |
| | | | | incorporated by |
| | | | | reference herein in |
| | | | | its entirety |
| MSCA1 | | | | See U.S. Pat. No. |
| | | | | 7,919,676 and WO |
| | | | | 2019/043082; each of |
| | | | | which is incorporated |
| | | | | by reference herein |
| | | | | in its entirety. |
| | | | | For example, the nucleic |
| | | | | acid sequence of SEQ |
| | | | | ID NO: 16 and the |
| | | | | polypeptide sequence of |
| | | | | SEQ ID NO: 17 of U.S. |
| | | | | Pat. No. 7,919,676 |

In some embodiments of any of the aspects, the MF gene is Mfw2. Genomic, coding, and polypeptide sequences for the three homoeologues of Mfw2 occurring in the Triticum aestivum variety “Fielder” genome are provided herein as SEQ ID Nos. 4-6, 10-12, 14, 16, 18, and/or 21. A Mfw2 gene or sequence can be a naturally-occurring Mfw2 gene or sequence occurring in a plant, e.g., wheat. In some embodiments of any of the aspects, a MF gene can be the gene from a species, cultivar, or variety which has the highest degree of homology and/or sequence identity of the genes in that species', cultivar's or variety's genome with an Mfw2 sequence provided herein.

In some embodiments of any of the aspects, a functional allele of a MF gene shares at least 80% sequence identity with at least one of SEQ ID NOs: 4-6, 10-12, 14, 16, 18, and/or 21. In some embodiments of any of the aspects, a functional allele of a MF gene displays the same type of activity and shares at least 80% sequence identity with at least one of SEQ ID NOs: 4-6, 10-12, 14, 16, 18, and/or 21. In some embodiments of any of the aspects, a functional allele of a MF gene shares at least 80%, at least 85%, at least 90%, at least 95%, at least 98%, or greater sequence identity with at least one of SEQ ID NOs: 4-6, 10-12, 14, 16, 18, and/or 21. In some embodiments of any of the aspects, a functional allele of a MF gene displays the same type of activity and shares at least 80%, at least 85%, at least 90%, at least 95%, at least 98%, or greater sequence identity with at least one of SEQ ID NOs: 4-6, 10-12, 14, 16, 18, and/or 21. In some embodiments of any of the aspects, a functional allele of a MF gene shares at least 95% sequence identity with at least one of SEQ ID NOs: 4-6, 10-12, 14, 16, 18, and/or 21. In some embodiments of any of the aspects a functional allele of a MF gene displays the same type of activity and shares at least 95% sequence identity with at least one of SEQ ID NOs: 4-6, 10-12, 14, 16, 18, and/or 21. In some embodiments of any of the aspects, the functional allele of a MF gene is one of SEQ ID NOs: 4-6, 10-12, 14, 16, 18, and/or 21.

In some embodiments of any of the aspects, the MF gene is Ms1. Although Ms1 expresses a protein which is vital for development of an independent haploid pollen grain/sperm cell through to its successful germination on and penetration of a stigma and finally fertilization of an ovule, it is expressed in the diploid phase before haploid phase microgametogenesis. Ms1 is understood to be expressed in microsporocytes and secondary sporogenous cells but not in pollen grains during microgametogeneis. Additionally, a single copy of Ms1 is sufficient to resuce an Ms1 knockout. For characterization and further information regarding Ms1, see Wang et al. PNAS 2017 114 (47) 12614-12619; which is incorporated by reference herein in its entirety. Genomic, coding, and polypeptide sequences for the three homologues of Ms1 occurring in the Chinese Spring genome are provided herein as SEQ ID Nos. 183-191 and SEQ ID NOs: 214-216 (B genome). A Ms1 gene or sequence can be a naturally-occurring Ms1 gene or sequence occurring in a plant, e.g., wheat. In some embodiments of any of the aspects, a Ms1 gene can be the gene from a species, cultivar, or variety which has the highest degree of homology and/or sequence identity of the genes in that species', cultivar's or variety's genome with an Ms1 sequence provided herein.

In some embodiments of any of the aspects, the MF gene shares at least 80% sequence identity with Ms1. In some embodiments of any of the aspects, the MF gene displays the same type of activity and shares at least 80% sequence identity with Ms1. In some embodiments of any of the aspects, the MF gene shares at least 80%, at least 85%, at least 90%, at least 95%, at least 98%, or greater sequence identity with Ms1. In some embodiments of any of the aspects, the MF gene displays the same type of activity and shares at least 80%, at least 85%, at least 90%, at least 95%, at least 98%, or greater sequence identity with Ms1. In some embodiments of any of the aspects, the MF gene shares at least 95% sequence identity with Ms1. In some embodiments of any of the aspects, the MF gene displays the same type of activity and shares at least 95% sequence identity with Ms1. In some embodiments of any of the aspects, the MF gene is Ms1.

In the first genome, the first chromosome can be engineered to comprise a functional allele(s) of a MF gene at the MF loci and at least one allele of a seed color gene (e.g., seed coat and/or seed endosperm gene) (or at least one functional ectopic allele of each member of a set of seed color genes) by at least two different methods. In a first method, the endogenous wild-type functional allele of the MF gene is not engineered or modified, and the seed color gene (e.g., seed coat and/or seed endosperm gene) (or at least one functional ectopic allele of each member of a set of seed color genes) is inserted and/or introgressed into the chromosome, e.g., at the MF locus. Accordingly, in some embodiments of any of the aspects, the at least one functional allele of a MF gene is the endogenous wild-type functional allele of the MF gene.

In a second method, the endogenous allele of the MF gene is engineered to a loss-of-function MF allele and then a functional allele(s) of the MF gene is inserted and/or introgressed, e.g., as part of a single construct that includes the seed color gene (e.g., seed coat and/or seed endosperm gene) (or at least one functional ectopic allele of each member of a set of seed color genes). Accordingly, in some embodiments of any of the aspects, the at least one functional allele of a MF gene is an ectopic copy of the MF gene. In some embodiments of any of the aspects, the at least one functional allele of a MF gene and the at least one allele of a seed color gene (or at least one allele of each of a set of seed color genes) (e.g., seed coat and/or seed endosperm gene) are part of single construct.

In some embodiments of any of the aspects, a male-sterile line may comprise the recited modifications/alleles of two or more MF genes, e.g., due to redundancy and/or leaky phenotypes. In such embodiments, the maintainer line will comprise the same arrangement of MF alleles described herein, but for both MF genes.

As used herein, “PV” or “pollen vital” gene is a gene which, when its expression is inhibited, decreases the rate and/or success of pollen development and which functions post-meiosis, e.g, including the haploid phases towards the end of meiosis. In some embodiments of any of the aspects, a PV gene, when fully deactivated in a plant, is sufficient to eliminate development and/or germination of mature pollen and/or pollen-tube extension/ovule fertilisation, e.g., the PV gene is strictly necessary for pollen development. PV genes for various species have been described in the art, and exemplary, but non-limiting PV genes include those described in Golovkin and Redd et al PNAS 100(18) 10558-10563 (2003), as well as the Ms genes (e.g., Ms26 and Ms45) described in Wang et al. PNAS 2017; Singh et al. PloS One 12(5) e0177632 (2017); Timofejva et al. G3: Genes-Genomes-Genetc 3:231-249 (2013); and Wu et al. Plant Biotechnology Journal 14:1046-1054 (2015); each of which is incorporated by reference herein in its entirety. In some embodiments of any of the aspects, the PV gene is a gene which has been identified to produce a pollen-death phenotype when a plant was modified to a knock-out for that gene.

The PV gene selected for use in the compositions and methods described herein can, e.g., have homology to a gene demonstrated to be vital for post-meiosis events such as pollen-grain development, germination, or pollen tube extension in a plant. A non-limiting list of exemplary PV genes is provided in Table 2. In some embodiments of any of the aspects, the PV gene is a gene selected from Table 2. In some embodiments of any of the aspects, the PV gene is a gene which displays the same type of activity, and/or shares at least 80%, at least 85%, at least 90%, at least 95%, at least 98%, or greater sequence identity with a PV gene of Table 2. In some embodiments of any of the aspects, a PV gene can be the gene from a species, cultivar, or variety which has the highest degree of homology and/or sequence identity of the genes in that species', cultivar's or variety's genome with a gene selected from Table 2.

In some embodiments of any of the aspects, a functional allele of a PV gene shares at least 80% sequence identity with at least one sequence of Table 2. In some embodiments of any of the aspects, a functional allele of a PV gene displays the same type of activity and shares at least 80% sequence identity with at least one sequence of Table 2. In some embodiments of any of the aspects, a functional allele of a PV gene shares at least 80%, at least 85%, at least 90%, at least 95%, at least 98%, or greater sequence identity with at least one sequence of Table 2. In some embodiments of any of the aspects, a functional allele of a PV gene displays the same type of activity and shares at least 80%, at least 85%, at least 90%, at least 95%, at least 98%, or greater sequence identity with at least one sequence of Table 2. In some embodiments of any of the aspects, a functional allele of a PV gene shares at least 95% sequence identity with at least one sequence of Table 2. In some embodiments of any of the aspects a functional allele of a PV gene displays the same type of activity and shares at least 95% sequence identity with at least one sequence of Table 2. In some embodiments of any of the aspects, the functional allele of a PV gene is a sequence of Table 2.

TABLE 2
Exemplary PV genes

| | | | TGAC v1 homoeologues* - | |
|---|---|---|---|---|
| | | | the copies on the other | |
| Assigned | | | subgenomes of wheat and | |
| Mfw | | TGAC v1 gene | their associated gene | RefSeq v1.1 |
| name | Blast hit | model* | models | Sequences |
| Mfw1-A | RPG1 | TRIAE_CS42_7AL_{—} | TRIAE_CS42_7BL_TGACV1_{—} | TraesCS7A02G533900 |
| | (RUPTURED | TGACv1_556969_{—} | 580455_AA1914070; | |
| | POLLEN | AA1774370 | TRIAE_CS42_7DL_TGACv1_{—} | |
| | GRAIN1) | | 603435_AA1983700 | |
| | like | | | |
| Mfw1-B | RPG1 | TRIAE_CS42_7BL_{—} | TRIAE_CS42_7AL_TGACv1_{—} | TraesCS7B02G451200 |
| | (RUPTURED | TGACv1_580455_{—} | 556969_AA1774370; | |
| | POLLEN | AA1914070 | TRIAE_CS42_7DL_TGACv1_{—} | |
| | GRAIN1) | | 603435_AA1983700 | |
| | like | | | |
| Mfw1-D | RPG1 | TRIAE_CS42_7DL_{—} | TRIAE_CS42_7AL_TGACv1_{—} | TraesCS7D02G521400 |
| | (RUPTURED | TGACv1_603435_{—} | 556969_AA1774370; | |
| | POLLEN | AA1983700 | TRIAE_CS42_7BL_TGACV1_{—} | |
| | GRAIN1) | | 580455_AA1914070 | |
| | like | | | |
| Mfw4-D | RPG1 | TRIAE_CS42_5BS_{—} | TRIAE_CS42_5AS_TGACv1_{—} | |
| | (RUPTURED | TGACv1_423307_{—} | 393366_AA1271880; | |
| | POLLEN | AA1373980; | TRIAE_CS42_5DS_TGACV1_{—} | |
| | GRAIN1) | | 457788_AA1489840 | |
| | like | | | |
| Ms26 | | TRIAE_CS42_4AS_{—} | | SEQ ID Nos: 192-200 |
| | | TGACv1_308399_{—} | | |
| | | AA1027760 | | |
| | | TRIAE_CS42_4BL_{—} | | |
| | | TGACv1_321123_{—} | | |
| | | AA1055760 | | |
| | | TRIAE_CS42_4DL_{—} | | |
| | | TGACv1_345634_{—} | | |
| | | AA1154040 | | |
| Ms45 | | TRIAE_CS42_4AS_{—} | | SEQ ID Nos: 201-209 |
| | | TGACv1_307709_{—} | | |
| | | AA1022920 | | |
| | | TRIAE_CS42_4BL_{—} | | |
| | | TGACv1_320775_{—} | | |
| | | AA1048430 | | |
| | | TRIAE_CS42_4DL_{—} | | |
| | | TGACv1_343561_{—} | | |
| | | AA1136570 | | |
| RPG1 | | | | NC_003076.8 |
| PV1 | | | | SEQ ID NOs: 174-182 |
| (NPG1) | | | | See also Golovkin, M. |
| | | | | PNAS. (2003) 100, |
| | | | | 10558-1056; which |
| | | | | is incorporated by |
| | | | | reference herein in |
| | | | | its entirety |
| Apv1 | | | | See, e.g., Wu et al. |
| | | | | Plant Biotechnology |
| | | | | Journal 14: 1046-1054 |
| | | | | (2015); which is |
| | | | | incorporated by |
| | | | | reference herein in |
| | | | | its entirety |
| Ipe1 | | | | See, e.g., Wu et al. |
| | | | | Plant Biotechnology |
| | | | | Journal 14: 1046-1054 |
| | | | | (2015); which is |
| | | | | incorporated by |
| | | | | reference herein in |
| | | | | its entirety |
| PV2 | | | | TraesCS4A02G001100 |
| (ANX1) | | | | TraesCS4B02G002300 |
| | | | | TraesCS4D02G001000 |
| PV3 | | | | TraesCS4A02G441600.1 |
| (RUP0) | | | | TraesCS7A02G055000.1 |
| | | | | TraesCS7D02G050100.1 |
| | | | | SEQ ID NOs: 222-230 |
| | | | | and 257 |
| | | | | See also Yang et al. |
| | | | | Joural of Experimental |
| | | | | Botany 2020 71: 2112- |
| | | | | 26; which is |
| | | | | incorporated by |
| | | | | reference herein in |
| | | | | its entirety. |

In some embodiments of any of the aspects, the PV gene is PV1, or pollen-grain-vital gene 1. PV1 expresses a protein which is vital for development of an independent haploid pollen grain/sperm cell through to its successful germination on and penetration of a stigma and finally fertilization of an ovule. PV1 is understood to be expressed in microsporocytes and secondary sporogenous cells. See, e.g., Golovkin, M. PNAS. (2003) 100, 10558-1056; which is incorporated by reference herein in its entirety. Additionally, a single copy of PV1 is sufficient to rescue an PV1 knockout. Genomic, coding, and polypeptide sequences for the three homoeologues' pairs of PV1 occurring in the Chinese Spring genome are provided herein as SEQ ID Nos. 174-182. A PV1 gene or sequence can be a naturally-occurring PV1 gene or sequence occurring in a plant, e.g., wheat. In some embodiments of any of the aspects, a PV1 gene can be the gene from a species, cultivar, or variety which has the highest degree of homology and/or sequence identity of the genes in that species', cultivar's or variety's genome with an PV1 sequence provided herein.

In some embodiments of any of the aspects, the PV gene shares at least 80% sequence identity with PV1. In some embodiments of any of the aspects, the PV gene displays the same type of activity and shares at least 80% sequence identity with PV1. In some embodiments of any of the aspects, the PV gene shares at least 80%, at least 85%, at least 90%, at least 95%, at least 97%, at least 98%, or greater sequence identity with PV1. In some embodiments of any of the aspects, the PV gene displays the same type of activity and shares at least 80%, at least 85%, at least 90%, at least 95%, at least 97%, at least 98%, or greater sequence identity with PV1. In some embodiments of any of the aspects, the PV gene shares at least 95% sequence identity with PV1. In some embodiments of any of the aspects, the PV gene displays the same type of activity and shares at least 95% sequence identity with PV1. In some embodiments of any of the aspects, the PV gene is PV1.

In some embodiments of any of the aspects, a functional allele of a PV gene shares at least 80% sequence identity with at least one of SEQ ID NOs: 1, 3, 4, 6, 7, and 9, or encodes a polypeptide with at least 80% sequence identity with at least one of SEQ ID NOs: 2, 5, and 8. In some embodiments of any of the aspects, a functional allele of a PV gene displays the same type of activity and shares at least 80% sequence identity with at least one of SEQ ID NOs: 1, 3, 4, 6, 7, and 9, or encodes a polypeptide with at least 80% sequence identity with at least one of SEQ ID NOs: 2, 5, and 8. In some embodiments of any of the aspects, a functional allele of a PV gene shares at least 80%, at least 85%, at least 90%, at least 95%, at least 97%, at least 98%, or greater sequence identity with at least one of SEQ ID NOs: 1, 3, 4, 6, 7, and 9, or encodes a polypeptide with at least 80% sequence identity with at least one of SEQ ID NOs: 2, 5, and 8. In some embodiments of any of the aspects, a functional allele of a PV gene displays the same type of activity and shares at least 80%, at least 85%, at least 90%, at least 95%, at least 97%, at least 98%, or greater sequence identity with at least one of SEQ ID NOs: 1, 3, 4, 6, 7, and 9, or encodes a polypeptide with at least 80% sequence identity with at least one of SEQ ID NOs: 2, 5, and 8. In some embodiments of any of the aspects, a functional allele of a PV gene shares at least 95% sequence identity with at least one of SEQ ID NOs: 1, 3, 4, 6, 7, and 9, or encodes a polypeptide with at least 80% sequence identity with at least one of SEQ ID NOs: 2, 5, and 8. In some embodiments of any of the aspects a functional allele of a PV gene displays the same type of activity and shares at least 95% sequence identity with at least one of SEQ ID NOs: 1, 3, 4, 6, 7, and 9, or encodes a polypeptide with at least 80% sequence identity with at least one of SEQ ID NOs: 2, 5, and 8. In some embodiments of any of the aspects, the functional allele of a PV gene is one of SEQ ID NOs: 1, 3, 4, 6, 7, and 9, or encodes a polypeptide of one of SEQ ID NOs: 2, 5, and 8.

In some embodiments of any of the aspects, the PV gene is PV2, or pollen-grain-vital gene 2. For further discussion of the activity and characterization of P2(ANX1) see, e.g., Boisson-Dernier A et al. Development (2009) 136:3279-3288; and Miyazaki S, eta 1. Curr Biol (2009) 19:1327-1331, each of which is incorporated by reference herein in its entirety. Genomic, coding, and polypeptide sequences for the three homologues of PV2 occurring in the Chinese Spring genome are provided herein as SEQ ID Nos. 157-165. A PV2 gene or sequence can be a naturally-occurring PV2 gene or sequence occurring in a plant, e.g., wheat. In some embodiments of any of the aspects, a PV2 gene can be the gene from a species, cultivar, or variety which has the highest degree of homology and/or sequence identity of the genes in that species', cultivar's or variety's genome with an PV2 sequence provided herein.

In some embodiments of any of the aspects, the PV gene shares at least 80% sequence identity with PV2. In some embodiments of any of the aspects, the PV gene displays the same type of activity and shares at least 80% sequence identity with PV2. In some embodiments of any of the aspects, the PV gene shares at least 80%, at least 85%, at least 90%, at least 95%, at least 98%, or greater sequence identity with PV2. In some embodiments of any of the aspects, the PV gene displays the same type of activity and shares at least 80%, at least 85%, at least 90%, at least 95%, at least 98%, or greater sequence identity with PV2. In some embodiments of any of the aspects, the PV gene shares at least 95% sequence identity with PV2. In some embodiments of any of the aspects, the PV gene displays the same type of activity and shares at least 95% sequence identity with PV2. In some embodiments of any of the aspects, the PV gene is PV2.

In some embodiments of any of the aspects, a functional allele of a PV gene shares at least 80% sequence identity with at least one of SEQ ID NOs: 157, 158, 160, 161, 163, and 164, or encodes a polypeptide with at least 80% sequence identity with at least one of SEQ ID NOs: 159, 162, or 165. In some embodiments of any of the aspects, a functional allele of a PV gene displays the same type of activity and shares at least 80% sequence identity with at least one of SEQ ID NOs: 157, 158, 160, 161, 163, and 164, or encodes a polypeptide with at least 80% sequence identity with at least one of SEQ ID NOs: 159, 162 and 165. In some embodiments of any of the aspects, a functional allele of a PV gene shares at least 80%, at least 85%, at least 90%, at least 95%, at least 98%, or greater sequence identity with at least one of SEQ ID NOs: 157, 158, 160, 161, 163, and 164, or encodes a polypeptide with at least 80% sequence identity with at least one of SEQ ID NOs: 159, 162, and 165. In some embodiments of any of the aspects, a functional allele of a PV gene displays the same type of activity and shares at least 80%, at least 85%, at least 90%, at least 95%, at least 98%, or greater sequence identity with at least one of SEQ ID NOs: 157, 158, 160, 161, 163, and 164, or encodes a polypeptide with at least 80% sequence identity with at least one of SEQ ID NOs: 159, 162, and 165. In some embodiments of any of the aspects, a functional allele of a PV gene shares at least 95% sequence identity with at least one of SEQ ID NOs: 157, 158, 160, 161, 163, and 164, or encodes a polypeptide with at least 80% sequence identity with at least one of SEQ ID NOs 159, 162, and 165. In some embodiments of any of the aspects a functional allele of a PV gene displays the same type of activity and shares at least 95% sequence identity with at least one of SEQ ID NOs: 157, 158, 160, 161, 163, and 164, or encodes a polypeptide with at least 80% sequence identity with at least one of SEQ ID NOs: 159, 162, and 165. In some embodiments of any of the aspects, the functional allele of a PV gene is one of SEQ ID NOs: 157, 158, 160, 161, 163, and 164, or encodes a polypeptide of one of SEQ ID NOs: 159, 162, and 165.

In some embodiments of any of the aspects, the PV gene is Pollen Vital 3 (PV3) or RUPO, or Ruptured Pollen Tube. For further discussion of the activity and characterization of PV3 (RUPO) see, e.g., Liu LPLoS Genet 2016 12(7): e1006085, which is incorporated by reference herein in its entirety. Genomic, coding, and polypeptide sequences for the three homoeologues' pairs of PV3 are provided herein as SEQ ID Nos. 222-230. A PV3 gene or sequence can be a naturally-occurring PV3 gene or sequence occurring in a plant, e.g., wheat. In some embodiments of any of the aspects, a PV3 gene can be the gene from a species, cultivar, or variety which has the highest degree of homology and/or sequence identity of the genes in that species', cultivar's or variety's genome with an PV3 sequence provided herein.

In some embodiments of any of the aspects, the PV gene shares at least 80% sequence identity with PV3. In some embodiments of any of the aspects, the PV gene displays the same type of activity and shares at least 80% sequence identity with PV3. In some embodiments of any of the aspects, the PV gene shares at least 80%, at least 85%, at least 90%, at least 95%, at least 97%, at least 98%, or greater sequence identity with PV3. In some embodiments of any of the aspects, the PV gene displays the same type of activity and shares at least 80%, at least 85%, at least 90%, at least 95%, at least 97%, at least 98%, or greater sequence identity with PV3. In some embodiments of any of the aspects, the PV gene shares at least 95% sequence identity with PV3. In some embodiments of any of the aspects, the PV gene displays the same type of activity and shares at least 95% sequence identity with PV3. In some embodiments of any of the aspects, the PV gene is PV3.

In some embodiments of any of the aspects, a functional allele of a PV gene shares at least 80% sequence identity with at least one of SEQ ID NOs: 222, 224, 225, 227, 228, 230, and 257 or encodes a polypeptide with at least 80% sequence identity with at least one of SEQ ID NOs: 223, 226, and 229. In some embodiments of any of the aspects, a functional allele of a PV gene displays the same type of activity and shares at least 80% sequence identity with at least one of SEQ ID NOs: 222, 224, 225, 227, 228, 230, and 257 or encodes a polypeptide with at least 80% sequence identity with at least one of SEQ ID NOs: 223, 226, and 229. In some embodiments of any of the aspects, a functional allele of a PV gene shares at least 80%, at least 85%, at least 90%, at least 95%, at least 97%, at least 98%, or greater sequence identity with at least one of SEQ ID NOs: 222, 224, 225, 227, 228, 230, and 257 or encodes a polypeptide with at least 80% sequence identity with at least one of SEQ ID NOs: 223, 226, and 229. In some embodiments of any of the aspects, a functional allele of a PV gene displays the same type of activity and shares at least 80%, at least 85%, at least 90%, at least 95%, at least 97%, at least 98%, or greater sequence identity with at least one of SEQ ID NOs: 222, 224, 225, 227, 228, 230, and 257 or encodes a polypeptide with at least 80% sequence identity with at least one of SEQ ID NOs: 223, 226, and 229. In some embodiments of any of the aspects, a functional allele of a PV gene shares at least 95% sequence identity with at least one of SEQ ID NOs: 222, 224, 225, 227, 228, 230, and 257 or encodes a polypeptide with at least 80% sequence identity with at least one of SEQ ID NOs: 223, 226, and 229. In some embodiments of any of the aspects a functional allele of a PV gene displays the same type of activity and shares at least 95% sequence identity with at least one of SEQ ID NOs: 222, 224, 225, 227, 228, 230, and 257 or encodes a polypeptide with at least 80% sequence identity with at least one of SEQ ID NOs: 223, 226, and 229. In some embodiments of any of the aspects, the functional allele of a PV gene is one of SEQ ID NOs: 222, 224, 225, 227, 228, 230, and 257 or encodes a polypeptide of one of SEQ ID NOs: 223, 226, and 229.

In some embodiments of any of the aspects, the endogenous MF and PV genes are located on the same arms of the same homologous pair of chromosomes in the wild-type genome.

A seed color gene is a gene or allele that, when at least one copy is present in the genome, will cause some or all of the tissue of the seed of the plant to have a different color than in the absence of the at least one copy of that gene or allele. In some embodiments of any of the aspects, the tissue is the seed coat. In some embodiments of any of the aspects, the tissue is the endosperm. In some embodiments of any of the aspects, the seed color gene is a seed color gene (e.g., seed coat and/or seed endosperm gene). A seed endosperm color gene is a gene or allele that, when at least one dominant expressed copy is present in the genome, will cause the endosperm of the seed of the plant to have a different color than in the absence of the at least one dominant copy of that gene or allele. The genome of an endosperm comprises two copies of the maternal genome (ie from the ovule) and only one from the paternal parent (ie the sperm cell). So embryos from, e.g., a heterozygous (MFW′:BA/PV′ maintainer as described herein will have either two copies of BA:MFW′ or two copies of PV′. With no BA allele from the sperm cell, in the maintainer described herein, seeds from the former will have a different (BA darker/blue) color seed and the latter (PV′) will have wildtype seed color; hence the two genotypes can be color-sorted with an optical sorter—a particular benefit for the production of the maintainer and male-sterile in the hybrid system described herein. The color can be in the visible or non-visible spectrum. Different color refers to a distinguishable difference in color, either by the human eye or a machine. The difference can be a difference in saturation, lightness, darkness, color, or hue. The color can be due to production of a pigment or any other change that impacts the light absorption, reflection, or refraction of the seed. In some embodiments of any of the aspects, a set of seed color genes, e.g, two or more different genes, are required to express the different color. In such embodiments, where ever a singular seed color gene or allele is referenced, embodiments comprising a set of seed color genes or a set of seed color gene alleles is specifically contemplated. In some embodiments, the plants, chromosomes, and/or cassettes described herein can comprise a set of seed color genes (or at least one allele of each member of a seed color gene set) in place of a singlular seed color gene or allele thereof. Suitable seed color genes (e.g., seed coat and/or seed endosperm gene) are known in the art and include, by way of non-limiting example, blue aleurone (BA) or deep-red (DsRed). Sequences for these seed color gene (e.g., seed coat and/or seed endosperm gene)s are known in the art, e.g., BA sequences are described in US Patent Publication US2020/0255856; Zheng et al. Euphytica 2006 152:51-60; Zeller et al. Theor Appl Genet. 19991 81:551-558; Li et al. PLoS One 2017 12:e0181116 (see, e.g, SEQ ID NO: 155); Zong et al. Plant Cell Rep 2019 38:1291-8; each of which is incorporated by reference herein in its entirety. As a further illustrative example, HvMYC2 is a suitable seed color gene in barley and is described in the art, e.g., at Strygina et al. BMC Plant Biology 2017 17:184, which is incorporated by reference herein in its entirety.

The BA gene's grain phenotype has been shown to be dose-related, but one allele's expression is enough for a darker-grained phenotype to be colour-selectable. In fact in the maintainer's endosperm there will be two alleles from the maternal side with BA and one from the paternal without it, providing double the amount of BA alleles needed for functional colour-sorting. In some embodiments of any of the aspects, the blue aleurone gene comprises, consists of, or consists essentially of a sequence of SEQ ID NO: 155. In some embodiments of any of the aspects, the blue aleurone gene comprises, consists of, or consists essentially of a sequence having at least 60%, at least 65%, at least 70%, at least 75%, at least 80%, at least 90%, at least 95%, or at least 98% sequence identity with SEQ ID NO: 155. In some embodiments of any of the aspects, the blue aleurone gene comprises, consists of, or consists essentially of a sequence having at least 60%, at least 65%, at least 70%, at least 75%, at least 80%, at least 90%, at least 95%, or at least 98% sequence identity with SEQ ID NO: 155 and which retains the wild-type activity of SEQ ID NO: 155 (e.g., causing blue seed endosperm color). In some embodiments of any of the aspects, the blue aleurone gene comprises, consists of, or consists essentially of a sequence having at least 90% sequence identity with SEQ ID NO: 155 and which retains the wild-type activity of SEQ ID NO: 155 (e.g., causing blue seed endosperm color). In some embodiments of any of the aspects, the blue aleurone gene comprises, consists of, or consists essentially of a sequence having at least 95% sequence identity with SEQ ID NO: 155 and which retains the wild-type activity of SEQ ID NO: 155 (e.g., causing blue seed endosperm color).

SEQ ID NO: 155
atgcgggaaatagctactcagcggtgtggtaatcgatcaatggcgctatcagctcctccc
agtcaggaacagccgtcggggaagcaattcggctaccagctcgctgctgctgtgaggagc
atcaactggacttatggcatattttggtccatttccgccagcccgcgcccaggccactcc
tcagttctggcgtggaaggatgggttctacaacggcgagataaagactagaaagattacc
ggctcgaccactacggagcttacagcggacgagcgcgtcatgcacagaagcaagcaactg
agggagctctacgaatcgctcttgcccggcaactccaacaaccgggcaaggcgaccaacc
gcctcactgtcaccggaggatctcggggacggcgagtggtattacaccataagcatgact
tacaccttccaccctaatcaagggttgccaggcaaaagctttgcgagcaatcaacatgtt
tggctgtacaacgctcaatacgcaaacaccagagttttcccccgcgcgctcttagcaaag
acaatcgtttgcattcccttcatgggcggtgtgcttgagctcggaacgtcggatcaggtg
ttggaggacccgagcatggtgaagcggatcagcacgtctttctgggagctgcacttgccg
tcatccttggagtcgaaggatccgagctccagcacatcagcaaacgataccagggaggcc
accgacatcatcttgttcgaggatttcgaccacaacgacacagttgagggggtgatctct
gagcaaagggaggtccagtgcccgtccaacgtcaatctggagcgcctcacaaagcagatg
gacgagttccacagccttctcggtggactggacgtgcatcctctcgaagacagatggatc
atggacgagccctttgagtttacgttttccccagaagtggcgccggctatggatatgccg
agcaccgacgatgtcatcgtcactttaagtaggtccgaaggctctcgtccatcctgcttc
acagcgtggaagggatcatccgagtcgaaatacgtggctggccaggtcgttggggagtca
cagaagttgctgaataaagttgtggctggtggtgcatgggcgagcaattatggcggtcgc
accatggtgagagctcagggaattaacagcaacacccatgtcatgacagagagaagacgc
cgggagaaactcaacgagatgttcctggttctcaagtcactggtcccgtccattcacaag
gtagacaaagcatccatcctcacagaaacgataggttatcttagagaactgaagcaaagg
gtagatcagctagaatccagccggtcaccgtctcacccaaaagaaacaacaggaccgagc
agaagccatgtcgtcggcgctaggaagaagatagtctcggccggatccaagaggaaggcg
ccagggctggagagcccgagcaatgtcgtgaacgtgacgatgctggacaaggtggtgctg
ttggaggtgcagtgcccgtggaaggagctgctgatgacacaagtgtttgacgccatcaag
agcctctgtctggacgttgtctccgtgcaggcatccacatcaggtggccgtcttgacctc
aagatacgagctaatcagcagcttgcggtcggttctgctatggtggcacctggggcaatc
accgaaacacttcagaaagctatatag

In some embodiments of any of the aspects, the at least one seed color gene (e.g., seed coat and/or seed endosperm gene)/allele (or set of seed color genes/alleles) is a sequence from a different line or variety of the same species as the plant/cell. In some embodiments of any of the aspects, the at least one seed color gene (e.g., seed coat and/or seed endosperm gene) allele (or set of seed color genes/alleles) is a sequence from the same genus as the plant/cell. In some embodiments of any of the aspects, the at least one seed color gene (e.g., seed coat and/or seed endosperm gene) allele (or set of seed color genes/alleles) comprises, consists of, or consists essentially of a sequence from T. aestivum, T durum or T. monococcum, or another Triticum aestivum-crossable species. In some embodiments of any of the aspects, the at least one seed color gene (e.g., seed coat and/or seed endosperm gene) (or set of seed color genes/alleles) is exogenous, e.g., the gene is not present in the relevant genome(s) except for the functional copy(ies) of the seed color gene (e.g., seed coat and/or seed endosperm gene) (or set of seed color genes/alleles) prior to the engineered modifications described herein. In some embodiments of any of the aspects, the the at least one allele of a seed color gene (e.g., seed coat and/or seed endosperm gene) is at least two copies and/or individual alleles of the seed color gene (e.g., seed coat and/or seed endosperm gene) In some embodiments of any of the aspects, the the at least one allele of a seed color gene (e.g., seed coat and/or seed endosperm gene) is at least three copies and/or individual alleles of the seed color gene (e.g., seed coat and/or seed endosperm gene). In some embodiments of any of the aspects, the the at least one allele of a seed color gene (e.g., seed coat and/or seed endosperm gene) is at least four copies and/or individual alleles of the seed color gene (e.g., seed coat and/or seed endosperm gene).

An allele or gene described herein can comprise both a coding sequence and one or more regulatory sequences operably linked to the coding sequence. Regulatory sequences can include but are not limited to promoters, enhancers, boundary elements, insulators, 5′ untranslated (5′UTR) or “leader” sequences, 3′ UTR or “trailer” sequences, etc. In some embodiments of any of the aspects, the regulatory sequences of an ectopic gene or allele are the regulatory sequences which are endogenous to that gene or allele in its wild-type context, e.g., an ectopic gene includes a coding sequence and one or more of its native regulatory sequences. In some embodiments of any of the aspects, the regulatory sequences of an exogenous gene or allele are the regulatory sequences which are endogenous to that gene or allele in its wild-type context, e.g., an exogenous gene includes a coding sequence and one or more of its native regulatory sequences (which are also exogenous to the plant/cell). In some embodiments of any of the aspects, the regulatory sequences of an exogenous or ectopic gene or allele are regulatory sequences which are endogenous to the plant/cell. In some embodiments of any of the aspects, the regulatory sequences of an exogenous or ectopic gene or allele are regulatory sequences which are endogenous to the plant/cell but not native to the gene or allele.

In some embodiments of any of the aspects, one or more functional alleles can comprise cDNA constructs derived from wild-type or functional alleles of the relevant gene(s) (e.g., introns are not present). In some embodiments of any of the aspects, functional alleles can comprise endogenous promoters, enhancers, and/or terminators in the normal sense orientation. In some embodiments, a functional allele and/or seed color gene (e.g., seed coat and/or seed endosperm gene) (or set of seed color genes/alleles) expression can be driven by exogenous and/or heterologous promoters, enhancers, and/or terminators. Exemplary promoters include OsU3, TaU3, TaU6 and OsU6 promoters.

As described herein, a functional allele(s) of a MF gene at the MF gene locus and at least one allele of a seed color gene (e.g., seed coat and/or seed endosperm gene) (or set of seed color genes/alleles) are present on a first chromosome of a pair of homologous chromosomes. In some embodiments, the at least one functional allele of the MF gene and the seed color gene (e.g., seed coat and/or seed endosperm gene) (or set of seed color genes/alleles) are located within 10 centimorgans (cM) of each other, e.g., within 10 cM, within 5 cM, within 2 cM, within 1 cM, within 0.75 cM, within 0.5 cM, or within 0.25 cM. In some embodiments, the seed color gene (e.g., seed coat and/or seed endosperm gene) (or set of seed color genes/alleles) is located within 10 centimorgans (cM) of the MF gene locus, e.g., within 10 cM, within 5 cM, within 2 cM, within 1 cM, within 0.75 cM, within 0.5 cM, or within 0.25 cM.

As described herein, a loss-of-function allele of the MF gene and at least one ectopic functional allele of a PV gene are present on a second chromosome of a pair of homologous chromosomes. In some embodiments, the at least one functional allele of the PV gene and the loss-of-function allele of the MF gene are located within 10 centimorgans (cM) of each other, e.g., within 10 cM, within 5 cM, within 2 cM, within 1 cM, within 0.75 cM, within 0.5 cM, or within 0.25 cM. In some embodiments, the at least one functional allele of the PV gene is located within 10 centimorgans (cM) of the MF gene locus, e.g., within 10 cM, within 5 cM, within 2 cM, within 1 cM, within 0.75 cM, within 0.5 cM, or within 0.25 cM.

In some embodiments of any of the aspects, a maintainer plant described herein comprises multiple functional alleles and/or seed color gene (e.g., seed coat and/or seed endosperm gene alleles (or set of seed color genes/alleles), e.g., multiple copies of the same relevant gene, e.g., arranged in series. Multiple copies of a gene can increase the strength or penetrance of the relevant phenotype and may therefore be desired to avoid intermediate phenotypes or failure to express the phenotype dictated by the relevant genes. This is sometimes referred to in the art as “gene stacking.” Multiple copies of the genes described herein can be inserted into a genome by multiple sequential steps using any appropriate technology described herein or known in the art, or using technologies that permit insertion of large constructs. By way of non-limiting example, GAANTRY technology can transfer multiple genes into a genome via a single construct (see Collier et al. The Plant Journal 2018 95:573-583) and alternative technology to transfer cassettes of at least 37 kb and likely as much as 100 kb, into wheat is also known in the art (see Luo et al. Nature Biotechnology 2021 39:561-566 doi: 10.1038/s41587-020-00770-x). The foregoing references are incorporated by reference herein in their entireties.

In some embodiments of any of the aspects, the maintainer plant does not comprise any genetic sequences which are exogenous to that plant species except for the allele(s) of the seed color gene (e.g., seed coat and/or seed endosperm gene) (or set of seed color genes/alleles). In some embodiments of any of the aspects, the maintainer plant does not comprise any genetic sequences which are exogenous to that plant genus except for the allele(s) of the seed color gene (e.g., seed coat and/or seed endosperm gene) (or set of seed color genes/alleles). In some embodiments of any of the aspects, the maintainer plant does not comprise any genetic sequences which are ectopic to that plant species except for the allele(s) of the seed color gene (e.g., seed coat and/or seed endosperm gene) (or set of seed color genes/alleles). In some embodiments of any of the aspects, the maintainer plant does not comprise any genetic sequences which are ectopic to that plant genus except for the allele(s) of the seed color gene (e.g., seed coat and/or seed endosperm gene) (or set of seed color genes/alleles).

In some embodiments of any of the aspects, the maintainer plant does not comprise any genetic sequences which are ectopic to that plant species except for the allele(s) of the seed color gene (e.g., seed coat and/or seed endosperm gene) (or set of seed color genes/alleles) and/or the at least one functional allele of the PV gene. In some embodiments of any of the aspects, the maintainer plant does not comprise any genetic sequences which are ectopic to that plant genus except for the allele(s) of the seed color gene (e.g., seed coat and/or seed endosperm gene) (or set of seed color genes/alleles) and/or the at least one functional allele of the PV gene.

The ectopic alleles and/or inserted alleles/genes/constructs can be inserted at target locus. In some embodiments, the target locus can be the MF or PV gene locus (e.g., the locus where the endogenous MF or PV gene is located) or the target locus can be a different locus that is not the MF or PV gene locus. In some embodiments of any of the aspects, the target locus can be a locus that is not the MF or PV gene locus. In some embodiments of any of the aspects, the ectopic alleles and/or inserted alleles/genes/constructs can be inserted downstream of an endogenous gene. In some embodiments of any of the aspects, the ectopic alleles and/or inserted alleles/genes/constructs does not disrupt the coding sequence and/or expression of an endogenous gene. In some embodiments of any of the aspects, the target locus can be on the same chromosome as the MF gene. In some embodiments of any of the aspects, the target locus can be on the same chromosome arm as the MF gene. In some embodiments of any of the aspects, the target locus can be on the same chromosome as the PV gene. In some embodiments of any of the aspects, the target locus can be on the same chromosome arm as the PV gene. In some embodiments of any of the aspects, the target locus can be on a different chromosome than the MF and PV genes. In some embodiments of any of the aspects, the target locus known in the art to permit expression of inserted genes/constructs. Such target loci are known in the art, e.g., the ANXA1 locus as described in WO 2013/169802, which is incorporated by reference herein in its entirety.

Where the specification refers to a maintainer line, it is meant that the line is a maintainer of the male-sterile genetics and that some of the maintainer's progeny from self-pollination will be male-sterile. The maintainer plant is not itself male-sterile.

In some embodiments of any of the aspects, the maintainer plant is substantially isogenic with the male-sterile plant with the exception of the engineered modifications in the first genome. In some embodiments of any of the aspects, the maintainer plant is substantially isogenic with the male-sterile plant with the exception of the engineered modifications on the first chromosome of the pair of homologous chromosomes in the first genome. In some embodiments of any of the aspects, the maintainer plant is substantially isogenic with the male-sterile plant with the exception of the first chromosome of the pair of homologous chromosomes in the first genome of the maintainer plant. In some embodiments of any of the aspects, the maintainer plant is substantially isogenic with the male-sterile plant with the exception of the seed color gene (e.g., seed coat and/or seed endosperm gene) (or set of seed color genes/alleles). In some embodiments of any of the aspects, the maintainer plant is substantially isogenic with the male-sterile plant with the exception of the at least one functional allele of the MF gene and the at least one allele of a seed color gene (e.g., seed coat and/or seed endosperm gene) (or set of seed color genes/alleles).

In some embodiments of any of the aspects, an ectopic allele or ectopic copy of a gene is a nuclease-null allele. As used herein, a “site-specific guided nuclease-null allele” (also referred to herein as a “nuclease-null allele”) refers to an engineered allele in which the sequence targeted by a selected site-specific guided nuclease (e.g, CRISPR-Cas9 guide sites) in the wild-type sequence have been engineered to comprise silent mutations that do not change the sequence of the polypeptide that the allele codes for, but which change the sequence targeted by the selected site-specific guided nuclease (e.g, CRISPR-Cas9 guide sites) into a sequence(s) which is not targeted the selected site-specific guided nuclease, e.g., is not a CRISPR-Cas9 guide site sequence. Such mutations are possible due to the fact that multiple codons can code for the same amino acid and appropriate mutations for a given sequence can be selected on the basis of known alternative codons. Silent mutations are also referred to herein as synonymous mutations.

In some embodiments of any of the aspects, a nuclease-null allele comprises 1 mutation, e.g., one nucleotide in the gene is mutated from the wild-type sequence. In some embodiments of any of the aspects, a nuclease-null allele comprises 2 mutations, e.g., two nucleotides in the gene are mutated from the wild-type sequence. In some embodiments of any of the aspects, a nuclease-null allele comprises at least 2 mutations, e.g., at least two nucleotides in the gene are mutated from the wild-type sequence. In some embodiments of any of the aspects, a nuclease-null allele comprises 2-5 mutations, e.g., two to five nucleotides in the gene are mutated from the wild-type sequence. In some embodiments of any of the aspects, a nuclease-null allele comprises 2-4 mutations, e.g., two to four nucleotides in the gene are mutated from the wild-type sequence.

In some embodiments of any of the aspects, a nuclease-null allele comprises mutations in at least two codons, e.g., at least two codons in the gene are mutated from the wild-type sequence. In some embodiments of any of the aspects, a nuclease-null allele comprises mutations in two codons, e.g., two codons in the gene are mutated from the wild-type sequence. In some embodiments of any of the aspects, a nuclease-null allele comprises mutations in 1-4 codons, e.g., 1-4 codons in the gene are mutated from the wild-type sequence. In some embodiments of any of the aspects, a nuclease-null allele comprises mutations in 2-4 codons, e.g., 2-4 codons in the gene are mutated from the wild-type sequence.

In some embodiments of any of the aspects, a nuclease-null allele comprises at least two mutations with each mutation occurring in a different codon. In some embodiments of any of the aspects, a nuclease-null allele comprises two mutations with each mutation occurring in a different codon.

In some embodiments of any of the aspects, the nuclease-null alleles is a CRISPR-null allele.

Exemplary but non-limiting CRISPR-null alleles are provided in Example 6, along with explanations of their design and production.

In some embodiments of any of the aspects, a nuclease-null allele of Mfw2 (e.g. a Mfw2′ allele) comprises a sequence comprising one or both of the T to C mutations of SEQ ID NO: 169, relative to SEQ ID NO: 168. In some embodiments of any of the aspects, a nuclease-null allele of Mfw2 (e.g. a Mfw2′ allele) comprises a Mfw2 sequence provided herein which has been modified to comprise one or two T to C mutations corresponding to one or both of the T to C mutations of SEQ ID NO: 169. In some embodiments of any of the aspects, a nuclease-null allele of Mfw2 (e.g. a Mfw2′ allele) comprises a sequence comprising both of the T to C mutations of SEQ ID NO: 169, relative to SEQ ID NO: 168. In some embodiments of any of the aspects, a nuclease-null allele of Mfw2 (e.g. a Mfw2′ allele) comprises a Mfw2 sequence provided herein which has been modified to comprise two T to C mutations corresponding to both of the T to C mutations of SEQ ID NO: 169.

In some embodiments of any of the aspects, a nuclease-null allele of Mfw2 (e.g., a Mfw2′ allele) comprises a a Mfw2 sequence provided herein which has been modified to comprise a G>A mutation as shown in SEQ ID NO: 239, relative to SEQ ID NO: 238 and 188:

Wild-type allele sequence
cccACGCGGCACTAACTACTATC (SEQ ID NO: 238, a
portion of SEQ ID NO: 188)
Nuclease-null allele sequence
(SEQ ID NO: 239)
cccACGCAGCACTAACTACTATC

Alternatively, the Mfw2′ allele comprises a naturally-occurring sequence comprising a G>A mutation as shown in SEQ ID NO: 239, relative to SEQ ID NO: 238 and 188. SEQ ID NO: 238 also presents a guide sequence that can be used to target Mfw2′ with a site-specific guided nuclease. This nuclease-null variant allele is known to naturally occur in certain wheat accessions, e.g., in Buck Meteoro which is available commercially from Buck Semillas S. A., (Necochea, Argentina) or Argenetics Seeds, S. A. (Colon, Argentina). An advantage of this nuclease-null allele is that it is a naturally-occurring allele. Other nuclease-null alleles of Mfw2 known in the art and plants comprising such alleles can be utilized in the methods and processes described herein.

In some embodiments of any of the aspects, a nuclease-null allele of PV1 (e.g. a PV1′ allele) comprises a sequence comprising one or both of the G to A mutations of SEQ ID NO: 167, relative to SEQ ID NO: 166. In some embodiments of any of the aspects, a nuclease-null allele of PV1 (e.g. a PV1′ allele) comprises a PV1 sequence provided herein which has been modified to comprise one or two G to A mutations corresponding to one or both of the G to A mutations of SEQ ID NO: 167. In some embodiments of any of the aspects, a nuclease-null allele of PV1 (e.g. a PV1′ allele) comprises a sequence comprising both of the G to A mutations of SEQ ID NO: 167, relative to SEQ ID NO: 166. In some embodiments of any of the aspects, a nuclease-null allele of PV1 (e.g. a PV1′ allele) comprises a PV1 sequence provided herein which has been modified to comprise two G to A mutations corresponding to both of the G to A mutations of SEQ ID NO: 167.

Use of nuclease-null ectopic alleles permits the introduction or insertion of the ectopic alleles before or during use of nuclease to knock-out endogenous alleles. This can be of particular use when introducing the genetic systems described herein into a new line while avoiding the need to insert ectopic allees or copies into the new line by molecular biology techniques. For instance, a wildtype elite line can be crossed and back-crossed onto an extant maintainer line, with selection for maximum elite line conformity (e.g., by genome wide SNPs and plant phenotype) as well as the necessary maintainer cassettes. Once suitable elite line conformity and the presence of the maintainer cassettes or systems described herein are present, the endogenous Mfw and PV alleles can be knocked out as a last stage of preparing the new maintainer lines without such knockout affecting the inserted nuclease-null alleles.

In some embodiments of any of the aspects, the at least one functional ectopic allele of a MF gene and at least one functional ectopic allele of a seed color gene (e.g., seed coat and/or seed endosperm gene) (or set of seed color genes/alleles) comprises a sequence with at least 80%, at least 85%, at least 90%, at least 95%, at least 98% or greater sequence identity to the sequence of SEQ ID NO: 173. In some embodiments of any of the aspects, the at least one functional ectopic allele of a PV gene comprises a sequence with at least 80%, at least 85%, at least 90%, at least 95%, at least 98% or greater sequence identity to the sequence of SEQ ID NO: 172. In some embodiments of any of the aspects, the at least one functional ectopic allele of a MF gene and at least one functional ectopic allele of a seed color gene (e.g., seed coat and/or seed endosperm gene) (or set of seed color genes/alleles) comprises a sequence with at least 80%, at least 85%, at least 90%, at least 95%, at least 98% or greater sequence identity to the sequence of SEQ ID NO: 173 and the at least one functional ectopic allele of a PV gene comprises a sequence with at least 80%, at least 85%, at least 90%, at least 95%, at least 98% or greater sequence identity to the sequence of SEQ ID NO: 172. In some embodiments of any of the aspects, the at least one functional ectopic allele of a MF gene and at least one functional ectopic allele of a seed color gene (e.g., seed coat and/or seed endosperm gene) (or set of seed color genes/alleles) comprises the sequence of SEQ ID NO: 173. In some embodiments of any of the aspects, the at least one functional ectopic allele of a PV gene comprises the sequence of SEQ ID NO: 172. In some embodiments of any of the aspects, the at least one functional ectopic allele of a MF gene and at least one functional ectopic allele of a seed color gene (e.g., seed coat and/or seed endosperm gene) (or set of seed color genes/alleles) comprises the sequence of SEQ ID NO: 173 and the at least one functional ectopic allele of a PV gene comprises the sequence of SEQ ID NO: 172.

The maintainer (for male-sterility) compositions and methods described herein, particularly those relating to nuclease-null (e.g., CRISPR-null) MF and PV alleles, are suitable for use with all small grains, e.g., wheat, triticale, canola/oilseed rape, indian mustard, barley, rice, oat, or rye. MF and PV genes endogenous to non-wheat small grain species can be readily identified as the homologs or orthologs of the wheat MF or PV genes provided herein. Homologs or orthologs of the MF and PV genes provided herein can be identified by, e.g., searching a plant's genomic sequence data using a MF or PV sequence provided herein and identifying gene in the plant's genome with the degree of homology (percent identity) as the homolog or ortholog. In some embodiments of any of the aspects, a homolog or ortholog of a MF or PV gene described herein is a gene with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or more, identical (e.g, at the genomic sequence level, coding sequence level, or amino acid sequence level) to a MF or PV gene described herein. In some embodiments of any of the aspects, a homolog or ortholog of a MF or PV gene described herein is a gene with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or more, identical at the genomic sequence level to a MF or PV gene described herein. In some embodiments of any of the aspects, a homolog or ortholog of a MF or PV gene described herein is a gene with at least 60% identical at the genomic sequence level to a MF or PV gene described herein. In some embodiments of any of the aspects, a homolog or ortholog of a MF or PV gene described herein is a gene with at least 80%, at least 85%, at least 90%, at least 91%, at least 92%, at least 93%, at least 94%, at least 95%, at least 96%, at least 97%, at least 98%, at least 99%, or more, identical at the coding sequence level to a MF or PV gene described herein. In some embodiments of any of the aspects, a homolog or ortholog of a MF or PV gene described herein is a gene with at least 80% identical at the coding sequence level to a MF or PV gene described herein. In some embodiments of any of the aspects, a homolog or ortholog of a MF or PV gene described herein is a gene with at least 80%, at least 85%, at least 90%, at least 91%, at least 92%, at least 93%, at least 94%, at least 95%, at least 96%, at least 97%, at least 98%, at least 99%, or more, identical at the amino acid sequence level to a MF or PV gene described herein. In some embodiments of any of the aspects, a homolog or ortholog of a MF or PV gene described herein is a gene with at least 80% identical at the amino acid sequence level to a MF or PV gene described herein. Sequence data for the plant species described herein is freely available at, e.g., in the Ensembl Plants database, available on the world wide web at plants.ensembl.org/index.html.

In some embodiments of any of the aspects, the plant is not wheat, the MF gene is the gene in the plant with the highest degree of homology (e.g., at least 90% homology) to a gene selected from Table 1, and the PV gene is the gene in the plant with the highest degree of homology (e.g., at least 90% homology) to PV1 or PV2. In some embodiments of any of the aspects, the plant is not wheat, the MF gene is the gene in the plant with the highest degree of homology (e.g., at least 90% homology) to Mfw2, and the PV gene is the gene in the plant with the highest degree of homology (e.g., at least 90% homology) to PV1 or PV2. In some embodiments of any of the aspects, the plant is not wheat, the MF gene is is the gene in the plant with the highest degree of homology (e.g., at least 90% homology) to Mfw2, and the PV gene is the gene in the plant with the highest degree of homology (e.g., at least 90% homology) to PV1. In some embodiments of any of the aspects, the plant is not wheat, the MF gene is the gene in the plant with the highest degree of homology (e.g., at least 90% homology) to Mfw2, and the PV gene is the gene in the plant with the highest degree of homology (e.g., at least 90% homology) to PV2. In some embodiments of any of the aspects, the plant is barley and the MF gene is HORVU7Hr1G029930, HORVU.MOREX.r3.7HG0658750.1 (a homolog of Mfw2), or HORVU.MOREX.r3.4HG0333500 (a homolog of Ms1), e.g., as provided in the Ensembl Plant database. In some embodiments of any of the aspects, the plant is barley and the PV gene is HORVU7Hr1G001280, HORVU.MOREX.r3.7HG0635710.1 (a homolog of PV1/NPG1), HORVU.MOREX.r3.4HG0331330.1 (a homolog of PV2/ANX1), or HORVU.MOREX.r3.7HG0642320.1 (a homolog of PV3/RUPO) e.g., as provided in the Ensembl Plant database.

In some embodiments of any of the aspects, a functional allele of HORVU7Hr1G029930 shares at least 80% sequence identity with SEQ ID NO: 170. In some embodiments of any of the aspects, a functional allele of HORVU7Hr1G029930 displays the same type of activity and shares at least 80% sequence identity with SEQ ID NO: 170. In some embodiments of any of the aspects a functional allele of HORVU7Hr1G029930 shares at least 80%, at least 85%, at least 90%, at least 95%, at least 98%, or greater sequence identity with SEQ ID NO: 170. In some embodiments of any of the aspects, a functional allele of HORVU7Hr1G029930 displays the same type of activity and shares at least 80%, at least 85%, at least 90%, at least 95%, at least 98%, or greater sequence identity with SEQ ID NO: 170. In some embodiments of any of the aspects, a functional allele of HORVU7Hr1G029930 shares at least 95% sequence identity with SEQ ID NO: 170. In some embodiments of any of the aspects a functional allele of HORVU7Hr1G029930 displays the same type of activity and shares at least 95% sequence identity with SEQ ID NO: 170. In some embodiments of any of the aspects, the functional allele of a functional allele of HORVU7Hr1G029930 is SEQ ID NO: 170. In some embodiments of any of the aspects, a MF gene can be the gene from a species, cultivar, or variety which has the highest degree of homology and/or sequence identity of the genes in that species', cultivar's or variety's genome with one of the foregoing sequences.

In some embodiments of any of the aspects, a functional allele of HORVU.MOREX.r3.7HG0658750.1 shares at least 80% sequence identity with SEQ ID NO: 247, 248, and/or 249. In some embodiments of any of the aspects, a functional allele of HORVU.MOREX.r3.7HG0658750.1 displays the same type of activity and shares at least 80% sequence identity with SEQ ID NO: 247, 248, and/or 249. In some embodiments of any of the aspects a functional allele of HORVU.MOREX.r3.7HG0658750.1 shares at least 80%, at least 85%, at least 90%, at least 95%, at least 98%, or greater sequence identity with SEQ ID NO: 247, 248, and/or 249. In some embodiments of any of the aspects, a functional allele of HORVU.MOREX.r3.7HG0658750.1 displays the same type of activity and shares at least 80%, at least 85%, at least 90%, at least 95%, at least 98%, or greater sequence identity with SEQ ID NO: 247, 248, and/or 249. In some embodiments of any of the aspects, a functional allele of HORVU.MOREX.r3.7HG0658750.1 shares at least 95% sequence identity with SEQ ID NO: 247, 248, and/or 249. In some embodiments of any of the aspects a functional allele of HORVU.MOREX.r3.7HG0658750.1 displays the same type of activity and shares at least 95% sequence identity with SEQ ID NO: 247, 248, and/or 249. In some embodiments of any of the aspects, the functional allele of a functional allele of HORVU.MOREX.r3.7HG0658750.1 is SEQ ID NO: 247, 248, and/or 249. In some embodiments of any of the aspects, a MF gene can be the gene from a species, cultivar, or variety which has the highest degree of homology and/or sequence identity of the genes in that species', cultivar's or variety's genome with one of the foregoing sequences.

In some embodiments of any of the aspects, a functional allele of HORVU.MOREX.r3.4HG0333500 shares at least 80% sequence identity with SEQ ID NO: 250, 251, and/or 252. In some embodiments of any of the aspects, a functional allele of HORVU.MOREX.r3.4HG0333500 displays the same type of activity and shares at least 80% sequence identity with SEQ ID NO: 250, 251, and/or 252. In some embodiments of any of the aspects a functional allele of HORVU.MOREX.r3.4HG0333500 shares at least 80%, at least 85%, at least 90%, at least 95%, at least 98%, or greater sequence identity with SEQ ID NO: 250, 251, and/or 252. In some embodiments of any of the aspects, a functional allele of HORVU.MOREX.r3.4HG0333500 displays the same type of activity and shares at least 80%, at least 85%, at least 90%, at least 95%, at least 98%, or greater sequence identity with SEQ ID NO: 250, 251, and/or 252. In some embodiments of any of the aspects, a functional allele of HORVU.MOREX.r3.4HG0333500 shares at least 95% sequence identity with SEQ ID NO: 250, 251, and/or 252. In some embodiments of any of the aspects a functional allele of HORVU.MOREX.r3.4HG0333500 displays the same type of activity and shares at least 95% sequence identity with SEQ ID NO: 250, 251, and/or 252. In some embodiments of any of the aspects, the functional allele of a functional allele of HORVU.MOREX.r3.4HG0333500 is SEQ ID NO: 250, 251, and/or 252. In some embodiments of any of the aspects, a MF gene can be the gene from a species, cultivar, or variety which has the highest degree of homology and/or sequence identity of the genes in that species', cultivar's or variety's genome with one of the foregoing sequences.

In some embodiments of any of the aspects, a functional allele of HORVU7Hr1G001280 shares at least 80% sequence identity with SEQ ID NO: 171. In some embodiments of any of the aspects, a functional allele of HORVU7Hr1G001280 displays the same type of activity and shares at least 80% sequence identity with SEQ ID NO: 171. In some embodiments of any of the aspects a functional allele of HORVU7Hr1G001280 shares at least 80%, at least 85%, at least 90%, at least 95%, at least 98%, or greater sequence identity with SEQ ID NO: 171. In some embodiments of any of the aspects, a functional allele of HORVU7Hr1G001280 displays the same type of activity and shares at least 80%, at least 85%, at least 90%, at least 95%, at least 98%, or greater sequence identity with SEQ ID NO: 171. In some embodiments of any of the aspects, a functional allele of HORVU7Hr1G001280 shares at least 95% sequence identity with SEQ ID NO: 171. In some embodiments of any of the aspects a functional allele of HORVU7Hr1G001280 displays the same type of activity and shares at least 95% sequence identity with SEQ ID NO: 171. In some embodiments of any of the aspects, the functional allele of a functional allele of HORVU7Hr1G001280 is SEQ ID NO: 171. In some embodiments of any of the aspects, a PV gene can be the gene from a species, cultivar, or variety which has the highest degree of homology and/or sequence identity of the genes in that species', cultivar's or variety's genome with one of the foregoing sequences.

In some embodiments of any of the aspects, a functional allele of HORVU.MOREX.r3.7HG0635710.1 shares at least 80% sequence identity with SEQ ID NO: 238, 239, and/or 240. In some embodiments of any of the aspects, a functional allele of HORVU.MOREX.r3.7HG0635710.1 displays the same type of activity and shares at least 80% sequence identity with SEQ ID NO: 238, 239, and/or 240. In some embodiments of any of the aspects a functional allele of HORVU.MOREX.r3.7HG0635710.1 shares at least 80%, at least 85%, at least 90%, at least 95%, at least 98%, or greater sequence identity with SEQ ID NO: 238, 239, and/or 240. In some embodiments of any of the aspects, a functional allele of HORVU.MOREX.r3.7HG0635710.1 displays the same type of activity and shares at least 80%, at least 85%, at least 90%, at least 95%, at least 98%, or greater sequence identity with SEQ ID NO: 238, 239, and/or 240. In some embodiments of any of the aspects, a functional allele of HORVU.MOREX.r3.7HG0635710.1 shares at least 95% sequence identity with SEQ ID NO: 238, 239, and/or 240. In some embodiments of any of the aspects a functional allele of HORVU.MOREX.r3.7HG0635710.1 displays the same type of activity and shares at least 95% sequence identity with SEQ ID NO: 238, 239, and/or 240. In some embodiments of any of the aspects, the functional allele of a functional allele of HORVU.MOREX.r3.7HG0635710.1 is SEQ ID NO: 238, 239, and/or 240. In some embodiments of any of the aspects, a PV gene can be the gene from a species, cultivar, or variety which has the highest degree of homology and/or sequence identity of the genes in that species', cultivar's or variety's genome with one of the foregoing sequences.

In some embodiments of any of the aspects, a functional allele of HORVU.MOREX.r3.4HG0331330.1 shares at least 80% sequence identity with SEQ ID NO: 241, 242, and/or 243. In some embodiments of any of the aspects, a functional allele of HORVU.MOREX.r3.4HG0331330.1 displays the same type of activity and shares at least 80% sequence identity with SEQ ID NO: 241, 242, and/or 243. In some embodiments of any of the aspects a functional allele of HORVU.MOREX.r3.4HG0331330.1 shares at least 80%, at least 85%, at least 90%, at least 95%, at least 98%, or greater sequence identity with SEQ ID NO: 241, 242, and/or 243. In some embodiments of any of the aspects, a functional allele of HORVU.MOREX.r3.4HG0331330.1 displays the same type of activity and shares at least 80%, at least 85%, at least 90%, at least 95%, at least 98%, or greater sequence identity with SEQ ID NO: 241, 242, and/or 243. In some embodiments of any of the aspects, a functional allele of HORVU.MOREX.r3.4HG0331330.1 shares at least 95% sequence identity with SEQ ID NO: 241, 242, and/or 243. In some embodiments of any of the aspects a functional allele of HORVU.MOREX.r3.4HG0331330.1 displays the same type of activity and shares at least 95% sequence identity with SEQ ID NO: 241, 242, and/or 243. In some embodiments of any of the aspects, the functional allele of a functional allele of HORVU.MOREX.r3.4HG0331330.1 is SEQ ID NO: 241, 242, and/or 243. In some embodiments of any of the aspects, a PV gene can be the gene from a species, cultivar, or variety which has the highest degree of homology and/or sequence identity of the genes in that species', cultivar's or variety's genome with one of the foregoing sequences.

In some embodiments of any of the aspects, a functional allele of HORVU.MOREX.r3.7HG0642320.1 shares at least 80% sequence identity with SEQ ID NO: 244, 245, and/or 246. In some embodiments of any of the aspects, a functional allele of HORVU.MOREX.r3.7HG0642320.1 displays the same type of activity and shares at least 80% sequence identity with SEQ ID NO: 244, 245, and/or 246. In some embodiments of any of the aspects a functional allele of HORVU.MOREX.r3.7HG0642320.1 shares at least 80%, at least 85%, at least 90%, at least 95%, at least 98%, or greater sequence identity with SEQ ID NO: 244, 245, and/or 246. In some embodiments of any of the aspects, a functional allele of HORVU.MOREX.r3.7HG0642320.1 displays the same type of activity and shares at least 80%, at least 85%, at least 90%, at least 95%, at least 98%, or greater sequence identity with SEQ ID NO: 244, 245, and/or 246. In some embodiments of any of the aspects, a functional allele of HORVU.MOREX.r3.7HG0642320.1 shares at least 95% sequence identity with SEQ ID NO: 244, 245, and/or 246. In some embodiments of any of the aspects a functional allele of HORVU.MOREX.r3.7HG0642320.1 displays the same type of activity and shares at least 95% sequence identity with SEQ ID NO: 244, 245, and/or 246. In some embodiments of any of the aspects, the functional allele of a functional allele of HORVU.MOREX.r3.7HG0642320.1 is SEQ ID NO: 244, 245, and/or 246. In some embodiments of any of the aspects, a PV gene can be the gene from a species, cultivar, or variety which has the highest degree of homology and/or sequence identity of the genes in that species', cultivar's or variety's genome with one of the foregoing sequences.

The methods and compositions described herein are particularly applicable to polyploidal plants. In some embodiments of any of the aspects, the plant or cell is polyploidal, e.g., tetraploid or hexaploid. In some embodiments of any of the aspects, the plant or cell is wheat, e.g., hexaploid wheat, tetraploid wheat, Triticum aestivum, or Triticum durum. In some embodiments of any of the aspects, the plant or cell is triticale, oat, canola/oilseed rape or indian mustard. In some embodiments of any of the aspects, the plant or cell is an elite breeding line.

In some embodiments of any of the aspects, the male-fertile maintainer plant or cell is tetraploid and the second genome comprises loss-of-function alleles of the MF gene at the native MF gene loci and loss-of-function alleles of the PV gene at the native PV gene loci. In some embodiments of any of the aspects, the male-fertile maintainer plant or cell is hexaploid and the second and third genomes both comprise loss-of-function alleles of the MF gene at the native MF gene loci and loss-of-function alleles of the PV gene at the native PV gene loci.

The plants and cells described herein comprise one or more of: certain loss-of-function alleles, at least one functional MF allele, at least one allele of a seed color gene (e.g., seed coat and/or seed endosperm gene) (or set of seed color genes/alleles), and at least one functional PV allele; which are engineered and are referred to collectively as “engineered modifications.” The engineered modifications described herein can be generated by any method known in the art, e.g., by homolgous recombination-mediated mutagenesis, random mutagenesis, or by using a site-specific guided nuclease. In some embodiments of any of the aspects, at least one copy of any of the engineered modifications is engineered by using a site-specific guided nuclease. In some embodiments of any of the aspects, the engineered modifications are engineered by using a site-specific guided nuclease.

Various site-specific guided nucleases are known in the art and can include, by way of non-limiting example, transcription activator-like effector nucleases (TALENs), oligonucleotides, meganucleases, and zinc-finger nucleases. Toolkits and services for zinc-finger nuclease mutagenesis are commercially available, for example EXZACT™ Precision Technology, marketed by Dow AgroSciences.

In some embodiments of any of the aspects, the site-specific guided nuclease is a CRISPR-associated (Cas) system such as CRISPR-Cas9 (e.g., Cas9, a Cas9-derived nickase, or a Cas9 homolog (e.g., Cpf1)). CRISPR is an acronym for clustered regularly interspaced short palindromic repeats. Briefly, in order for a Cas nuclease (or related nuclease) to recognize and cleave a target nucleic acid molecule, a CRISPR RNA (crRNA) and trans-activating crRNA (tracrRNA) must be present. crRNAs hybridize with tracrRNA to form a guide RNA (sgRNA) which then associates with the Cas nuclease. Alternatively, the sgRNA can be provided as a single contiguous sgRNA. Once the sgRNA is complexed with Cas, the complex can bind to a target nucleic acid molecule. The sgRNA binds specifically to a complementary target sequence via a target-specific sequence in the crRNA portion (e.g., the spacer sequence), while Cas itself binds to a protospacer adjacent motif (CRISPR/Cas protospacer-adjacent motif; PAM). The Cas nuclease then mediates cleavage of the target nucleic acid to create a double-stranded break within the sequence bound by the sgRNA. Deletions can be generated by, e.g., using the nuclease to cut a genome at two specific locations targeted with two sgRNAs each specific to one of the two locations concerned, thereby excising the sequence between the two double-strand breaks. CRISPR-Cas technology for editing of plant genomes is fully described in Belhaj et al. (2015). This is a practicable, convenient and flexible method of gene editing. It has been shown to work well in plants, see for example in Belhaj et al. (2015); Wang et al. (2014; Nature Biotechnology32:947-951); and Shan et al. (2014). The latter paper gives full protocols to enable the system to be applied to modify plant genomes (including wheat) as desired.

As described herein, an engineered modification can be introduced by utilizing the CRISPR/Cas system. In some embodiments of any of the aspects, the site-specific guided nuclease is a form of CRISPR-Cas, e.g., CRISPR-Cas9. In some embodiments of any of the aspects, the engineered modifications are created using a site-specific guided nuclease and a multi-guide construct.

In some embodiments of any of the aspects, a plant or plant cell described herein can further comprise an exogenous or introduced endonuclease or a nucleic acid encoding such an endonuclease (e.g., Cas9, a Cas9-derived nickase, or a Cas9 homolog (e.g., Cpf1)). In some embodiments of any of the aspects, a plant or seed as described herein can further comprise a CRISPR RNA sequence designed to target an endonuclease to the gene, e.g. (a crRNA and trans-activating crRNA (tracrRNA) and/or a guide RNA (sgRNA)). In some embodiments of any of the aspects, the sgRNA is provided as a single continuous nucleic acid molecule. In some embodiments of any of the aspects, the sgRNA is provided as a set of hybridized molecules, e.g., a crRNA and tracrRNA. In some embodiments of any of the aspects, the sgRNA is provided as a DNA molecule encoding a sgRNA and/or a crRNA and tracrRNA. Design of sgRNAs, crRNAs, and tracrRNAs are known in the art and described elsewere herein. Exemplary sgRNA sequences are provided elsewhere herein (e.g., SEQ ID NOs: 22-25 or SEQ ID NO: 156 for Mfwl, SEQ ID NOs: 26-29 for Mfw2, SEQ ID NOs: 131-134 for Mfw3-A, SEQ ID NOs: 135-138 for Mfw3-B, SEQ ID NOs: 139-142 for Mfw3-D, SEQ ID NOs: 143-146 for Mfw5-A, SEQ ID NOs: 147-150 for Mfw5-B, SEQ ID NOs: 151-154 for Mfw5-D) and described in detail in International Patent Publication WO 2018/022410 and Milner et al. Plant Direct 2020 4(3):e00201; each of which are incorporated by reference herein in their entireties. In some embodiments of any of the aspects, a multi-guide construct is provided, e.g., multiple sgRNA are provided in a single construct and/or nucleic acid molecule such that multiple target sequences are cleaved in the presence of a Cas enzyme and the multi-guide construct.

sgRNA for Mfw1
SEQ ID NO: 156
GGGGGATGGGGGCTTACGTAGGG

As used herein, “target sequence” within the context of a site-specific guided nuclease refers to a sequence in the relevant genome which is to be used to specify where the nuclease will generate a break or nick in the genome at a desired location. In the case of Cas (and related) nucleases, the guide RNA is designed to specifically hybridize to the target sequence, or in the case of multi-guide constructs, multiple guide RNAs are provided, each of which specifically hybrizes to a target sequence. Target sequences can be identified using the publicly available program DREG (available on the world wide web at emboss.sourceforge.net/apps/cvs/emboss/apps/dreg.html) to find sequences that match either ANNNNNNNNNNNNNNNNNNNNGG or GNNNNNNNNNNNNNNNNNNNNGG in both directions of the genomic sequence. As an illustrative example, guides can be selected from the results based on the following criteria: that the target sequence is conserved in all homoeologues which are to be modified, that it has a restriction enzyme site near the site of the protospacer associated motif (PAM) but in the sequence of the guide RNA and finally, prioritizing guides near the start of the coding sequences of each gene. An additional consideration can be to select sequences with either AN20GG and GN20GG as this stabilizes the construct for transformation in the plant.

By way of non-limiting example, exemplary guide sequences for generating mutations in a target sequence include SEQ ID NOs: 22-25 or SEQ ID NO: 156 for Mfwl, SEQ ID NOs: 26-29, and 238-239 for Mfw2, SEQ ID NOs: 131-134 for Mfw3-A, SEQ ID NOs: 135-138 for Mfw3-B, SEQ ID NOs: 139-142 for Mfw3-D, SEQ ID NOs: 143-146 for Mfw5-A, SEQ ID NOs: 147-150 for Mfw5-B, and/or SEQ ID NOs: 151-154 for Mfw5-D.

Guide sequence expression can be driven by individual and/or shared promoters. Exemplary promoters include OsU3, TaU3, TaU6 and OsU6 promoters. Guide constructs, expressing one or more sgRNA sequences, can be cloned into a vector suitable for expressing the sgRNAs in the plant, e.g., a binary vector containing a wheat-optimized Cas9 enzyme driven by the rice actin promoter can be used in wheat. Vectors can be introduced into the plant or plant cell by any means known in the art, e.g. by Agrobacterium. Alternatively, the sgRNAs can be expressed in vitro and introduced into cells by, e.g., microinjection.

Cas9 and sgRNA sequences can be expressed either stably or transiently in a cell in order to generate the engineered modifications described herein. In one aspect of any of the embodiments, described herein is a plant cell comprising 1) an exogenous Cas9 protein and/or an exogenous nucleic acid encoding a Cas9 protein; and 2) at least one sgRNA capable of specifically hybridizing with at least one target sequence of a gene described herein under cellular conditions or a nucleic acid encoding such an sgRNA. In some embodiments of any of the aspects, the 1) exogenous nucleic acid encoding a Cas9 protein; and 2) the nucleic acid encoding at least one sgRNA capable of specifically hybridizing with the target sequence(s) under cellular conditions are provided in a vector or vector(s). In some embodiments of any of the aspects, the vectors are transient expression vectors. In some embodiments of any of the aspects, the 1) exogenous nucleic acid encoding a Cas9 protein; and 2) the nucleic acid encoding at least one sgRNA are integrated into the genome. It is contemplated herein that similar approaches to vector delivery, transient expression, and/or stable integration can also be utilized in embodiments relating to, e.g., TALENs, and/or ZFNs.

The Cas enzyme and guide sequences can be provided in non-integrating vectors, e.g., to avoid incorporation of these sequences in the genome of the plant.

In one aspect of any of the embodiments, described herein is a nucleic acid encoding at least one sgRNA capable of specifically hybridizing with at least one gene sequence described herein, e.g., under cellular conditions. In one aspect of any of the embodiments, described herein is a nucleic acid encoding at least one sgRNA capable of targeting Cas9 or a related endonuclease to at least one gene described herein, e.g., under cellular conditions. In some embodiments of any of the aspects, the nucleic acid further encodes a Cas9 protein. In some embodiments of any of the aspects, the nucleic acid is provided in a vector. In some embodiments of any of the aspects, the vector is a transient expression vector.

Following contact with a site-specific nuclease, e.g., a Cas (or related) enzyme and at least one guide RNA, plants can be screened for deactivating modifications, e.g., utilizing a PCR based method where the PCR product is digested with an appropriate enzyme previously identified to cut the DNA at a site near the PAM. PCR products which are not cut therefore contain a modification induced by the CRISPR construct.

In some embodiments of any of the aspects, a loss-of-function or knockout allele of a gene can comprise a deletion generated by CRISPR/Cas. In some embodiments of any of the aspects, a loss-of-function or knockout allele of a gene can be made/engineered/mutated/created by contacting a plant/plant cell with CRISPR/Cas and at least one sgRNA capable of targeting the gene, thereby creating a deletion in or of the gene.

In some embodiments of any of the aspects, a loss-of-function or knockout allele of a gene can comprise a “prime edit” generated by CRISPR/Cas (e.g., a Cas-reverse transcriptase fusion). Prime editing is a technique in which Cas is fused to a reverse transcriptase and the guide RNA further comprises an edit-containing RNA template. When the edit-containing RNA template comprises a template for a premature stop codon, the combined activity of the Cas-reverse transcriptase fusion introduces a premature stop codon in the targeted gene. In some embodiments of any of the aspects, a loss-of-function or knockout allele of a gene can be made/engineered/mutated/created by contacting a plant/plant cell with CRISPR/Cas and at least one guide RNA further comprising an edit-containing RNA template and capable of targeting the gene, thereby creating a prime edit in or of the gene. In some embodiments of any of the aspects, the prime edit comprises a premature stop codon. Prime editing techniques are well known in the art and are further discussed, e.g., in Scholefield et al. Gene Therapy volume 28, pages 396-401 (2021); and Anzalone et al. Nature volume 576, pages 149-157 (2019); each of which is incorporated by reference herein in its entirety.

In any of the methods described herein, nucleases, guide RNAs, sgRNA, and/or nuclease fusion proteins can be introduced or inserted by any method known in the art, e.g., biolistic delivery, or vector delivery (e.g., viral vectors or T-DNA vectors). Methods of transforming plants/plant cells are well known in the art. In some embodiments of any of the aspects, contacting a plant/cell with a nuclease/guide RNA/sgRNA/nuclease fusion protein comprises contacting the plant/plant cell(s) with a viral vector comprising the nuclease/guide RNA/sgRNA/nuclease fusion protein. In some embodiments of any of the aspects, contacting a plant/cell with a nuclease/guide RNA/sgRNA/nuclease fusion protein comprises contacting the plant/plant cell(s) with a T-DNA vector comprising the nuclease/guide RNA/sgRNA/nuclease fusion protein. In some embodiments of any of the aspects, introducing or inserting a nuclease/guide RNA/sgRNA/nuclease fusion protein into a plant/plant cell comprises contacting the plant/plant cell(s) with a viral vector comprising the nuclease/guide RNA/sgRNA/nuclease fusion protein. In some embodiments of any of the aspects, introducing or inserting a nuclease/guide RNA/sgRNA/nuclease fusion protein into a plant/plant cell comprises contacting the plant/plant cell(s) with a T-DNA vector comprising the nuclease/guide RNA/sgRNA/nuclease fusion protein.

In any of the aspects of the methods described herein contacting the plant/plant cell(s) with a nuclease and/or nuclease fusion protein can comprise contacting the plant/plant cell(s) with a recombinase polypeptide, or with a nucleic acid (e.g., a vector) encoding the nuclease and/or nuclease fusion protein. In any of the aspects of the methods described herein contacting the plant/plant cell(s) with a nuclease and/or nuclease fusion protein can comprise introducing into the plant/plant cell(s) a recombinase polypeptide, or a nucleic acid (e.g., a vector) encoding the nuclease and/or nuclease fusion protein. In embodiments relating to a nucleic acid (e.g., a vector) encoding the nuclease and/or nuclease fusion protein, a step of removing or selecting out the nucleic acid encoding the nuclease and/or nuclease fusion protein after the relevant knock-out or loss-of-function allles are created/mutated/engineered.

In alternative embodiments, an engineered modification can be introduced by utilizing TALENs or ZFN technology, which are known in the art. Methods of engineering nucleases to achieve a desired sequence specificity are known in the art and are described, e.g., in Kim (2014); Kim (2012); Belhaj et al. (2013); Urnov et al. (2010); Bogdanove et al. (2011); Jinek et al. (2012) Silva et al. (2011); Ran et al. (2013); Carlson et al. (2012); Guerts et al. (2009); Taksu et al. (2010); and Watanabe et al. (2012); each of which is incorporated by reference herein in its entirety.

In some embodiments of any of the aspects, modifications can be introduced using any of homolgous recombination-mediated mutagenesis, random mutagenesis, or site-specific guided nuclease methods described elsewhere herein, combined with providing one or more template nucleic acids comprising the sequence/gene/allele/construct to be introduced. The template nucleic acids can comprise one or more regions of homology to the target loci in the first genome to direct their introduction at the target loci. Such technologies, and the design of such constructs are known in the art.

In one aspect of any of the embodiments, described herein is a plant comprising a first genome comprising: on a first chromosome of a pair of homologous chromosomes, at a single target locus, at least one functional ectopic allele of a MF gene and at least one functional ectopic allele of a seed color gene (e.g., seed coat and/or seed endosperm gene) (or at least one functional ectopic allele of each member of a set of seed color genes). In one aspect of any of the embodiments, described herein is a plant comprising a first genome comprising: on a second chromosome of the pair of homologous chromosomes, at the target locus corresponding to the target locus of the first chromosome of the pair of homologous chromosomes, at least one functional ectopic allele of a PV gene.

In one aspect of any of the embodiments, described herein is a plant comprising a first genome comprising: on a first chromosome of a pair of homologous chromosomes, at a single target locus, at least one functional ectopic allele of a MF gene and at least one functional ectopic allele of a seed color gene (e.g., seed coat and/or seed endosperm gene) (or at least one functional ectopic allele of each member of a set of seed color genes) and loss-of-function alleles of the endogenous MF genes at the native MF gene loci. In one aspect of any of the embodiments, described herein is a plant comprising a first genome comprising: on a first chromosome of a pair of homologous chromosomes, at a single target locus, at least one functional ectopic allele of a MF gene and at least one functional ectopic allele of a seed color gene (e.g., seed coat and/or seed endosperm gene) (or at least one functional ectopic allele of each member of a set of seed color genes) and loss-of-function alleles of the endogenous PV genes at the native PV gene loci. In one aspect of any of the embodiments, described herein is a plant comprising a first genome comprising: on a first chromosome of a pair of homologous chromosomes, at a single target locus, at least one functional ectopic allele of a MF gene and at least one functional ectopic allele of a seed color gene (e.g., seed coat and/or seed endosperm gene) (or at least one functional ectopic allele of each member of a set of seed color genes) and loss-of-function alleles of the endogenous MF genes at the native MF gene loci and loss-of-function alleles of the endogenous PV genes at the native PV gene loci.

In one aspect of any of the embodiments, described herein is a plant comprising a first genome comprising: on a second chromosome of the pair of homologous chromosomes, at the target locus corresponding to the target locus of the first chromosome of the pair of homologous chromosomes, at least one functional ectopic allele of a PV gene and loss-of-function alleles of the endogenous PV genes at the native PV gene loci. In one aspect of any of the embodiments, described herein is a plant comprising a first genome comprising: on a second chromosome of the pair of homologous chromosomes, at the target locus corresponding to the target locus of the first chromosome of the pair of homologous chromosomes, at least one functional ectopic allele of a PV gene and loss-of-function alleles of the endogenous MF genes at the native MF gene loci. In one aspect of any of the embodiments, described herein is a plant comprising a first genome comprising: on a second chromosome of the pair of homologous chromosomes, at the target locus corresponding to the target locus of the first chromosome of the pair of homologous chromosomes, at least one functional ectopic allele of a PV gene and loss-of-function alleles of the endogenous MF genes at the native MF gene loci and loss-of-function alleles of the endogenous PV genes at the native PV gene loci.

In one aspect of any of the embodiments, provided herein is a method of producing a male-fertile maintainer plant as described herein, wherein the method comprises:
b. engineering the seed endosperm gene into the first chromosome of a homologous pair in the first genome and engineering at least one functional allele of a PV gene into the second chromosome of the homologous pair in the first genome;
c. engineering loss-of-function alleles in/at each allele of a MF gene in the second and any subsequent genomes, and at the allele on the second chromosome of the homologous pair in the first genome;
d. engineering loss-of-function alleles in/at each native allele of a PV gene in all genomes.
   In some embodiments of any of the aspects, steps b and c can be conducted simultaneously. In some embodiments of any of the aspects, step b is conducted before step c. In some embodiments of any of the aspects, step c is conducted before step b.

In one aspect of any of the embodiments, provided herein is a method of producing a male-fertile maintainer plant and cognate male-sterile plant as described herein, wherein the method comprises:
a. engineering the seed endosperm gene into the first chromosome of a homologous pair in the first genome and engineering at least one functional allele of a PV gene into the second chromosome of the homologous pair in the first genome of the maintainer line;
b. engineering loss-of-function alleles in/at each allele of a MF gene in the second and any subsequent genomes, and at the allele on the second chromosome of the homologous pair in the first genome of the maintainer line;
c. engineering loss-of-function alleles in/at each native allele of a PV gene in all genomes of the maintainer line; and
d. engineering loss-of-function alleles in/at each native allele of the MF and PV genes in all genomes of the male-sterile line.
   In some embodiments of any of the aspects, steps b and c can be conducted simultaneously. In some embodiments of any of the aspects, step b is conducted before step c. In some embodiments of any of the aspects, step c is conducted before step b. In some embodiments of any of the aspects, steps b and c can be conducted simultaneously with step d. In some embodiments of any of the aspects, step b and c are conducted before step d. In some embodiments of any of the aspects, step d is conducted before steps b and c.

In one aspect of any of the embodiments, provided herein is a method of producing a male-fertile maintainer plant as described herein, wherein the method comprises:
a. engineering at least one functional nuclease-null (e.g., CRISPR-null) allele of a MF gene and a seed endosperm gene (optionally a nuclease-null (e.g., CRISPR-null) allele of a seed endosperm gene) into the first chromosome of a homologous pair in the first genome and engineering at least one functional nuclease-null (e.g., CRISPR-null) allele of a PV gene into the second chromosome of the homologous pair in the first genome;
b. engineering loss-of-function alleles in/at each allele of a MF gene in all genomes using the nuclease;
c. engineering loss-of-function alleles in/at each native allele of a PV gene in all genomes using the nuclease.
   In some embodiments of any of the aspects, steps b and c can be conducted simultaneously. In some embodiments of any of the aspects, step b is conducted before step c. In some embodiments of any of the aspects, step c is conducted before step b.

In one aspect of any of the embodiments, described herein is a method of preparing a male-fertile maintainer plant (or seed thereof) for a male-sterile polyploid plant, the method comprising engineering a plant to comprise: in a first genome: i) on a first chromosome of a pair of homologous chromosomes, at a single target locus, at least one functional ectopic allele of a MF gene and at least one functional ectopic allele of a seed color gene (e.g., seed coat and/or seed endosperm gene) (or set of seed color genes/alleles); ii) on a second chromosome of the pair of homologous chromosomes, at the target locus corresponding to the target locus of the first chromosome of the pair of homologous chromosomes, at least one functional ectopic allele of a PV gene; and iii) loss-of-function alleles of the endogenous MF genes at the native MF gene loci and loss-of-function alleles of the endogenous PV genes at the native PV gene loci. In some embodiments of any of the aspects, the plant further comprises at least one further genome, and the method further comprises engineering loss-of-function alleles of the endogenous MF genes at the native MF gene loci and loss-of-function alleles of the PV gene at the native PV gene loci in each of the at least one further genomes. Methods for engineering such alleles are described elsewhere herein. The engineering of the individual alleles can be done consecutively in any order or contemporaneously.

In the foregoing methods, the i) MF gene and seed color gene (or set of seed color genes/alleles) and ii) PV gene insertions are made separately. However, further contemplated herein are methods of preparing a male-fertile maintainter plant for a male-sterile polyploid plant in which a construct comprising both i) the MF gene and seed color gene (or set of seed color genes/alleles) and ii) the PV gene is inserted and then either i) or ii) are removed from individual alleles to provide a maintainer plant with the structure described herein. This approach ensures that in the maintainer line, i) the MF gene and seed color gene (or set of seed color genes/alleles) and ii) the PV gene are both located at the same locus and reduces the number of insertions and subsequent screenings necessary. This method has the further advantage that, after the initial cassette insertion at random loci in different transformant plants, the plant with the highest level of expression from the inserted cassette can be selected for the next-stage excisions. Additionally, this method provides a fundamentally different system to set up an allelic pair of genes/alleles which is an alternative to precisely targeted insertions (e.g. targeted to one of the homoeologues of a MF gene as described elsewhere herein). This approach gives users the option to use a different technology which does not rely on precision-targeting mutagenesis technologies.

Accordingly, in one aspect of any of the embodiments, described herein is a method of preparing a male-fertile maintainer plant for a male-sterile polyploid plant, the method comprising:
i) inserting, on a first chromosome of a pair of homologous chromosomes in a first genome, at a single target locus, a cassette comprising in 5′ to 3′ or 3′ to 5′ order:
   a first recognition site for a first recombinase;
   at least one functional ectopic nuclease null allele of a MF gene and at least one functional ectopic allele of a seed color gene (e.g., seed coat and/or seed endosperm gene) (or at least one functional ectopic allele of each member of a set of seed color genes) in either relative order;
   a first recognition site for a second recombinase;
   a selection gene;
   a second recognition site for the first recombinase;
   at least one functional ectopic nuclease-null allele of a PV gene;
   a second recognition site for the second recombinase;
thereby providing a full-cassette insertion plant;
ii) contacting a first progeny of the full-cassette insertion plant, or a cell thereof, with the first recombinase, thereby excising:
   one recognition site for the first recombinase, the at least one functional ectopic nuclease null allele of a MF gene and at least one functional ectopic allele of a seed color gene (e.g., seed coat and/or seed endosperm gene) (or at least one functional ectopic allele of each member of a set of seed color genes), the first recognition site for the second recombinase, and the selection gene from the genome of the first progeny and
thereby providing an excised first progeny comprising:
   one recognition site for the first recombinase, the at least one functional ectopic nuclease-null allele of a PV gene, and the second recognition site for the second recombinase portions of the construct;
iii) contacting a second progeny of the full-cassette insertion plant, or a cell thereof, with the second recombinase,
thereby excising:
   one recognition site for the second recombinase, the selection gene, the second recognition site for the first recombinase and at least one functional ectopic nuclease-null allele of a PV gene, and
thereby providing an excised second progeny comprising:
   one recognition site for the second recombinase, the first recognition site for the first recombinase, and the at least one functional ectopic nuclease null allele of a MF gene and at least one functional ectopic allele of a seed color gene (e.g., seed coat and/or seed endosperm gene) (or at least one functional ectopic allele of each member of a set of seed color genes) portions of the construct;
iv) crossing the excised first progeny provided in step ii) and the excised second progeny provided in step iii), thereby providing a third progeny comprising, in a first genome,
   on a first chromosome of a pair of homologous chromosomes, at a single target locus, the at least one functional ectopic nuclease-null allele of a MF gene and the at least one functional ectopic allele of a seed color gene (e.g., seed coat and/or seed endosperm gene) (or at least one functional ectopic allele of each member of a set of seed color genes);
   on a second chromosome of the pair of homologous chromosomes, at the target locus corresponding to the target locus of the first chromosome of the pair of homologous chromosomes, the at least one functional ectopic nuclease-null allele of a PV gene; and
v) mutating the the endogenous MF genes at the native MF gene loci and the endogenous PV genes at the native PV gene loci to create loss-of-function alleles, optionally wherein the loss-of-function alleles are caused by contacting the genome with a site-specific guided nuclease (e.g., CRISPR) and one or more guide RNA sequences or multi-guide constructs, thereby providing male-fertile maintainer plant.

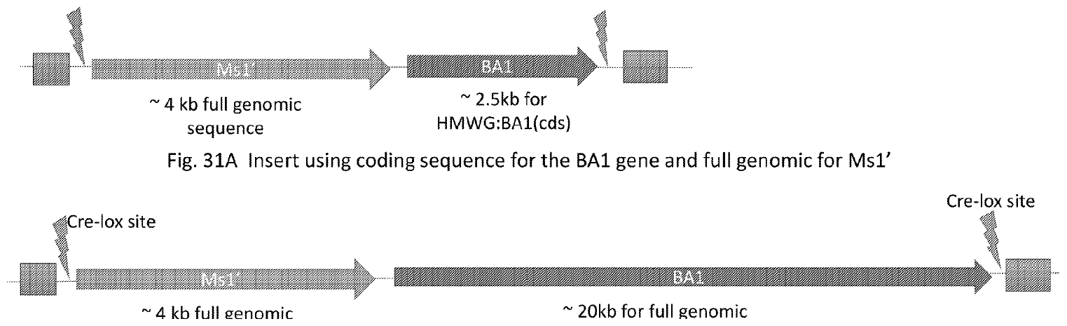

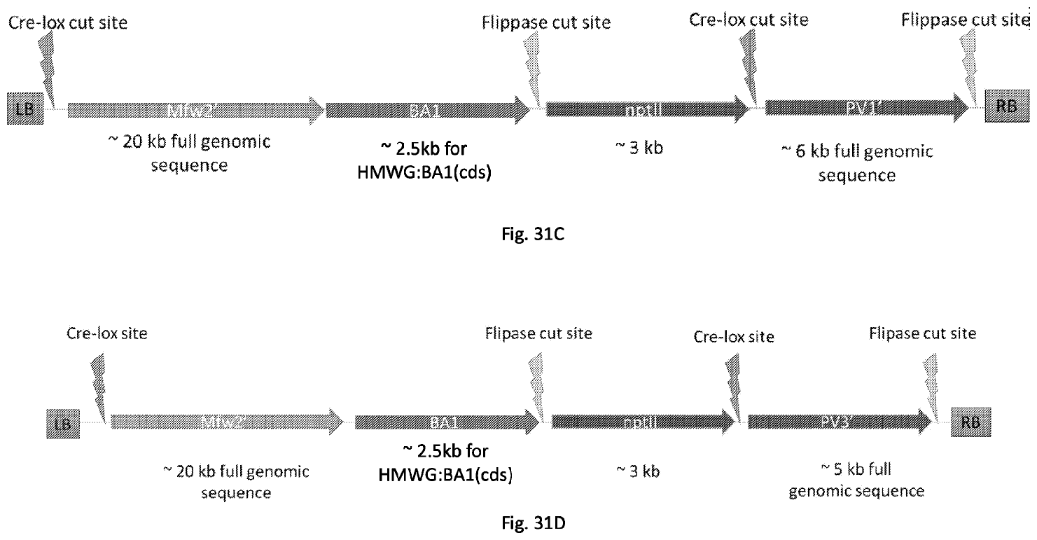

A cassette comprising all of:
a first recognition site for a first recombinase;
at least one functional ectopic nuclease null allele of a MF gene and at least one functional ectopic allele of a seed color gene (e.g., seed coat and/or seed endosperm gene) (or at least one functional ectopic allele of each member of a set of seed color genes) in either relative order;
a first recognition site for a second recombinase;
a selection gene;
a second recognition site for the first recombinase;
at least one functional ectopic nuclease-null allele of a PV gene;
a second recognition site for the second recombinase;
   is referred to herein as a “full cassette.” FIG. 31 provides exemplary embodiments of such a full cassette. As noted above, the full cassette can be provided in a number of possible configurations where all the recited elements are present. In particular, the above list of the elements of the construct can comprise a 5′ to 3′ order or a 3′ to 5′ order of the elements. It is specifically contemplated that the at least one functional ectopic nuclease null allele of a MF gene and at least one functional ectopic allele of a seed color gene (or at least one functional ectopic allele of each member of a set of seed color genes) can be in either 5′ to 3′ order relative to each other. A configuration is acceptable as long as:
the selection gene is located between a) the at least one functional ectopic nuclease-null allele of a PV gene and b) at least one functional ectopic nuclease null allele of a MF gene and at least one functional ectopic allele of a seed color gene (e.g., seed coat and/or seed endosperm gene) (or at least one functional ectopic allele of each member of a set of seed color genes); the first and second recognition sites for the first recombinase flank the at least one functional ectopic nuclease null allele of a MF gene, at least one functional ectopic allele of a seed color gene and the selection gene (or at least one functional ectopic allele of each member of a set of seed color genes);
the first and second recognition sites for the second recombinase flank the selection gene and the at least one functional ectopic nuclease-null allele of a PV gene.

The insertion can be efficiently selected for using the selection gene. As used herein, “selection gene” refers to a gene that confers a trait not endogenous to the plant/cell and which is readily selected for, e.g., herbicide or antibiotic resistance. Non-limiting examples of selection genes include nptII and nptII which confer resistance to kanamycin, beta-lactamase which confers resistance to certain penicillins like ampicillin, the ble genes that confers resistance to zeocin, the acetolactate synthase (ALS) gene (herbicide resistance, see e.g., Zong et al. Nature Plants 2019 5:480-5, which is incorporated by reference herein in its entirety), the acetyl-coenzyme A carboxylase gene, and the hygromycin resistance gene, More examples of resistance genes are also readily found in relevant databases, e.g, the Antibiotic Resistance Genes Database found at ardb.cbcb.umd.edu. In some embodiments of any of the aspects, the selection gene is nptII.

Once the full cassette is inserted into the genome, the resulting plant is referred to as a full-cassette insertion plant. It is contemplated herein that the plant could be a cell(s) of a plant and selection/screening/recombination occurs in cell culture. The full-cassette insertion plant can comprise the full-cassette in all somatic and germline cells (e.g., the plant is prepared or grown from a cell(s) comprising the full cassette), or the full-cassette insertion plant can comprise the full-cassette in at least some germline cells (e.g, if the cassette is introduced by Agrobacterium into cell in a flower). Two excising steps are then performed, respectively, on a first and second progeny of the full-cassette insertion plant. The progeny can be a plant descended from the full-cassette insertion plant, a cell(s) thereof, or a cell of the full-cassette insertion plant comprising the full-cassette.

The full cassette plant, first progeny, and second progeny can be heterozygous, hemizygous, or homozygous for the full cassette, depending on the methods utilized, the parameters of the screening, the propagation techniques utilized, and the number of generations separating the full-cassette insertion plant and the progeny. The first and second progeny are preferably genetically identical prior to the following excision steps and are differentiated by being physically separated and then subjected to different excision steps, rather than “first” and “second” implying any reference to, e.g., a first and second generation.

The first progeny is contacted with the first recombinase, which will cleave the genome at its first and second recognition sites, thereby excising the intervening sequence from the genome of the first progeny. That is, contacting the first progeny with the first recombinase will excise: one recognition site for the first recombinase, the at least one functional ectopic nuclease null allele of a MF gene and at least one functional ectopic allele of a seed color gene (e.g., seed coat and/or seed endosperm gene) (or at least one functional ectopic allele of each member of a set of seed color genes), the first recognition site for the second recombinase, and the selection gene from the genome of the first progeny. This excising step thereby provides an excised first progeny comprising: one recognition site for the first recombinase, the at least one functional ectopic nuclease-null allele of a PV gene, and the second recognition site for the second recombinase portions of the construct.

The second progeny is contacted with the second recombinase, which will cleave the genome at its first and second recognition sites, thereby excising the intervening sequence from the genome of the second progeny. That is, contacting the second progeny with the second recombinase will excise: one recognition site for the second recombinase, the selection gene, the second recognition site for the first recombinase and at least one functional ectopic nuclease-null allele of a PV gene. This excising step hereby provides an excised second progeny comprising: one recognition site for the second recombinase, the first recognition site for the first recombinase, and the at least one functional ectopic nuclease null allele of a MF gene and at least one functional ectopic allele of a seed color gene (e.g., seed coat and/or seed endosperm gene) (or at least one functional ectopic allele of each member of a set of seed color genes) portions of the construct.

After each of the excising steps, the first and second excised progeny can be selected by screening for the excision, e.g, by PCR screening. The excised first progeny and excised second progeny can be heterozygous, hemizygous, or homozygous for the excised cassette, depending on the methods utilized and the parameters of the screening.

The excised first progeny and excised second progeny are then crossed to produce a third progeny. The resulting third progeny comprises, in a first genome,
on a first chromosome of a pair of homologous chromosomes, at a single target locus, the at least one functional ectopic nuclease-null allele of a MF gene and the at least one functional ectopic allele of a seed color gene (e.g., seed coat and/or seed endosperm gene) (or at least one functional ectopic allele of each member of a set of seed color genes); and
on a second chromosome of the pair of homologous chromosomes, at the target locus corresponding to the target locus of the first chromosome of the pair of homologous chromosomes, the at least one functional ectopic nuclease-null allele of a PV gene.
   The third progeny (or a descendant or cell thereof) is mutated or engineered such that the endogenous MF genes at the native MF gene loci and the endogenous PV genes at the native PV gene loci are mutated or engineered to provide loss-of-function alleles. When all of the endogenous MF and PV alleles are mutated or engineered ot provide loss-of-function alleles, a male-fertile maintainer plant as described has been produced.

Alternatively, a male-fertile maintainer plant for a male-sterile polyploid plant can be prepared by a method comprising a first step of contacting a cell comprising a PV locus in a first chromosome and a second chromosome of a pair of homologous chromosomes in a first genome, with: 1) a site-specific guided nuclease (e.g., CRISPR); 2) one or more guide RNA sequences or multi-guide constructs specific to one or more sequences at the PV locus; and 3) a targeting insertion cassette comprising in 5′ to 3′ or 3′ to 5′ order: a first recognition site for a first recombinase and a second recognition site for the first recombinase; thereby providing a targeting insertion plant. In some embodiments of any of the aspects, the contacting of the first step comprises biolistic delivery or integration. The site-specific guided nuclease and guide sequences/constructs introduce the targeting insertion cassette into the PV locus. Selection of the guide sequence/constructs can provide a loss-of-function allele of the PV locus through the insertion of the targeting insertion cassette, or the insertion of the targeting insertion cassette may not interfere with the PV locus's expression or function. The targeting insertion plant is therefore available for targeted insertion of a desired second cassette by use of a recombinase that recognize's the targeting insertion cassette's recombinase sites. The insertion can be specific to the first genome, e.g., by selecting guide sequences/constructs specific to the sequence in a first genome, or can be made in multiple genomes and subject to later selection or engineering as described below.

In a second step, the targeting insertion plant, or first progeny of the targeting insertion plant, or a cell thereof is contacted with the first recombinase and a cassette comprising in 5′ to 3′ or 3′ to 5′ order: 1) a first recombination site for the first recombinase; 2) at least one functional ectopic nuclease null allele of a MF gene and at least one functional ectopic allele of a seed color gene (e.g., seed coat and/or seed endosperm gene) (or at least one functional ectopic allele of each member of a set of seed color genes) in either relative order; and 3) a second recombination site for the first recombinase; thereby providing a cassette insertion plant. The cassette insertion plant comprises the foregoing cassette inserted at at least one, and optionally, two alleles of the PV locus in the first genome. In some embodiments of any of the aspects, the contacting of the second step comprises transforming the plant, progeny, or cell thereof with one or more T-DNAs comprising the recombinase and cassette. In a third step, a cassette insertion plant comprising a cassette insertion at one allele of the PV locus is selected, or a cassette insertion plant comprising a cassette insertion at both alleles of the PV locus is crossed with a plant with a functional PV allele at the PV locus, thereby providing a cassette insertion plant with a cassette insertion at one PV allele in the first genome and a functional PV allele at the second PV allele in the first genome.

In a fourth step, the cassette insertion plant selected or provided by crossing in the third step, or a first progeny or cell thereof, is contacted with: 1) a site-specific guided nuclease (e.g., CRISPR); 2) one or more guide RNA sequences or multi-guide constructs flanking the insertion sites, thereby excising the inserted recombination sites; and 3) one or more guide RNA sequences or multi-guide constructs specific to the functional alleles of the endogenous MF gene and/or flanking the functional alleles of the endogenous MF gene, thereby mutating the functional alleles of the endogenous MF genes at the functional native MF gene loci to create loss-of-function alleles; thereby providing the male-fertile maintainer plant. In some embodiments of any of the aspects, the functional alleles of the MF gene comprise all alleles of the MF gene, e.g, two alleles in a diploid, four alleles in a tetraploid, or six alleles in a hexaploid. Accordingly, in some embodiments the fourth step comprises contacting the cassette insertion plant selected or provided by crossing in the third step, or a first progeny or cell thereof, with: 1) a site-specific guided nuclease (e.g., CRISPR); 2) one or more guide RNA sequences or multi-guide constructs flanking the insertion sites, thereby excising the inserted recombination sites; and 3) one or more guide RNA sequences or multi-guide constructs specific to the alleles of the endogenous MF gene and/or flanking the alleles of the endogenous MF gene, thereby mutating the alleles of the endogenous MF genes at the native MF gene loci to create loss-of-function alleles; thereby providing the male-fertile maintainer plant. For example, in some embodiments the fourth step comprises contacting the cassette insertion plant selected or provided by crossing in the third step, or a first progeny or cell thereof, with: 1) a site-specific guided nuclease (e.g., CRISPR); 2) one or more guide RNA sequences or multi-guide constructs flanking the insertion sites, thereby excising the inserted recombination sites; and 3) one or more guide RNA sequences or multi-guide constructs specific to all the alleles of the endogenous MF gene and/or flanking all the alleles of the endogenous MF gene, thereby mutating all the alleles of the endogenous MF genes at all the native MF gene loci to create loss-of-function alleles; thereby providing the potential for the male-fertile maintainer plant. In the latter embodiment, the inserted MF′ variant is not targeted by the one or more guide sequences because it has at least one SNP difference to the normal endogenous MF allele and so is not recognized for annealing/contacting by the guide. Exemplary MF′ alleles, e.g, Mfw2′ alleles are provided elsewhere herein.

In some embodiments of any of the aspects, the method further comprises a fifth step of segregating remaining T-DNA out of the plant or plant cells.

To create PV knock-out alleles as described herein, e.g., when the PV gene is endogenously expressed from the unmutated allele in the first genome and at least one further genome in the fourth step the plant, first progeny, or cell thereof can be further contacted with one or more guide RNA sequences or multi-guide constructs specific to the further genomes' endogenous PV genes and/or flanking the endogenous PV genes, thereby mutating the endogenous PV genes at the native PV gene loci of the further genomes to create loss-of-function alleles where they are not required for the hybrid system's allelic pair.

It is further contemplated herein that in some embodiments of any of the aspects, the MF gene is endogenously expressed from only some of the genomes. In such embodiments, it is not necessary to engineer loss of function alleles of the MF gene in the genomes which do not endogenously express the MF gene. For example, in some embodiments of any of the aspects, the MF gene is endogenously expressed only from the first genome. Unexpressed alleles can be hypermethylated alleles and/or alleles comprising a loss of function mutation. In such embodiments, it is not necessary to engineer loss of function alleles of the MF gene in the remaining genomes. For example, the MF gene can be Ms1, which is expressed only from the B genome of wheat. When the MF gene is Ms1, the gRNA sequences or constructs can be or comprise one or more of the three gRNA sequences of SEQ ID NOs: 253, 254, and 267. To create MF knock-out alleles as described herein, e.g., when the MF gene is endogenously expressed from the first genome and at least one further genome in the fourth step the plant, first progeny, or cell thereof can be further contacted with one or more guide RNA sequences or multi-guide constructs specific to the endogenous MF genes and/or flanking the endogenous MF genes, thereby mutating the endogenous MF genes at the native MF gene loci to create loss-of-function alleles.

In some embodiments of any of the aspects, the mutating or engineering to provide loss-of-function alleles can comprise one step, e.g, following by selection or screening. In some embodiments of any of the aspects, the selection or screening can comprise PCR screening for the desired excision. In some embodiments of any of the aspects, the mutating or engineering to provide loss-of-function alleles can comprise multiple steps, until all of the alleles are mutated or engineered. In some embodiments of any of the aspects, the loss-of-function alleles are caused by contacting the genome with a site-specific guided nuclease (e.g., CRISPR) and one or more guide RNA sequences or multi-guide constructs.

It is contemplated that the inserting and excision steps can be performed sequentially or concurrently. It is contemplated that the excision and the mutating/engineering steps can be performed sequentially or concurrently.

In some embodiments of any of the aspects, male-sterile plants can also be provided, produced, selected, or identified during the mutating or engineering of the third progeny. As illustrated in Example 9, when the first and second progeny are hemizygous, some of the third progeny will be heterzogyous MF′:seed color/PV′ and after the mutating or engineering to produce knockout or loss-of-function alleles of the endogenous MF and PV alleles, will be the maintainer plants as described herein. However, some of the progeny will be homozygous null for the insert or hemizygous PV′/− and after the mutating or engineering to produce knockout or loss-of-function alleles of the endogenous MF and PV alleles, will be the male-sterile plants as described herein.

A “recombinase,” as used herein, is a site-specific enzyme that recognizes short DNA sequence(s), which sequence(s) are typically between about 30 base pairs (bp) and 40 bp, and that mediates the recombination between these recombinase recognition sequences, which results in the excision, integration, inversion, or exchange of DNA fragments between the recombinase recognition sequences.

The outcome of the recombination reaction mediated by a recombinase depends, in part, on the location and orientation of two short repeated DNA sequences (e.g., RRS) that are to be recombined, typically less than 30 bp long. Recombinases bind to these repeated sequences, which are specific to each recombinase, and are herein referred to as “recombinase recognition sequences” or “recombinase recognition sites” or “RRS”. Thus, as used herein, a recombinase is “specific for” a recombinase recognition site when the recombinase can mediate inversion or excision between the repeat DNA sequences. As used herein, a recombinase may also be said to recognize its “cognate recombinase recognition sites,” which flank an intervening genetic element (e.g., a gene or genes). A genetic element is said to be “flanked” by recombinase recognition sites when the element is located between and immediately adjacent to two repeated DNA sequences.

In some embodiments of any of the aspects, the first and second recognition for a recombinase are provided or are in the same orientation, such that excision rather than inversion is performed by the recombinase.

The first and second recombinases are recombinases that recognized and cause recombination at different recognition sites. The first and second recombinases can be related, but must not utilize each other's recognition sites. Numerous recombinases and their cognate recognition sites are known in the art. Exemplary recombinases for use in the methods and compositions as described herein, include, but are not limited to, Flp, Dre, SCre, VCre, Vika, B2, B3, KD, ΦC31, Bxb1, λ, HK022, HP1, γδ, ParA, Tn3, Gin, R4, TP901-1, TG1, PhiRvl, PhiBT1, SprA, XisF, TnpX, R, BxB1, A118, spoIVCA, PhiMR11, SCCmec, TndX, XerC, XerD, XisA, Hin, Cin, mrpA, beta, PhiFC1, Fre, Clp, sTre, FimE, and HbiF. In some embodiments of any of the aspects, the recombinase is a tyrosine recombinase. In some embodiments of any of the aspects, the tyrosine recombinase is Cre, VCre, SCre, Flippase (Flp) XerA, XerC, or XerD. In some embodiments of any of the aspects, the first and second recombinase are Cre and Flp, or Flp and Cre respectively.

The sequences of recombinases and their recognition sites are well known in the art, for example, VCre's amino acid sequence is available in Genbank as ABX77110.1 and SCre's amino acid sequence is available in Genbank as ABK50591.1. Further discussion of VCre and SCre can be found, e.g., in Suzuki. Nucleic Acids Res 2011 39:e49; which is incorporated by reference herein in its entirey.

In any of the methods described herein, casettes, constructs, and genes can be introduced or inserted by any method known in the art, e.g., biolistic delivery, or vector delivery (e.g., viral vectors or T-DNA vectors). Methods of transforming plants/plant cells are well known in the art. In some embodiments of any of the aspects, contacting a plant/cell with a cassette/construct/or gene(s) comprises contacting the plant/plant cell(s) with a viral vector comprising the cassette/construct/gene(s). In some embodiments of any of the aspects, contacting a plant/cell with a cassette/construct/or gene(s) comprises contacting the plant/plant cell(s) with a T-DNA vector comprising the cassette/construct/gene(s). In some embodiments of any of the aspects, introducing or inserting a cassette/construct/or gene(s) into a plant/plant cell comprises contacting the plant/plant cell(s) with a viral vector comprising the cassette/construct/gene(s). In some embodiments of any of the aspects, introducing or inserting a cassette/construct/or gene(s) into a plant/plant cell comprises contacting the plant/plant cell(s) with a T-DNA vector comprising the cassette/construct/gene(s).

In any of the aspects of the methods described herein contacting the plant/plant cell(s) with a recombinase can comprise contacting the plant/plant cell(s) with a recombinase polypeptide, or with a nucleic acid (e.g., a vector) encoding the recombinase. In any of the aspects of the methods described herein contacting the plant/plant cell(s) with a recombinase can comprise introducing into the plant/plant cell(s) a recombinase polypeptide, or a nucleic acid (e.g., a vector) encoding the recombinase. In embodiments relating to a nucleic acid (e.g., a vector) encoding the recombinase, a step of removing or selecting out the nucleic acid encoding the recombinase after the relevant excision step.

Introducing, contacting, or inserting a polypeptide or nucleic acid can comprise transformation, transduction, and/or transfection according to any method known in the art.

In one aspect of any of the embodiments, described herein is a male-sterile plant or maintainer plant (or seed thereof), obtained by a method described herein.

In one aspect of any of the embodiments, described herein is a method of providing a male sterile plant seed, the method comprising selecting, from seed produced by selfing a maintainer plant as described herein, seed not displaying a phenotype provided by the seed endosperm gene. The selecting can be done manually or by a machine or device, e.g., a device that can sort based on seed color. Such devices are known in the art and suitable exemplary thresholds and sorting mechanisms are described in the examples herein. In one aspect of any of the embodiments, described herein is a method of providing male sterile plant seed, the method comprising selfing a maintainer plant as described herein, whereby the resulting seed not displaying a phenotype provided by the seed endosperm gene is the male sterile plant seed. Selfing a maintainer plant can include, but is not limited to, growing the maintainer plant under circumstances where cross-pollination with pollination-capable plants that are not maintainer plants is not likely to occur and/or will not occur, e.g., growing the maintainer plant in a greenhouse or other controlled environment lacking pollination-capable plants that are not maintainer plants, growing the maintainer plant in a field where pollination-capable plants that are not maintainer plants are not within pollination range (this will vary depending on e.g., the identity of the plant, local environmental conditions, and the existence and characteristics of intervening plants or structures and can readily be determined by one of ordinary skill in the art for an individual set of conditions), or growing the maintainer plant in or partially inside a device that isolates the reproductive portions of the plant and prevents or reduces cross-pollination (e.g., a pollination bag).

In one aspect of any of the embodiments, described herein is a method of providing a F1 hybrid seed for crop production, the method comprising collecting the seed produced by a male-sterile plant pollinated by a male-fertile plant, wherein the male-sterile plant is a) a plant grown from male sterile plant seed obtained by the method described herein; and/or b) comprises: i) loss-of-function alleles of an endogenous MF gene at each of the native MF gene loci; ii) loss-of-function alleles of an endogenous PV gene at each of the native PV gene loci; and iii) two ectopic alleles of the PV gene at a target locus. In one aspect of any of the embodiments, described herein is a method of providing a F1 hybrid seed for crop production, the method comprising crossing a a male-sterile plant with a male-fertile plant, wherein the male-sterile plant is a) a plant grown from male sterile plant seed obtained by the method described herein; and/or b) comprises: i) loss-of-function alleles of an endogenous MF gene at each of the native MF gene loci; ii) loss-of-function alleles of an endogenous PV gene at each of the native PV gene loci; and iii) two ectopic alleles of the PV gene at a target locus. In one aspect of any of the embodiments, described herein is a method of providing a F1 hybrid seed for crop production, the method comprising planting a male-sterile plant within pollination range of a male-fertile plant, wherein the male-sterile plant is a) a plant grown from male sterile plant seed obtained by a method described herein; and/or b) comprises: i) loss-of-function alleles of an endogenous MF gene at each of the native MF gene loci; ii) loss-of-function alleles of an endogenous PV gene at each of the native PV gene loci; and iii) two ectopic alleles of the PV gene at a target locus; and whereby the male-fertile plant pollinates the male-sterile plant and F1 hybrid seed is produced. As described above, the pollination range will vary depending on the species of plant and the growing conditions. One of ordinary skill in the art can determine the pollination range for a selected species and site. In some embodiments of any of the aspects, the pollination range of wheat is 200 meteres or less. In some embodiments of any of the aspects, the pollination range of wheat is 100 meteres or less. In some embodiments of any of the aspects, the pollination range of wheat is 50 meteres or less. In some embodiments of any of the aspects, the pollination range of wheat is 300 meteres or less. In some embodiments of any of the aspects, the pollination range of wheat is 400 meteres or less. In some embodiments of any of the aspects, the male-sterile plant and male fertile plant are different lines. In some embodiments of any of the aspects, the male-sterile plant and male fertile plant are different elite lines.

In one aspect of any of the embodiments, described herein is a method of producing a plant crop (e.g., a commodity or cash crop, or a crop for consumption, or a crop for industrial use and not for use as planting seed), the method comprising: a) planting and/or harvesting a plant or portion thereof, wherein the plant i) is plant grown from F1 hybrid seed obtained by a method described herein; and/or ii) comprises: 1) in each genome of the plant, at a native MF gene locus, one functional endogenous allele of the endogenous MF gene and one loss-of-function allele of the endogenous MF gene; 2) in each genome of the plant, at a native PV gene locus, one functional endogenous allele of the endogenous PV gene and one loss-of-function allele of the endogenous PV gene; 3) one ectopic allele of the PV gene at a target locus.

The engineered alleles described herein can be engineered by any single methodology or technology known in the art (which are described elsewhere herein) or a combination of any of those methodologies or technologies. In some embodiments of any of the apects, the method comprises engineering one or more modifications, e.g., by contacting a plant cell with a site-specific guided nuclease. In some embodiments of any of the apects, the method comprises engineering one or more modifications, e.g., by contacting a plant cell with a site-specific guided nuclease and at least one multi-guide construct. In some embodiments of any of the apects, step b, c, or d of the foregoing method comprises a single step of contacting a plant cell with a site-specific guided nuclease (e.g., a Cas enzyme) and one or more multi-guide constructs that target each allele of a MF and/or PV gene in the indicated genomes.

In one aspect of any of the embodiments, provided herein is a method of producing a male-fertile maintainer plant comprising nuclease-null (e.g., CRISPR-null) MF and PV alleles as described herein in a second plant line, wherein the method comprises:
a. crossing an extant male-fertile maintainer plant of a first line with a second plant of a second line to obtain a F1 generation, wherein the extant male-fertile maintainer plant comprises the alleles and/or modifications described herein;
b. selfing the F1 plant to obtain a F2 generation,
c. selecting for a plant or seed in the F2 generation with the greatest degree of conformity with the second line (e.g., by genetic sequence (e.g., SNPs) or by phenotype) and comprising the ectopic alleles and/or seed endosperm genes of the male-fertile maintainer plant as described herein;
d. optionally, backcrossing the plant selected in step b with the second line to obtain a further generation and then repeating the selection in the further generation as above,
e. optionally, repeating step d iteratively.

In one aspect of any of the embodiments, provided herein is a method of producing a male-fertile maintainer plant comprising nuclease-null (e.g., CRISPR-null) MF and PV alleles as described herein in a second plant line, wherein the method comprises:
a. crossing an extant male-fertile maintainer plant of a first line with a second plant of a second line to obtain a F1 generation, wherein the extant male-fertile maintainer plant comprises the alleles and/or modifications described herein;
b. selfing the F1 plant to obtain a F2 generation, selecting for a plant or seed in the F2 generation with the greatest degree of conformity with the second line (e.g., by genetic sequence (e.g., SNPs) or by phenotype) and comprising the ectopic alleles and/or seed endosperm genes of the male-fertile maintainer plant as described herein;
c. optionally, backcrossing the plant selected in step b with plant of the second line to obtain a further generation and then repeating the selection in the further generation as above,
d. optionally, repeating step c iteratively,
e. engineering:
   loss-of-function alleles in/at each native or endogenous allele of a MF gene in the second and any subsequent genomes, and at the allele on the second chromosome of the homologous pair in the first genome; and loss-of-function alleles in/at each native allele of a PV gene in all genomes.

In some embodiments of any of the aspects, each step of engineering a loss-of-function allele utilizes a guided nuclease (e.g., Cas9) and one, two, three, or more targeted sequences per gene. In some embodiments of any of the aspects, each step of engineering a loss-of-function allele utilizes a targeted nuclease (e.g., Cas9) and three targeted sequences per gene. In some embodiments of any of the aspects, the step of engineering a loss-of-function allele in the MF and PV genes in the indicated genomes comprises concurrent or simultaneous knock-out modifications generated by contacting a cell with a guided nuclease (e.g., Cas9) and three guide RNA sequences for each target, e.g., six guide RNA sequences total.

Selection and screening of plants which comprise the engineered alleles or modification(s) and/or progeny which comprise a combination of engineered alleles or modifications can be performed by any method known in the art, e.g., by phenotype screening or selection, genetic analysis (e.g. PCR or sequencing to detect the modifications), analysis of gene expression products, and the like. In some embodiments of any of the aspects, PCR screening can comprise PCR utilizing KASP primers. Such methods are known to one of skill in the art and can be used in any combination as desired. In some embodiments of any of the aspects, the engineered modifications do not comprise introduction of an exogenous marker gene (e.g., a selectable marker or screenable marker such as herbicide resistance or fluorsence or color-altering genes), and any selection or screening step does not rely upon the use of a selectable marker gene.

In one aspect of any of the embodiments, provided herein is a method of propagating a male-fertile maintainer plant as described herein, wherein the method comprises:
a. Permitting a male-fertile maintainer plant as described herein to self-fertilize;
b. Sorting the seed resulting from the self-fertilization to retain the seed expressing the seed endosperm gene's phenotype (e.g., the color produced by the seed endosperm gene's expression).

With homozygous MF′ being impossible due to pollen grains incorporating MF′ having no PV allele vital for pollen germination, the sorted seed (incorporating, e.g., BA with MF′) resulting from this method will have the same heterozygous genotype (e.g., MF′:BA/PV′) as the parental male-fertile maintainer plant (e.g, the plant that self-fertilized in step a).

In one aspect of any of the embodiments, provided herein is a method of propagating a male-sterile plant as described herein (e.g., having a mfiv×3, PV′/PV′, pv×3 genotype or a mf vx2, mfwv:PV pv×3 genotype) wherein the method comprises:
a. Permitting a male-fertile maintainer plant as described herein to pollinate the male-sterile plant;
b. collecting the seed produced by the male-sterile plant.

The seed resulting from this method will have the same genotype as the male-sterile parent plant (e.g. the male-sterile plant pollinated in step a).

In one aspect of any of the embodiments, provided herein is a method of producing F1 crop seed, the method comprising:
c. Permitting a male-fertile breeding line (e.g., an elite breeding line) to pollinate a male-sterile plant as described herein (e.g., having a mfv×3, PV′/PV′, pv×3 genotype or a mf vx2 mfw:PV pv×3 genotype);
d. collecting the seed produced by the male-sterile plant.
   The seed resulting from this method will be F1 seed.

In some embodiments of any of the aspects, a loss-of-function allele can comprise a “deactivating modification.” The phrase “deactivating modification” refers to a modification of an individual nucleic acid sequence and/or copy of a gene, resulting in deactivation of the allele. In some embodiments, deactivating modifications at all alleles of a given gene may be necessary to deactivate the gene. Furthermore, it is contemplated herein that the deactivating modification found at any given copy of a gene may or may not be identical to the deactivating modification found at the remaining copies of that gene. In some embodiments of any of the aspects, a knock-out or nonfunctional allele of a gene can comprise a deactivating modification at that allele.

As used herein, a “deactivated” gene is one that, due to engineering and/or modification of the genome (both chromosomal and/or extrachromosomal) of the cell in which the gene is found, is expressed at less than 35% of the wild-type level of functional polypeptide. In some embodiments of any of the aspects, a deactivated gene is expressed at less than 30% of the wild-type level of functional polypeptide. In some embodiments of any of the aspects, a deactivated gene is expressed at less than 25% of the wild-type level of functional polypeptide. In some embodiments of any of the aspects, a deactivated gene is expressed at less than 20% of the wild-type level of functional polypeptide. In some embodiments of any of the aspects, a deactivated gene is expressed at less than 15% of the wild-type level of functional polypeptide.

The wild-type level of functional polypeptide can be the level of functional polypeptide found in the same type of cell not comprising the modification. In some embodiments of any of the aspects, the level of functional polypeptide can be the level of full-length polypeptide with a wild-type sequence.

In some embodiments of any of the aspects, deactivation of a gene can comprise engineering, modifying, and/or altering the genome of the cell in which the gene is found such that the cell expresses no more than 35% of the wild-type level of the polypeptide, inclusive of both full-length and partial sequences of the gene. In some embodiments of any of the aspects, a deactivated gene is expressed at less than 30% of the wild-type level of polypeptide, inclusive of both full-length and partial sequences of the gene. In some embodiments of any of the aspects, a deactivated gene is expressed at less than 25% of the wild-type level of polypeptide, inclusive of both full-length and partial sequences of the gene. In some embodiments of any of the aspects, a deactivated gene is expressed at less than 20% of the wild-type level of polypeptide, inclusive of both full-length and partial sequences of the gene. In some embodiments of any of the aspects, a deactivated gene is expressed at less than 15% of the wild-type level of polypeptide, inclusive of both full-length and partial sequences of the gene.

In some embodiments of any of the aspects, deactivation of a gene can comprise engineering, modifying, and/or altering the genome of the cell in which the gene is found such that the cell expresses polypeptides comprising no more than 35% of the wild-type sequence of the polypeptide. In some embodiments of any of the aspects, deactivation of a gene can comprise engineering, modifying, and/or altering the genome of the cell in which the gene is found such that the cell expresses polypeptides comprising no more than 30% of the wild-type sequence of the polypeptide. In some embodiments of any of the aspects, deactivation of a gene can comprise engineering, modifying, and/or altering the genome of the cell in which the gene is found such that the cell expresses polypeptides comprising no more than 25% of the wild-type sequence of the polypeptide. In some embodiments of any of the aspects, deactivation of a gene can comprise engineering, modifying, and/or altering the genome of the cell in which the gene is found such that the cell expresses polypeptides comprising no more than 20% of the wild-type sequence of the polypeptide. In some embodiments of any of the aspects, deactivation of a gene can comprise engineering, modifying, and/or altering the genome of the cell in which the gene is found such that the cell expresses polypeptides comprising no more than 15% of the wild-type sequence of the polypeptide. In some embodiments of any of the aspects, deactivation of a gene can comprise engineering, modifying, and/or altering the genome of the cell in which the gene is found such that the cell expresses polypeptides comprising no more than 10% of the wild-type sequence of the polypeptide.

The whole wheat genome has previously been sequenced and published. Sequences are given in International Wheat Genome Sequencing Consortium (IWGSC) TIWGSC, IWGSC RefSeq principal investigators: R, Appels R, Eversole K, Feuillet C, Keller B, et al. Shifting the limits in wheat research and breeding using a fully annotated reference genome. Science. American Association for the Advancement of Science; 2018; 361:eaar7191; Chapman et al (2014) and Clavijo et al, (2016) and were downloadable from, e.g., TGAC, The Genome Analysis Centre, Norwich in January 2016 and subsequently published in October 2016 as part of Clavijo et al., 2016. (available on the world wide web at ftp.ensemblgenomes.org/pub/plants/pre/fasta/triticum_aestivum/dna/). In the case of wheat, selecting sequences of targeted genes for use in the present invention, suitable coding sequences can be selected from Appels et al. (2018), Clavijo et al, (2016), Chapman et al (2014) or TGAC (or any other academic publication).

In some embodiments, alleles may be deactivated by editing or deleting their associated promoter sequences or inserting a premature stop codon so that it no longer fulfils its function (‘gene knockout’). A variety of general methods are known for such gene editing. Such editing may involve additions to or deletions from the gene coding sequence or from control (regulatory) sequences upstream or downstream of the coding sequence, but in any case is such as to inhibit production of functional RNA transcript. For example, a gene might be knocked out by inserting one or more additional base pairs of DNA resulting in coding for one or more unsuitable amino-acids, or by creating a premature stop codon so as to substantially shorten the resulting RNA transcript. In some embodiments of any of the aspects, such “gene editing” modifications comprise only deletion of DNA base sequence and not insertion of exogenous sequence. Such editing by deletion, because it contains no additional or heterogenous DNA, is often regarded as environmentally safer and so may require less extensive, and hence less expensive and time-consuming, regulation. Accordingly, in some embodiments of any of the aspects, a deactivating modification can be a modification that interrupts and/or alters the wild-type coding sequence of the gene, e.g., by deletions which generate a stop codon, transposon, deletion, or frameshift in the coding sequence of the gene. Methods of performing such modifications are described elsewhere herein.

In some embodiments of any of the aspects, engineered modifications, including deactivating modifications, can be introduced by means of a mutagen, e.g., ethyl methane sulphonate (EMS), radiation, UV light, aflatoxin B1, nitrosoguanidine (NG), formaldehyde, acetaldehyde, diepoxyoctane (DEO), depoxybutane (DEB), diethyl sulphate (DES), methylnitrontrosoguanidine (NTG), N-ethyl-N-nitrosourea (ENU), and trimethylpsoralen (TMP). In some embodiments of any of the aspects, engineered modifications can be introduced, selected, and/or identified by means of TILLING (Targeted Induced Local Lesions IN Genomes) which uses mutagens to generate mutations. TILLING is described in detail, e.g., in Kurowska et al. J Appl Genet 2011 52:371-390 and McCallum et al. Plant Physiol 2000 123:439-442, which are incorporated by reference herein in their entireties.

In some embodiments of any of the aspects, engineered modifications can be introduced by non-transgenic mutagenesis, e.g., by a method which causes mutations of the nucleic acid sequences of the plant genome without introducing foreign and/or exogenous nucleic acid molecules into the plant cell. In some embodiments of any of the aspects, non-transgenic mutagenesis can comprise insertions and/or deletions due to mutagenic activity, e.g., indels arising from damage and/or repair processes in the cell. Non-transgenic mutagenesis can utilize, e.g., chemical mutagens (e.g., mutagens not comprising a nucleic acid sequence) and/or radiation sources (e.g., UV light). Non-transgenic mutagenesis excludes the use of, e.g., transposon insertions and/or RNAi. In some embodiments of any of the aspects, non-transgenic mutagenesis does not comprise the use of a site-specific nuclease, e.g., CRISPR-Cas. In some embodiments of any of the aspects, non-transgenic mutagenesis can be used in, e.g., TILLING approaches to generate and/or identify engineered modifications.

In some embodiments of any of the aspects, the engineered modification is not a naturally occurring modification, mutation, and/or allele.

In some embodiments of any of the aspects, the deactivating modification is excision of at least part of a coding or regulatory sequence; or the deactivated gene is deactivated by excision of at least part of a coding or regulatory sequence. In some embodiments of any of the aspects, the deactivating modification is non-transgenic mutagenesis; or the deactivated gene is deactivated by non-transgenic mutagenesis.

In some embodiments of any of the aspects, genes can be deactivated by utilizing a CRISPR/Cas system to introduce deactivating mutations at these loci. For example, PV1 can be targeted with four guide RNAs for each of the three sets of homoeologues and exemplary sets of such guide sequences are provided herein, e.g., guides having the sequences of SEQ ID Nos:210-213 can be used to target PV1. Exemplary guide sequences for targeting MF and PV alleles are described herein. Exemplary guide sequences for targeting Mfw alleles (either for knock-outs or simultaneous knockout/knock-ins) can also be found in International Patent Application PCT/US2017/043009, e.g., as SEQ ID NOs; 22-29 and 131-154 therein. A further exemplary guide sequence for targeting Mfw2 is SEQ ID NO: 238. The contents of International Patent Application PCT/US2017/043009 are incorporated by reference herein in their entirety.

PV1 guides (the fourth guide is in the reverse direction relative to the coding sequence)

SEQ ID NO: 210
GCATGGCGGAGCCGGAGGACGG
SEQ ID NO: 211
GTCGCCCCTCCTGAGGCGGCGG
SEQ ID NO: 212
AAGGAGGAGCCGGCGGCAGCGG
SEQ ID NO: 213
GAGACCGCCTCGCCGGAGCCGG

In some embodiments of any of the aspects, the deactivating modification is a site-directed mutagenic event resulting from the activity of a site-specific nuclease; or the at least one gene is deactivated by site-directed mutagenesis resulting from the activity of a site-specific nuclease. In some embodiments of any of the aspects, the site-specific nuclease is CRISPR-Cas.

In order for a gene to be deactivated, it is necessary to reduce the expression from multiple alleles or copies, e.g., wheat is a hexaploid genome and it may be necessary to reduce expression from all six copies of a given gene. Accordingly, in some embodiments of any of the aspects, a deactivating modification is present at all six copies of a given deactivated gene. The individual deactivating modifications can be identical or they can vary.

In some embodiments of any of the aspects, the deactivation of a first gene can further comprise deactivation of one or more further related genes which display functional redundancy with the first gene. In some embodiments of any of the aspects, a plant or cell in which a given gene is deactivated can comprise deactivating modification(s) that deactivate all members of that gene's family. In some embodiments of any of the aspects, a plant or cell in which a given gene is deactivated can comprise deactivating modification(s) that deactivate all genes with at least 30% sequence identity at the amino acid level to the gene. In some embodiments of any of the aspects, a plant or cell in which a given gene is deactivated can comprise deactivating modification(s) that deactivate all genes with at least 40% sequence identity at the amino acid level to the gene. In some embodiments of any of the aspects, a plant or cell in which a given gene is deactivated can comprise deactivating modification(s) that deactivate all genes with at least 50% sequence identity at the amino acid level to the gene. In some embodiments of any of the aspects, a plant or cell in which a given gene is deactivated can comprise deactivating modification(s) that deactivate all genes with at least 60% sequence identity at the amino acid level to the gene. In some embodiments of any of the aspects, a plant or cell in which a given gene is deactivated can comprise deactivating modification(s) that deactivate all genes with at least 70% sequence identity at the amino acid level to the gene. In some embodiments of any of the aspects, a plant or cell in which a given gene is deactivated can comprise deactivating modification(s) that deactivate all genes with at least 80% sequence identity at the amino acid level to the gene. In some embodiments of any of the aspects, a plant or cell in which a given gene is deactivated can comprise deactivating modification(s) that deactivate all genes with at least 90% sequence identity at the amino acid level to the gene.

In some embodiments of any of the aspects, a plant or cell in which a given gene is deactivated can comprise deactivating modification(s) that deactivate all genes with at least 30% sequence identity at the nucleotide level to the gene. In some embodiments of any of the aspects, a plant or cell in which a given gene is deactivated can comprise deactivating modification(s) that deactivate all genes with at least 40% sequence identity at the nucleotide level to the gene. In some embodiments of any of the aspects, a plant or cell in which a given gene is deactivated can comprise deactivating modification(s) that deactivate all genes with at least 50% sequence identity at the nucleotide level to the gene. In some embodiments of any of the aspects, a plant or cell in which a given gene is deactivated can comprise deactivating modification(s) that deactivate all genes with at least 60% sequence identity at the nucleotide level to the gene. In some embodiments of any of the aspects, a plant or cell in which a given gene is deactivated can comprise deactivating modification(s) that deactivate all genes with at least 70% sequence identity at the nucleotide level to the gene. In some embodiments of any of the aspects, a plant or cell in which a given gene is deactivated can comprise deactivating modification(s) that deactivate all genes with at least 80% sequence identity at the nucleotide level to the gene. In some embodiments of any of the aspects, a plant or cell in which a given gene is deactivated can comprise deactivating modification(s) that deactivate all genes with at least 90% sequence identity at the nucleotide level to the gene.

It is contemplated herein that such further related gene(s) can be deactivated by the same type of modification (e.g., the first gene is deactivated by modifying the gene with CRISPR/Cas and the further related gene(s) are deactivated by modifying the further related genes(s) with CRISPR/Cas); with the same modification step (e.g., the first gene is deactivated by modifying the gene with CRISPR/Cas and the further related gene(s) are simultaneously deactivated by modifying the further related genes(s) with the same CRISPR/Cas array, wherein the array targets sequences shared between the first and further genes); or by separate types of modifications.

In embodiments where multiple genes are to be deactivated, e.g., multiple members of a gene family, deactivating modifications can be targeted to shared sequences to minimize the number of modifications and/or individual reagents. Alternatively, deactivating modifications can be targeted to areas that are unique to each gene and a multiplexed approach can be taken. By way of non-limiting example, a gene family can be deactivated utilizing a single CRISPR sgRNA (or equivalent) if the sgRNA is targeted to a sequence found in all members of the gene family; or the gene family can be deactivated utilizing multiple CRISPR sgRNAs (or equivalents) if the sgRNAs are each targeted to sequences not found in each member of the gene family.

In some embodiments of any of the aspects, the engineered modifications described herein can be made directly in an elite breeding line. In some embodiments of any of the aspects, the engineered modifications described herein can be made in a first line or cultivar and then transferred to elite standard lines by traditional or ‘conventional’ crossing and selection.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

As used herein, a first plant which is a “maintainer” of a second male-sterile plant is a plant which is itself male-fertile but which when permitted to fertilize the male-sterile plant, will result in male-sterile plants in the next generation.

As used herein, a plant which is “male-sterile” is a plant in which less than 1% of pollen grains are viable, e.g., in which there are no detectable viable pollen grains. This is distinguished from uses in the art in which plants are referred to as male-sterile when they only have reduced male fertility, but still produce significant amounts of viable pollen and exhibit substantial rates of seed set. In some embodiments of any of the aspects, a male-sterile plant described herein is “stringently male-sterile”, i.e., no viable pollen grains can be detected and/or no seed set from natural self-fertilization is observed. In some embodiments of any of the aspects, a stringently male-sterile wheat gene is selected from the group consisting of Mfw1, Mfw2, and PV1. In some embodiments of any of the aspects, a plant which is “male-sterile” is not photo or thermo sensitive in its male-sterility. That is, the male-sterile phenotype is not dependent on light or temperature levels or changes. In some embodiments of any of the aspects, a “male-sterile” plant is one in which less than 1% of pollen grains are viable regardless of changes in light or temperature. In some embodiments of any of the aspects, a “male-sterile” plant is one in which less than 1% of pollen grains are viable regardless of changes in light or temperature that are within the range of light and temperature levels that permit growth and viable pollen production in a plant that is isogenic except for the MF mutation(s) that convey male sterility.

Plants can be polyploid, e.g., they contain multiple genomes. Accordingly, the plants and plant cells are described herein with reference to a first genome and further genomes (e.g., a second genome, a third genome, etc). When engineering the plants/cells described herein, the selection or designation of one genome as the first genome is at the discretion of the user. That is, there is not an inherent feature of one of the genomes that designates it as the “first” genome. Each genome comprises pairs of homologous chromosomes. When engineering the plants/cells described herein, the selection or designation of one chromosome of a pair of homologous chromosomes as the first member of the pair is at the discretion of the user. That is, there is not an inherent feature of one of the chromosomes that designates it as the “first” chromosome.

As used herein, “locus” refers to a fixed position on a chromosome, e.g., the location of a gene or marker and its immediately neighbouring sequence on a chromosome as it exists prior to engineering or modification. Thus, reference to a “MF locus” refers to the physical position of a given MF gene on a particular chromosome prior to any engineering or modification.

As used herein, “allele” refers to an individual copy of a gene. In a diploid organism, two alleles of a gene are typically present in the genome and the two alleles may not have identical sequences. Multiple different alleles can be present in a single organism, in a single population, or a single species.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given element or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level.

The terms “increased”, “increase”, “enhance”, or “activate” are all used herein to mean an increase by a statistically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker, an “increase” is a statistically significant increase in such level.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to a suitable assay for gene activity, e.g., pollen viability and/or seed set. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, Jan. 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

As used herein, a “transgenic” organism or cell is one in which exogenous DNA from another source (natural, from a second non-crossable species, or synthetic) has been introduced.

The term “exogenous” refers to a substance present in a cell other than its native source. The term “exogenous” when used herein can refer to a nucleic acid (e.g., a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism.

“Ectopic” refers to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels; or which has been introduced by a process involving the hand of man into a different location within the same biological system (such as a cell or organism) in which the nucleic acid or polypeptide naturally occurs.

In contrast, the term “endogenous” refers to a substance that is native to the biological system or cell in both location and amount.

As used herein, “cognate” with respect to the maintainer line and its phenotypic relative (e.g., a male-sterile line), refers to the two plants carrying recessive alleles (e.g., loss-of-function alleles) of the same phenotype-controlling gene(s) of interest according to the schemes described herein. For example, a male-sterile plant which comprises only recessive non-functional alleles of a first MF gene is not cognate with a maintainer line which carries recessive non-functional alleles of a second MF gene. It is noted that the recessive alleles need not be identical in sequence in order for a maintainer and the phenotypic relative to be cognate.

In some embodiments, a nucleic acid encoding a DNA or an RNA molecule or a polypeptide as described herein can be introduced into a cell by, e.g., biolistic delivery.

In some embodiments, a nucleic acid encoding an RNA or polypeptide as described herein is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc. Exemplary vectors are known in the art and can include, by way of non-limiting example, pBR322 and related plasmids, pACYC and related plasmids, transcription vectors, expression vectors, phagemids, yeast expression vectors, plant expression vectors, pDONR201 (Invitrogen), pBIl21, pBIN20, pEarleyGate100 (ABRC), pEarleyGate102 (ABRC), pCAMBIA, pUC-derived vectors, pSK-derived vectors, pGEM-derived vectors, pSP-derived vectors, pBS-derived vectors, the binary Ti plasmid (see, e.g., U.S. Pat. No. 4,940,838; which is incorporated by reference herein in its entirety), T-DNA, transposons, and artificial chromosomes.

As used herein, the term “expression vector” refers to a vector that directs expression of an RNA or polypeptide from sequences operably linked to transcriptional regulatory sequences on the vector. The term “operably linked” as used herein refers to a functional linkage between a regulatory element and a second sequence, wherein the regulatory element influences the expression and/or processing of the second sequence. Generally, “operably linked” means that the nucleic acid sequences being linked are contiguous or near contiguous and, where necessary to join two protein coding regions, contiguous and in the same reading frame. The regulatory sequence, e.g., a promoter, can be a constitutive, tissue-specific, and/or inducible promoter. The sequences expressed will often, but not necessarily, be heterologous to the cell. An expression vector may comprise additional elements, for example, the expression vector may have two replication systems, thus allowing it to be maintained in two organisms, for example in plant cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

In the context of this invention, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” refers to a sufficient degree of complementarity or precise pairing such that stable and specific binding occurs between the two nucleic acid sequences under the relevantly strigent conditions, e.g., in this case, in a plant cell. As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

As used herein, “contacting” refers to any suitable means for delivering, or exposing, an agent to at least one cell. The cell can be ex vivo or in vitro. In some embodiments, a cell is contacted. In some embodiments, at least one cell in a culture or tissue is contact. In some embodiments, at least one cell in a plant is contacted. Exemplary delivery methods include, but are not limited to, direct delivery to cell culture medium, perfusion, injection, transfection, ballistic delivery, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The invention can be further described with reference to the accompanying sequences, wherein:
SEQ ID NO 1 is the amino-acid sequence for which Mfw1-A codes
SEQ ID NO 2 is the amino-acid sequence for which Mfw1-B codes
SEQ ID NO 3 is the amino-acid sequence for which Mfw1-D codes
SEQ ID NO 4 is the amino-acid sequence for which Mfw2-A codes
SEQ ID NO 5 is the amino-acid sequence for which Mfw2-B codes
SEQ ID NO 6 is the amino-acid sequence for which Mfw2-D codes
SEQ ID NO 7 is the DNA coding sequence (from start codon to stop codon inclusive) of Mfw1-A from wheat (Triticum aestivum, variety ‘Fielder’)
SEQ ID NO 8 is the DNA coding sequence (from start codon to stop codon inclusive) of Mfw1-B from wheat (Triticum aestivum, variety ‘Fielder’)
SEQ ID NO 9 is the DNA coding sequence (from start codon to stop codon inclusive) of Mfw1-D from wheat (Triticum aestivum, variety ‘Fielder’)
SEQ ID NO 10 is the DNA coding sequence (from start codon to stop codon inclusive) of Mfw2-A from wheat (Triticum aestivum, variety ‘Fielder’)
SEQ ID NO 11 is the DNA coding sequence (from start codon to stop codon inclusive) of Mfw2-B from wheat (Triticum aestivum, variety ‘Fielder’)
SEQ ID NO 12 is the DNA coding sequence (from start codon to stop codon inclusive) of Mfw2-D from wheat (Triticum aestivum, variety ‘Fielder’)
SEQ ID NO 13 is a partial sequence of chromosome 7A of wheat (Triticum aestivum, variety ‘Chinese Spring’) including Mfw1-A
SEQ ID NO 14 is a partial sequence chromosome 7A of wheat (Triticum aestivum, variety ‘Chinese Spring’) including Mfw2-A
SEQ ID NO 15 is a partial sequence of chromosome 7B of wheat (Triticum aestivum, variety ‘Chinese Spring’) including Mfw1-B
SEQ ID NO 16 is a partial sequence of chromosome 7B of wheat (Triticum aestivum, variety ‘Chinese Spring’) including Mfw2-B
SEQ ID NO 17 is a partial sequence of chromosome 7D of wheat (Triticum aestivum, variety ‘Chinese Spring’) including Mfw1-D
SEQ ID NO 18 is a partial sequence of chromosome 7D of wheat (Triticum aestivum, variety ‘Chinese Spring’) including Mfw2-D
SEQ ID NO 19 is a DNA sequence that can be used in a hairpin described in International Patent Publication WO 2018/022410.
SEQ ID NO: 20 is a sequence of Mfw1 and SEQ ID NOs: 22-25 are guide targeting sequences for SEQ ID NO: 20.
SEQ ID NO: 21 is a sequence of Mfw2 and SEQ ID NOs: 26-29 are guide targeting sequences for SEQ ID NO: 21.
SEQ ID NO 30 is the amino-acid sequence for which Mfw3-A codes.
SEQ ID NO 31 is the amino-acid sequence for which Mfw3-B codes.
SEQ ID NO 32 is the amino-acid sequence for which Mfw3-D codes.
SEQ ID NO 33 is the amino-acid sequence for which Mfw5-A codes.
SEQ ID NO 34 is the amino-acid sequence for which Mfw5-B codes.
SEQ ID NO 35 is the amino-acid sequence for which Mfw5-D codes.
SEQ ID NO 36 is the DNA coding sequence (from start-codon to stop-codon inclusive) of Mfw3-A from wheat (Triticum aestivum, variety ‘Fielder’). SEQ ID NOs: 131-134 are guide targeting sequences for SEQ ID NO: 36. SEQ ID NO: 54 is a portion of SEQ ID NO: 36 that can be used in a Mfw-3/Mfw-5 hairpin described in International Patent Publication WO 2018/022410.
SEQ ID NO 37 is the DNA coding sequence (from start-codon to stop-codon inclusive) of Mfw3-B from wheat (Triticum aestivum, variety ‘Fielder’). SEQ ID NOs: 135-138 are guide targeting sequences for SEQ ID NO: 37. SEQ ID NO: 55 is a portion of SEQ ID NO: 37 that can be used in a Mfw-3/Mfw-5 hairpin described in International Patent Publication WO 2018/022410.
SEQ ID NO 38 is the DNA coding sequence (from start-codon to stop-codon inclusive) of Mfw3-D from wheat (Triticum aestivum, variety ‘Fielder’). SEQ ID NOs: 139-142 are guide targeting sequences for SEQ ID NO: 38. SEQ ID NO: 56 is a portion of SEQ ID NO: 38 that can be used in a Mfw-3/Mfw-5 hairpin described in International Patent Publication WO 2018/022410.
SEQ ID NO 39 is the DNA coding sequence (from start-codon to stop-codon inclusive) of Mfw5-A from wheat (Triticum aestivum, variety ‘Fielder’).
SEQ ID NO 40 is the DNA coding sequence (from start-codon to stop-codon inclusive) of Mfw5-B from wheat (Triticum aestivum, variety ‘Fielder’).
SEQ ID NO 41 is the DNA coding sequence (from start-codon to stop-codon inclusive) of Mfw5-D from wheat (Triticum aestivum, variety ‘Fielder’).
SEQ ID NO 42 is a partial sequence of chromosome 6A of wheat (Triticum aestivum, variety ‘Chinese Spring’) including Mfw3-A.
SEQ ID NO 43 is a partial sequence of chromosome 6B of wheat (Triticum aestivum, variety ‘Chinese Spring’) including Mfw3-B.
SEQ ID NO 44 is a partial sequence of chromosome 6D of wheat (Triticum aestivum, variety ‘Chinese Spring’) including Mfw3-D.
SEQ ID NO 45 is a partial sequence of chromosome 2A of wheat (Triticum aestivum, variety ‘Chinese Spring’) including Mfw5-A.
SEQ ID NO 46 is a partial sequence of chromosome 2B of wheat (Triticum aestivum, variety ‘Chinese Spring’) including Mfw5-B.
SEQ ID NO 47 is a partial sequence of chromosome 2D of wheat (Triticum aestivum, variety ‘Chinese Spring’) including Mfw5-D.
SEQ ID NO 48 is a DNA sequence that can be used in a Mfw-3/Mfw-5 hairpin described in International Patent Publication WO 2018/022410.
SEQ ID NO 60 is the amino-acid sequence for which Mfw4-A codes.
SEQ ID NO 61 is the DNA coding sequence (from start-codon to stop-codon inclusive) of Mfw4-A from wheat (Triticum aestivum, variety ‘Fielder’).
SEQ ID NO 62 is a partial sequence of the wheat (Triticum aestivum, variety ‘Chinese Spring’) genomic sequence including Mfw4-A.
SEQ ID NO 63 is the amino-acid sequence for which Mfw4-B codes.
SEQ ID NO 64 is the DNA coding sequence (from start-codon to stop-codon inclusive) of Mfw4-B from wheat (Triticum aestivum, variety ‘Fielder’).
SEQ ID NO 65 is a partial sequence of the wheat (Triticum aestivum, variety ‘Chinese Spring’) genomic sequence including Mfw4-B.
SEQ ID NO 66 is the amino-acid sequence for which Mfw4-D codes.
SEQ ID NO 67 is the DNA coding sequence (from start-codon to stop-codon inclusive) of Mfw4-D from wheat (Triticum aestivum, variety ‘Fielder’).
SEQ ID NO 68 is a partial sequence of the wheat (Triticum aestivum, variety ‘Chinese Spring’) genomic sequence including Mfw4-D.
SEQ ID NO 69 is the amino-acid sequence for which Mfw6-A codes.
SEQ ID NO 70 is the DNA coding sequence (from start-codon to stop-codon inclusive) of Mfw6-A from wheat (Triticum aestivum, variety ‘Fielder’).
SEQ ID NO 71 is a partial sequence of the wheat (Triticum aestivum, variety ‘Chinese Spring’) genomic sequence including Mfw6-A.
SEQ ID NO 72 is the amino-acid sequence for which Mfw6-D codes.
SEQ ID NO 73 is the DNA coding sequence (from start-codon to stop-codon inclusive) of Mfw6-D from wheat (Triticum aestivum, variety ‘Fielder’).
SEQ ID NO 74 is a partial sequence of the wheat (Triticum aestivum, variety ‘Chinese Spring’) genomic sequence including Mfw6-D.
SEQ ID NO 75 is the amino-acid sequence for which Mfw7-A codes.
SEQ ID NO 76 is the DNA coding sequence (from start-codon to stop-codon inclusive) of Mfw7-A from wheat (Triticum aestivum, variety ‘Fielder’).
SEQ ID NO 77 is a partial sequence of the wheat (Triticum aestivum, variety ‘Chinese Spring’) genomic sequence including Mfw7-A.
SEQ ID NO 78 is the amino-acid sequence for which Mfw7-B codes.
SEQ ID NO 79 is the DNA coding sequence (from start-codon to stop-codon inclusive) of Mfw7-B from wheat (Triticum aestivum, variety ‘Fielder’).
SEQ ID NO 80 is a partial sequence of the wheat (Triticum aestivum, variety ‘Chinese Spring’) genomic sequence including Mfw7-B.
SEQ ID NO 81 is the amino-acid sequence for which Mfw7-D codes.
SEQ ID NO 82 is the DNA coding sequence (from start-codon to stop-codon inclusive) of Mfw7-D from wheat (Triticum aestivum, variety ‘Fielder’).
SEQ ID NO 83 is a partial sequence of the wheat (Triticum aestivum, variety ‘Chinese Spring’) genomic sequence including Mfw7-D.
SEQ ID NO 84 is the amino-acid sequence for which Mfw8-A codes.
SEQ ID NO 85 is the DNA coding sequence (from start-codon to stop-codon inclusive) of Mfw8-A from wheat (Triticum aestivum, variety ‘Fielder’).
SEQ ID NO 86 is a partial sequence of the wheat (Triticum aestivum, variety ‘Chinese Spring’) genomic sequence including Mfw8-A.
SEQ ID NO 87 is the amino-acid sequence for which Mfw8-B codes.
SEQ ID NO 88 is the DNA coding sequence (from start-codon to stop-codon inclusive) of Mfw8-B from wheat (Triticum aestivum, variety ‘Fielder’).
SEQ ID NO 89 is a partial sequence of the wheat (Triticum aestivum, variety ‘Chinese Spring’) genomic sequence including Mfw8-B.
SEQ ID NO 90 is the amino-acid sequence for which Mfw8-D codes.
SEQ ID NO 91 is the DNA coding sequence (from start-codon to stop-codon inclusive) of Mfw8-D from wheat (Triticum aestivum, variety ‘Fielder’).
SEQ ID NO 92 is a partial sequence of the wheat (Triticum aestivum, variety ‘Chinese Spring’) genomic sequence including Mfw8-D.
SEQ ID NO 93 is the amino-acid sequence for which Mfw9-A codes.
SEQ ID NO 94 is the DNA coding sequence (from start-codon to stop-codon inclusive) of Mfw9-A from wheat (Triticum aestivum, variety ‘Fielder’).
SEQ ID NO 95 is a partial sequence of the wheat (Triticum aestivum, variety ‘Chinese Spring’) genomic sequence including Mfw9-A.
SEQ ID NO 96 is the amino-acid sequence for which Mfw9-B codes.
SEQ ID NO 97 is the DNA coding sequence (from start-codon to stop-codon inclusive) of Mfw9-B from wheat (Triticum aestivum, variety ‘Fielder’).
SEQ ID NO 98 is a partial sequence of the wheat (Triticum aestivum, variety ‘Chinese Spring’) genomic sequence including Mfw9-B.
SEQ ID NO 99 is the amino-acid sequence for which Mfw9-D codes.
SEQ ID NO 100 is the DNA coding sequence (from start-codon to stop-codon inclusive) of Mfw9-D from wheat (Triticum aestivum, variety ‘Fielder’).
SEQ ID NO 101 is a partial sequence of the wheat (Triticum aestivum, variety ‘Chinese Spring’) genomic sequence including Mfw9-D.
SEQ ID NO 102 is the amino-acid sequence for which Mfw10-A codes.
SEQ ID NO 103 is the DNA coding sequence (from start-codon to stop-codon inclusive) of Mfw10-A from wheat (Triticum aestivum, variety ‘Fielder’).
SEQ ID NO 104 is a partial sequence of the wheat (Triticum aestivum, variety ‘Chinese Spring’) genomic sequence including Mfw10-A.
SEQ ID NO 105 is the amino-acid sequence for which Mfw10-B codes.
SEQ ID NO 106 is the DNA coding sequence (from start-codon to stop-codon inclusive) of Mfw10-B from wheat (Triticum aestivum, variety ‘Fielder’).
SEQ ID NO 107 is a partial sequence of the wheat (Triticum aestivum, variety ‘Chinese Spring’) genomic sequence including Mfw11-U.
SEQ ID NO 108 is the amino-acid sequence for which Mfw11-U codes.
SEQ ID NO 109 is the DNA coding sequence (from start-codon to stop-codon inclusive) of Mfw11-U from wheat (Triticum aestivum, variety ‘Fielder’).
SEQ ID NO 110 is a partial sequence of the wheat (Triticum aestivum, variety ‘Chinese Spring’) genomic sequence including Mfw11-U.
SEQ ID NO 111 is the amino-acid sequence for which Mfw12-A codes.
SEQ ID NO 112 is the DNA coding sequence (from start-codon to stop-codon inclusive) of Mfw12-A from wheat (Triticum aestivum, variety ‘Fielder’).
SEQ ID NO 113 is a partial sequence of the wheat (Triticum aestivum, variety ‘Chinese Spring’) genomic sequence including Mfw12-A.
SEQ ID NO 114 is the amino-acid sequence for which Mfw12-B codes.
SEQ ID NO 115 is the DNA coding sequence (from start-codon to stop-codon inclusive) of Mfw12-B from wheat (Triticum aestivum, variety ‘Fielder’).
SEQ ID NO 116 is a partial sequence of the wheat (Triticum aestivum, variety ‘Chinese Spring’) genomic sequence including Mfw12-B.
SEQ ID NO 117 is the amino-acid sequence for which Mfw12-D codes.
SEQ ID NO 118 is the DNA coding sequence (from start-codon to stop-codon inclusive) of Mfw12-D from wheat (Triticum aestivum, variety ‘Fielder’).
SEQ ID NO 119 is a partial sequence of the wheat (Triticum aestivum, variety ‘Chinese Spring’) genomic sequence including Mfw12-D.
SEQ ID NO 120 is the amino-acid sequence for which Mfw13-A codes.
SEQ ID NO 121 is the DNA coding sequence (from start-codon to stop-codon inclusive) of Mfw13-A from wheat (Triticum aestivum, variety ‘Fielder’).
SEQ ID NO 122 is a partial sequence of the wheat (Triticum aestivum, variety ‘Chinese Spring’) genomic sequence including Mfw13-A.
SEQ ID NO 123 is the amino-acid sequence for which Mfw13-B codes.
SEQ ID NO 124 is the DNA coding sequence (from start-codon to stop-codon inclusive) of Mfw13-B from wheat (Triticum aestivum, variety ‘Fielder’).
SEQ ID NO 125 is a partial sequence of the wheat (Triticum aestivum, variety ‘Chinese Spring’) genomic sequence including Mfw13-D.
SEQ ID NO 126 is the amino-acid sequence for which Mfw13-B codes.
SEQ ID NO 127 is the DNA coding sequence (from start-codon to stop-codon inclusive) of Mfw13-D from wheat (Triticum aestivum, variety ‘Fielder’).
SEQ ID NO 128 is a partial sequence of the wheat (Triticum aestivum, variety ‘Chinese Spring’) genomic sequence including Mfw13-D.
SEQ ID NO: 129 is the coding sequence of Mfw5-A. SEQ ID NOs: 143-146 are guide targeting sequences for SEQ ID NO: 129. SEQ ID NO: 57 is a portion of SEQ ID NO: 129 that can be used in a Mfw-3/Mfw-5 hairpin described in International Patent Publication WO 2018/022410.
SEQ ID NO: 130 is the coding sequence of Mfw5-B. SEQ ID NOs: 147-150 are guide targeting sequences for SEQ ID NO: 130. SEQ ID NO: 58 is a portion of SEQ ID NO: 130 that can be used in a Mfw-3/Mfw-5 hairpin described in International Patent Publication WO 2018/022410.
SEQ ID NO: 41 is the coding sequence of Mfw5-D. SEQ ID NOs: 151-154 are guide targeting sequences for SEQ ID NO: 41. SEQ ID NO: 57 is a portion of SEQ ID NO: 59 that can be used in a Mfw-3/Mfw-5 hairpin described in International Patent Publication WO 2018/022410.

Further description of certain sequences:
SEQ ID NO 13 is a partial sequence of that part of chromosome 7A of wheat (Triticum aestivum, variety ‘Chinese Spring’) that, from the start codon starting at 6072 bp to the end of the TAA stop codon at 8122 bp, includes the DNA coding sequence for Mfw1-A as well as flanking sequences upstream of the start codon and downstream of the stop codon. These flanking sequences may be expected to include regulatory sequences, such as, in the upstream flanking sequence, the promoter.
SEQ ID NO 14 is a partial sequence of that part of chromosome 7B of wheat (Triticum aestivum, variety ‘Chinese Spring’) that, from the start codon starting at 2076 bp to the end of the TAA stop codon at 3844 bp, includes the DNA coding sequence for Mfw2-A as well as flanking sequences upstream of the start codon and downstream of the stop codon. These flanking sequences may be expected to include regulatory sequences, such as, in the upstream flanking sequence, the promoter.
SEQ ID NO 15 is a partial sequence of that part of chromosome 7D of wheat (Triticum aestivum, variety ‘Chinese Spring’) that, from the start codon starting at 7957 bp to the end of the TAA stop codon at 9960 bp, includes the DNA coding sequence for Mfw1-B as well as flanking sequences upstream of the start codon and downstream of the stop codon. These flanking sequences may be expected to include regulatory sequences, such as, in the upstream flanking sequence, the promoter.
SEQ ID NO 16 is a partial sequence of that part of chromosome 7A of wheat (Triticum aestivum, variety ‘Chinese Spring’) that, from the start codon starting at 2949 bp to the end of the TGA stop codon at 16953 bp, includes the DNA coding sequence for Mfw2-B as well as flanking sequences upstream of the start codon and downstream of the stop codon. These flanking sequences may be expected to include regulatory sequences, such as, in the upstream flanking sequence, the promoter.
SEQ ID NO 17 is a partial sequence of that part of chromosome 7B of wheat (Triticum aestivum, variety ‘Chinese Spring’) that, from the start codon starting at 249 bp to the end of the TGA stop codon at 17681 bp, includes the DNA coding sequence for Mfw1-D as well as flanking sequences upstream of the start codon and downstream of the stop codon. These flanking sequences may be expected to include regulatory sequences, such as, in the upstream flanking sequence, the promoter.
SEQ ID NO 18 is a partial sequence of that part of chromosome 7D of wheat (Triticum aestivum, variety ‘Chinese Spring’) that, from the start codon starting at 1255 bp to the end of the TGA stop codon at 18448 bp, includes the DNA coding sequence for Mfw2-D as well as flanking sequences upstream of the start codon and downstream of the stop codon. These flanking sequences may be expected to include regulatory sequences, such as, in the upstream flanking sequence, the promoter.
SEQ ID NO 42 is a partial sequence of that part of chromosome 6A of wheat (Triticum aestivum, variety ‘Chinese Spring’) that, from the start codon starting at 2130 bp to the end of the TGA stop codon at 4398 bp, includes the DNA coding sequence for Mfw3-A as well as flanking sequences upstream of the start codon and downstream of the stop codon. These flanking sequences may be expected to include regulatory sequences, such as, in the upstream flanking sequence, the promoter.
SEQ ID NO 43 is a partial sequence of that part of chromosome 6B of wheat (Triticum aestivum, variety ‘Chinese Spring’) that, from the start codon starting at 1884 bp to the end of the TGA stop codon at 4144 bp, includes the DNA coding sequence for Mfw3-B as well as flanking sequences upstream of the start codon and downstream of the stop codon. These flanking sequences may be expected to include regulatory sequences, such as, in the upstream flanking sequence, the promoter.
SEQ ID NO 44 is a partial sequence of that part of chromosome 6D of wheat (Triticum aestivum, variety ‘Chinese Spring’) that, from the start codon starting at 2078 bp to the end of the TGA stop codon at 4269 bp, includes the DNA coding sequence for Mfw3-D as well as flanking sequences upstream of the start codon and downstream of the stop codon. These flanking sequences may be expected to include regulatory sequences, such as, in the upstream flanking sequence, the promoter.
SEQ ID NO 45 is a partial sequence of that part of chromosome 2A of wheat (Triticum aestivum, variety ‘Chinese Spring’) that, from the start codon starting at 1395 bp to the end of the TGA stop codon at 3650 bp, includes the DNA coding sequence for Mfw5-A as well as flanking sequences upstream of the start codon and downstream of the stop codon. These flanking sequences may be expected to include regulatory sequences, such as, in the upstream flanking sequence, the promoter.
SEQ ID NO 46 is a partial sequence of that part of chromosome 2B of wheat (Triticum aestivum, variety ‘Chinese Spring’) that, from the start codon starting at 2360 bp to the end of the TGA stop codon at 4734 bp, includes the DNA coding sequence for Mfw5-B as well as flanking sequences upstream of the start codon and downstream of the stop codon. These flanking sequences may be expected to include regulatory sequences, such as, in the upstream flanking sequence, the promoter.
SEQ ID NO 47 is a partial sequence of that part of chromosome 2D of wheat (Triticum aestivum, variety ‘Chinese Spring’) that, from the start codon starting at 1501 bp to the end of the TGA stop codon at 3579 bp, includes the DNA coding sequence for Mfw5-D as well as flanking sequences upstream of the start codon and downstream of the stop codon. These flanking sequences may be expected to include regulatory sequences, such as, in the upstream flanking sequence, the promoter.
SEQ ID NO 62 is a partial sequence of that part of the genomic sequence of wheat (Triticum aestivum, variety ‘Chinese Spring’) that, from the start codon starting at 1374 bp to the end of the TGA stop codon at 4938 bp, includes the DNA coding sequence for Mfw4-A as well as flanking sequences upstream of the start codon and downstream of the stop codon. These flanking sequences may be expected to include regulatory sequences, such as, in the upstream flanking sequence, the promoter.
SEQ ID NO 65 is a partial sequence of that part of the genomic sequence of wheat (Triticum aestivum, variety ‘Chinese Spring’) that, from the start codon starting at 1309 bp to the end of the TGA stop codon at 4637 bp, includes the DNA coding sequence for Mfw4-B as well as flanking sequences upstream of the start codon and downstream of the stop codon. These flanking sequences may be expected to include regulatory sequences, such as, in the upstream flanking sequence, the promoter.
SEQ ID NO 68 is a partial sequence of that part of the genomic sequence of wheat (Triticum aestivum, variety ‘Chinese Spring’) that, from the start codon starting at 1309 bp to the end of the TGA stop codon at 4637 bp, includes the DNA coding sequence for Mfw4-D as well as flanking sequences upstream of the start codon and downstream of the stop codon. These flanking sequences may be expected to include regulatory sequences, such as, in the upstream flanking sequence, the promoter.
SEQ ID NO 71 is a partial sequence of that part of the genomic sequence of wheat (Triticum aestivum, variety ‘Chinese Spring’) that, from the start codon starting at 1605 bp to the end of the TGA stop codon at 3022 bp, includes the DNA coding sequence for Mfw6-A as well as flanking sequences upstream of the start codon and downstream of the stop codon. These flanking sequences may be expected to include regulatory sequences, such as, in the upstream flanking sequence, the promoter.
SEQ ID NO 74 is a partial sequence of that part of the genomic sequence of wheat (Triticum aestivum, variety ‘Chinese Spring’) that, from the start codon starting at 1560 bp to the end of the TGA stop codon at 2980 bp, includes the DNA coding sequence for Mfw6-D as well as flanking sequences upstream of the start codon and downstream of the stop codon. These flanking sequences may be expected to include regulatory sequences, such as, in the upstream flanking sequence, the promoter.
SEQ ID NO 77 is a partial sequence of that part of the genomic sequence of wheat (Triticum aestivum, variety ‘Chinese Spring’) that, from the start codon starting at 1318 bp to the end of the TGA stop codon at 3470 bp, includes the DNA coding sequence for Mfw7-A as well as flanking sequences upstream of the start codon and downstream of the stop codon. These flanking sequences may be expected to include regulatory sequences, such as, in the upstream flanking sequence, the promoter.
SEQ ID NO 80 is a partial sequence of that part of the genomic sequence of wheat (Triticum aestivum, variety ‘Chinese Spring’) that, from the start codon starting at 1229 bp to the end of the TGA stop codon at 3369 bp, includes the DNA coding sequence for Mfw7-B as well as flanking sequences upstream of the start codon and downstream of the stop codon. These flanking sequences may be expected to include regulatory sequences, such as, in the upstream flanking sequence, the promoter.
SEQ ID NO 83 is a partial sequence of that part of the genomic sequence of wheat (Triticum aestivum, variety ‘Chinese Spring’) that, from the start codon starting at 1413 bp to the end of the TGA stop codon at 3588 bp, includes the DNA coding sequence for Mfw7-D as well as flanking sequences upstream of the start codon and downstream of the stop codon. These flanking sequences may be expected to include regulatory sequences, such as, in the upstream flanking sequence, the promoter.
SEQ ID NO 86 is a partial sequence of that part of the genomic sequence of wheat (Triticum aestivum, variety ‘Chinese Spring’) that, from the start codon starting at 1340 bp to the end of the TGA stop codon at 3407 bp, includes the DNA coding sequence for Mfw8-A as well as flanking sequences upstream of the start codon and downstream of the stop codon. These flanking sequences may be expected to include regulatory sequences, such as, in the upstream flanking sequence, the promoter.
SEQ ID NO 87 is a partial sequence of that part of the genomic sequence of wheat (Triticum aestivum, variety ‘Chinese Spring’) that, from the start codon starting at 1349 bp to the end of the TGA stop codon at 3422 bp, includes the DNA coding sequence for Mfw8-B as well as flanking sequences upstream of the start codon and downstream of the stop codon. These flanking sequences may be expected to include regulatory sequences, such as, in the upstream flanking sequence, the promoter.
SEQ ID NO 92 is a partial sequence of that part of the genomic sequence of wheat (Triticum aestivum, variety ‘Chinese Spring’) that, from the start codon starting at 1331 bp to the end of the TGA stop codon at 3401 bp, includes the DNA coding sequence for Mfw8-D as well as flanking sequences upstream of the start codon and downstream of the stop codon. These flanking sequences may be expected to include regulatory sequences, such as, in the upstream flanking sequence, the promoter.
SEQ ID NO 95 is a partial sequence of that part of the genomic sequence of wheat (Triticum aestivum, variety ‘Chinese Spring’) that, from the start codon starting at 1248 bp to the end of the TGA stop codon at 2849 bp, includes the DNA coding sequence for Mfw9-A as well as flanking sequences upstream of the start codon and downstream of the stop codon. These flanking sequences may be expected to include regulatory sequences, such as, in the upstream flanking sequence, the promoter.
SEQ ID NO 98 is a partial sequence of that part of the genomic sequence of wheat (Triticum aestivum, variety ‘Chinese Spring’) that, from the start codon starting at 393 bp to the end of the TGA stop codon at 32502 bp, includes the DNA coding sequence for Mfw9-B as well as flanking sequences upstream of the start codon and downstream of the stop codon. These flanking sequences may be expected to include regulatory sequences, such as, in the upstream flanking sequence, the promoter.
SEQ ID NO 101 is a partial sequence of that part of the genomic sequence of wheat (Triticum aestivum, variety ‘Chinese Spring’) that, from the start codon starting at 1273 bp to the end of the TGA stop codon at 2831 bp, includes the DNA coding sequence for Mfw9-D as well as flanking sequences upstream of the start codon and downstream of the stop codon. These flanking sequences may be expected to include regulatory sequences, such as, in the upstream flanking sequence, the promoter.
SEQ ID NO 104 is a partial sequence of that part of the genomic sequence of wheat (Triticum aestivum, variety ‘Chinese Spring’) that, from the start codon starting at 1398 bp to the end of the TGA stop codon at 3217 bp, includes the DNA coding sequence for Mfw10-A as well as flanking sequences upstream of the start codon and downstream of the stop codon. These flanking sequences may be expected to include regulatory sequences, such as, in the upstream flanking sequence, the promoter.
SEQ ID NO 107 is a partial sequence of that part of the genomic sequence of wheat (Triticum aestivum, variety ‘Chinese Spring’) that, from the start codon starting at 1407 bp to the end of the TGA stop codon at 3217 bp, includes the DNA coding sequence for Mfw10-B as well as flanking sequences upstream of the start codon and downstream of the stop codon. These flanking sequences may be expected to include regulatory sequences, such as, in the upstream flanking sequence, the promoter.
SEQ ID NO 110 is a partial sequence of that part of the genomic sequence of wheat (Triticum aestivum, variety ‘Chinese Spring’) that, from the start codon starting at 1553 bp to the end of the TGA stop codon at 2940 bp, includes the DNA coding sequence for Mfw11-U as well as flanking sequences upstream of the start codon and downstream of the stop codon. These flanking sequences may be expected to include regulatory sequences, such as, in the upstream flanking sequence, the promoter.
SEQ ID NO 113 is a partial sequence of that part of the genomic sequence of wheat (Triticum aestivum, variety ‘Chinese Spring’) that, from the start codon starting at 1309 bp to the end of the TGA stop codon at 3246 bp, includes the DNA coding sequence for Mfw12-A as well as flanking sequences upstream of the start codon and downstream of the stop codon. These flanking sequences may be expected to include regulatory sequences, such as, in the upstream flanking sequence, the promoter.
SEQ ID NO 116 is a partial sequence of that part of the genomic sequence of wheat (Triticum aestivum, variety ‘Chinese Spring’) that, from the start codon starting at 1281 bp to the end of the TGA stop codon at 3169 bp, includes the DNA coding sequence for Mfw12-B as well as flanking sequences upstream of the start codon and downstream of the stop codon. These flanking sequences may be expected to include regulatory sequences, such as, in the upstream flanking sequence, the promoter.
SEQ ID NO 119 is a partial sequence of that part of the genomic sequence of wheat (Triticum aestivum, variety ‘Chinese Spring’) that, from the start codon starting at 1300 bp to the end of the TGA stop codon at 3086 bp, includes the DNA coding sequence for Mfw12-D as well as flanking sequences upstream of the start codon and downstream of the stop codon. These flanking sequences may be expected to include regulatory sequences, such as, in the upstream flanking sequence, the promoter.
SEQ ID NO 122 is a partial sequence of that part of the genomic sequence of wheat (Triticum aestivum, variety ‘Chinese Spring’) that, from the start codon starting at 1308 bp to the end of the TGA stop codon at 3251 bp, includes the DNA coding sequence for Mfw13-A as well as flanking sequences upstream of the start codon and downstream of the stop codon. These flanking sequences may be expected to include regulatory sequences, such as, in the upstream flanking sequence, the promoter.
SEQ ID NO 125 is a partial sequence of that part of the genomic sequence of wheat (Triticum aestivum, variety ‘Chinese Spring’) that, from the start codon starting at 1259 bp to the end of the TGA stop codon at 3233 bp, includes the DNA coding sequence for Mfw13-B as well as flanking sequences upstream of the start codon and downstream of the stop codon. These flanking sequences may be expected to include regulatory sequences, such as, in the upstream flanking sequence, the promoter.
SEQ ID NO 128 is a partial sequence of that part of the genomic sequence of wheat (Triticum aestivum, variety ‘Chinese Spring’) that, from the start codon starting at 1446 bp to the end of the TGA stop codon at 3418 bp, includes the DNA coding sequence for Mfw13-D as well as flanking sequences upstream of the start codon and downstream of the stop codon. These flanking sequences may be expected to include regulatory sequences, such as, in the upstream flanking sequence, the promoter.
SEQ ID NO: 174 is the coding sequence of PV1-A
SEQ ID NO: 175 is the polypeptide sequence of PV1-A
SEQ ID NO: 176 is the genomic sequence of PV1-A. Start codon at bases 3,142-3,144. Stop codon at bases 9,522-9,524
SEQ ID NO: 177 is the coding sequence of PV1-B
SEQ ID NO: 178 is the polypeptide sequence of PV1-B
SEQ ID NO: 179 is the genomic sequence of PV1-B. Start codon at bases 3,000-3,002. Stop codon at bases 6,086-6,088.
SEQ ID NO: 180 is the coding sequence of PV1-D
SEQ ID NO: 181 is the polypeptide sequence of PV1-D
SEQ ID NO: 182 is the genomic sequence of PV1-D. Start codon at bases 3,201-3,203. Stop codon at bases 7,078-7,080.
SEQ ID NO: 183 is the predicted coding sequence of Ms1-A
SEQ ID NO: 184 is the predicted polypeptide sequence of Ms1-A.
SEQ ID NO: 185 is the genomic sequence of Ms1-A.
SEQ ID NO: 186 is the coding sequence of Ms1-B
SEQ ID NO: 187 is the polypeptide sequence of Ms1-B.
SEQ ID NO: 188 is the genomic sequence of Ms1-B.
SEQ ID NO: 189 is the predicted coding sequence of Ms1-D
SEQ ID NO: 190 is the predicted polypeptide sequence of Ms1-D.
SEQ ID NO: 191 is the genomic sequence of Ms1-D.
SEQ ID NO: 192 is the coding sequence of Ms26-A
SEQ ID NO: 193 is the polypeptide sequence of Ms26-A.
SEQ ID NO: 194 is the genomic sequence of Ms26-A.
SEQ ID NO: 195 is the coding sequence of Ms26-B
SEQ ID NO: 196 is the polypeptide sequence of Ms26-B.
SEQ ID NO: 197 is the genomic sequence of Ms26-B.
SEQ ID NO: 198 is the coding sequence of Ms26-D.
SEQ ID NO: 199 is the polypeptide sequence of Ms26-D.
SEQ ID NO: 200 is the genomic sequence of Ms26-D.
SEQ ID NO: 201 is the coding sequence of Ms45-A.
SEQ ID NO: 202 is the polypeptide sequence of Ms45-A.
SEQ ID NO: 203 is the genomic sequence of Ms45-A.
SEQ ID NO: 204 is the coding sequence of Ms45-B.
SEQ ID NO: 205 is the polypeptide sequence of Ms45-B.
SEQ ID NO: 206 is the genomic sequence of Ms45-B.
SEQ ID NO: 207 is the coding sequence of Ms45-D.
SEQ ID NO: 208 is the polypeptide sequence of Ms45-D.
SEQ ID NO: 209 is the genomic sequence of Ms45-D.
SEQ ID NO: 214 is the Chinese Spring genomic sequence of Ms1-B.
SEQ ID NO: 215 is the Chinese Spring coding sequence of Ms1-B.
SEQ ID NO: 216 is the Chinese Spring amino acid sequence of Ms1-B.
SEQ ID NO: 217 is a guide sequence for targeting Mfw2.
SEQ ID NO: 218 is a Mfw2′.1 genomic sequence. The altered guide RNA target sequence (SEQ ID NO: 217) is found at nucleotides 2,014-2,036 of SEQ ID NO: 218.

In some embodiments, the present technology may be defined in any of the following numbered paragraphs:
1. A male-fertile maintainer plant for a male-sterile polyploid plant comprising:
   a first genome comprising:
      on a first chromosome of a pair of homologous chromosomes, at a single target locus, at least one functional ectopic allele of a MF gene and at least one functional allele of a seed endosperm color gene;
      on a second chromosome of the pair of homologous chromosomes, at the target locus corresponding to the target locus of the first chromosome of the pair of homologous chromosomes, at least one functional ectopic allele of a PV gene;
      loss-of-function alleles of the endogenous MF genes at the native MF gene loci and loss-of-function alleles of the endogenous PV genes at the native PV gene loci.
2. The male-fertile maintainer plant of paragraph 1, comprising at least one further genome, each of the further genomes comprising loss-of-function alleles of the endogenous MF genes at the native MF gene loci and loss-of-function alleles of the PV gene at the native PV gene loci.
3. The male-fertile maintainer plant of paragraph 1 or 2, wherein the target locus is the native MF gene locus.
4. The male-fertile maintainer plant of paragraph 1 or 2, wherein the target locus is the native PV gene locus.
5. The male-fertile maintainer plant of paragraph 1 or 2, wherein the target locus is not the native MF gene locus or the native PV gene locus.
6. The male-fertile maintainer plant of any one of the preceding paragraphs, wherein the ectopic allele of the MF gene and/or the ectopic allele of the PV gene is a nuclease-null allele.
7. The male-fertile maintainer plant of any one of the preceding paragraphs, wherein the ectopic allele of the MF gene and/or the ectopic allele of the PV gene is a CRISPR-null allele.
8. A male-fertile maintainer plant for a male-sterile polyploid plant comprising:
   a first genome comprising:
      on a first chromosome of a pair of homologous chromosomes, at least one functional allele of a MF gene at the MF gene locus and at least one allele of a seed endosperm color gene;
      on a second chromosome of the pair of homologous chromosomes, a loss-of-function allele of the MF gene at the MF gene locus and at least one ectopic functional allele of a PV gene;
      and loss-of-function alleles of the PV gene at the native PV gene loci; and
   at least one further genome, each of the further genomes comprising loss-of-function alleles of the MF gene at the native MF gene loci and loss-of-function alleles of the PV gene at the native PV gene loci.
9. The plant of any one of the preceding paragraphs, wherein the at least one functional allele of a MF gene is the endogenous wild-type functional allele of the MF gene.
10. The plant of any one of the preceding paragraphs, wherein the at least one functional allele of a MF gene is an ectopic copy of the MF gene.
11. The plant of paragraph 10, wherein the at least one functional allele of a MF gene and the at least one allele of a seed endosperm color gene are part of single construct.
12. The plant of any one of the preceding paragraphs, wherein an ectopic allele or ectopic copy of a gene is a nuclease-null or CRISPR-null allele.
13. The plant of any one of the preceding paragraphs, wherein the MF gene displays the same type of activity and shares at least 80%, at least 85%, at least 90%, at least 95%, or greater sequence identity with one or more of the genes of Table 1.
14. The plant of paragraph 13, wherein the MF gene is selected from Table 1.
15. The plant of any one of the preceding paragraphs, wherein the MF gene displays the same type of activity and shares at least 80%, at least 85%, at least 90%, at least 95%, or greater sequence identity with Mfw2.
16. The plant of any one of the preceding paragraphs, wherein the MF gene is Mfw2.
17. The plant of any one of the preceding paragraphs, wherein the PV gene displays the same type of activity and shares at least 80%, at least 85%, at least 90%, at least 95%, or greater sequence identity with PV1 or PV2.
18. The plant of any one of the preceding paragraphs, wherein the PV gene is PV1 or PV2.
19. The plant of any one of the preceding paragraphs, wherein the at least one allele of a seed endosperm color gene is exogenous.
20. The plant of any one of the preceding paragraphs, wherein the at least one allele of a seed endosperm color gene is blue aleurone (BA).
21. The plant of any one of the preceding paragraphs, wherein the at least one allele of a seed endosperm color gene comprises sequences obtained from a species within the same genus as the plant.
22. The plant of any one of the preceding paragraphs, wherein the at least one allele of a seed endosperm color gene is located within 10 cM of the MF gene loci.
23. The plant of any one of the preceding paragraphs, wherein the at least one allele of a seed endosperm color gene is located within 1 cM of the MF gene loci.
24. The plant of any one of the preceding paragraphs, wherein the at least one ectopic functional allele of a PV gene is located within 10 cM of the MF gene loci.
25. The plant of any one of the preceding paragraphs, wherein the at least one ectopic functional allele of a PV gene is located within 1 cM of the MF gene loci.
26. The plant of any one of the preceding paragraphs, wherein the only exogenous sequence in the genomes is the at least one allele of a seed endosperm color gene.
27. The plant of any one of the preceding paragraphs, wherein the only ectopic sequence in the genomes is the at least one ectopic functional allele of a PV gene.
28. The plant of any one of the preceding paragraphs, wherein the plant is tetraploid and the second genome comprises loss-of-function alleles of the MF gene at the native MF gene loci and loss-of-function alleles of the PV gene at the native PV gene loci.
29. The plant of any one of the preceding paragraphs, wherein the plant is hexaploid and the second and third genomes both comprise loss-of-function alleles of the MF gene at the native MF gene loci and loss-of-function alleles of the PV gene at the native PV gene loci.
30. The plant of any one of the preceding paragraphs, wherein a loss-of-function allele comprises an engineered knock-out modification.
31. The plant of any one of the preceding paragraphs, wherein a loss-of-function allele comprises an engineered excision of at least part of a coding or regulatory sequence.
32. The plant of any one of paragraphs 30-31, wherein the loss-of-function allele is engineered using a site-specific guided nuclease.
33. The plant of paragraph 32, wherein the site-specific guided nuclease is a form of CRISPR-Cas (such as CRISPR-Cas9).
34. The plant of any one of the preceding paragraphs, wherein the plant is wheat, triticale, canola/oilseed rape, indian mustard, barley, rice, oat, or rye.
35. The plant of any one of the preceding paragraphs, wherein the plant is wheat.
36. The plant of paragraph 35, wherein the at least one allele of a seed endosperm color gene comprises a sequence from T. aestivum, T durum, T. monococcum or another Triticum aestivum-crossable species.
37. The plant of any one of the preceding paragraphs, wherein the plant is hexaploid wheat or tetraploid wheat, Triticum aestivum, or Triticum durum.
38. A method of preparing a male-fertile maintainer plant for a male-sterile polyploid plant, the method comprising engineering a plant to comprise:
   in a first genome:
      on a first chromosome of a pair of homologous chromosomes, at a single target locus, at least one functional ectopic allele of a MF gene and at least one functional allele of a seed endosperm color gene;
      on a second chromosome of the pair of homologous chromosomes, at the target locus corresponding to the target locus of the first chromosome of the pair of homologous chromosomes, at least one functional ectopic allele of a PV gene; and
      loss-of-function alleles of the endogenous MF genes at the native MF gene loci and loss-of-function alleles of the endogenous PV genes at the native PV gene loci.
39. A method of preparing a male-fertile maintainer plant for a male-sterile polyploid plant, the method comprising, simultaneously or sequentially:
   inserting, on a first chromosome of a pair of homologous chromosomes in a first genome, at a single target locus, a construct comprising at least one functional ectopic allele of a MF gene and at least one functional allele of a seed endosperm color gene, optionally wherein the inserting comprises nuclease cleavage of the target locus (e.g., zinc-finger nuclease or CRISPR nuclease cleavage) and recombination or end-joining of the construct;
   inserting, on a second chromosome of the pair of homologous chromosomes in the first genome, at the target locus corresponding to the target locus of the first chromosome of the pair of homologous chromosomes, a construct comprising at least one functional ectopic allele of a PV gene, optionally wherein the inserting comprises nuclease cleavage of the target locus (e.g., zinc-finger nuclease or CRISPR nuclease cleavage) and/or recombination or end-joining of the construct; and
   mutating the the endogenous MF genes at the native MF gene loci and the endogenous PV genes at the native PV gene loci to create loss-of-function alleles, optionally wherein the loss-of-function alleles are caused by contacting the genome with a site-specific guided nuclease (e.g., CRISPR) and one or more guide RNA sequences or multi-guide constructs.
40. The method of paragraph 38 or 39, wherein the plant further comprises at least one further genome, and the method further comprises engineering loss-of-function alleles of the endogenous MF genes at the native MF gene loci and loss-of-function alleles of the PV gene at the native PV gene loci in each of the at least one further genomes.
41. The method of any one of paragraphs 38-40, wherein the target locus is the native MF gene locus.
42. The method of any one of paragraphs 38-40, wherein the target locus is the native PV gene locus.
43. The method of any one of paragraphs 38-40, wherein the target locus is not the native MF gene locus or the native PV gene locus.
44. The method of any one of the preceding paragraphs, wherein the ectopic allele of the MF gene and/or the ectopic allele of the PV gene is a nuclease-null allele.
45. The method of any of the preceding paragraphs, wherein the ectopic allele of the MF gene and/or the ectopic allele of the PV gene is a CRISPR-null allele.
46. The method of any of the preceding paragraphs, wherein the at least one functional allele of a MF gene is the endogenous wild-type functional allele of the MF gene.
47. The method of any of the preceding paragraphs, wherein the at least one functional allele of a MF gene is an ectopic copy of the MF gene.
48. The method of paragraph 46, wherein the at least one functional allele of a MF gene and the at least one allele of a seed endosperm color gene are part of single construct.
49. The method of any of the preceding paragraphs, wherein an ectopic allele or ectopic copy of a gene is a nuclease-null or CRISPR-null allele.
50. The method of any of the preceding paragraphs, wherein the MF gene displays the same type of activity and shares at least 80%, at least 85%, at least 90%, at least 95%, or greater sequence identity with one or more of the genes of Table 1.
51. The method of paragraph 50, wherein the MF gene is selected from Table 1.
52. The method of any of the preceding paragraphs, wherein the MF gene displays the same type of activity and shares at least 80%, at least 85%, at least 90%, at least 95%, or greater sequence identity with Mfw2.
53. The method of any of the preceding paragraphs, wherein the MF gene is Mfw2.
54. The method of any of the preceding paragraphs, wherein the PV gene displays the same type of activity and shares at least 80%, at least 85%, at least 90%, at least 95%, or greater sequence identity with PV1 or PV2.
55. The method of any of the preceding paragraphs, wherein the PV gene is PV1 or PV2.
56. The method of any of the preceding paragraphs, wherein the at least one allele of a seed endosperm color gene is exogenous.
57. The method of any of the preceding paragraphs, wherein the at least one allele of a seed endosperm color gene is blue aleurone (BA).
58. The method of any of the preceding paragraphs, wherein the at least one allele of a seed endosperm color gene comprises sequences obtained from a species within the same genus as the plant.
59. The method of any of the preceding paragraphs, wherein the at least one allele of a seed endosperm color gene is located within 10 cM of the MF gene loci.
60. The method of any of the preceding paragraphs, wherein the at least one allele of a seed endosperm color gene is located within 1 cM of the MF gene loci.
61. The method of any of the preceding paragraphs, wherein the at least one ectopic functional allele of a PV gene is located within 10 cM of the MF gene loci.
62. The method of any of the preceding paragraphs, wherein the at least one ectopic functional allele of a PV gene is located within 1 cM of the MF gene loci.
63. The method of any of the preceding paragraphs, wherein the only exogenous sequence in the genomes is the at least one allele of a seed endosperm color gene.
64. The method of any of the preceding paragraphs, wherein the only ectopic sequence in the genomes is the at least one ectopic functional allele of a PV gene.
65. The method of any of the preceding paragraphs, wherein the plant is tetraploid and the second genome comprises loss-of-function alleles of the MF gene at the native MF gene loci and loss-of-function alleles of the PV gene at the native PV gene loci.
66. The method of any of the preceding paragraphs, wherein the plant is hexaploid and the second and third genomes both comprise loss-of-function alleles of the MF gene at the native MF gene loci and loss-of-function alleles of the PV gene at the native PV gene loci.
67. The method of any of the preceding paragraphs, wherein a loss-of-function allele comprises an engineered knock-out modification.
68. The method of any of the preceding paragraphs, wherein a loss-of-function allele comprises an engineered excision of at least part of a coding or regulatory sequence.
69. The method of any of paragraphs 67-68, wherein the loss-of-function allele is engineered using a site-specific guided nuclease.
70. The method of paragraph 69, wherein the site-specific guided nuclease is a form of CRISPR-Cas (such as CRISPR-Cas9).
71. The method of any of the preceding paragraphs, wherein the plant is wheat, triticale, canola/oilseed rape, indian mustard, barley, rice, oat, or rye.
72. The method of any of the preceding paragraphs, wherein the plant is wheat.
73. The method of paragraph 72, wherein the at least one allele of a seed endosperm color gene comprises a sequence from T. aestivum, T durum, T. monococcum or another Triticum aestivum-crossable species.
74. The method of any of the preceding paragraphs, wherein the plant is hexaploid wheat or tetraploid wheat, Triticum aestivum, or Triticum durum.
75. The method of any of the preceding paragraphs, wherein the at least one functional ectopic allele of a MF gene and at least one functional allele of a seed endosperm color gene comprises the sequence of SEQ ID NO: 173 or a sequence with at least 80%, 85%, 90%, or 95% sequence identity thereto; and/or wherein the at least one functional ectopic allele of a PV gene comprises the sequence of SEQ ID NO: 172 or a sequence with at least 80%, 85%, 90%, or 95% sequence identity thereto.
76. The method of any of the preceding paragraphs, wherein the guide RNA sequences and/or multi-guide constructs comprise one or more of SEQ ID NOs: 22-29 or 131-156.
77. A method of providing a male sterile plant seed, the method comprising selecting, from seed produced by selfing a plant of any one of paragraphs 1-37, seed not displaying a phenotype provided by the seed endosperm gene.
78. A method of providing male sterile plant seed, the method comprising selfing a plant of any one of paragraphs 1-37, whereby the resulting seed not displaying a phenotype provided by the seed endosperm gene is the male sterile plant seed.
79. A method of providing a F1 hybrid seed for crop production, the method comprising collecting the seed produced by a male-sterile plant pollinated by a male-fertile plant, wherein the male-sterile plant is
   a) a plant grown from male sterile plant seed obtained by the method of paragraph 77 or 78; and/or
   b) comprises:
      i) loss-of-function alleles of an endogenous MF gene at each of the native MF gene loci;
      ii) loss-of-function alleles of an endogenous PV gene at each of the native PV gene loci; and
      iii) two ectopic alleles of the PV gene at a target locus.
80. A method of providing a F1 hybrid seed for crop production, the method comprising crossing a a male-sterile plant with a male-fertile plant, wherein the male-sterile plant is
   a) a plant grown from male sterile plant seed obtained by the method of paragraph 77 or 78; and/or
   b) comprises:
      i) loss-of-function alleles of an endogenous MF gene at each of the native MF gene loci;
      ii) loss-of-function alleles of an endogenous PV gene at each of the native PV gene loci; and
      iii) two ectopic alleles of the PV gene at a target locus.
81. A method of providing a F1 hybrid seed for crop production, the method comprising planting a male-sterile plant within pollination range of a male-fertile plant, wherein the male-sterile plant is
   a) a plant grown from male sterile plant seed obtained by the method of paragraph 77 or 78; and/or
   b) comprises:
      i) loss-of-function alleles of an endogenous MF gene at each of the native MF gene loci;
      ii) loss-of-function alleles of an endogenous PV gene at each of the native PV gene loci; and
      iii) two ectopic alleles of the PV gene at a target locus; and whereby the male-fertile plant pollinates the male-sterile plant and F1 hybrid seed is produced.
82. The method of any of paragraphs 79-81, wherein the pollination range is 200 metres.
83. The method of any of paragraphs 79-82, wherein the male-sterile plant and male fertile plant are different lines.
84. A method of producing a plant crop, the method comprising:
   a) planting and/or harvesting a plant or portion thereof, wherein the plant:
      i) is plant grown from F1 hybrid seed obtained by the method of any of paragraphs 79-83; and/or
      ii) comprises:
         1) in each genome of the plant, at a native MF gene locus, one functional endogenous allele of the endogenous MF gene and one loss-of-function allele of the endogenous MF gene;
         2) in each genome of the plant, at a native PV gene locus, one functional endogenous allele of the endogenous PV gene and one loss-of-function allele of the endogenous PV gene;
         3) one ectopic allele of the PV gene at a target locus.

In some embodiments, the present technology may be defined in any of the following numbered paragraphs:
1. A male-fertile maintainer plant for a male-sterile polyploid plant comprising:
   a first genome comprising:
      on a first chromosome of a pair of homologous chromosomes, at a single target locus, at least one functional ectopic allele of a MF gene and at least one functional ectopic allele of a seed color gene (e.g., seed coat and/or seed endosperm gene) (or at least one functional ectopic allele of each member of a set of seed color genes);
      on a second chromosome of the pair of homologous chromosomes, at the target locus corresponding to the target locus of the first chromosome of the pair of homologous chromosomes, at least one functional ectopic allele of a PV gene;
      loss-of-function alleles of the endogenous MF genes at the native MF gene loci and loss-of-function alleles of the endogenous PV genes at the native PV gene loci.
2. The male-fertile maintainer plant of paragraph 1, comprising at least one further genome, each of the further genomes comprising loss-of-function alleles of the endogenous MF genes at the native MF gene loci and loss-of-function alleles of the PV gene at the native PV gene loci.
3. The male-fertile maintainer plant of paragraph 1 or 2, wherein the target locus is the native MF gene locus.
4. The male-fertile maintainer plant of paragraph 1 or 2, wherein the target locus is the native PV gene locus.
5. The male-fertile maintainer plant of paragraph 1 or 2, wherein the target locus is not the native MF gene locus or the native PV gene locus.
6. The male-fertile maintainer plant of any one of the preceding paragraphs, wherein the ectopic allele of the MF gene and/or the ectopic allele of the PV gene is a nuclease-null allele.
7. The male-fertile maintainer plant of any one of the preceding paragraphs, wherein the ectopic allele of the MF gene and/or the ectopic allele of the PV gene is a CRISPR-null allele.
8. A male-fertile maintainer plant for a male-sterile polyploid plant comprising:
   a first genome comprising:
      on a first chromosome of a pair of homologous chromosomes, at least one functional allele of a MF gene at the MF gene locus and at least one allele of a seed color gene (e.g., seed coat and/or seed endosperm gene) (or at least one functional ectopic allele of each member of a set of seed color genes);
      on a second chromosome of the pair of homologous chromosomes, a loss-of-function allele of the MF gene at the MF gene locus and at least one ectopic functional allele of a PV gene;
      and loss-of-function alleles of the PV gene at the native PV gene loci; and
   at least one further genome, each of the further genomes comprising loss-of-function alleles of the MF gene at the native MF gene loci and loss-of-function alleles of the PV gene at the native PV gene loci.
9. The plant of any one of the preceding paragraphs, wherein the at least one functional allele of a MF gene is the endogenous wild-type functional allele of the MF gene.
10. The plant of any one of the preceding paragraphs, wherein the at least one functional allele of a MF gene is an ectopic copy of the MF gene.
11. The plant of paragraph 10, wherein the at least one functional allele of a MF gene and the at least one allele of a seed color gene (e.g., seed coat and/or seed endosperm gene) (or at least one functional ectopic allele of each member of a set of seed color genes) are part of single construct.
12. The plant of any one of the preceding paragraphs, wherein an ectopic allele or ectopic copy of a gene is a nuclease-null or CRISPR-null allele.
13. The plant of any one of the preceding paragraphs, wherein the MF gene displays the same type of activity and shares at least 80%, at least 85%, at least 90%, at least 95%, or greater sequence identity with one or more of the genes of Table 1.
14. The plant of paragraph 13, wherein the MF gene is selected from Table 1.
15. The plant of any one of the preceding paragraphs, wherein the MF gene displays the same type of activity and shares at least 80%, at least 85%, at least 90%, at least 95%, or greater sequence identity with Mfw2.
16. The plant of any one of the preceding paragraphs, wherein the MF gene is Mfw2.
17. The plant of any one of the preceding paragraphs, wherein the PV gene displays the same type of activity and shares at least 80%, at least 85%, at least 90%, at least 95%, or greater sequence identity with one or more of the genes of Table 2.
18. The plant of paragraph 17, wherein the PV gene is selected from Table 2.
19. The plant of any one of the preceding paragraphs, wherein the PV gene displays the same type of activity and shares at least 80%, at least 85%, at least 90%, at least 95%, or greater sequence identity with PV1 or PV2.
20. The plant of any one of the preceding paragraphs, wherein the PV gene is PV1 or PV2.
21. The plant of any one of the preceding paragraphs, wherein the PV gene displays the same type of activity and shares at least 80%, at least 85%, at least 90%, at least 95%, or greater sequence identity with Ms1.
22. The plant of any one of the preceding paragraphs, wherein the PV gene is Ms1.
23. The plant of any one of the preceding paragraphs, wherein the PV gene displays the same type of activity and shares at least 80%, at least 85%, at least 90%, at least 95%, or greater sequence identity with PV3.
24. The plant of any one of the preceding paragraphs, wherein the PV gene is PV3.
25. The plant of any one of the preceding paragraphs, wherein the MF gene displays the same type of activity and shares at least 80%, at least 85%, at least 90%, at least 95%, or greater sequence identity with Mfw2 and the PV gene displays the same type of activity and shares at least 80%, at least 85%, at least 90%, at least 95%, or greater sequence identity with Ms1.
26. The plant of any one of the preceding paragraphs, wherein the MF gene is Mfw2 and the PV gene is Ms1.
27. The plant of any one of the preceding paragraphs, wherein the MF gene displays the same type of activity and shares at least 80%, at least 85%, at least 90%, at least 95%, or greater sequence identity with Mfw2 and the PV gene displays the same type of activity and shares at least 80%, at least 85%, at least 90%, at least 95%, or greater sequence identity with PV1.
28. The plant of any one of the preceding paragraphs, wherein the MF gene is Mfw2 and the PV gene is PV1.
29. The plant of any one of the preceding paragraphs, wherein the MF gene displays the same type of activity and shares at least 80%, at least 85%, at least 90%, at least 95%, or greater sequence identity with Mfw2 and the PV gene displays the same type of activity and shares at least 80%, at least 85%, at least 90%, at least 95%, or greater sequence identity with PV3.
30. The plant of any one of the preceding paragraphs, wherein the MF gene is Mfw2 and the PV gene is PV3.
31. The plant of any one of the preceding paragraphs, wherein the at least one allele of a seed color gene (e.g., seed coat and/or seed endosperm gene) (or at least one functional ectopic allele of each member of a set of seed color genes) is exogenous.
32. The plant of any one of the preceding paragraphs, wherein the at least one allele of a seed color gene (e.g., seed coat and/or seed endosperm gene) is blue aleurone (BA).
33. The plant of any one of the preceding paragraphs, wherein the at least one allele of a seed color gene (e.g., seed coat and/or seed endosperm gene) (or at least one functional ectopic allele of each member of a set of seed color genes) comprises sequences obtained from a species within the same genus as the plant.
34. The plant of any one of the preceding paragraphs, wherein the at least one allele of a seed color gene (e.g., seed coat and/or seed endosperm gene) (or at least one functional ectopic allele of each member of a set of seed color genes) is located within 10 cM of the MF gene loci.
35. The plant of any one of the preceding paragraphs, wherein the at least one allele of a seed color gene (e.g., seed coat and/or seed endosperm gene) (or at least one functional ectopic allele of each member of a set of seed color genes) is located within 1 cM of the MF gene loci.
36. The plant of any one of the preceding paragraphs, wherein the at least one ectopic functional allele of a PV gene is located within 10 cM of the MF gene loci.
37. The plant of any one of the preceding paragraphs, wherein the at least one ectopic functional allele of a PV gene is located within 1 cM of the MF gene loci.
38. The plant of any one of the preceding paragraphs, wherein the only exogenous sequence in the genomes is the at least one allele of a seed color gene (e.g., seed coat and/or seed endosperm gene) (or at least one functional ectopic allele of each member of a set of seed color genes).
39. The plant of any one of the preceding paragraphs, wherein the only ectopic sequence in the genomes is the at least one ectopic functional allele of a PV gene.
40. The plant of any one of the preceding paragraphs, wherein the plant is tetraploid and the second genome comprises loss-of-function alleles of the MF gene at the native MF gene loci and loss-of-function alleles of the PV gene at the native PV gene loci.
41. The plant of any one of the preceding paragraphs, wherein the plant is hexaploid and the second and third genomes both comprise loss-of-function alleles of the MF gene at the native MF gene loci and loss-of-function alleles of the PV gene at the native PV gene loci.
42. The plant of any one of the preceding paragraphs, wherein a loss-of-function allele comprises an engineered knock-out modification.
43. The plant of any one of the preceding paragraphs, wherein a loss-of-function allele comprises an engineered excision of at least part of a coding or regulatory sequence.
44. The plant of any one of paragraphs 42-43, wherein the loss-of-function allele is engineered using a site-specific guided nuclease.
45. The plant of paragraph 44, wherein the site-specific guided nuclease is a form of CRISPR-Cas (such as CRISPR-Cas9).
46. The plant of any one of the preceding paragraphs, wherein the plant is wheat, triticale, canola/oilseed rape, indian mustard, barley, rice, oat, or rye.
47. The plant of any one of the preceding paragraphs, wherein the plant is wheat.
48. The plant of paragraph 41, wherein the at least one allele of a seed color gene (e.g., seed coat and/or seed endosperm gene) (or at least one functional ectopic allele of each member of a set of seed color genes) comprises a sequence from T. aestivum, T durum, T. monococcum or another Triticum aestivum-crossable species.
49. The plant of any one of the preceding paragraphs, wherein the plant is hexaploid wheat or tetraploid wheat, Triticum aestivum, or Triticum durum.
50. A method of preparing a male-fertile maintainer plant for a male-sterile polyploid plant, the method comprising engineering a plant to comprise:
   in a first genome:
      on a first chromosome of a pair of homologous chromosomes, at a single target locus, at least one functional ectopic allele of a MF gene and at least one functional ectopic allele of a seed color gene (e.g., seed coat and/or seed endosperm gene) (or at least one functional ectopic allele of each member of a set of seed color genes);
      on a second chromosome of the pair of homologous chromosomes, at the target locus corresponding to the target locus of the first chromosome of the pair of homologous chromosomes, at least one functional ectopic allele of a PV gene; and
      loss-of-function alleles of the endogenous MF genes at the native MF gene loci and loss-of-function alleles of the endogenous PV genes at the native PV gene loci.
51. A method of preparing a male-fertile maintainer plant for a male-sterile polyploid plant, the method comprising, simultaneously or sequentially:
   inserting, on a first chromosome of a pair of homologous chromosomes in a first genome, at a single target locus, a construct comprising at least one functional ectopic allele of a MF gene and at least one functional ectopic allele of a seed color gene (e.g., seed coat and/or seed endosperm gene) (or at least one functional ectopic allele of each member of a set of seed color genes), optionally wherein the inserting comprises nuclease cleavage of the target locus (e.g., zinc-finger nuclease or CRISPR nuclease cleavage) and recombination or end-joining of the construct;
   inserting, on a second chromosome of the pair of homologous chromosomes in the first genome, at the target locus corresponding to the target locus of the first chromosome of the pair of homologous chromosomes, a construct comprising at least one functional ectopic allele of a PV gene, optionally wherein the inserting comprises nuclease cleavage of the target locus (e.g., zinc-finger nuclease or CRISPR nuclease cleavage) and/or recombination or end-joining of the construct; and
   mutating the the endogenous MF genes at the native MF gene loci and the endogenous PV genes at the native PV gene loci to create loss-of-function alleles, optionally wherein the loss-of-function alleles are caused by contacting the genome with a site-specific guided nuclease (e.g., CRISPR) and one or more guide RNA sequences or multi-guide constructs.
52. A method of preparing a male-fertile maintainer plant for a male-sterile polyploid plant, the method comprising:
   i) inserting, on a first chromosome of a pair of homologous chromosomes in a first genome, at a single target locus, a cassette comprising in 5′ to 3′ or 3′ to 5′ order:
      a first recognition site for a first recombinase;
      at least one functional ectopic nuclease null allele of a MF gene and at least one functional ectopic allele of a seed color gene (e.g., seed coat and/or seed endosperm gene) (or at least one functional ectopic allele of each member of a set of seed color genes) in either relative order;
      a first recognition site for a second recombinase;
      a selection gene;
      a second recognition site for the first recombinase;
      at least one functional ectopic nuclease-null allele of a PV gene;
      a second recognition site for the second recombinase;
   thereby providing a full-cassette insertion plant;
   ii) contacting a first progeny of the full-cassette insertion plant, or a cell thereof, with the first recombinase,
   thereby excising:
      one recognition site for the first recombinase, the at least one functional ectopic nuclease null allele of a MF gene and at least one functional ectopic allele of a seed color gene (e.g., seed coat and/or seed endosperm gene) (or at least one functional ectopic allele of each member of a set of seed color genes), the first recognition site for the second recombinase, and the selection gene from the genome of the first progeny and
   thereby providing an excised first progeny comprising:
      one recognition site for the first recombinase, the at least one functional ectopic nuclease-null allele of a PV gene, and the second recognition site for the second recombinase portions of the construct;
   iii) contacting a second progeny of the full-cassette insertion plant, or a cell thereof, with the second recombinase, thereby excising:
      one recognition site for the second recombinase, the selection gene, the second recognition site for the first recombinase and at least one functional ectopic nuclease-null allele of a PV gene, and
   thereby providing an excised second progeny comprising:
      one recognition site for the second recombinase, the first recognition site for the first recombinase, and the at least one functional ectopic nuclease null allele of a MF gene and at least one functional ectopic allele of a seed color gene (e.g., seed coat and/or seed endosperm gene) (or at least one functional ectopic allele of each member of a set of seed color genes) portions of the construct;
   iv) crossing the excised first progeny provided in step ii) and the excised second progeny provided in step iii), thereby providing a third progeny comprising, in a first genome,
      on a first chromosome of a pair of homologous chromosomes, at a single target locus, the at least one functional ectopic nuclease-null allele of a MF gene and the at least one functional ectopic allele of a seed color gene (e.g., seed coat and/or seed endosperm gene) (or at least one functional ectopic allele of each member of a set of seed color genes);
      on a second chromosome of the pair of homologous chromosomes, at the target locus corresponding to the target locus of the first chromosome of the pair of homologous chromosomes, the at least one functional ectopic nuclease-null allele of a PV gene; and
   v) mutating the the endogenous MF genes at the native MF gene loci and the endogenous PV genes at the native PV gene loci to create loss-of-function alleles, optionally wherein the loss-of-function alleles are caused by contacting the genome with a site-specific guided nuclease (e.g., CRISPR) and one or more guide RNA sequences or multi-guide constructs, thereby providing the male-fertile maintainer plant.
53. The method of paragraph 52, wherein one of first recombinase and second recombinase is Cre and the other recombinase is Flp.
54. The method of any one of paragraphs 52-53, wherein the construct is a T-DNA construct.
55. The method of any one of paragraphs 52-54, wherein one or more of the steps further comprise selection of the provided plants or cells, optionally wherein the selection is PCR selection.
56. The method of any one of paragraphs 52-55, wherein the plant further comprises at least one further genome, and the method further comprises engineering loss-of-function alleles of the endogenous MF genes at the native MF gene loci and loss-of-function alleles of the PV gene at the native PV gene loci in each of the at least one further genomes.
57. The method of any one of paragraphs 52-56, wherein the target locus is the native MF gene locus.
58. The method of any one of paragraphs 52-57, wherein the target locus is the native PV gene locus.
59. The method of any one of paragraphs 52-56, wherein the target locus is not the native MF gene locus or the native PV gene locus.
60. The method of any one of the preceding paragraphs, wherein the ectopic allele of the MF gene and/or the ectopic allele of the PV gene is a nuclease-null allele.
61. The method of any of the preceding paragraphs, wherein the ectopic allele of the MF gene and/or the ectopic allele of the PV gene is a CRISPR-null allele.
62. The method of any of the preceding paragraphs, wherein the at least one functional allele of a MF gene is the endogenous wild-type functional allele of the MF gene.
63. The method of any of the preceding paragraphs, wherein the at least one functional allele of a MF gene is an ectopic copy of the MF gene.
64. The method of any of the preceding paragraphs, wherein the at least one functional allele of a MF gene and the at least one allele of a seed color gene (e.g., seed coat and/or seed endosperm gene) (or at least one functional ectopic allele of each member of a set of seed color genes) are part of single construct.
65. The method of any of the preceding paragraphs, wherein an ectopic allele or ectopic copy of a gene is a nuclease-null or CRISPR-null allele.
66. The method of any of the preceding paragraphs, wherein the MF gene displays the same type of activity and shares at least 80%, at least 85%, at least 90%, at least 95%, or greater sequence identity with one or more of the genes of Table 1.
67. The method of paragraph 66, wherein the MF gene is selected from Table 1.
68. The method of any of the preceding paragraphs, wherein the MF gene displays the same type of activity and shares at least 80%, at least 85%, at least 90%, at least 95%, or greater sequence identity with Mfw2.
69. The method of any of the preceding paragraphs, wherein the MF gene is Mfw2.
70. The method of any one of the preceding paragraphs, wherein the PV gene displays the same type of activity and shares at least 80%, at least 85%, at least 90%, at least 95%, or greater sequence identity with one or more of the genes of Table 2.
71. The method of paragraph 70, wherein the PV gene is selected from Table 2.
72. The method of any of the preceding paragraphs, wherein the PV gene displays the same type of activity and shares at least 80%, at least 85%, at least 90%, at least 95%, or greater sequence identity with PV1 or PV2.
73. The method of any of the preceding paragraphs, wherein the PV gene is PV1 or PV2.
74. The method of any one of the preceding paragraphs, wherein the PV gene displays the same type of activity and shares at least 80%, at least 85%, at least 90%, at least 95%, or greater sequence identity with Ms1.
75. The method of any one of the preceding paragraphs, wherein the PV gene is Ms1.
76. The method of any one of the preceding paragraphs, wherein the PV gene displays the same type of activity and shares at least 80%, at least 85%, at least 90%, at least 95%, or greater sequence identity with PV3.
77. The method of any one of the preceding paragraphs, wherein the PV gene is PV3.
78. The method of any one of the preceding paragraphs, wherein the MF gene displays the same type of activity and shares at least 80%, at least 85%, at least 90%, at least 95%, or greater sequence identity with Mfw2 and the PV gene displays the same type of activity and shares at least 80%, at least 85%, at least 90%, at least 95%, or greater sequence identity with Ms1.
79. The method of any one of the preceding paragraphs, wherein the MF gene is Mfw2 and the PV gene is Ms1.
80. The method of any one of the preceding paragraphs, wherein the MF gene displays the same type of activity and shares at least 80%, at least 85%, at least 90%, at least 95%, or greater sequence identity with Mfw2 and the PV gene displays the same type of activity and shares at least 80%, at least 85%, at least 90%, at least 95%, or greater sequence identity with PV1.
81. The method of any one of the preceding paragraphs, wherein the MF gene is Mfw2 and the PV gene is PV1.
82. The method of any one of the preceding paragraphs, wherein the MF gene displays the same type of activity and shares at least 80%, at least 85%, at least 90%, at least 95%, or greater sequence identity with Mfw2 and the PV gene displays the same type of activity and shares at least 80%, at least 85%, at least 90%, at least 95%, or greater sequence identity with PV3.
83. The method of any one of the preceding paragraphs, wherein the MF gene is Mfw2 and the PV gene is PV3.
84. The method of any of the preceding paragraphs, wherein the at least one allele of a seed color gene (e.g., seed coat and/or seed endosperm gene) (or at least one functional ectopic allele of each member of a set of seed color genes) is exogenous.
85. The method of any of the preceding paragraphs, wherein the at least one allele of a seed color gene (e.g., seed coat and/or seed endosperm gene) is blue aleurone (BA).
86. The method of any of the preceding paragraphs, wherein the at least one allele of a seed color gene (e.g., seed coat and/or seed endosperm gene) (or at least one functional ectopic allele of each member of a set of seed color genes) comprises sequences obtained from a species within the same genus as the plant.
87. The method of any of the preceding paragraphs, wherein the at least one allele of a seed color gene (e.g., seed coat and/or seed endosperm gene) (or at least one functional ectopic allele of each member of a set of seed color genes) is located within 10 cM of the MF gene loci.
88. The method of any of the preceding paragraphs, wherein the at least one allele of a seed color gene (e.g., seed coat and/or seed endosperm gene) (or at least one functional ectopic allele of each member of a set of seed color genes) is located within 1 cM of the MF gene loci.
89. The method of any of the preceding paragraphs, wherein the at least one ectopic functional allele of a PV gene is located within 10 cM of the MF gene loci.
90. The method of any of the preceding paragraphs, wherein the at least one ectopic functional allele of a PV gene is located within 1 cM of the MF gene loci.
91. The method of any of the preceding paragraphs, wherein the only exogenous sequence in the genomes is the at least one allele of a seed color gene (e.g., seed coat and/or seed endosperm gene) (or at least one functional ectopic allele of each member of a set of seed color genes).
92. The method of any of the preceding paragraphs, wherein the only ectopic sequence in the genomes is the at least one ectopic functional allele of a PV gene.
93. The method of any of the preceding paragraphs, wherein the plant is tetraploid and the second genome comprises loss-of-function alleles of the MF gene at the native MF gene loci and loss-of-function alleles of the PV gene at the native PV gene loci.
94. The method of any of the preceding paragraphs, wherein the plant is hexaploid and the second and third genomes both comprise loss-of-function alleles of the MF gene at the native MF gene loci and loss-of-function alleles of the PV gene at the native PV gene loci.
95. The method of any of the preceding paragraphs, wherein a loss-of-function allele comprises an engineered knock-out modification.
96. The method of any of the preceding paragraphs, wherein a loss-of-function allele comprises an engineered excision of at least part of a coding or regulatory sequence.
97. The method of any of paragraphs 95-96, wherein the loss-of-function allele is engineered using a site-specific guided nuclease.
98. The method of paragraph 97, wherein the site-specific guided nuclease is a form of CRISPR-Cas (such as CRISPR-Cas9).
99. The method of any of the preceding paragraphs, wherein the plant is wheat, triticale, canola/oilseed rape, indian mustard, barley, rice, oat, or rye.
100. The method of any of the preceding paragraphs, wherein the plant is wheat.
101. The method of paragraph 100, wherein the at least one allele of a seed color gene (e.g., seed coat and/or seed endosperm gene) (or at least one functional ectopic allele of each member of a set of seed color genes) comprises a sequence from T. aestivum, T durum, T. monococcum or another Triticum aestivum-crossable species.
102. The method of any of the preceding paragraphs, wherein the plant is hexaploid wheat or tetraploid wheat, Triticum aestivum, or Triticum durum.
103. The method of any of the preceding paragraphs, wherein the at least one functional ectopic allele of a MF gene and at least one functional ectopic allele of a seed color gene (e.g., seed coat and/or seed endosperm gene) (or at least one functional ectopic allele of each member of a set of seed color genes) comprises the sequence of SEQ ID NO: 173 or a sequence with at least 80%, 85%, 90%, or 95% sequence identity thereto; and/or wherein the at least one functional ectopic allele of a PV gene comprises the sequence of SEQ ID NO: 172 or 218 or a sequence with at least 80%, 85%, 90%, or 95% sequence identity thereto.
104. The method of any of the preceding paragraphs, wherein the guide RNA sequences and/or multi-guide constructs comprise one or more of SEQ ID NOs: 22-29, 131-154, 156, 210-213, or 217.
105. A method of providing a male sterile plant seed, the method comprising selecting, from seed produced by selfing a plant of any one of paragraphs 1-49, seed not displaying a phenotype provided by the seed endosperm gene.
106. A method of providing male sterile plant seed, the method comprising selfing a plant of any one of paragraphs 1-49, whereby the resulting seed not displaying a phenotype provided by the seed endosperm gene is the male sterile plant seed.
107. A method of providing a F1 hybrid seed for crop production, the method comprising collecting the seed produced by a male-sterile plant pollinated by a male-fertile plant, wherein the male-sterile plant is
   a) a plant grown from male sterile plant seed obtained by the method of paragraph 105 or 106; and/or
   b) comprises:
      i) loss-of-function alleles of an endogenous MF gene at each of the native MF gene loci;
      ii) loss-of-function alleles of an endogenous PV gene at each of the native PV gene loci; and
      iii) two ectopic alleles of the PV gene at a target locus.
108. A method of providing a F1 hybrid seed for crop production, the method comprising crossing a a male-sterile plant with a male-fertile plant, wherein the male-sterile plant is
a) a plant grown from male sterile plant seed obtained by the method of paragraph 105 or 106; and/or
b) comprises:
   i) loss-of-function alleles of an endogenous MF gene at each of the native MF gene loci;
   ii) loss-of-function alleles of an endogenous PV gene at each of the native PV gene loci; and
   iii) two ectopic alleles of the PV gene at a target locus.
109. A method of providing a F1 hybrid seed for crop production, the method comprising planting a male-sterile plant within pollination range of a male-fertile plant, wherein the male-sterile plant is
   a) a plant grown from male sterile plant seed obtained by the method of paragraph 105 or 106; and/or
   b) comprises:
      i) loss-of-function alleles of an endogenous MF gene at each of the native MF gene loci;
      ii) loss-of-function alleles of an endogenous PV gene at each of the native PV gene loci; and
      iii) two ectopic alleles of the PV gene at a target locus; and
   whereby the male-fertile plant pollinates the male-sterile plant and F1 hybrid seed is produced.
110. The method of paragraph 105-109, wherein the pollination range is 200 metres.
111. The method of any of paragraphs 105-110, wherein the male-sterile plant and male fertile plant are different lines.
112. A method of producing a plant crop, the method comprising:
   a) planting and/or harvesting a plant or portion thereof, wherein the plant:
      i) is plant grown from F1 hybrid seed obtained by the method of any of paragraphs 107-111; and/or
      ii) comprises:
         1) in each genome of the plant, at a native MF gene locus, one functional endogenous allele of the endogenous MF gene and one loss-of-function allele of the endogenous MF gene;
         2) in each genome of the plant, at a native PV gene locus, one functional endogenous allele of the endogenous PV gene and one loss-of-function allele of the endogenous PV gene;
         3) one ectopic allele of the PV gene at a target locus.

In some embodiments, the present technology may be defined in any of the following numbered paragraphs:
1. A male-fertile maintainer plant for a male-sterile polyploid plant comprising:
   a first genome comprising:
      on a first chromosome of a pair of homologous chromosomes, at a single target locus, at least one functional ectopic allele of a MF gene and at least one functional ectopic allele of a seed color gene (e.g., seed coat and/or seed endosperm gene) (or at least one functional ectopic allele of each member of a set of seed color genes);
      on a second chromosome of the pair of homologous chromosomes, at the target locus corresponding to the target locus of the first chromosome of the pair of homologous chromosomes, at least one functional ectopic allele of a PV gene;
      loss-of-function alleles of the endogenous MF genes at the native MF gene loci and loss-of-function alleles of the endogenous PV genes at the native PV gene loci.
2. The male-fertile maintainer plant of paragraph 1, comprising at least one further genome, each of the further genomes comprising loss-of-function alleles of the endogenous MF genes at the native MF gene loci and loss-of-function alleles of the PV gene at the native PV gene loci.
3. The male-fertile maintainer plant of paragraph 1 or 2, wherein the target locus is the native MF gene locus.
4. The male-fertile maintainer plant of paragraph 1 or 2, wherein the target locus is the native PV gene locus.
5. The male-fertile maintainer plant of paragraph 1 or 2, wherein the target locus is not the native MF gene locus or the native PV gene locus.
6. The male-fertile maintainer plant of any one of the preceding paragraphs, wherein the ectopic allele of the MF gene and/or the ectopic allele of the PV gene is a nuclease-null allele.
7. The male-fertile maintainer plant of any one of the preceding paragraphs, wherein the ectopic allele of the MF gene and/or the ectopic allele of the PV gene is a CRISPR-null allele.
8. A male-fertile maintainer plant for a male-sterile polyploid plant comprising: a first genome comprising:
   on a first chromosome of a pair of homologous chromosomes, at least one functional allele of a MF gene at the MF gene locus and at least one allele of a seed color gene (e.g., seed coat and/or seed endosperm gene) (or at least one functional ectopic allele of each member of a set of seed color genes);
   on a second chromosome of the pair of homologous chromosomes, a loss-of-function allele of the MF gene at the MF gene locus and at least one ectopic functional allele of a PV gene;
   and loss-of-function alleles of the PV gene at the native PV gene loci; and
at least one further genome, each of the further genomes comprising loss-of-function alleles of the MF gene at the native MF gene loci and loss-of-function alleles of the PV gene at the native PV gene loci.
9. The plant of any one of the preceding paragraphs, wherein the at least one functional allele of a MF gene is the endogenous wild-type functional allele of the MF gene.
10. The plant of any one of the preceding paragraphs, wherein the at least one functional allele of a MF gene is an ectopic copy of the MF gene.
11. The plant of paragraph 10, wherein the at least one functional allele of a MF gene and the at least one allele of a seed color gene (e.g., seed coat and/or seed endosperm gene) (or at least one functional ectopic allele of each member of a set of seed color genes) are part of single construct.
12. The plant of any one of the preceding paragraphs, wherein an ectopic allele or ectopic copy of a gene is a nuclease-null or CRISPR-null allele.
13. The plant of any one of the preceding paragraphs, wherein the MF gene displays the same type of activity and shares at least 80%, at least 85%, at least 90%, at least 95%, or greater sequence identity with one or more of the genes of Table 1.
14. The plant of paragraph 13, wherein the MF gene is selected from Table 1.
15. The plant of any one of the preceding paragraphs, wherein the MF gene displays the same type of activity and shares at least 80%, at least 85%, at least 90%, at least 95%, or greater sequence identity with Mfw2.
16. The plant of any one of the preceding paragraphs, wherein the MF gene is Mfw2.
17. The plant of any one of the preceding paragraphs, wherein the PV gene displays the same type of activity and shares at least 80%, at least 85%, at least 90%, at least 95%, or greater sequence identity with one or more of the genes of Table 2.
18. The plant of paragraph 17, wherein the PV gene is selected from Table 2.
19. The plant of any one of the preceding paragraphs, wherein the PV gene displays the same type of activity and shares at least 80%, at least 85%, at least 90%, at least 95%, or greater sequence identity with PV1 or PV2.
20. The plant of any one of the preceding paragraphs, wherein the PV gene is PV1 or PV2.
21. The plant of any one of the preceding paragraphs, wherein the PV gene displays the same type of activity and shares at least 80%, at least 85%, at least 90%, at least 95%, or greater sequence identity with Ms1.
22. The plant of any one of the preceding paragraphs, wherein the PV gene is Ms1.
23. The plant of any one of the preceding paragraphs, wherein the PV gene displays the same type of activity and shares at least 80%, at least 85%, at least 90%, at least 95%, or greater sequence identity with PV3.
24. The plant of any one of the preceding paragraphs, wherein the PV gene is PV3.
25. The plant of any one of the preceding paragraphs, wherein the MF gene displays the same type of activity and shares at least 80%, at least 85%, at least 90%, at least 95%, or greater sequence identity with Mfw2 and the PV gene displays the same type of activity and shares at least 80%, at least 85%, at least 90%, at least 95%, or greater sequence identity with Ms1.
26. The plant of any one of the preceding paragraphs, wherein the MF gene is Mfw2 and the PV gene is Ms1.
27. The plant of any one of the preceding paragraphs, wherein the MF gene displays the same type of activity and shares at least 80%, at least 85%, at least 90%, at least 95%, or greater sequence identity with Mfw2 and the PV gene displays the same type of activity and shares at least 80%, at least 85%, at least 90%, at least 95%, or greater sequence identity with PV1.
28. The plant of any one of the preceding paragraphs, wherein the MF gene is Mfw2 and the PV gene is PV1.
29. The plant of any one of the preceding paragraphs, wherein the MF gene displays the same type of activity and shares at least 80%, at least 85%, at least 90%, at least 95%, or greater sequence identity with Mfw2 and the PV gene displays the same type of activity and shares at least 80%, at least 85%, at least 90%, at least 95%, or greater sequence identity with PV3.
30. The plant of any one of the preceding paragraphs, wherein the MF gene is Mfw2 and the PV gene is PV3.
31. The plant of any one of the preceding paragraphs, wherein the at least one allele of a seed color gene (e.g., seed coat and/or seed endosperm gene) (or at least one functional ectopic allele of each member of a set of seed color genes) is exogenous.
32. The plant of any one of the preceding paragraphs, wherein the at least one allele of a seed color gene (e.g., seed coat and/or seed endosperm gene) is blue aleurone (BA).
33. The plant of any one of the preceding paragraphs, wherein the at least one allele of a seed color gene (e.g., seed coat and/or seed endosperm gene) (or at least one functional ectopic allele of each member of a set of seed color genes) comprises sequences obtained from a species within the same genus as the plant.
34. The plant of any one of the preceding paragraphs, wherein the at least one allele of a seed color gene (e.g., seed coat and/or seed endosperm gene) (or at least one functional ectopic allele of each member of a set of seed color genes) is located within 10 cM of the MF gene loci.
35. The plant of any one of the preceding paragraphs, wherein the at least one allele of a seed color gene (e.g., seed coat and/or seed endosperm gene) (or at least one functional ectopic allele of each member of a set of seed color genes) is located within 1 cM of the MF gene loci.
36. The plant of any one of the preceding paragraphs, wherein the at least one ectopic functional allele of a PV gene is located within 10 cM of the MF gene loci.
37. The plant of any one of the preceding paragraphs, wherein the at least one ectopic functional allele of a PV gene is located within 1 cM of the MF gene loci.
38. The plant of any one of the preceding paragraphs, wherein the only exogenous sequence in the genomes is the at least one allele of a seed color gene (e.g., seed coat and/or seed endosperm gene) (or at least one functional ectopic allele of each member of a set of seed color genes).
39. The plant of any one of the preceding paragraphs, wherein the only ectopic sequence in the genomes is the at least one ectopic functional allele of a PV gene.
40. The plant of any one of the preceding paragraphs, wherein the plant is tetraploid and the second genome comprises loss-of-function alleles of the MF gene at the native MF gene loci and loss-of-function alleles of the PV gene at the native PV gene loci.
41. The plant of any one of the preceding paragraphs, wherein the plant is hexaploid and the second and third genomes both comprise loss-of-function alleles of the MF gene at the native MF gene loci and loss-of-function alleles of the PV gene at the native PV gene loci.
42. The plant of any one of the preceding paragraphs, wherein a loss-of-function allele comprises an engineered knock-out modification.
43. The plant of any one of the preceding paragraphs, wherein a loss-of-function allele comprises an engineered excision of at least part of a coding or regulatory sequence.
44. The plant of any one of paragraphs 42-43, wherein the loss-of-function allele is engineered using a site-specific guided nuclease.
45. The plant of paragraph 44, wherein the site-specific guided nuclease is a form of CRISPR-Cas (such as CRISPR-Cas9).
46. The plant of any one of the preceding paragraphs, wherein the plant is wheat, triticale, canola/oilseed rape, indian mustard, barley, rice, oat, or rye.
47. The plant of any one of the preceding paragraphs, wherein the plant is wheat.
48. The plant of paragraph 41, wherein the at least one allele of a seed color gene (e.g., seed coat and/or seed endosperm gene) (or at least one functional ectopic allele of each member of a set of seed color genes) comprises a sequence from T. aestivum, T durum, T. monococcum or another Triticum aestivum-crossable species.
49. The plant of any one of the preceding paragraphs, wherein the plant is hexaploid wheat or tetraploid wheat, Triticum aestivum, or Triticum durum.
50. A method of preparing a male-fertile maintainer plant for a male-sterile polyploid plant, the method comprising engineering a plant to comprise:
   in a first genome:
      on a first chromosome of a pair of homologous chromosomes, at a single target locus, at least one functional ectopic allele of a MF gene and at least one functional ectopic allele of a seed color gene (e.g., seed coat and/or seed endosperm gene) (or at least one functional ectopic allele of each member of a set of seed color genes);
      on a second chromosome of the pair of homologous chromosomes, at the target locus corresponding to the target locus of the first chromosome of the pair of homologous chromosomes, at least one functional ectopic allele of a PV gene; and
      loss-of-function alleles of the endogenous MF genes at the native MF gene loci and loss-of-function alleles of the endogenous PV genes at the native PV gene loci.
51. A method of preparing a male-fertile maintainer plant for a male-sterile polyploid plant, the method comprising, simultaneously or sequentially:
   inserting, on a first chromosome of a pair of homologous chromosomes in a first genome, at a single target locus, a construct comprising at least one functional ectopic allele of a MF gene and at least one functional ectopic allele of a seed color gene (e.g., seed coat and/or seed endosperm gene) (or at least one functional ectopic allele of each member of a set of seed color genes), optionally wherein the inserting comprises nuclease cleavage of the target locus (e.g., zinc-finger nuclease or CRISPR nuclease cleavage) and recombination or end-joining of the construct;
   inserting, on a second chromosome of the pair of homologous chromosomes in the first genome, at the target locus corresponding to the target locus of the first chromosome of the pair of homologous chromosomes, a construct comprising at least one functional ectopic allele of a PV gene, optionally wherein the inserting comprises nuclease cleavage of the target locus (e.g., zinc-finger nuclease or CRISPR nuclease cleavage) and/or recombination or end-joining of the construct; and
   mutating the the endogenous MF genes at the native MF gene loci and the endogenous PV genes at the native PV gene loci to create loss-of-function alleles, optionally wherein the loss-of-function alleles are caused by contacting the genome with a site-specific guided nuclease (e.g., CRISPR) and one or more guide RNA sequences or multi-guide constructs.
52. A method of preparing a male-fertile maintainer plant for a male-sterile polyploid plant, the method comprising:
   i) inserting, on a first chromosome of a pair of homologous chromosomes in a first genome, at a single target locus, a cassette comprising in 5′ to 3′ or 3′ to 5′ order:
      a first recognition site for a first recombinase;
      at least one functional ectopic nuclease null allele of a MF gene and at least one functional ectopic allele of a seed color gene (e.g., seed coat and/or seed endosperm gene) (or at least one functional ectopic allele of each member of a set of seed color genes) in either relative order;
      a first recognition site for a second recombinase;
      a selection gene;
      a second recognition site for the first recombinase;
      at least one functional ectopic nuclease-null allele of a PV gene;
      a second recognition site for the second recombinase;
   thereby providing a full-cassette insertion plant;
   ii) contacting a first progeny of the full-cassette insertion plant, or a cell thereof, with the first recombinase,
   thereby excising:
      one recognition site for the first recombinase, the at least one functional ectopic nuclease null allele of a MF gene and at least one functional ectopic allele of a seed color gene (e.g., seed coat and/or seed endosperm gene) (or at least one functional ectopic allele of each member of a set of seed color genes), the first recognition site for the second recombinase, and the selection gene from the genome of the first progeny and
   thereby providing an excised first progeny comprising:
      one recognition site for the first recombinase, the at least one functional ectopic nuclease-null allele of a PV gene, and the second recognition site for the second recombinase portions of the construct;
   iii) contacting a second progeny of the full-cassette insertion plant, or a cell thereof, with the second recombinase,
   thereby excising:
      one recognition site for the second recombinase, the selection gene, the second recognition site for the first recombinase and at least one functional ectopic nuclease-null allele of a PV gene, and
   thereby providing an excised second progeny comprising:
      one recognition site for the second recombinase, the first recognition site for the first recombinase, and the at least one functional ectopic nuclease null allele of a MF gene and at least one functional ectopic allele of a seed color gene (e.g., seed coat and/or seed endosperm gene) (or at least one functional ectopic allele of each member of a set of seed color genes) portions of the construct;
   iv) crossing the excised first progeny provided in step ii) and the excised second progeny provided in step iii), thereby providing a third progeny comprising, in a first genome,
      on a first chromosome of a pair of homologous chromosomes, at a single target locus, the at least one functional ectopic nuclease-null allele of a MF gene and the at least one functional ectopic allele of a seed color gene (e.g., seed coat and/or seed endosperm gene) (or at least one functional ectopic allele of each member of a set of seed color genes);
      on a second chromosome of the pair of homologous chromosomes, at the target locus corresponding to the target locus of the first chromosome of the pair of homologous chromosomes, the at least one functional ectopic nuclease-null allele of a PV gene; and
   v) mutating the the endogenous MF genes at the native MF gene loci and the endogenous PV genes at the native PV gene loci to create loss-of-function alleles, optionally wherein the loss-of-function alleles are caused by contacting the genome with a site-specific guided nuclease (e.g., CRISPR) and one or more guide RNA sequences or multi-guide constructs, thereby providing the male-fertile maintainer plant.
53. The method of paragraph 52, wherein one of first recombinase and second recombinase is Cre and the other recombinase is Flp.
54. The method of any one of paragraphs 52-53, wherein the construct is a T-DNA construct.
55. The method of any one of paragraphs 52-54, wherein one or more of the steps further comprise selection of the provided plants or cells, optionally wherein the selection is PCR selection.
56. The method of any one of paragraphs 52-55, wherein the plant further comprises at least one further genome, and the method further comprises engineering loss-of-function alleles of the endogenous MF genes at the native MF gene loci and loss-of-function alleles of the PV gene at the native PV gene loci in each of the at least one further genomes.
57. The method of any one of paragraphs 52-56, wherein the target locus is the native MF gene locus.
58. The method of any one of paragraphs 52-57, wherein the target locus is the native PV gene locus.
59. The method of any one of paragraphs 52-56, wherein the target locus is not the native MF gene locus or the native PV gene locus.
60. A method of preparing a male-fertile maintainer plant for a male-sterile polyploid plant, the method comprising:
   i) contacting a cell comprising a PV locus in a first chromosome and a second chromosome of a pair of homologous chromosomes in a first genome, with:
      1) a site-specific guided nuclease (e.g., CRISPR);
      2) one or more guide RNA sequences or multi-guide constructs specific to one or more sequences at the PV locus; and
      3) an targeting insertion cassette comprising in 5′ to 3′ or 3′ to 5′ order: a first recognition site for a first recombinase and a second recognition site for the first recombinase;
   thereby providing a targeting insertion plant;
   ii) contacting the targeting insertion plant, or first progeny of the targeting insertion plant, or a cell thereof with the first recombinase and a cassette comprising in 5′ to 3′ or 3′ to 5′ order:
      1) a first recombination site for the first recombinase;
      2) at least one functional ectopic nuclease null allele of a MF gene and at least one functional ectopic allele of a seed color gene (e.g., seed coat and/or seed endosperm gene) (or at least one functional ectopic allele of each member of a set of seed color genes) in either relative order; and
      3) a second recombination site for the first recombinase;
   thereby providing a cassette insertion plant;
   iii) selecting a cassette insertion plant comprising a cassette insertion at one allele of the PV locus, or crossing a cassette insertion plant comprising a cassette insertion at both alleles of the PV locus with a plant with a functional PV allele at the PV locus,
   thereby providing a cassette insertion plant with a cassette insertion at one PV allele in the first genome and a functional PV allele at the second PV allele in the first genome,
   iv) contacting the cassette insertion plant selected in iii), or a first progeny or cell thereof, with:
      1) a site-specific guided nuclease (e.g., CRISPR);
      2) one or more guide RNA sequences or multi-guide constructs flanking the insertion sites, thereby excising the inserted recombination sites;
      3) one or more guide RNA sequences or multi-guide constructs specific to the functional endogenous MF genes and/or flanking the functional endogenous MF genes, thereby mutating the functional endogenous MF genes at the functional native MF gene loci to create loss-of-function alleles;
thereby providing the male-fertile maintainer plant.
61. The method of paragraph 60, wherein the contacting of step i) comprises biolistic delivery or integration.
62. The method of any of paragraphs 60-61, wherein the contacting of step ii) comprises transforming the plant, progeny, or cell thereof with one or more T-DNAs comprising the recombinase and cassette.
63. The method of paragraph 62, wherein the method further comprises a step v) of segregating remaining T-DNA out of the plant or plant cells.
64. The method of any of paragraphs 60-64, wherein the PV gene is endogenously expressed only from the first genome.
65. The method of paragraph 64, wherein the PV gene is Ms1.
66. The method of paragraph 65, wherein the one or more sequences at the PV locus are one or more of the three gRNA sequences of SEQ ID NOs: 235-237.
67. The method of any of paragraphs 60-63, wherein the PV genes is endogenously expressed from the first genome and at least one further genome and in step iv) the plant, first progeny, or cell thereof is further contacted with one or more guide RNA sequences or multi-guide constructs specific to the endogenous PV genes and/or flanking the endogenous PV genes, thereby mutating the endogenous PV genes at the native PV gene loci to create loss-of-function alleles.
68. The method of any one of the preceding paragraphs, wherein the ectopic allele of the MF gene and/or the ectopic allele of the PV gene is a nuclease-null allele.
69. The method of any of the preceding paragraphs, wherein the ectopic allele of the MF gene and/or the ectopic allele of the PV gene is a CRISPR-null allele.
70. The method of any of the preceding paragraphs, wherein the at least one functional allele of a MF gene is the endogenous wild-type functional allele of the MF gene.
71. The method of any of the preceding paragraphs, wherein the at least one functional allele of a MF gene is an ectopic copy of the MF gene.
72. The method of any of the preceding paragraphs, wherein the at least one functional allele of a MF gene and the at least one allele of a seed color gene (e.g., seed coat and/or seed endosperm gene) (or at least one functional ectopic allele of each member of a set of seed color genes) are part of single construct.
73. The method of any of the preceding paragraphs, wherein an ectopic allele or ectopic copy of a gene is a nuclease-null or CRISPR-null allele.
74. The method of any of the preceding paragraphs, wherein the MF gene displays the same type of activity and shares at least 80%, at least 85%, at least 90%, at least 95%, or greater sequence identity with one or more of the genes of Table 1.
75. The method of paragraph 74, wherein the MF gene is selected from Table 1.
76. The method of any of the preceding paragraphs, wherein the MF gene displays the same type of activity and shares at least 80%, at least 85%, at least 90%, at least 95%, or greater sequence identity with Mfw2.
77. The method of any of the preceding paragraphs, wherein the MF gene is Mfw2.
78. The method of any one of the preceding paragraphs, wherein the PV gene displays the same type of activity and shares at least 80%, at least 85%, at least 90%, at least 95%, or greater sequence identity with one or more of the genes of Table 2.
79. The method of paragraph 78, wherein the PV gene is selected from Table 2.
80. The method of any of the preceding paragraphs, wherein the PV gene displays the same type of activity and shares at least 80%, at least 85%, at least 90%, at least 95%, or greater sequence identity with PV1 or PV2.
81. The method of any of the preceding paragraphs, wherein the PV gene is PV1 or PV2.
82. The method of any one of the preceding paragraphs, wherein the PV gene displays the same type of activity and shares at least 80%, at least 85%, at least 90%, at least 95%, or greater sequence identity with Ms1.
83. The method of any one of the preceding paragraphs, wherein the PV gene is Ms1.
84. The method of any one of the preceding paragraphs, wherein the PV gene displays the same type of activity and shares at least 80%, at least 85%, at least 90%, at least 95%, or greater sequence identity with PV3.
85. The method of any one of the preceding paragraphs, wherein the PV gene is PV3.
86. The method of any one of the preceding paragraphs, wherein the MF gene displays the same type of activity and shares at least 80%, at least 85%, at least 90%, at least 95%, or greater sequence identity with Mfw2 and the PV gene displays the same type of activity and shares at least 80%, at least 85%, at least 90%, at least 95%, or greater sequence identity with Ms1.
87. The method of any one of the preceding paragraphs, wherein the MF gene is Mfw2 and the PV gene is Ms1.
88. The method of any one of the preceding paragraphs, wherein the MF gene displays the same type of activity and shares at least 80%, at least 85%, at least 90%, at least 95%, or greater sequence identity with Mfw2 and the PV gene displays the same type of activity and shares at least 80%, at least 85%, at least 90%, at least 95%, or greater sequence identity with PV1.
89. The method of any one of the preceding paragraphs, wherein the MF gene is Mfw2 and the PV gene is PV1.
90. The method of any one of the preceding paragraphs, wherein the MF gene displays the same type of activity and shares at least 80%, at least 85%, at least 90%, at least 95%, or greater sequence identity with Mfw2 and the PV gene displays the same type of activity and shares at least 80%, at least 85%, at least 90%, at least 95%, or greater sequence identity with PV3.
91. The method of any one of the preceding paragraphs, wherein the MF gene is Mfw2 and the PV gene is PV3.
92. The method of any of the preceding paragraphs, wherein the at least one allele of a seed color gene (e.g., seed coat and/or seed endosperm gene) (or at least one functional ectopic allele of each member of a set of seed color genes) is exogenous.
93. The method of any of the preceding paragraphs, wherein the at least one allele of a seed color gene (e.g., seed coat and/or seed endosperm gene) is blue aleurone (BA).
94. The method of any of the preceding paragraphs, wherein the at least one allele of a seed color gene (e.g., seed coat and/or seed endosperm gene) (or at least one functional ectopic allele of each member of a set of seed color genes) comprises sequences obtained from a species within the same genus as the plant.
95. The method of any of the preceding paragraphs, wherein the at least one allele of a seed color gene (e.g., seed coat and/or seed endosperm gene) (or at least one functional ectopic allele of each member of a set of seed color genes) is located within 10 cM of the MF gene loci.
96. The method of any of the preceding paragraphs, wherein the at least one allele of a seed color gene (e.g., seed coat and/or seed endosperm gene) (or at least one functional ectopic allele of each member of a set of seed color genes) is located within 1 cM of the MF gene loci.
97. The method of any of the preceding paragraphs, wherein the at least one ectopic functional allele of a PV gene is located within 10 cM of the MF gene loci.
98. The method of any of the preceding paragraphs, wherein the at least one ectopic functional allele of a PV gene is located within 1 cM of the MF gene loci.
99. The method of any of the preceding paragraphs, wherein the only exogenous sequence in the genomes is the at least one allele of a seed color gene (e.g., seed coat and/or seed endosperm gene) (or at least one functional ectopic allele of each member of a set of seed color genes).
100. The method of any of the preceding paragraphs, wherein the only ectopic sequence in the genomes is the at least one ectopic functional allele of a PV gene.
101. The method of any of the preceding paragraphs, wherein the plant is tetraploid and the second genome comprises loss-of-function alleles of the MF gene at the native MF gene loci and loss-of-function alleles of the PV gene at the native PV gene loci.
102. The method of any of the preceding paragraphs, wherein the plant is hexaploid and the second and third genomes both comprise loss-of-function alleles of the MF gene at the native MF gene loci and loss-of-function alleles of the PV gene at the native PV gene loci.
103. The method of any of the preceding paragraphs, wherein a loss-of-function allele comprises an engineered knock-out modification.
104. The method of any of the preceding paragraphs, wherein a loss-of-function allele comprises an engineered excision of at least part of a coding or regulatory sequence.
105. The method of any of paragraphs 103-104, wherein the loss-of-function allele is engineered using a site-specific guided nuclease.
106. The method of paragraph 105, wherein the site-specific guided nuclease is a form of CRISPR-Cas (such as CRISPR-Cas9).
107. The method of any of the preceding paragraphs, wherein the plant is wheat, triticale, canola/oilseed rape, indian mustard, barley, rice, oat, or rye.
108. The method of any of the preceding paragraphs, wherein the plant is wheat.
109. The method of paragraph 108, wherein the at least one allele of a seed color gene (e.g., seed coat and/or seed endosperm gene) (or at least one functional ectopic allele of each member of a set of seed color genes) comprises a sequence from T. aestivum, T durum, T. monococcum or another Triticum aestivum-crossable species.
110. The method of any of the preceding paragraphs, wherein the plant is hexaploid wheat or tetraploid wheat, Triticum aestivum, or Triticum durum.
111. The method of any of the preceding paragraphs, wherein the at least one functional ectopic allele of a MF gene and at least one functional ectopic allele of a seed color gene (e.g., seed coat and/or seed endosperm gene) (or at least one functional ectopic allele of each member of a set of seed color genes) comprises the sequence of SEQ ID NO: 173 or a sequence with at least 80%, 85%, 90%, or 95% sequence identity thereto; and/or wherein the at least one functional ectopic allele of a PV gene comprises the sequence of SEQ ID NO: 172 or 218 or a sequence with at least 80%, 85%, 90%, or 95% sequence identity thereto.
112. The method of any of the preceding paragraphs, wherein the guide RNA sequences and/or multi-guide constructs comprise one or more of SEQ ID NOs: 22-29, 131-154, 156, 210-213, 217, or 235-238.
113. A method of providing a male sterile plant seed, the method comprising selecting, from seed produced by selfing a plant of any one of paragraphs 1-49, seed not displaying a phenotype provided by the seed endosperm gene.
114. A method of providing male sterile plant seed, the method comprising selfing a plant of any one of paragraphs 1-49, whereby the resulting seed not displaying a phenotype provided by the seed endosperm gene is the male sterile plant seed.
115. A method of providing a F1 hybrid seed for crop production, the method comprising collecting the seed produced by a male-sterile plant pollinated by a male-fertile plant, wherein the male-sterile plant is
   a) a plant grown from male sterile plant seed obtained by the method of paragraph 105 or 106; and/or
   b) comprises:
      i) loss-of-function alleles of an endogenous MF gene at each of the native MF gene loci;
      ii) loss-of-function alleles of an endogenous PV gene at each of the native PV gene loci; and
      iii) two ectopic alleles of the PV gene at a target locus.
116. A method of providing a F1 hybrid seed for crop production, the method comprising crossing a a male-sterile plant with a male-fertile plant, wherein the male-sterile plant is
a) a plant grown from male sterile plant seed obtained by the method of paragraph 113 or 114; and/or
b) comprises:
   i) loss-of-function alleles of an endogenous MF gene at each of the native MF gene loci;
   ii) loss-of-function alleles of an endogenous PV gene at each of the native PV gene loci; and
   iii) two ectopic alleles of the PV gene at a target locus.
117. A method of providing a F1 hybrid seed for crop production, the method comprising planting a male-sterile plant within pollination range of a male-fertile plant, wherein the male-sterile plant is
   a) a plant grown from male sterile plant seed obtained by the method of paragraph 113 or 114; and/or
   b) comprises:
      i) loss-of-function alleles of an endogenous MF gene at each of the native MF gene loci;
      ii) loss-of-function alleles of an endogenous PV gene at each of the native PV gene loci; and
      iii) two ectopic alleles of the PV gene at a target locus; and whereby the male-fertile plant pollinates the male-sterile plant and F1 hybrid seed is produced.
118. The method of paragraph 113-117, wherein the pollination range is 200 metres.
119. The method of any of paragraphs 113-118, wherein the male-sterile plant and male fertile plant are different lines.
120. A method of producing a plant crop, the method comprising:
   a) planting and/or harvesting a plant or portion thereof, wherein the plant:
      i) is plant grown from F1 hybrid seed obtained by the method of any of paragraphs 115-119; and/or
      ii) comprises:
         1) in each genome of the plant, at a native MF gene locus, one functional endogenous allele of the endogenous MF gene and one loss-of-function allele of the endogenous MF gene;
         2) in each genome of the plant, at a native PV gene locus, one functional endogenous allele of the endogenous PV gene and one loss-of-function allele of the endogenous PV gene;
         3) one ectopic allele of the PV gene at a target locus.

In some embodiments, the present technology may be defined in any of the following numbered paragraphs:
1. A male-fertile maintainer plant for a male-sterile polyploid plant comprising: a first genome comprising:
   on a first chromosome of a pair of homologous chromosomes, at a single target locus, at least one functional ectopic allele of a MF gene and at least one functional ectopic allele of a seed color gene (e.g., seed coat and/or seed endosperm gene) (or at least one functional ectopic allele of each member of a set of seed color genes);
   on a second chromosome of the pair of homologous chromosomes, at the target locus corresponding to the target locus of the first chromosome of the pair of homologous chromosomes, at least one functional ectopic allele of a PV gene;
   loss-of-function alleles of the endogenous MF genes at the native MF gene loci and loss-of-function alleles of the endogenous PV genes at the native PV gene loci.
2. The male-fertile maintainer plant of paragraph 1, comprising at least one further genome, each of the further genomes comprising loss-of-function alleles of the endogenous MF genes at the native MF gene loci and loss-of-function alleles of the PV gene at the native PV gene loci.
3. The male-fertile maintainer plant of paragraph 1 or 2, wherein the target locus is the native MF gene locus.
4. The male-fertile maintainer plant of paragraph 1 or 2, wherein the target locus is the native PV gene locus.
5. The male-fertile maintainer plant of paragraph 1 or 2, wherein the target locus is not the native MF gene locus or the native PV gene locus.
6. The male-fertile maintainer plant of any one of the preceding paragraphs, wherein the ectopic allele of the MF gene and/or the ectopic allele of the PV gene is a nuclease-null allele.
7. The male-fertile maintainer plant of any one of the preceding paragraphs, wherein the ectopic allele of the MF gene and/or the ectopic allele of the PV gene is a CRISPR-null allele.
8. A male-fertile maintainer plant for a male-sterile polyploid plant comprising:
   a first genome comprising:
      on a first chromosome of a pair of homologous chromosomes, at least one functional allele of a MF gene at the MF gene locus and at least one allele of a seed color gene (e.g., seed coat and/or seed endosperm gene) (or at least one functional ectopic allele of each member of a set of seed color genes);
      on a second chromosome of the pair of homologous chromosomes, a loss-of-function allele of the MF gene at the MF gene locus and at least one ectopic functional allele of a PV gene;
      and loss-of-function alleles of the PV gene at the native PV gene loci; and
   at least one further genome, each of the further genomes comprising loss-of-function alleles of the MF gene at the native MF gene loci and loss-of-function alleles of the PV gene at the native PV gene loci.
9. The plant of any one of the preceding paragraphs, wherein the at least one functional allele of a MF gene is the endogenous wild-type functional allele of the MF gene.
10. The plant of any one of the preceding paragraphs, wherein the at least one functional allele of a MF gene is an ectopic copy of the MF gene.
11. The plant of paragraph 10, wherein the at least one functional allele of a MF gene and the at least one allele of a seed color gene (e.g., seed coat and/or seed endosperm gene) (or at least one functional ectopic allele of each member of a set of seed color genes) are part of single construct.
12. The plant of any one of the preceding paragraphs, wherein an ectopic allele or ectopic copy of a gene is a nuclease-null or CRISPR-null allele.
13. The plant of any one of the preceding paragraphs, wherein the MF gene displays the same type of activity and shares at least 80%, at least 85%, at least 90%, at least 95%, or greater sequence identity with one or more of the genes of Table 1.
14. The plant of paragraph 13, wherein the MF gene is selected from Table 1.
15. The plant of any one of the preceding paragraphs, wherein the MF gene displays the same type of activity and shares at least 80%, at least 85%, at least 90%, at least 95%, or greater sequence identity with Mfw2.
16. The plant of any one of the preceding paragraphs, wherein the MF gene is Mfw2.
17. The plant of any one of the preceding paragraphs, wherein the MF gene displays the same type of activity and shares at least 80%, at least 85%, at least 90%, at least 95%, or greater sequence identity with Ms1.
18. The plant of any one of the preceding paragraphs, wherein the MF gene is Ms1.
19. The plant of any one of the preceding paragraphs, wherein the PV gene displays the same type of activity and shares at least 80%, at least 85%, at least 90%, at least 95%, or greater sequence identity with one or more of the genes of Table 2.
20. The plant of paragraph 17, wherein the PV gene is selected from Table 2.
21. The plant of any one of the preceding paragraphs, wherein the PV gene displays the same type of activity and shares at least 80%, at least 85%, at least 90%, at least 95%, or greater sequence identity with PV1 or PV2.
22. The plant of any one of the preceding paragraphs, wherein the PV gene is PV1 or PV2.
23. The plant of any one of the preceding paragraphs, wherein the PV gene displays the same type of activity and shares at least 80%, at least 85%, at least 90%, at least 95%, or greater sequence identity with PV3.
24. The plant of any one of the preceding paragraphs, wherein the PV gene is PV3.
25. The plant of any one of the preceding paragraphs, wherein the MF gene displays the same type of activity and shares at least 80%, at least 85%, at least 90%, at least 95%, or greater sequence identity with Mfw2 and the PV gene displays the same type of activity and shares at least 80%, at least 85%, at least 90%, at least 95%, or greater sequence identity with PV1.
26. The plant of any one of the preceding paragraphs, wherein the MF gene is Mfw2 and the PV gene is PV1.
27. The plant of any one of the preceding paragraphs, wherein the MF gene displays the same type of activity and shares at least 80%, at least 85%, at least 90%, at least 95%, or greater sequence identity with Ms1 and the PV gene displays the same type of activity and shares at least 80%, at least 85%, at least 90%, at least 95%, or greater sequence identity with PV1.
28. The plant of any one of the preceding paragraphs, wherein the MF gene is Ms1 and the PV gene is PV1.
29. The plant of any one of the preceding paragraphs, wherein the MF gene displays the same type of activity and shares at least 80%, at least 85%, at least 90%, at least 95%, or greater sequence identity with Mfw2 and the PV gene displays the same type of activity and shares at least 80%, at least 85%, at least 90%, at least 95%, or greater sequence identity with PV3.
30. The plant of any one of the preceding paragraphs, wherein the MF gene is Mfw2 and the PV gene is PV3.
31. The plant of any one of the preceding paragraphs, wherein the at least one allele of a seed color gene (e.g., seed coat and/or seed endosperm gene) (or at least one functional ectopic allele of each member of a set of seed color genes) is exogenous.
32. The plant of any one of the preceding paragraphs, wherein the at least one allele of a seed color gene (e.g., seed coat and/or seed endosperm gene) is blue aleurone (BA).
33. The plant of any one of the preceding paragraphs, wherein the at least one allele of a seed color gene (e.g., seed coat and/or seed endosperm gene) (or at least one functional ectopic allele of each member of a set of seed color genes) comprises sequences obtained from a species within the same genus as the plant.
34. The plant of any one of the preceding paragraphs, wherein the at least one allele of a seed color gene (e.g., seed coat and/or seed endosperm gene) (or at least one functional ectopic allele of each member of a set of seed color genes) is located within 10 cM of the MF gene loci.
35. The plant of any one of the preceding paragraphs, wherein the at least one allele of a seed color gene (e.g., seed coat and/or seed endosperm gene) (or at least one functional ectopic allele of each member of a set of seed color genes) is located within 1 cM of the MF gene loci.
36. The plant of any one of the preceding paragraphs, wherein the at least one ectopic functional allele of a PV gene is located within 10 cM of the MF gene loci.
37. The plant of any one of the preceding paragraphs, wherein the at least one ectopic functional allele of a PV gene is located within 1 cM of the MF gene loci.
38. The plant of any one of the preceding paragraphs, wherein the only exogenous sequence in the genomes is the at least one allele of a seed color gene (e.g., seed coat and/or seed endosperm gene) (or at least one functional ectopic allele of each member of a set of seed color genes).
39. The plant of any one of the preceding paragraphs, wherein the only ectopic sequence in the genomes is the at least one ectopic functional allele of a PV gene.
40. The plant of any one of the preceding paragraphs, wherein the plant is tetraploid and the second genome comprises loss-of-function alleles of the MF gene at the native MF gene loci and loss-of-function alleles of the PV gene at the native PV gene loci.
41. The plant of any one of the preceding paragraphs, wherein the plant is hexaploid and the second and third genomes both comprise loss-of-function alleles of the MF gene at the native MF gene loci and loss-of-function alleles of the PV gene at the native PV gene loci.
42. The plant of any one of the preceding paragraphs, wherein a loss-of-function allele comprises an engineered knock-out modification.
43. The plant of any one of the preceding paragraphs, wherein a loss-of-function allele comprises an engineered excision of at least part of a coding or regulatory sequence.
44. The plant of any one of paragraphs 42-43, wherein the loss-of-function allele is engineered using a site-specific guided nuclease.
45. The plant of paragraph 44, wherein the site-specific guided nuclease is a form of CRISPR-Cas (such as CRISPR-Cas9).
46. The plant of any one of the preceding paragraphs, wherein the plant is wheat, triticale, canola/oilseed rape, indian mustard, barley, rice, oat, or rye.
47. The plant of any one of the preceding paragraphs, wherein the plant is wheat.
48. The plant of paragraph 41, wherein the at least one allele of a seed color gene (e.g., seed coat and/or seed endosperm gene) (or at least one functional ectopic allele of each member of a set of seed color genes) comprises a sequence from T. aestivum, T durum, T. monococcum or another Triticum aestivum-crossable species.
49. The plant of any one of the preceding paragraphs, wherein the plant is hexaploid wheat or tetraploid wheat, Triticum aestivum, or Triticum durum.
50. A method of preparing a male-fertile maintainer plant for a male-sterile polyploid plant, the method comprising engineering a plant to comprise:
   in a first genome:
      on a first chromosome of a pair of homologous chromosomes, at a single target locus, at least one functional ectopic allele of a MF gene and at least one functional ectopic allele of a seed color gene (e.g., seed coat and/or seed endosperm gene) (or at least one functional ectopic allele of each member of a set of seed color genes);
      on a second chromosome of the pair of homologous chromosomes, at the target locus corresponding to the target locus of the first chromosome of the pair of homologous chromosomes, at least one functional ectopic allele of a PV gene; and
      loss-of-function alleles of the endogenous MF genes at the native MF gene loci and loss-of-function alleles of the endogenous PV genes at the native PV gene loci.
51. A method of preparing a male-fertile maintainer plant for a male-sterile polyploid plant, the method comprising, simultaneously or sequentially:
   inserting, on a first chromosome of a pair of homologous chromosomes in a first genome, at a single target locus, a construct comprising at least one functional ectopic allele of a MF gene and at least one functional ectopic allele of a seed color gene (e.g., seed coat and/or seed endosperm gene) (or at least one functional ectopic allele of each member of a set of seed color genes), optionally wherein the inserting comprises nuclease cleavage of the target locus (e.g., zinc-finger nuclease or CRISPR nuclease cleavage) and recombination or end-joining of the construct;
   inserting, on a second chromosome of the pair of homologous chromosomes in the first genome, at the target locus corresponding to the target locus of the first chromosome of the pair of homologous chromosomes, a construct comprising at least one functional ectopic allele of a PV gene, optionally wherein the inserting comprises nuclease cleavage of the target locus (e.g., zinc-finger nuclease or CRISPR nuclease cleavage) and/or recombination or end-joining of the construct; and
   mutating the the endogenous MF genes at the native MF gene loci and the endogenous PV genes at the native PV gene loci to create loss-of-function alleles, optionally wherein the loss-of-function alleles are caused by contacting the genome with a site-specific guided nuclease (e.g., CRISPR) and one or more guide RNA sequences or multi-guide constructs.
52. A method of preparing a male-fertile maintainer plant for a male-sterile polyploid plant, the method comprising:
   i) inserting, on a first chromosome of a pair of homologous chromosomes in a first genome, at a single target locus, a cassette comprising in 5′ to 3′ or 3′ to 5′ order:
      a first recognition site for a first recombinase;
      at least one functional ectopic nuclease null allele of a MF gene and at least one functional ectopic allele of a seed color gene (e.g., seed coat and/or seed endosperm gene) (or at least one functional ectopic allele of each member of a set of seed color genes) in either relative order;
      a first recognition site for a second recombinase;
      a selection gene;
      a second recognition site for the first recombinase;
      at least one functional ectopic nuclease-null allele of a PV gene;
      a second recognition site for the second recombinase;
   thereby providing a full-cassette insertion plant;
   ii) contacting a first progeny of the full-cassette insertion plant, or a cell thereof, with the first recombinase,
   thereby excising:
      one recognition site for the first recombinase, the at least one functional ectopic nuclease null allele of a MF gene and at least one functional ectopic allele of a seed color gene (e.g., seed coat and/or seed endosperm gene) (or at least one functional ectopic allele of each member of a set of seed color genes), the first recognition site for the second recombinase, and the selection gene from the genome of the first progeny and
   thereby providing an excised first progeny comprising:
      one recognition site for the first recombinase, the at least one functional ectopic nuclease-null allele of a PV gene, and the second recognition site for the second recombinase portions of the construct;
   iii) contacting a second progeny of the full-cassette insertion plant, or a cell thereof, with the second recombinase,
   thereby excising:
      one recognition site for the second recombinase, the selection gene, the second recognition site for the first recombinase and at least one functional ectopic nuclease-null allele of a PV gene, and
   thereby providing an excised second progeny comprising:
      one recognition site for the second recombinase, the first recognition site for the first recombinase, and the at least one functional ectopic nuclease null allele of a MF gene and at least one functional ectopic allele of a seed color gene (e.g., seed coat and/or seed endosperm gene) (or at least one functional ectopic allele of each member of a set of seed color genes) portions of the construct;
   iv) crossing the excised first progeny provided in step ii) and the excised second progeny provided in step iii), thereby providing a third progeny comprising, in a first genome,
      on a first chromosome of a pair of homologous chromosomes, at a single target locus, the at least one functional ectopic nuclease-null allele of a MF gene and the at least one functional ectopic allele of a seed color gene (e.g., seed coat and/or seed endosperm gene) (or at least one functional ectopic allele of each member of a set of seed color genes);
      on a second chromosome of the pair of homologous chromosomes, at the target locus corresponding to the target locus of the first chromosome of the pair of homologous chromosomes, the at least one functional ectopic nuclease-null allele of a PV gene; and
   v) mutating the the endogenous MF genes at the native MF gene loci and the endogenous PV genes at the native PV gene loci to create loss-of-function alleles, optionally wherein the loss-of-function alleles are caused by contacting the genome with a site-specific guided nuclease (e.g., CRISPR) and one or more guide RNA sequences or multi-guide constructs, thereby providing the male-fertile maintainer plant.
53. The method of paragraph 52, wherein one of first recombinase and second recombinase is Cre and the other recombinase is Flp.
54. The method of any one of paragraphs 52-53, wherein the construct is a T-DNA construct.
55. The method of any one of paragraphs 52-54, wherein one or more of the steps further comprise selection of the provided plants or cells, optionally wherein the selection is PCR selection.
56. The method of any one of paragraphs 52-55, wherein the plant further comprises at least one further genome, and the method further comprises engineering loss-of-function alleles of the endogenous MF genes at the native MF gene loci and loss-of-function alleles of the PV gene at the native PV gene loci in each of the at least one further genomes.
57. The method of any one of paragraphs 52-56, wherein the target locus is the native MF gene locus.
58. The method of any one of paragraphs 52-57, wherein the target locus is the native PV gene locus.
59. The method of any one of paragraphs 52-56, wherein the target locus is not the native MF gene locus or the native PV gene locus.
60. A method of preparing a male-fertile maintainer plant for a male-sterile polyploid plant, the method comprising:
   i) contacting a cell comprising a PV locus in a first chromosome and a second chromosome of a pair of homologous chromosomes in a first genome, with:
      1) a site-specific guided nuclease (e.g., CRISPR);
      2) one or more guide RNA sequences or multi-guide constructs specific to one or more sequences at the PV locus; and
      3) an targeting insertion cassette comprising in 5′ to 3′ or 3′ to 5′ order: a first recognition site for a first recombinase and a second recognition site for the first recombinase;
   thereby providing a targeting insertion plant;
   ii) contacting the targeting insertion plant, or first progeny of the targeting insertion plant, or a cell thereof with the first recombinase and a cassette comprising in 5′ to 3′ or 3′ to 5′ order:
      4) a first recombination site for the first recombinase;
      5) at least one functional ectopic nuclease null allele of a MF gene and at least one functional ectopic allele of a seed color gene (e.g., seed coat and/or seed endosperm gene) (or at least one functional ectopic allele of each member of a set of seed color genes) in either relative order; and
      6) a second recombination site for the first recombinase;
   thereby providing a cassette insertion plant;
   iii) selecting a cassette insertion plant comprising a cassette insertion at one allele of the PV locus, or crossing a cassette insertion plant comprising a cassette insertion at both alleles of the PV locus with a plant with a functional PV allele at the PV locus,
   thereby providing a cassette insertion plant with a cassette insertion at one PV allele in the first genome and a functional PV allele at the second PV allele in the first genome,
   iv) contacting the cassette insertion plant selected in iii), or a first progeny or cell thereof, with:
      1) a site-specific guided nuclease (e.g., CRISPR);
      2) one or more guide RNA sequences or multi-guide constructs flanking the insertion sites, thereby excising the inserted recombination sites;
      3) one or more guide RNA sequences or multi-guide constructs specific to the functional endogenous MF genes and/or flanking the functional endogenous MF genes, thereby mutating the functional endogenous MF genes at the functional native MF gene loci to create loss-of-function alleles;
thereby providing the male-fertile maintainer plant.
61. The method of paragraph 60, wherein the contacting of step i) comprises biolistic delivery or integration.
62. The method of any of paragraphs 60-61, wherein the contacting of step ii) comprises transforming the plant, progeny, or cell thereof with one or more T-DNAs comprising the recombinase and cassette.
63. The method of paragraph 62, wherein the method further comprises a step v) of segregating remaining T-DNA out of the plant or plant cells.
64. The method of any of paragraphs 60-64, wherein the MF gene is endogenously expressed only from the first genome.
65. The method of paragraph 64, wherein the MF gene is Ms1.
66. The method of paragraph 65, wherein the one or more sequences at the MF locus are the gRNA sequences or constructs can be or comprise one or more of the three gRNA sequences of SEQ ID NOs: 253, 254, and 267.
67. The method of any of paragraphs 60-63, wherein the PV gene is endogenously expressed from the first genome and at least one further genome and in step iv) the plant, first progeny, or cell thereof is further contacted with one or more guide RNA sequences or multi-guide constructs specific to the endogenous PV genes and/or flanking the endogenous PV genes, thereby mutating the endogenous PV genes at the native PV gene loci to create loss-of-function alleles.
68. The method of any one of the preceding paragraphs, wherein the ectopic allele of the MF gene and/or the ectopic allele of the PV gene is a nuclease-null allele.
69. The method of any of the preceding paragraphs, wherein the ectopic allele of the MF gene and/or the ectopic allele of the PV gene is a CRISPR-null allele.
70. The method of any of the preceding paragraphs, wherein the at least one functional allele of a MF gene is the endogenous wild-type functional allele of the MF gene.
71. The method of any of the preceding paragraphs, wherein the at least one functional allele of a MF gene is an ectopic copy of the MF gene.
72. The method of any of the preceding paragraphs, wherein the at least one functional allele of a MF gene and the at least one allele of a seed color gene (e.g., seed coat and/or seed endosperm gene) (or at least one functional ectopic allele of each member of a set of seed color genes) are part of single construct.
73. The method of any of the preceding paragraphs, wherein an ectopic allele or ectopic copy of a gene is a nuclease-null or CRISPR-null allele.
74. The method of any of the preceding paragraphs, wherein the MF gene displays the same type of activity and shares at least 80%, at least 85%, at least 90%, at least 95%, or greater sequence identity with one or more of the genes of Table 1.
75. The method of paragraph 74, wherein the MF gene is selected from Table 1.
76. The method of any of the preceding paragraphs, wherein the MF gene displays the same type of activity and shares at least 80%, at least 85%, at least 90%, at least 95%, or greater sequence identity with Mfw2.
77. The method of any of the preceding paragraphs, wherein the MF gene is Mfw2.
78. The method of any of the preceding paragraphs, wherein the MF gene displays the same type of activity and shares at least 80%, at least 85%, at least 90%, at least 95%, or greater sequence identity with Ms1.
79. The method of any of the preceding paragraphs, wherein the MF gene is Ms1.
80. The method of any one of the preceding paragraphs, wherein the PV gene displays the same type of activity and shares at least 80%, at least 85%, at least 90%, at least 95%, or greater sequence identity with one or more of the genes of Table 2.
81. The method of paragraph 78, wherein the PV gene is selected from Table 2.
82. The method of any of the preceding paragraphs, wherein the PV gene displays the same type of activity and shares at least 80%, at least 85%, at least 90%, at least 95%, or greater sequence identity with PV1 or PV2.
83. The method of any of the preceding paragraphs, wherein the PV gene is PV1 or PV2.
84. The method of any one of the preceding paragraphs, wherein the PV gene displays the same type of activity and shares at least 80%, at least 85%, at least 90%, at least 95%, or greater sequence identity with PV3.
85. The method of any one of the preceding paragraphs, wherein the PV gene is PV3.
86. The method of any one of the preceding paragraphs, wherein the MF gene displays the same type of activity and shares at least 80%, at least 85%, at least 90%, at least 95%, or greater sequence identity with Mfw2 and the PV gene displays the same type of activity and shares at least 80%, at least 85%, at least 90%, at least 95%, or greater sequence identity with PV1.
87. The method of any one of the preceding paragraphs, wherein the MF gene is Mfw2 and the PV gene is PV1.
88. The method of any one of the preceding paragraphs, wherein the MF gene displays the same type of activity and shares at least 80%, at least 85%, at least 90%, at least 95%, or greater sequence identity with Ms1 and the PV gene displays the same type of activity and shares at least 80%, at least 85%, at least 90%, at least 95%, or greater sequence identity with PV1.
89. The method of any one of the preceding paragraphs, wherein the MF gene is Ms1 and the PV gene is PV1.
90. The method of any one of the preceding paragraphs, wherein the MF gene displays the same type of activity and shares at least 80%, at least 85%, at least 90%, at least 95%, or greater sequence identity with Mfw2 and the PV gene displays the same type of activity and shares at least 80%, at least 85%, at least 90%, at least 95%, or greater sequence identity with PV3.
91. The method of any one of the preceding paragraphs, wherein the MF gene is Mfw2 and the PV gene is PV3.
92. The method of any of the preceding paragraphs, wherein the at least one allele of a seed color gene (e.g., seed coat and/or seed endosperm gene) (or at least one functional ectopic allele of each member of a set of seed color genes) is exogenous.
93. The method of any of the preceding paragraphs, wherein the at least one allele of a seed color gene (e.g., seed coat and/or seed endosperm gene) is blue aleurone (BA).
94. The method of any of the preceding paragraphs, wherein the at least one allele of a seed color gene (e.g., seed coat and/or seed endosperm gene) (or at least one functional ectopic allele of each member of a set of seed color genes) comprises sequences obtained from a species within the same genus as the plant.
95. The method of any of the preceding paragraphs, wherein the at least one allele of a seed color gene (e.g., seed coat and/or seed endosperm gene) (or at least one functional ectopic allele of each member of a set of seed color genes) is located within 10 cM of the MF gene loci.
96. The method of any of the preceding paragraphs, wherein the at least one allele of a seed color gene (e.g., seed coat and/or seed endosperm gene) (or at least one functional ectopic allele of each member of a set of seed color genes) is located within 1 cM of the MF gene loci.
97. The method of any of the preceding paragraphs, wherein the at least one ectopic functional allele of a PV gene is located within 10 cM of the MF gene loci.
98. The method of any of the preceding paragraphs, wherein the at least one ectopic functional allele of a PV gene is located within 1 cM of the MF gene loci.
99. The method of any of the preceding paragraphs, wherein the only exogenous sequence in the genomes is the at least one allele of a seed color gene (e.g., seed coat and/or seed endosperm gene) (or at least one functional ectopic allele of each member of a set of seed color genes).
100. The method of any of the preceding paragraphs, wherein the only ectopic sequence in the genomes is the at least one ectopic functional allele of a PV gene.
101. The method of any of the preceding paragraphs, wherein the plant is tetraploid and the second genome comprises loss-of-function alleles of the MF gene at the native MF gene loci and loss-of-function alleles of the PV gene at the native PV gene loci.
102. The method of any of the preceding paragraphs, wherein the plant is hexaploid and the second and third genomes both comprise loss-of-function alleles of the MF gene at the native MF gene loci and loss-of-function alleles of the PV gene at the native PV gene loci.
103. The method of any of the preceding paragraphs, wherein a loss-of-function allele comprises an engineered knock-out modification.
104. The method of any of the preceding paragraphs, wherein a loss-of-function allele comprises an engineered excision of at least part of a coding or regulatory sequence.
105. The method of any of paragraphs 103-104, wherein the loss-of-function allele is engineered using a site-specific guided nuclease.
106. The method of paragraph 105, wherein the site-specific guided nuclease is a form of CRISPR-Cas (such as CRISPR-Cas9).
107. The method of any of the preceding paragraphs, wherein the plant is wheat, triticale, canola/oilseed rape, indian mustard, barley, rice, oat, or rye.
108. The method of any of the preceding paragraphs, wherein the plant is wheat.
109. The method of paragraph 108, wherein the at least one allele of a seed color gene (e.g., seed coat and/or seed endosperm gene) (or at least one functional ectopic allele of each member of a set of seed color genes) comprises a sequence from T. aestivum, T durum, T. monococcum or another Triticum aestivum-crossable species.
110. The method of any of the preceding paragraphs, wherein the plant is hexaploid wheat or tetraploid wheat, Triticum aestivum, or Triticum durum.
111. The method of any of the preceding paragraphs, wherein the at least one functional ectopic allele of a MF gene and at least one functional ectopic allele of a seed color gene (e.g., seed coat and/or seed endosperm gene) (or at least one functional ectopic allele of each member of a set of seed color genes) comprises the sequence of SEQ ID NO: 173 or a sequence with at least 80%, 85%, 90%, or 95% sequence identity thereto; and/or wherein the at least one functional ectopic allele of a PV gene comprises or encodes the sequence of SEQ ID NO: 172 or 258 or a sequence with at least 80%, 85%, 90%, or 95% sequence identity thereto.
112. The method of any of the preceding paragraphs, wherein the guide RNA sequences and/or multi-guide constructs comprise one or more of SEQ ID NOs: 22-29, 131-154, 156, 210-213, 217, 235-238, 253-255, and 266-267.
113. A method of providing a male sterile plant seed, the method comprising selecting, from seed produced by selfing a plant of any one of paragraphs 1-49, seed not displaying a phenotype provided by the seed endosperm gene.
114. A method of providing male sterile plant seed, the method comprising selfing a plant of any one of paragraphs 1-49, whereby the resulting seed not displaying a phenotype provided by the seed endosperm gene is the male sterile plant seed.
115. A method of providing a F1 hybrid seed for crop production, the method comprising collecting the seed produced by a male-sterile plant pollinated by a male-fertile plant, wherein the male-sterile plant is
   a) a plant grown from male sterile plant seed obtained by the method of paragraph 105 or 106; and/or
   b) comprises:
      i) loss-of-function alleles of an endogenous MF gene at each of the native MF gene loci;
      ii) loss-of-function alleles of an endogenous PV gene at each of the native PV gene loci; and
      iii) two ectopic alleles of the PV gene at a target locus.
116. A method of providing a F1 hybrid seed for crop production, the method comprising crossing a a male-sterile plant with a male-fertile plant, wherein the male-sterile plant is
   a) a plant grown from male sterile plant seed obtained by the method of paragraph 113 or 114; and/or
   b) comprises:
      i) loss-of-function alleles of an endogenous MF gene at each of the native MF gene loci;
      ii) loss-of-function alleles of an endogenous PV gene at each of the native PV gene loci; and
      iii) two ectopic alleles of the PV gene at a target locus.
117. A method of providing a F1 hybrid seed for crop production, the method comprising planting a male-sterile plant within pollination range of a male-fertile plant, wherein the male-sterile plant is
   a) a plant grown from male sterile plant seed obtained by the method of paragraph 113 or 114; and/or
   b) comprises:
      i) loss-of-function alleles of an endogenous MF gene at each of the native MF gene loci;
      ii) loss-of-function alleles of an endogenous PV gene at each of the native PV gene loci; and
      iii) two ectopic alleles of the PV gene at a target locus; and
   whereby the male-fertile plant pollinates the male-sterile plant and F1 hybrid seed is produced.
118. The method of paragraph 113-117, wherein the pollination range is 200 metres.
119. The method of any of paragraphs 113-118, wherein the male-sterile plant and male fertile plant are different lines.
120. A method of producing a plant crop, the method comprising:
   a) planting and/or harvesting a plant or portion thereof, wherein the plant:
      i) is plant grown from F1 hybrid seed obtained by the method of any of paragraphs 115-119; and/or
      ii) comprises:
         1) in each genome of the plant, at a native MF gene locus, one functional endogenous allele of the endogenous MF gene and one loss-of-function allele of the endogenous MF gene;
         2) in each genome of the plant, at a native PV gene locus, one functional endogenous allele of the endogenous PV gene and one loss-of-function allele of the endogenous PV gene;
         3) one ectopic allele of the PV gene at a target locus.

The technology described herein is further illustrated by the following examples which in no way should be construed as being further limiting.

### EXAMPLES

### Example 1

In an exemplary embodiment, provided herein is a cis-genic maintainer based on a pre-meiosis-expressed wheat male-fertility gene, MF, (to restore male-fertility to a male-sterile plant where other copies of that gene have been knocked-out), and an allelic knocked-in Pollen Vital (PV) gene (to cause pollen grain viability where other PV copies have been knocked out) and a coloured-grain (e.g., a blue aleurone layer grain (BA) gene (to permit positive selection, in the maintainer-line seed production, of the 50% progeny which have inherited the necessary MF and pv genes in the maintainer-line's female gametes/ovules). In some embodiments of any of the aspects, the MF is Mfw2 and the PV gene is PV1, which is expressed at pollen germination. All endogenous copies of these genes are knocked-out/mutated, with the exception of a single endogenous copy of MF which is retained, linked with the coloured-grain gene.

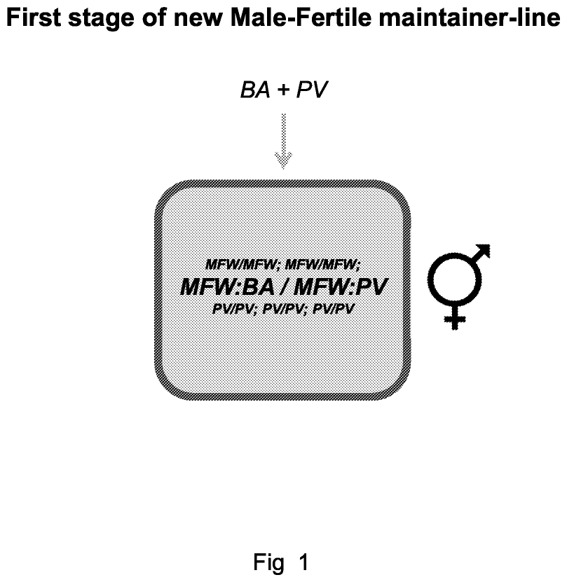

An exemplary embodiment of this system is depicted in FIGS. 1-7. FIG. 1 depicts a first step in producing a maintainer line. A wild-type elite wheat line is selected for transformation with two genes which will be ‘knocked in’ to the identical locus extremely close to MFW so that an ‘allelic’ pair can be selected. The two genes, using cis-genes from Triticum, will be BA (or another endosperm-expressed grain colour gene) and PV (the pollen vital gene fully-expressed). After the insertions, complementary chromosomes/alleles are PCR-selected so that a new line is heterozygous BA/PV at one genome's MFW-linked locus shown as MFWBA and MFW:PV (right).

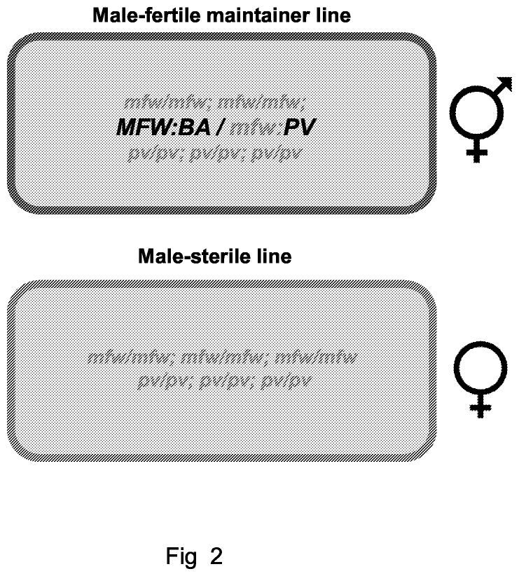

In FIG. 2, the maintainer line and cognate male sterile lines are created in parallel. The endogenous MFW and PV gene copies are mutated/knocked-out. The exception is that a genotype with just a single remaining unmutated wt MFW on the chromosome with the BA insertion is selected (see FIG. 2 top right). Thus, the fertility/sterility phenotype of the maintainer plants and their gametes will be completely dependent on the new two-gene allelic ‘constructs’: MFWBA and mfW:PV.

As shown in the top of FIG. 2, the MFW gene will not be present in the maintainer plant's viable pollen as pollen grains with the MFW gene will abort due to having no copy of the vital PV gene needed for pollen germination. Hence no transfer of male-fertility to the male-sterile. In parallel with this, a wt of the same line (with no inserted BA or PV genes) is similarly mutated to produce a genotype with MFW and PV fully knocked out. This is the male-sterile line (FIG. 2, bottom). (In this figure and subsequent ones, unexpressed genes are shown in grey and smaller; the larger/black/bold genes are the expressed ones.) By doing these experiments at the same time, the male-fertile maintainer is immediately ready to pollinate and maintain the male-sterile.

FIGS. 3-4 depict how the maintainer line is maintained. During meiosis (e.g., during the later phases of meiosis and/or after meiosis), pollen grains reciting the MFW:BA chromosome will not be viable, as they lack a functional PV gene. Therefore, the only viable grains produced have a mfw:PV genotype. When these pollen grains self-fertilize the ovules made by the maintainer line, they can fertilize one of two ovule genotypes. As shown, the two genotypes comprise either mfw:PV or MFW:BA. As depicted in FIG. 4, the resulting seed will be either 1) male-sterile and uncolored, or 2) male-fertile, colored, with the original maintainer line genotype. Thus, sorting the seeds by color will provide a pure population of the maintainer line seeds.

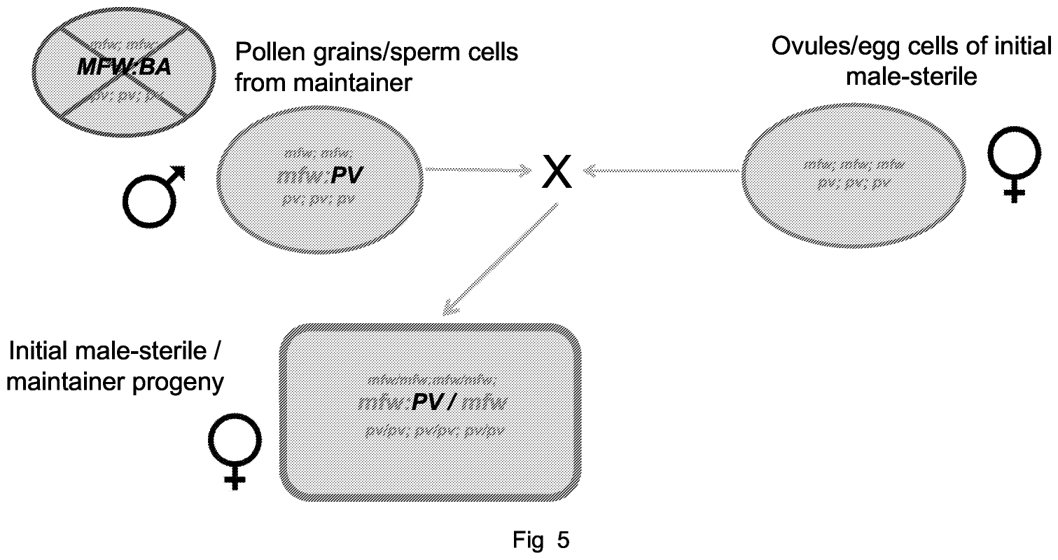

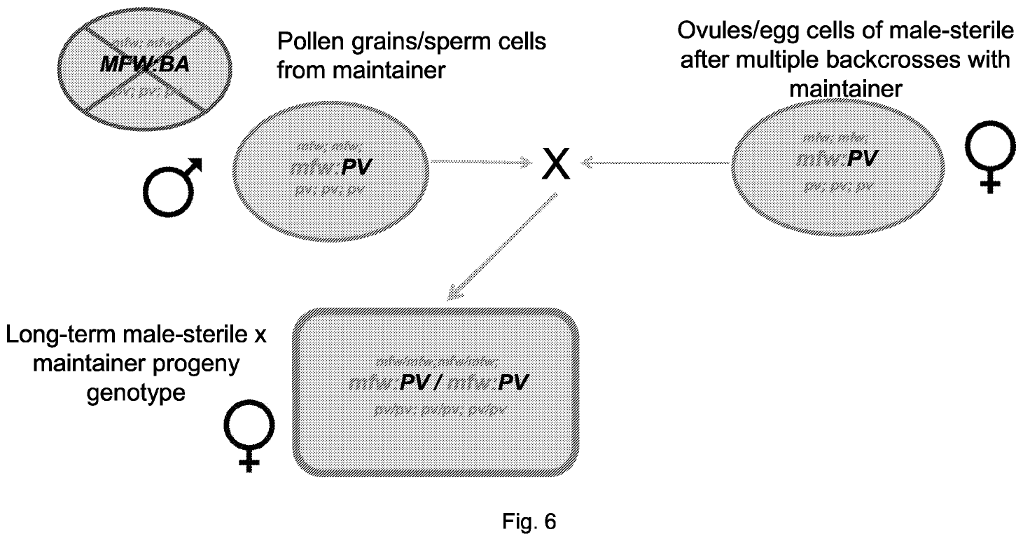

FIGS. 5-6 demonstrate how the maintainer line is used to maintainer the male-sterile plants. The maintainer line is used to fertilize a mfw×3, pv×3 initial male-sterile plant, providing male-sterile progeny with the indicated genotype. As depicted in FIG. 5, no viable MFW gene is transferred to the male-sterile as in top left pollen grain as contains no PV gene to give its pollen viability; the PV gene in the other pollen grain is irrelevant as its potentially viable pollen is never produced by the pre-meiosis male-sterility of a full set of knocked out MFWs—mfw×3. The progeny resulting from the cross shown in FIG. 5 can be the male-sterile line or further backcrossed with the maintainer to produce a final male-sterile line. FIG. 6 depicts how the male-sterile line is then propagated by crossing with the maintainer line. Male-sterility is maintained, in spite of the increased presence of the PV gene from the maintainer line, due to the complete knockout of all the pre-meiosis MFW genes
that is, the male-sterile plant and the progeny of a cross with the maintainer line are mfw/mfw in each of the three genomes.

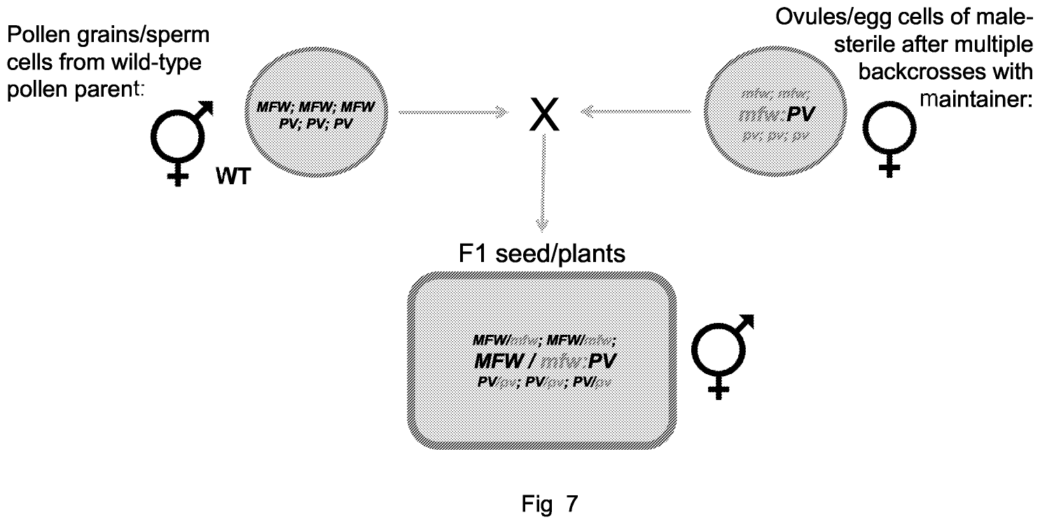

FIG. 7 depicts how the male-sterile plants are used for F1 seed production. It is contemplated that during the depicted crossing event, the pollen parent will be physically mixed with the male-sterile for maximum field pollination, seed yield reliability, and least cost. The pollen parent can be any male-fertile genotype or line, e.g., an elite breeding line.

The cross provides male-fertile F1 progeny suitable for use as a wheat crop plant. The flowers of all these F1 progeny will be pre-meiosis-male-fertile as all have a copy of MFW from the wild-type parent. 1/16* or 6.25% of pollen grains will have no PV allele so that proportion of pollen grains will be non-viable. With the huge ‘excess’ of pollen production in a wheat flower, having ˜6% non viable/non-germinating pollen grains will not be a problem for crop production. The 94% which are viable due to the presence of a PV allele will be ample for fertilization.

*Each pollen grain needs only one PV allele to be viable at the pollen germination stage. Each normal PV locus is heterozygous in the parent so, in the haploid pollen grain genome, there is a ½ probability of having thepv allele in each genome. So ⅛ have no normal-locus PV allele. Then there is a ½ chance of these also having a PV allele from the MFW-linked locus, so only 1/16 pollen grains have no PV allele.

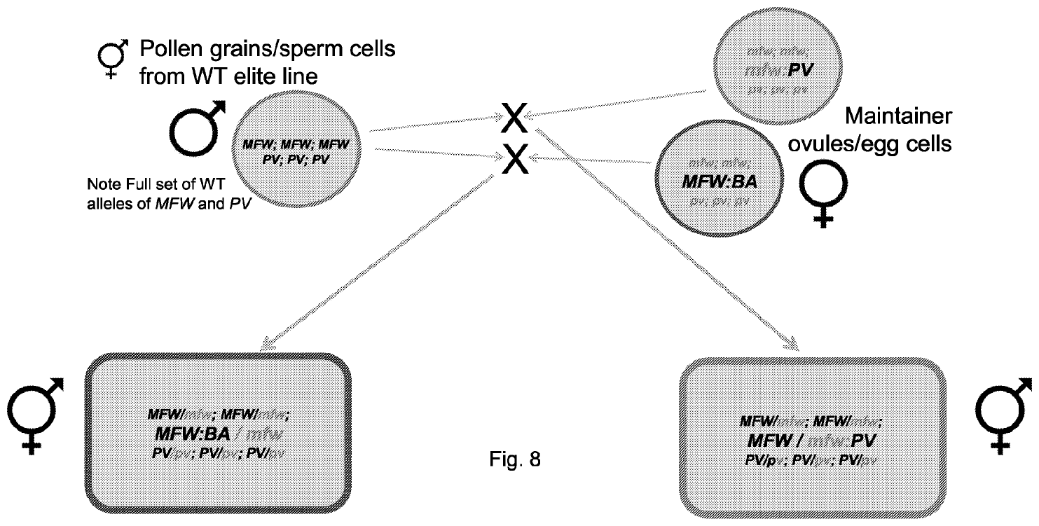

Notably, as is easily discerned from FIGS. 7 and 8, the plants involved directly in F1 seed production and crop growth do not contain exogenous genetic material, only loss-of-function alleles and an ectopic copy of PV gene, the sequence of which endogenous to the plant with only the location making the allele ectopic. That is, the plants involved directly in F1 seed production and crop growth are not transgenic.

### Example 2

The maintainer/male-sterile characteristics/genetic elements described herein can be introgressed into a new elite line by ‘conventional’ breeding. One approach to such a procedure is provided herein and illustrated in FIGS. 8-10. First, an elite wildtype (wt) line is crossed with a maintainer (of male-sterility) plant as described herein (FIG. 8). Seed harvested from the cross (ex maintainer) will be a 50% mix of the two genotypes depicted in FIG. 8. This is colour-sorted, separating the 50% with darker-coloured (BA) grains (and MFW male-fertility), (FIG. 8, bottom right), from the non-coloured plants (no BA), (FIG. 8, bottom left).

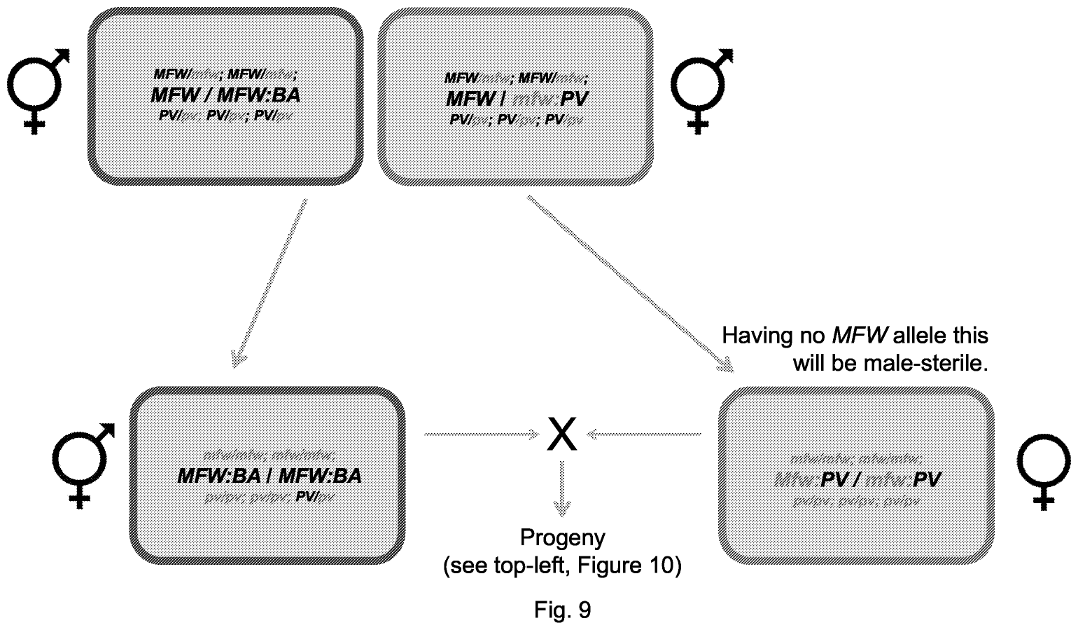

These two populations are planted, allowed to self-fertizile, and in the ensuing generation, individuals which are mfw/mfw×2 and mfw:PV/mfw:PV and pv/pv×3 (FIG. 9 left, providing male-sterile individuals) and mfW/mfw×2 and MFW:BA/MFW:BA and PV/PVx3 (FIG. 9 right) are selected by PCR analysis. These individuals are also selected for having an overall phenotype which is closest to the WT elite parent. These two selected individual plants or populations are then crossed.

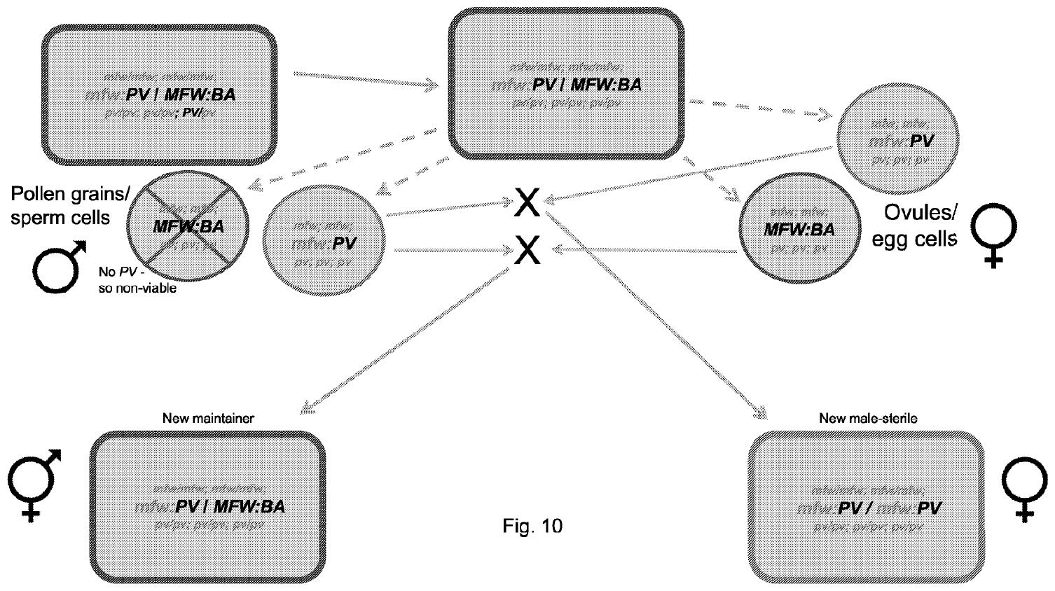

The plants from this cross are grown (FIG. 10, top left) and, from their progeny, PCR analysis is used to select those plants with a mfW/mfw×2+mfwv:PV/MFW:BA+pv/pv×3 genotype (no wt PV allele) and maximum WT elite line genotype (FIG. 10, top center). These plants are allowed to self-fertilize. Harvested seed will be a 50% mix of the two genotypes indicated at the bottom of FIG. 10. This seed is colour-sorted, selecting the 50% with darker-coloured (BA) grains and so MFW male-fertility, (FIG. 10, bottom right), to become the new maintainer line and separately, the non-coloured seeds, (FIG. 10, bottom left), which become the new male-sterile line. The seed/plants can be subject to standard selection in recurrent pollinator/maintainer for a further five generations to achieve introgression in the elite line.

### Example 3

This example presents the production and use of maintainer (of male-sterility) lines comprising nuclease-null alleles. Merely for illustrative purposes, this example utilizes wheat as the plant and CRISPR as the site-specific guided nuclease, but other plants and nucleases described herein can be used in alternative embodiments.

### Making the Maintainer

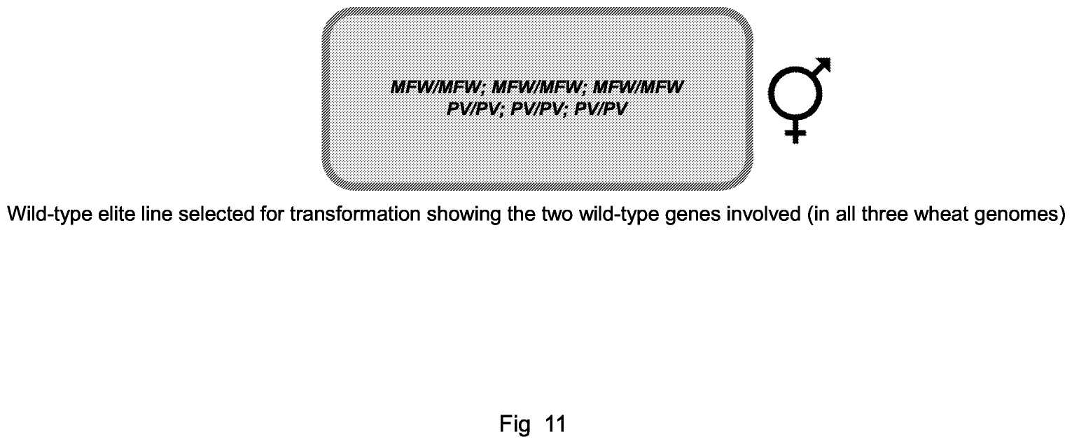

Step 1: A wild-type elite wheat line is selected for conversion/transformation to become/generate both a male-sterile and its male-fertile ‘maintainer’. Two endogenous genes vital for wheat reproduction that occur naturally in all Triticum aestivum wheat lines are at the heart of this system: MFW for initial, pre-meiosis, initiation of pollen and PV for pollen development, germination, and growth on the stigma. Both genes are found as single copies on each of wheat's three genomes; and just one wild-type copy is sufficient to generate the necessary fertile phenotype. These are the focus of this system and are shown here/hereon as in FIG. 11. The present male-sterility/maintainer system uses these two wheat genes (or others instead), subtly edited (or using a subtle natural variant selected from a rare line), with a rare ‘blue aleurone’ (or other endosperm-expressed trait) wheat gene, fused to MFW, as a selectable marker.

The selected wild-type elite wheat line can be transformed with two expression cassettes containing three genes which are ‘knocked in’ to one genome's MFW. The two cassettes comprise MFW (pre-meiosis-conclusion-expressed wheat male-fertility gene, e.g., expressed before the conclusion of meiosis, diploid) fused to BA for one cassette, and PV (the pollen vital gene fully-expressed) for the other.

The MFW and PV genes within the two maintainer cassettes can be designed to have synonymous edits (compared to wild-type) at the CRISPR/Cas9 knockout guide sites (or they can use a subtle natural variant selected from a rare line). This is so that they are not recognised by the CRISPR knockout guides at the later male-sterility-creating stage, and/or so that these and other traits can be selected in a fully-fertile form before native MFW or PV genes are knocked out without affecting the inserted genes (Step 2) yet their mRNA and amino acid sequences are unchanged so they code for/produce normal fertile phenotypes. (These subtly different or edited sequences are denoted MFW′ and PV′ hereafter.) The knockin site can be down-stream/3′ of the knockout site so that it is unaffected by the knockout process.

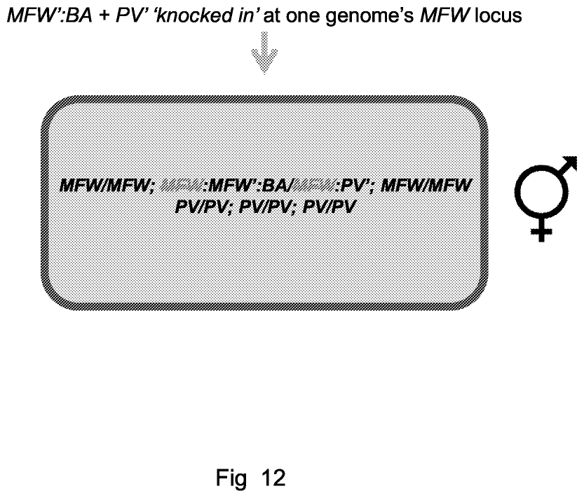

Genotypes can be selected where MFW′:BA and PV′ are introgressed into both of the homologous MFW loci. They become, in effect, a pair of ‘alleles’ at this locus. Integration of the MFW′:BA and PV′ expression cassettes at the one MFW locus results in disruption of the two endogenous gene copies at this locus (denoted MFW), as shown in FIG. 12.

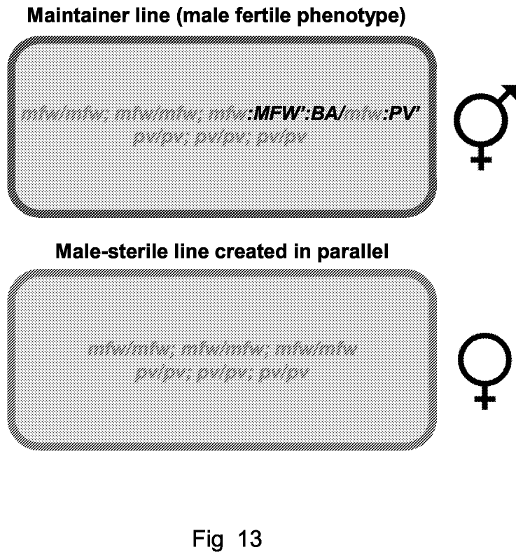

Step 2(a): Creating the maintainer and male-sterile lines in coordination. At this stage, all endogenous copies of MFW and PV are now mutated/knocked-out by CRISPR/Cas9. The MFW′ and PV′ expression cassettes inserted in Step 1 will not be CRISPR-targeted/mutated due to the CRISPR editing guides not recognising/targeting the slight/synonymous DNA sequence differences or changes at the CRISPR/Cas9 guide sites of the inserted genes. The resulting maintainer line is shown in FIG. 13.

The fertility/sterility phenotype of the maintainer plants and their gametes is now dependent on the two inserted allelic ‘constructs’: MFW′:BA and mfw:PV′. As shown in FIG. 13, these plants' viable pollen will not carry the MFW gene: the 50% of the (haploid) pollen grains which carry the MFW′ gene have mutated pv genes only and so are non-viable (wild-type PV required for pollen germination). Hence no transfer of MFW male-fertility to the male-sterile.

In parallel with the above, a wild-type of the same line (with no inserted MFW′, BA or PV′ genes) can be similarly mutated to produce a genotype with MFW and PV fully knocked out. This is the male-sterile line, as shown in FIG. 13. By doing these experiments in coordination, the male-fertile maintainer will be ready to pollinate and maintain the male-sterile. Alternatively some progeny from the making of the maintainer line above may have no successful inserts and these can be the male-sterile.

Step 2(b): Creating the maintainer and male-sterile lines together once a maintainer has already been created in a program. In an established breeding programme which already has a useable maintainer line with this system, the process described in Steps 1 and 2(a) can be accelerated and simplified as follows.

A new elite line can be back-crossed onto the established maintainer plant (with significant numbers and marker-assisted selection it should be possible to achieve near isogenic lines in ˜three generations). Then, taking embryos from a few plants with the target genotype (ie must include one genome with allelic MFW′:BA/mfW/PV′) all endogenous copies of MFW and PV can be mutated/knocked-out by CRISPR/Cas9. If no appropriate heterozygote is available at this stage, then two complementary homozygotes (most plants will be homozygous by this stage) can be crossed and the F1's embryos mutated (and a null insert plant mutated for the male sterile).

Again, the inserted MFW′ and PV′ expression cassettes will not be CRISPR-targeted/mutated and express the necessary fertility proteins. Plants for the new male-sterile can be selected from embryos which have homozygous mfw/PV′/mfw PV′ and a full set of endogenous knockouts. Plants for the new maintainer can be selected from embryos with the heterozygous maintainer combination MFW′:BA/mfw/PV′ and a full set of endogenous knockouts. Thus both a new maintainer and new male-sterile can be produced from the same experiment in the same new genetic background (see FIG. 13 for resulting genetics).

Alternative Step 1: Producing a maintainer/male-sterile using a single-genome Male-Fertility gene. The selected wild-type elite wheat line can be transformed with two expression cassettes containing three genes which are ‘knocked in’ to the one genome's MFW site The two inserted cassettes comprise MFW′ (single-genome pre-meiosis-expressed wheat male-fertility gene) fused to BA for one cassette, and PV′ (the pollen vital gene fully-expressed in all genomes) for the other.

The MFW′ and PV′ genes within the two maintainer cassettes can be designed to have (or naturally have) synonymous differences/edits (compared to wild-type) at the CRISPR/Cas9 guide sites used for knockout (at sites downstream/3′ side of the knockin sites). This is so that they are not recognised by the CRISPR knockout guides at the later male-sterility-creating stage (Step 2) yet their mRNA and amino acid sequences are unchanged so they code for/produce normal fertile phenotypes.

Genotypes can be selected where MFW′:BA and PV′ are introgressed into both of the homologous MFW loci. They become, in effect, a pair of ‘alleles’ at this locus. Integration of the MFW′:BA and PV′ expression cassettes at the one MFW locus results in disruption of the two endogenous gene copies at this locus (so here denoted MFW) as shown in FIG. 12.

### Maintaining/Using the Maintainer Line and Corresponding Male-Sterile Plants

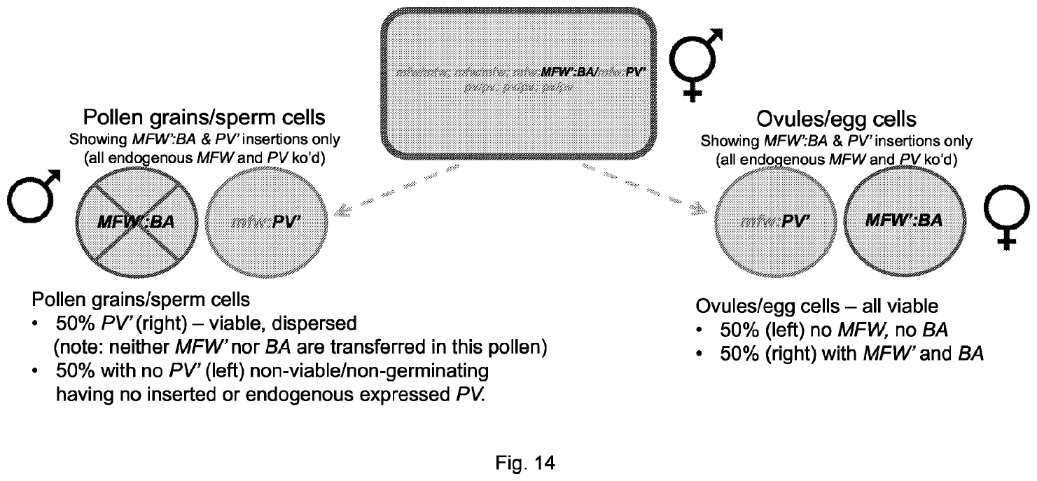

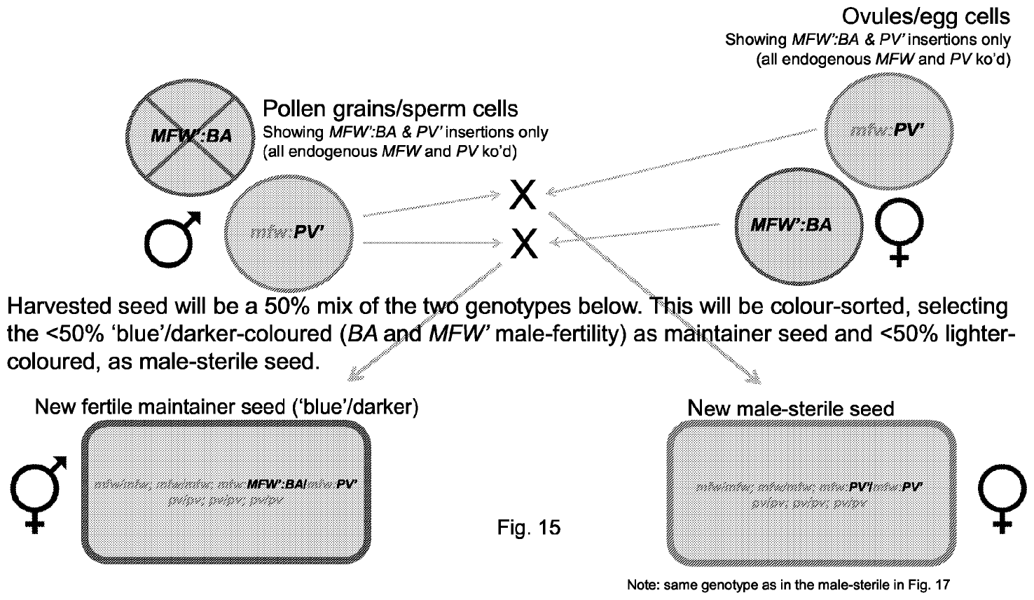

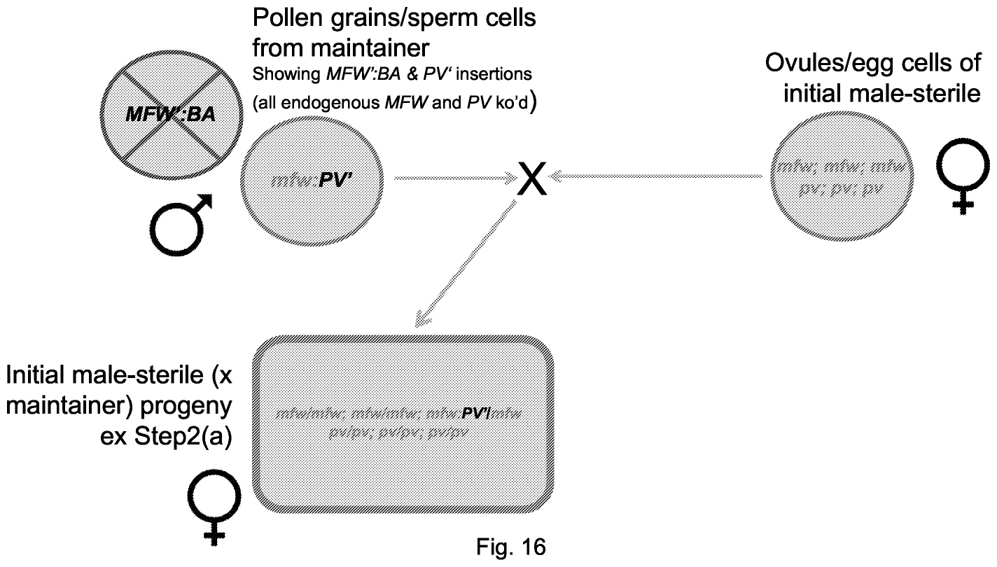

The maintainer plant can self-fertilize. The pollen and ovules produced by the maintainer are shown in FIG. 14. The far left pollen genotype is disabled at pollen germination as it has no PV′ allele. The two right hand ovule genotypes are enabled for fertilization by the pre-meiosis, heterozygote expression of the single MFW′ allele in the far right genotype (preceding heterozygote shown in FIG. 12). These pollen and ovules will fertilize and produce F1 seed according to FIG. 15, thereby maintaining the maintainer and producing male-sterile seed at the same time.

Where male-sterile production from maintainer maintenance shown in FIGS. 14-15 will be insufficient, male sterile plants can be produced or maintained as shown in FIG. 16. No viable MFW gene is transferred to the male-sterile; the PV′ gene is irrelevant as its potentially viable pollen is never produced by the pre-meiosis male-sterility of a full set of knocked out MFWs—mfv/mfw in all genomes.

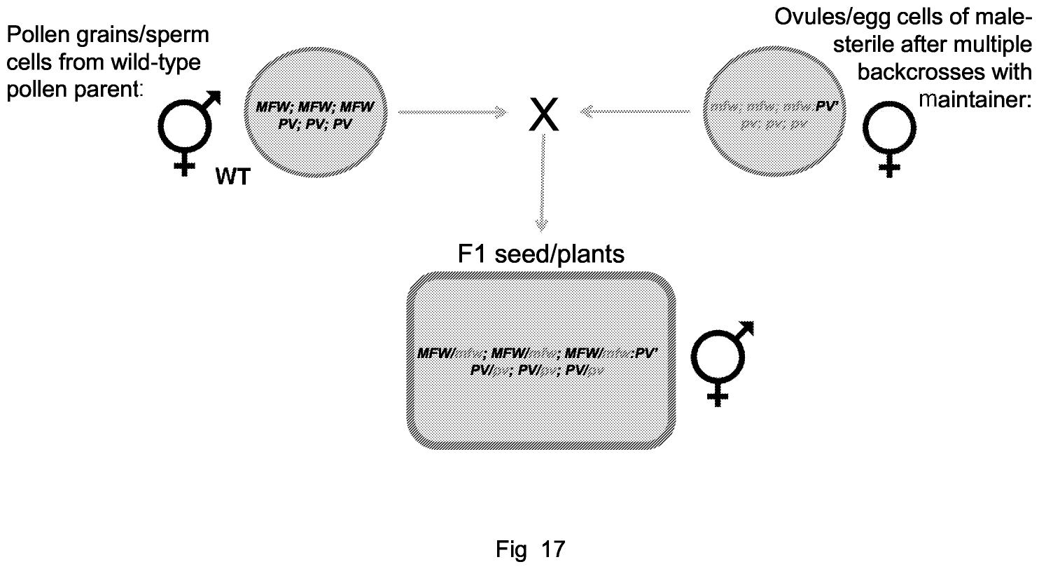

Final F1 seed production can proceed as shown in FIG. 17. It is contemplated that in F1 seed production, the pollen parent will be physically mixed with the male-sterile for maximum field pollination, maximum seed yield reliability, and least cost.

### Advantages

The male-sterile and maintainer lines described above provide lowered costs of F1 hybrid seed production. With the BA/darker-grain phenotype being colour-sortable in many seed plants, savings can be made to the final cost of male sterile-line production as well as the final F1 seed-production and provide to more easily sub-contract bioprocessing the stages to the final seed-producing company/facility.
a. The darker-grain maintainer pollinator seed can be physically mixed with the ms seed (e.g., at 1:10) to field-produce the final-stage ms seed and more maintainer seed.
b. After harvest from such a crop, the 50% of the darker-grained pollinator's seed (˜5% of the total) which is male-fertile (self-maintained maintainer) can be colour-sorted out of the ms grain. With a three-chute colour-sorter tuned to discard any ‘borderline’ grain, the darker-grain part becomes recycled maintainer seed.
c. The other non-borderline 50% seed from the maintainer (similarly ˜5% of the total) is male-sterile with effectively the same genotype as the ms itself so and efficiently adds to the ms yield as part of the same process. (The difference is that, compared to the main ms, it only has one PV allele so, come the final farmer customer's field with the wt/ms F1 heterozygote, this small proportion of the total plants, ˜ 1/19, will have ⅙ less viable pollen than other plants—there is such pollen ‘surplus’, that this is not a problem.)
d. In the final F1 seed production field (FIG. 17), the wild-type pollinator could be mixed with the ms line (e.g., at 1:15 to keep below 10% in the final seed) and the seed crop harvested as this mix. All plants' flowers will be pre-meiosis-male-fertile as all have a copy of MFW from the wild-type parent. 1/16* or 6.25% of pollen grains will have no PV allele so that proportion of pollen grains will be non-viable. With the huge ‘excess’ of pollen production in a wheat flower, having ˜6% non viable/non-germinating pollen grains will be no problem. The 94% which are viable due to the presence of a PV allele will be ample.
   *Each pollen grain needs only one PV allele to be viable. Each normal PV locus is heterozygous in the parent so, in the haploid pollen grain genome, there is a ½ probability of having the pv allele in each genome. So ⅛ have no normal-locus PV allele. Then there is a ½ chance of these also having a PV allele from the MFW-linked locus, so only 1/16 pollen grains have no PV allele.

This system has exceptional advantages for integration into breeding programmes with least disruption and least loss of focus on agronomic and other traits' improvement. Crucially the two maintainer constructs can be crossed, ‘bred in’ and selected—for along with other traits selection for all other traits only ‘converting’ the selected parent to be a male-sterile/maintainer when such progress has been achieved.

With this system being based on the use of highly conserved genes associated with male-fertility, it can also be used in other cereal grain species. Highly homologous orthologues of MFW and PV are endogenous in barley, rice, and other cereals. The concept of fine editing of the fertility gene inserts to avoid them being targeted at knockout (for male-sterility) while maintaining the identical aa sequences and proteins for full expression from the new inserts means this system is readily applicable to diploid cereals as well as hexaploid wheat.

For the first time, this offers the opportunity for a range of cereal crops to have a common hybrid system across them all with consequent advantages for costs, efficiency and ease of management.

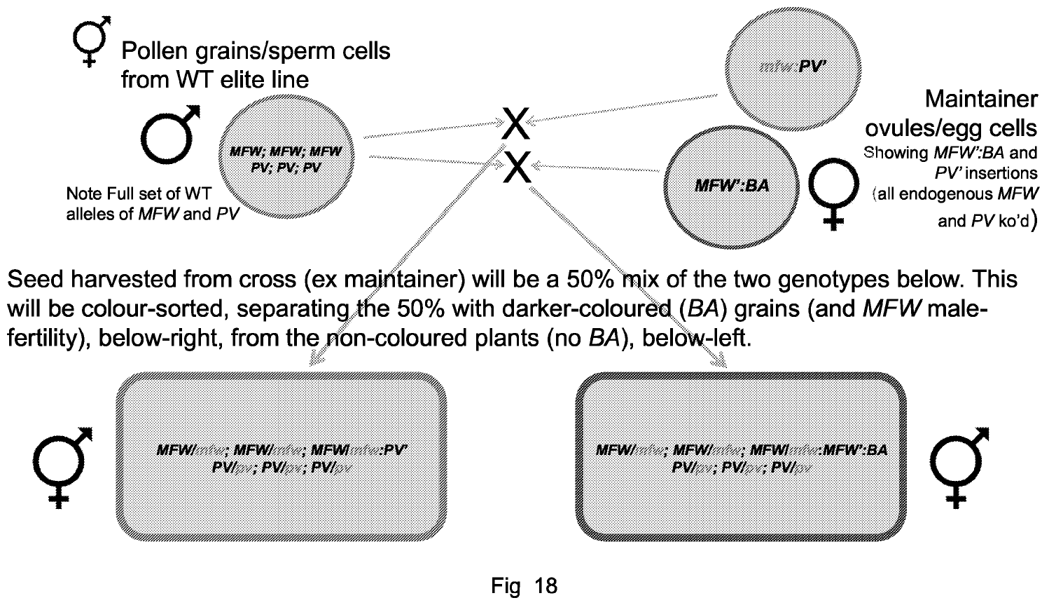

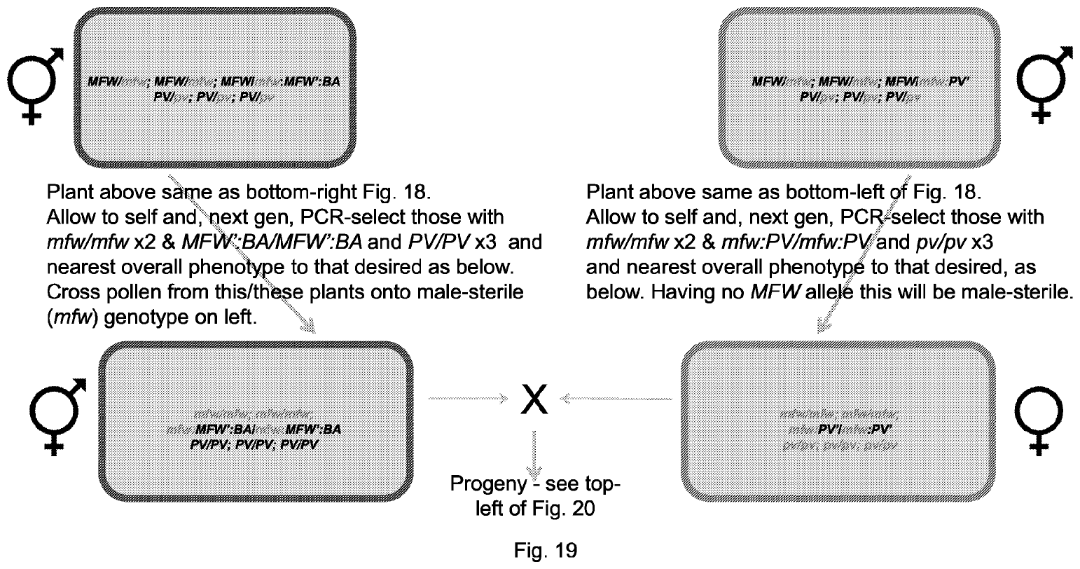

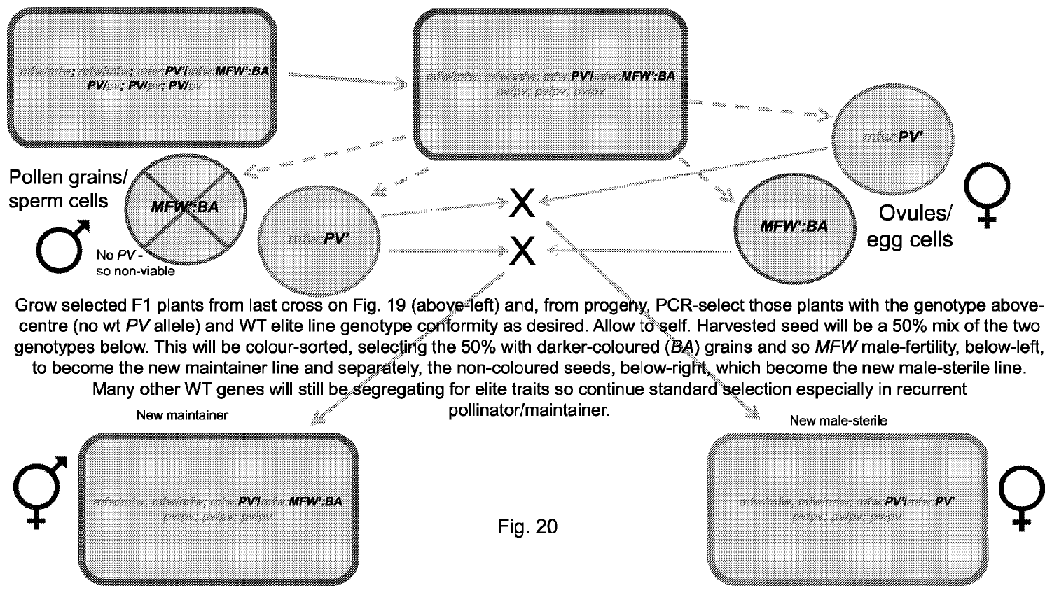

Creating new maintainer and male-sterile plants. The maintainer line described above can be crossed with an improved elite WT line, and improved new maintainer and male-sterile lines selected out from the resulting progeny (FIGS. 18-20). After about five to six generations, maintainer and male-sterile will have introgressed into new elite material. With Single Seed Descent/Speed-Breeding (see, e.g., Watson et al. Nature Plants 2018 4:23-29, which is incorporated by reference herein in its entirety) on this material and selection for maximum conformity with the elite parent, fully useable new lines can be provided.

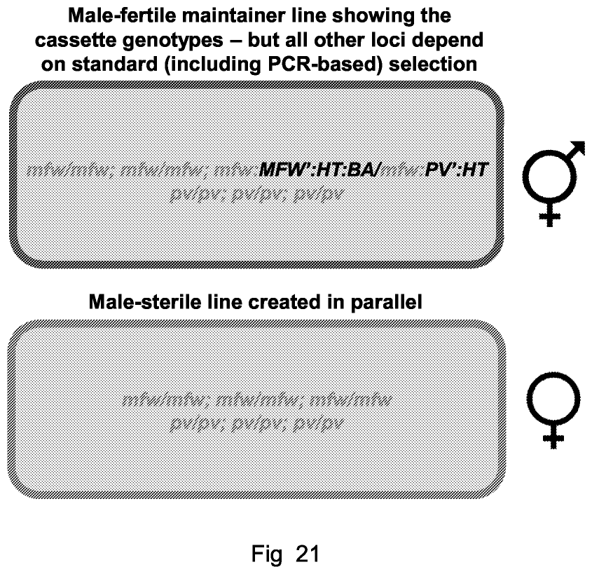

Herbicide tolerance. An addition to the processes described above can be to add a herbicide tolerance gene to each of the two cassettes, either in initial creation of a maintainer line or introducing the maintainer traits into a new elite line. This would allow, for example, an easier and far larger-scale selection for the new elite parent genotype. A field or greenhouse spray of, for example, jiffy pot plants would then allow the selected plants to be planted in the field as a uniformly-spaced population for field selection. This could be important as a means to increase the elite-line conformity in what is the recurrent pollen parent and thus the source of the long-term genotype. (FIG. 21).

Maximum breeding gain from standard breeding progress can also be achieved by just introgressing the maintainer cassettes into new lines and then doing CRISPR knockout of the endogenous MFW and PV genes.

### Example 4

### Producing a Maintainer

A selected wild-type elite wheat line can be transformed with two expression cassettes containing three genes which are ‘knocked in’: Mfw and BA in a single cassette (MFW:BA, total of ˜42 Kbp cassette or could be ˜24 Kbp as in FIG. 31B), the other cassette with PV (˜10 kb). This can be done using, e.g., a Zinc-Finger Nuclease to make the insertion at a selected ‘landing site’ (unrelated to either endogenous gene locus). Exemplary landing sites are known in the art, e.g., the ANXA1 locus as described in WO 2013/169802, which is incorporated by reference herein in its entirety.

The MFW and PV genes within the two maintainer cassettes are designed to have (or naturally have) synonymous differences/edits (compared to wild-type) at the planned CRISPR/Cas9 knockout guide sites. This is so that they are not recognised by the CRISPR knockout guides at the later male-sterility-creating stage (Step 2); their mRNA and amino acid sequences are unchanged so they code for/produce normal fertile phenotypes. (These subtly different or edited sequences are denoted MFW′ and PV′ hereafter).

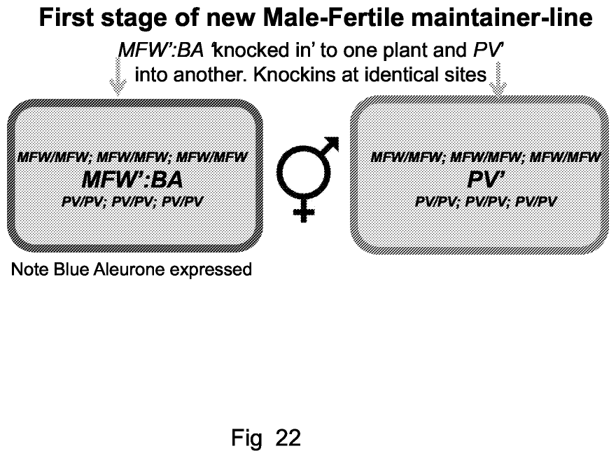

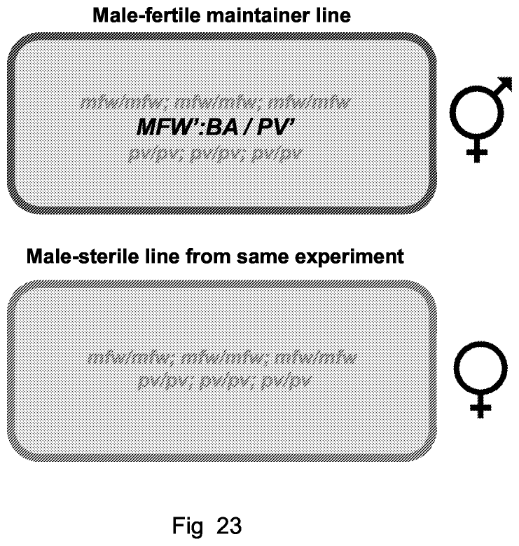

T0 plants from each experiment are identified which have successful insertions of MFW′:BA and PV′ respectively, each in a hemizygous form (FIG. 22). A plant of each is crossed. Embryos on the resultant F1 plants (so F2) are then subjected to CRISPR-Cas9 knockout of all endogenous copies of MFW and PV—see FIG. 23.

### Producing the Maintainer and Male-Sterile Lines Together

All endogenous copies of MFW and PV can be mutated/knocked-out by CRISPR/Cas9. The inserted MFW′ and PV′ expression cassettes (FIG. 22) will not be CRISPR-targeted/mutated due to the CRISPR editing guides not recognising/targeting the slight/synonymous DNA sequence changes/differences at the CRISPR/Cas9 guide sites of the inserted genes.

Being F2, ˜25% of the embryos targeted are heterozygous MFW′:BA/PV′; plants of these become the new maintainer (upper portion of FIG. 23). ˜50% of the embryos targeted are homozygous null insert or hemizygous PV′/−; plants of these, having no inserted MFW′ and successful knockouts of all endogenous MFW and PV genes, become the new male-sterile (lower portion of FIG. 23).

The fertility/sterility phenotype of the maintainer plants and their gametes is now dependent on the two inserted allelic ‘constructs’: MFW′:BA and PV′ (so hereon in this Example knocked out genes are shown in grey/smaller as they are not relevant to the phenotype). Male-fertility is expressed strongly by MFW′ to such an extent that one allele can restore full fertility, resulting in good maintainer pollen production and seed yields. This is demonstrated experimentally below herein.

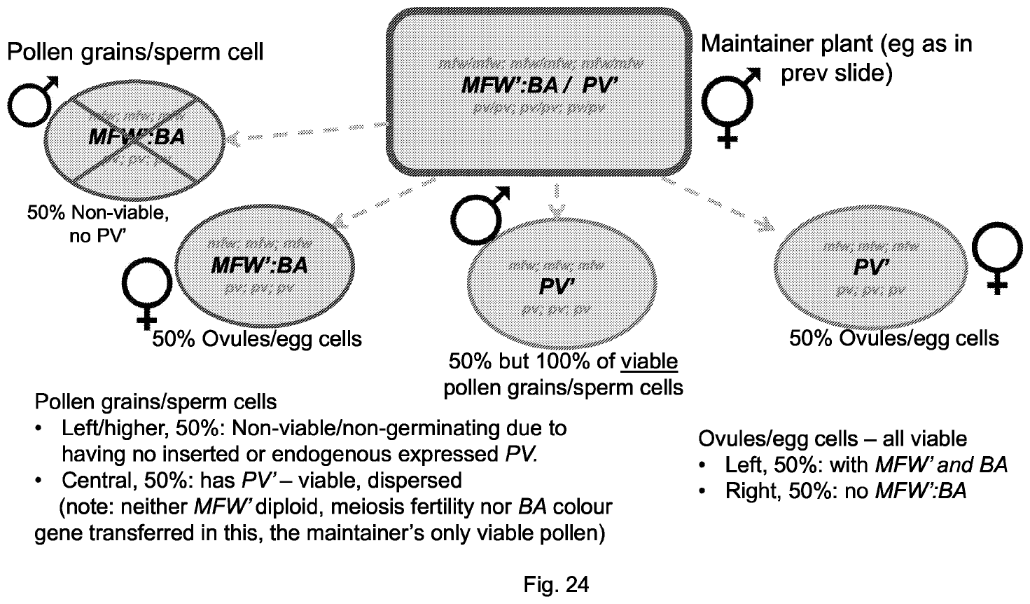

As shown in FIG. 24, these plants' viable pollen will not carry the MFW′ gene: the 50% of the (haploid) pollen grains which carry the MFW′ gene have mutated pv genes only and so are non-viable (wild-type PV required for pollen germination). Hence no transfer of MFW male-fertility to the male-sterile.

### Maintaining the Maintainer (1)

In FIG. 24, the far left pollen genotype is disabled at pollen germination as it has no PV′ allele. The two right hand ovule genotypes have been enabled for fertilisation by pre-meiosis MFW′ as in the central heterozygote at the top of FIG. 24.

Creating the Maintainer and Male-Sterile Lines Together Once a Maintainer has Already been Created in a Program

In an established breeding program which already has a useable maintainer line with this system, the process shown in FIGS. 23 and 24 can be accelerated and simplified as follows.

A new elite line can be back-crossed onto the established maintainer plant (with significant numbers and marker-assisted selection it should be possible to achieve near isogenic lines (to the elite parent) in ˜three generations. In F2 and on, selection can proceed on strict agronomic traits just ensuring that there is >1 plant in the progeny which has PV′ and >1 which has MFW′/BA. When selection is near completion a selected plant with MFW′:BA can be crossed with a near isogenic selected plant with PV′. If available, one parent can be hemizygous in order to bring in a null locus for the male-sterile. If neither of these is present in hemizygous form, an initial cross can be done with a null-inserted-gene plant and the next stage can be performed a generation later.

Then, taking embryos from a few plants with the target genotype (ie it must include one genome with allelic MFW′:BA/PV′) all endogenous copies of MFW and PV are mutated/knocked-out by CRISPR/Cas9. If there is no appropriate heterozygote, then two complementary homozygote (most plants will be homozygous by this stage) can be crossed and the F1's embryos mutated (and a null insert plant mutated for the male-sterile plant to be obtained).

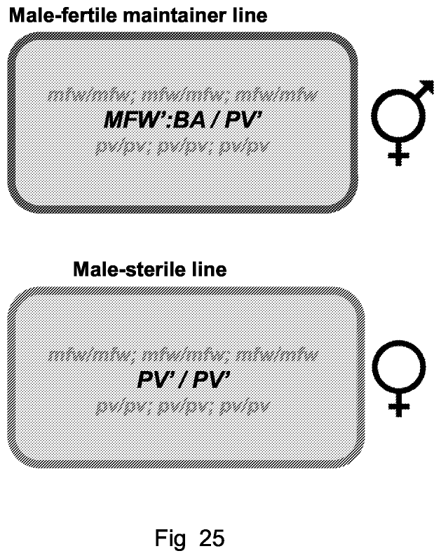

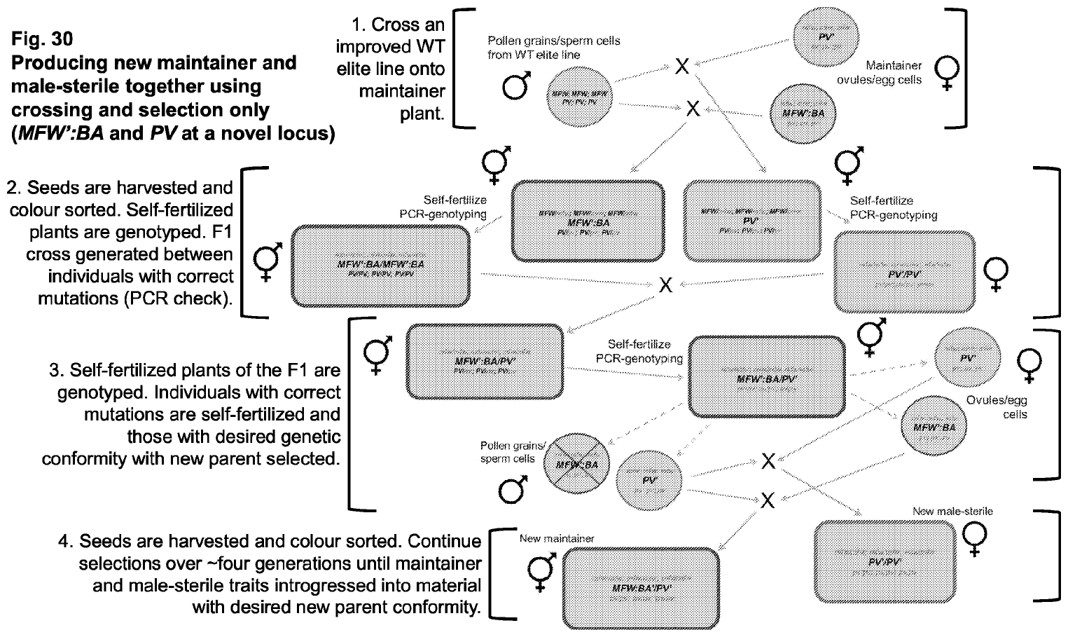

Again, the inserted MFW′ and PV′ expression cassettes will not be CRISPR-targeted/mutated and express the necessary fertility proteins. Plants for the new male-sterile can be selected from embryos which have homozygous PV′/PV′ and a full set of endogenous knockouts. Plants for the new maintainer can be selected from embryos with the heterozygous maintainer combination MFW′:BA/PV′ and a full set of endogenous knockouts. In other words, To plants with a full-set of knocked out endogenous genes are selected to become the new male-sterile: heterozygous MFW′:BA/PV′ (upper portion of FIG. 25) for the maintainer and homozygous PV′/PV′ (lower portion of FIG. 25)—or PV′/− or −/− after above secondary options. Thus both a new maintainer and new male-sterile are produced from the same experiment in the same new genetic background (FIG. 25 and FIG. 30). Further ‘fine-tuning’, for example, for certification criteria can be effected by selection for relevant traits within the maintainer line—improvements will be passed on to the male-sterile by recurrent fertilisation with the maintainer.

Creating the Maintainer and Male-Sterile Lines Together Once a Maintainer has Already been Created in a Program—by Normal Crossing

In an established breeding program which already has a useable maintainer line with this system, the process shown in FIGS. 23 and 24 can be accelerated and simplified as follows.

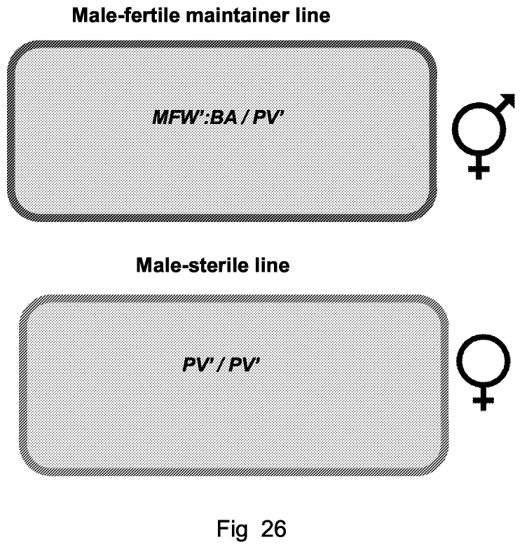

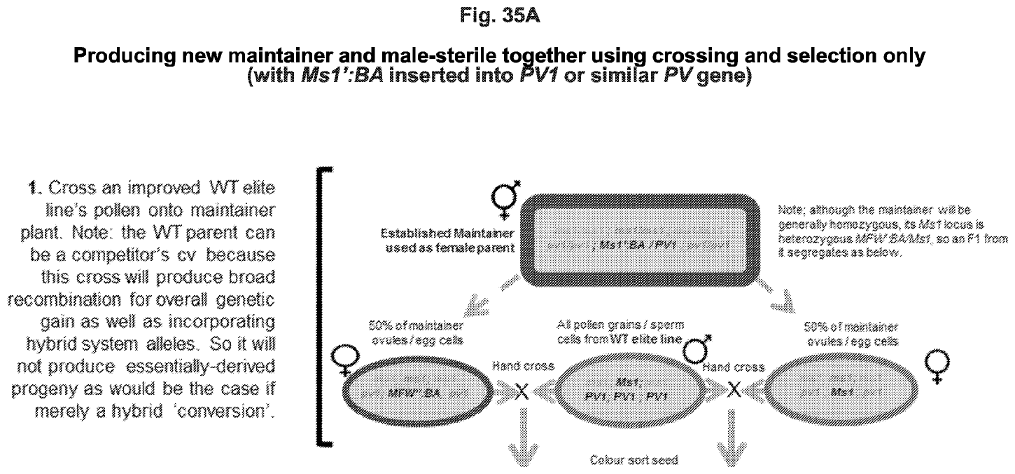

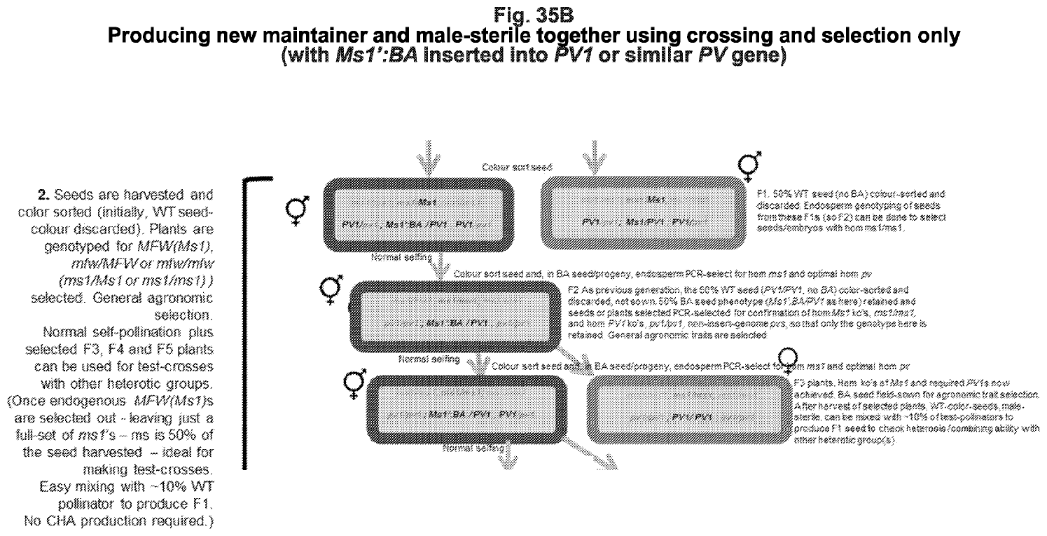

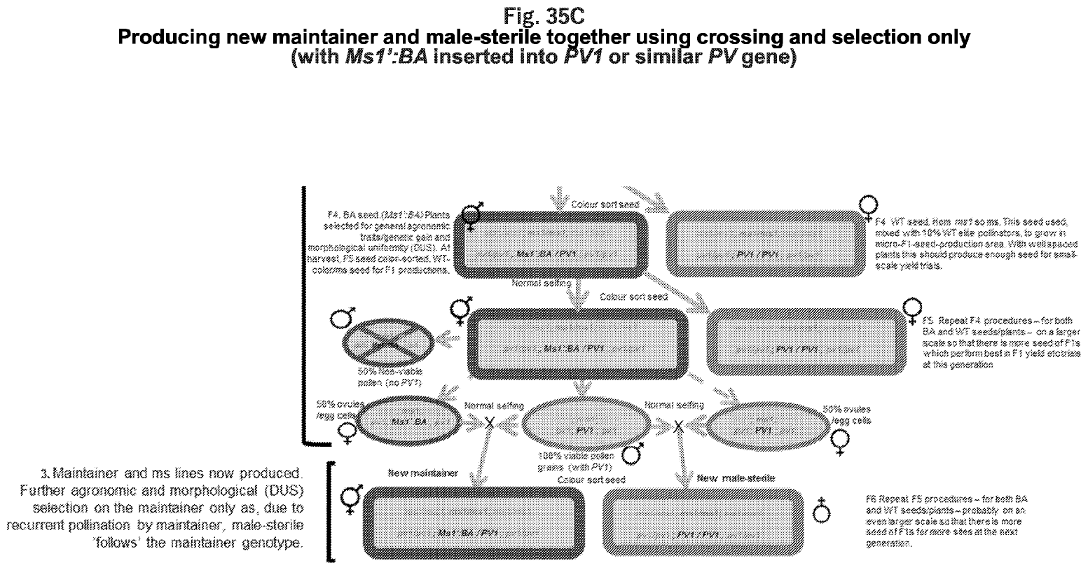

A new elite line can be back-crossed onto the established maintainer plant. The first F1. generation will be heterozygous at all endogenous loci/hemizygous at the new locus. In the F2, all loci have a ¼ chance of being homozygous for the desired knockout so with six loci there is a 1/4096 chance of all endogenous MF and PV loci being homozygous knockouts. Of those, ¼ (˜ 1/16000) will be MF′:BA/PV′ (for the maintainer) and ½ (˜ 1/8000) will be PV′/− or PV′/PV′ (for the male-sterile). Then there is the primary need to have a large enough remaining population to be able to select for the segregants with the best agronomic phenotype like the elite cv parent. An alternative strategy would be to select initially for agronomic elite type and then select for those segregates with the best combination of homozygous and heterozygous maintainer alleles (FIG. 26). Particularly if Ms1 (expressed only from the B genome thus reducing the rate of deselection needed) is used as the MF gene, a further alternative strategy would be to PCR-select initially (in F2 and F3 seeds) for the necessary combination of hybrid system alleles (FIGS. 35A-35C) leaving agronomic and other traits for selection in later generations.

Where regulatory approval for genome edited plants is/becomes reasonably easy and with increasingly widespread ability to use CRISPR-Cas technology, the preceding strategy may be most efficient.

### Maintaining the Maintainer—and Producing Male-Sterile Seed at the Same Time

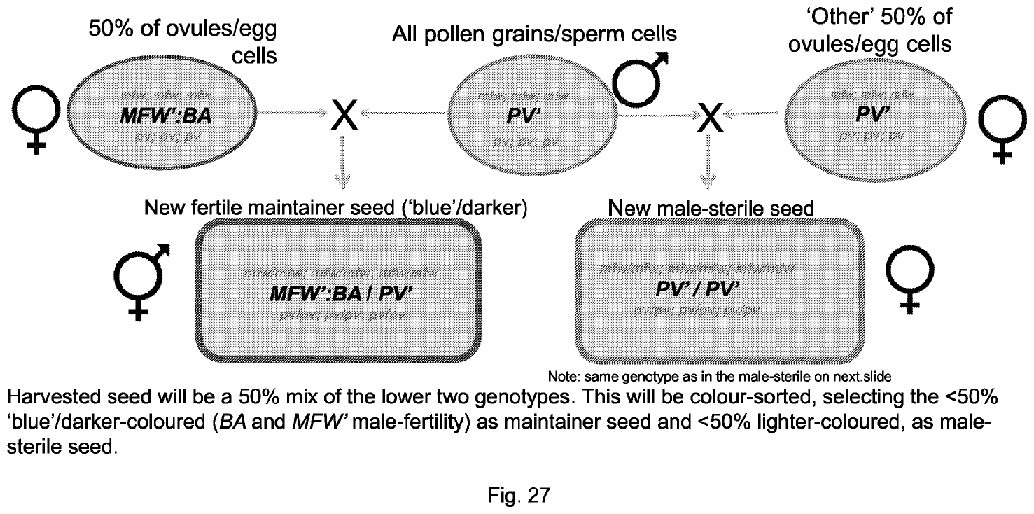

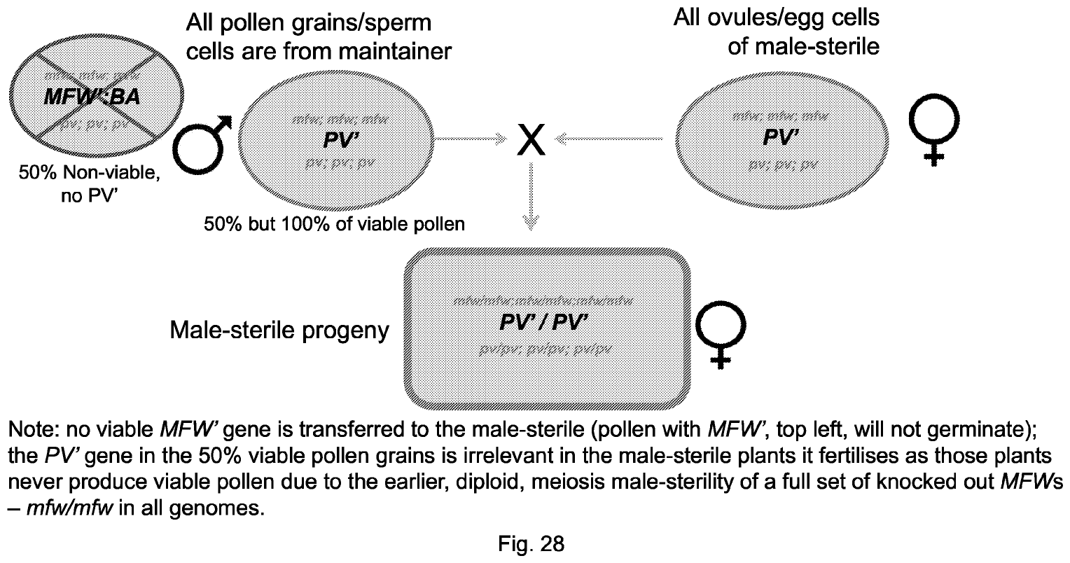

FIG. 27 depicts an approach for maintaining the maintainer line and generating male-sterile seed at the same time (the production of these gametes is shown in FIG. 24). FIG. 28 depicts an approach for male-sterile production from the maintainer line of FIG. 27 when the approach of FIG. 27 will not provide sufficient male-sterile progeny.

### Advantages of Described System

The BA gene's grain phenotype has been shown to be dose-related, but one allele's expression is enough for a darker-grained phenotype to be colour-selectable. In fact in the maintainer's endosperm there will be two BA alleles from the ovule/maternal side and null from the pollen/paternal side. Being from the same bread wheat species all the introduced genes described herein will be from the same species which will promote regulatory approval and end-market acceptability.

After harvest of a male-sterile seed production crop, the 50% of the darker-grained mainainer/pollinator's seed (˜5% of the total) which is male-fertile (self-maintained maintainer) can be colour-sorted out of the male-sterile grain. With a three-chute colour-sorter tuned to discard any ‘borderline’ grain, the darker-grain part becomes recycled maintainer seed.

The other non-borderline 50% seed from the maintainer (similarly ˜5% of the total) is male-sterile with effectively the same genotype as the male-sterile itself so (see FIG. 24) and efficiently adds to the male-sterile yield as part of the same process. (The difference is that, compared to the main male-sterile, it only has one PV allele so in the final farmer customer's field with the wt/ms F1 heterozygote, this small proportion of the total plants, ˜ 1/19, will have ⅙ less viable pollen than other plants. Given the pollen surplus present in the field, this decrease of pollen production will not negatively impact crop production.

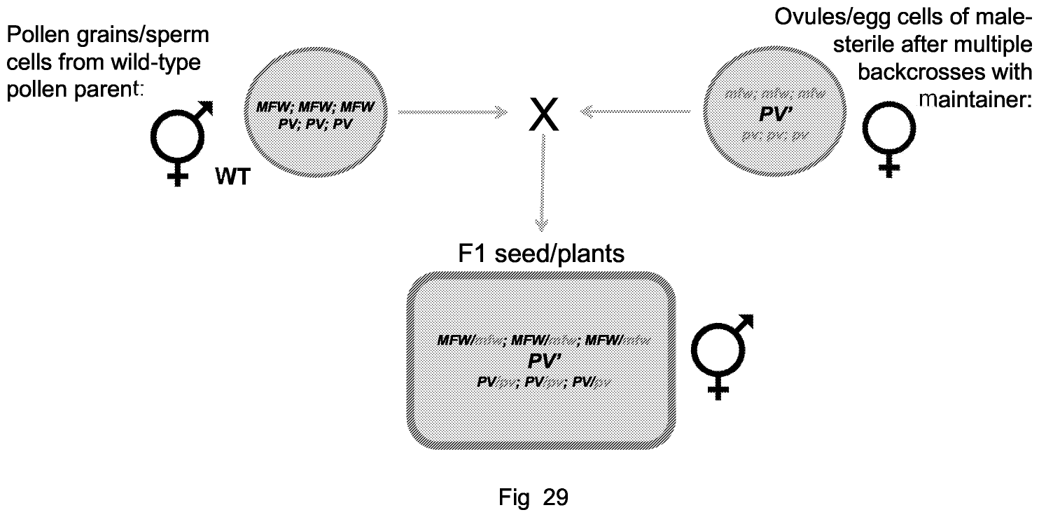

In the final F1 seed production field (FIG. 29), the wild-type pollinator can be be mixed with the male-sterile line for an effective spread of pollen within the F1 seed crop. A ratio of 1:15 (˜7%), for example, will keep the pollinator seed below 10% of the plants in the final F1 seed crop and the seed crop can be harvested as this mix.

When the F1 plants produced according to FIG. 29 are grown, all plants' flowers will be pre-meiosis-male-fertile as all have a copy of MFW from the wild-type parent. In this population, 1/16* or 6.25% of pollen grains will have no PV allele so that proportion of pollen grains will be non-viable. With the huge ‘excess’ of pollen production in a wheat flower, having ˜6% non viable/non-germinating pollen grains will not negatively effect crop yields. The 94% which are viable due to the presence of a PV allele will be ample.

*Each pollen grain needs only one PV allele to be viable. Each normal PV locus is heterozygous in the parent so, in the haploid pollen grain genome, there is a ½ probability of having thepv allele in each genome. So ⅛ have no normal-locus PV allele. Then there is a ½ chance of these also having a PV allele from the MFW-linked locus, so only 1/16 pollen grains have no PV allele.

### Example 5

PV1/NPG1 was deleted using CRISPR and guide RNAs as described herein. The genome and phenotypes of the resulting plants were examined and are presented in Table 3. As evidenced by AK30A.1.2, when each of the six alleles of PV1 in the genome are loss-of-function alleles, the plant displays a complete male-sterility phenotype.

TABLE 3
Genotypes are indicated as wild-type [WT}, het [indicating one mutant/one WT) or
−xbp [showing the number of base-pairs deleted where that data is available]. The
Tiller number is an indication of plant growth, with >7 tillers/plant indicative of well-grown plants.

| | A Genome | | B Genome | | D Genome | | | Seed | Seed | Tiller |
|---|---|---|---|---|---|---|---|---|---|---|
| Sample | guide 1 | guide 2 | guide 1 | guide 2 | guide 1 | guide 2 | guide 3 | number | weight | number |
| AK30A.1.1 | WT | bp +1/WT | bp +29 | bp +1 | WT | bp +1 | WT | 228 | 9 | 10 |
| AK30A.1.2 | WT | bp +1/bp −7 | bp +29 | bp +1 | WT | bp −172 | bp −172 | 0 | 0 | 11 |
| AK30A.1.3 | WT | bp +1 | WT/bp +29 | bp +1 | WT/bp −13 | bp +1 | WT | 276 | 12 | 8 |
| AK30A.3.1 | WT | bp +1 | WT | bp +1 | WT | het | WT | 332 | 14.4 | 7 |
| AK30A.3.2 | ? | het | WT | bp +1 | WT | bp +1 | WT | 425 | 15.2 | 11 |
| AK30A.3.3 | WT | bp +1/bp −10 | bp −1/WT | bp +1/WT | WT | bp −512 | bp −512 | 360 | 16.7 | 8 |
| AK30A.3.4 | WT | bp +1 | het | bp +1 | WT | het | WT | 319 | 14.7 | 7 |
| AK30A.3.5 | WT | bp +1 | WT | bp +1 | WT | het | WT | 333 | 14.5 | 8 |
| AK30B.3.1 | WT | bp +1 | het | het | Het | bp+1 | WT | 277 | 10.8 | 9 |
| AK33.5.1 | WT | bp +1 | ? | het | WT | het | WT | 335 | 15.3 | 7 |
| AK33.5.2 | WT | bp −1 | WT | het | WT | bp +1 | WT | 294 | 13.6 | 8 |

### Example 6

PV1 and Mfw2:BA1 Knocked in at a Non-Mfw2 or -PV1 Loci in Wheat in Order to Produce, after Appropriate Crossing and Selection, a PV1 Knock-In in One of the Two Homologous Loci and Mfw2:BA1 in the Other Homologous Locus so that they Become Sister Alleles at that Locus.

To produce plants with targeted insertion of PV1 and Mfw2:BA1, a CRISPR CAS system or ZFN or other site-directed nuclease system can be employed to introduce these gene transfers at a desired location in wheat plants to introduce the genes PV1 and Mfw2.

For the insertion of PV1, a construct can be made with the wheat PV1 genomic sequence driven by ˜1.5 kb of its own promoter and ˜1 kb of its terminator. This DNA sequence is changed minimally (2 bp) (or a rare natural variant is chosen)—enough sequence variation to disrupt the possibility of a future guide RNA targeted at endogenous PV1 from editing this sequence once it is introgressed into the wheat genome and enough to be able to PCR-select for it but not for endogenous PV1. This will include changing the DNA sequence from GTCGCCCCTCCTGAGGCGGCGG (SEQ ID NO: 166) which is the nuclease target in the native PV1 sequence to GTCGCCCCTCCTGAGGCAGCAG (SEQ ID NO: 167) which will not change the amino acid sequence of the protein but not allow the guide to target the introgressed PV1. This different/adapted PV1 is titled PV1′.1 hereafter and the complete sequence of PV1′.1 is provided in SEQ ID NO: 172. SEQ ID NO: 172 provides a construct for PV1 genomic introgression, the construct comprising PV1′.1 with the endogenous PV1 promoter. The altered guide RNA target sequence (SEQ ID NO: 167) is found at nucleotides 2,169-2,190 of SEQ ID NO: 172. PV1′.1 DNA along with a binary vector containing a wheat optimized Cas9 driven by the maize ubiquitin promoter and guide RNA driven by a TaU6 PollIl promoter targeting PV1 can be introduced into wheat embryos either by biolistics or agrobacterium mediated transformation. An alternative strategy would be to find, e.g. from an exome sequence database (see, e.g., He, F. et al. Nat. Genet. 2019 515, 51, 896-904; which is incorporated by reference herein in its entirety) a rare natural/endogenous variant whose sequence would then become MF′ or PV′.

Plants can then be screened for insertion of the gene using a PCR based method where the PCR product is amplified for each homoeologue anchored to the possible insertion and sequenced to verify insertion. Plants can be selected which have the PV1′.1 insertion or the insertion of Mfw2 (as follows).

For the insertion of Mfw2, again an intermediate construct can be made with Mfw2 cDNA driven by 1.5 kb of its own promoter and 1 kb of its terminator followed by BA1 driven by the high molecular weight glutenin promoter and 1 kb of its native terminator. This DNA sequence is changed minimally (2 bp)—enough to disrupt the possibility of a future guide RNA targeted at Mfw2 from editing the Mfw2 sequence once it is introgressed into the wheat genome and enough to be able to PCR-select for it but not for endogenous Mfw2. This can include changing the DNA sequence from GGATGGCCAATGCGAGATGATGG (SEQ ID NO: 168) which targets the native Mfw2 sequence to GGATGGCCAATGCGAGACGACGG (SEQ ID NO: 169) which will not change the amino acid sequence of the protein but not allow the guide to target the introgressed Mfw2. This different/adapted Mfw2 is titled Mfw2′.1 hereafter and the complete sequence of Mfw2′.1 is provided in SEQ ID NO: 173. SEQ ID NO: 173 provides a construct for Mfw2 genomic introgression, the construct comprising Mfw2′.1 with the endogenous Mfw2 promoter, and followed by BA with wheat HMWG promoter, HMWG::TaBA1. The altered guide RNA target sequence (SEQ ID NO: 169) is found at nucleotides 7,257-7,279 of SEQ ID NO: 173 and the HMWG promoter is found at nucleotides 20,748-21,165 of SEQ ID NO: 173. This DNA along with a binary vector containing a wheat optimized Cas9 driven by the maize ubiquitin promoter and guide RNA driven by a TaU6 PolIII promoter targeting Mfw2′.1 can be introduced into wheat embryos either by biolistics or agrobacterium mediated transformation.

Plants can then be screened for insertion of the DNA sequence using a PCR based method where the PCR product is amplified for each homoeologue anchored to the possible insertion and sequenced to verify insertion of Mfw2′0.1. Plants can be selected which had the Mfw2′0.1:BA1 insertion on the same homoeologue as the PV1 insertion above. Plants with an insertion of either PV1′.1 or Mfw2′.1 can then be crossed to combine the inserted sequences in the same plant.

Immature embryos from plants from the previous cross would then have their endogenous Mfw2, and PV1 genes knocked out in all native loci except Mfw2 on the chromosomes containing the above constructs, this is the basis of the maintainer line. As only the chromosomes with the above knockins have a functional Mfw2 expressing Mfw2 protein, the other six homoeologous alleles will be knocked out.

In another embodiment for the production of the maintainer line the native Mfw2 and PV1 homoeologues can be knocked out first and the resulting sterile plant could be rescued by either a WT plant containing fully fertile pollen or a WT plant containing one of the knocked in DNA sequences. This cross could then be further crossed to combine both the knocked in DNA sequences and track the mutated PV1 and Mfw2 alleles to select for a plant in which the native Mfw2 and PV1 sequences are knocked out and a single copy of the inserted Mfw2′0.1:BA1 and PV1′.1 sequences are in the wheat genome.

### Example 7

A wheat plant in which all six copies of Mfw2 were knocked-out, by CRISPR-Cas targeting as described elsewhere herein. The plant was also confirmed to have all T-DNA, including Cas9, segregated out. This male-sterile plant was crossed with wild-type Fielder male-fertile plants to produce seed. The F1 progengy were heterozygous at all loci. The F2 progeny, with a total population of 69 plants, showed every potential combination of alleles across the three sub-genomes.

In particular, two plants with complete homozygous knockouts of all alleles of Mfw2 displayed complete male-sterility. All other plants/genotypes were fully fertile. This demonsrates that Mfw2 provides recessive male-sterility.

These two diploid plants must have been fertilised by viable haploid pollen grains to produce their homozygous genotype. The pollen grains concerned could not have been viable haploids if Mfw2 were post-meiosis as the lack of Mfw2 at that stage would have made them non-viable as independent haploid cells. They had the ‘protection’ of a single wild-type MW2 allele being expressed during diploid meiosis to pass through that stage. This provides proof of diploid-stage expression of Mfw2—to give vital meiosis-stage viability for the post-meiosis independent haploid pollen grains with knocked-out MW2 alleles which do not transfer Mfw2. (The ‘other allele’ pollen grains, with Mfw2, have no post-meiosis PV gene so are never viable to transfer their MW2 alleles.)

There were seven plants with one genome only having a MFW2/mfw2 heterozygous genotypes with other genomes having full knockouts; average seed set in these seven plants was actually higher than in wild type plants. This is proof of the effectiveness of Mfw2 from expression of a single wild type Mfw2 allele. It is further contemplated herein that embodiments relating to Mfw2 have higher seed yields than some other WT MFW/Ms genes.

TABLE 4
Mfw2 genotype expression. Demonstrates thte ability of
a single wild-type Mfw2 allele to maintain fertility.

| | Summarised | avg. seed weight | avg. seed number |
|---|---|---|---|
| Genotype details | Genotype | (g) per plant | per plant |
| Single Wild-type Mfw2/mfw2 het in | Aabbdd | 13.05 | 308.5 |
| A genome, all others knocked out | | | |
| Single Wild-type Mfw2/mfw2 het in | aaBbdd | 9.5 | 236.5 |
| B genome, all others knocked-out | | | |
| Single Wild-type Mfwe/mfw2 het in | aabbDd | 11.37 | 303.67 |
| D genome, all others knocked-out | | | |
| All alleles wild-type | AABBDD | 9.24 | 248 |
| (de facto control) | | | |
| All alleles knocked out | aabbdd | 0 | 0 |

### Example 8

PV1 and MsI:BA1 Knocked in at a Non-Ms1 or -PV1 Loci in Wheat in Order to Produce, after Appropriate Crossing and Selection, a PV1 Knock-In in One of the Two Homologous Loci and MsI:BA1 in the Other Homologous Locus so that they Become Sister Alleles at that Locus.

To produce plants with targeted insertion of PV1 and Ms1:BA1, a CRISPR CAS system or ZFN or other site-directed nuclease system can be employed to introduce these gene transfers at a desired location in wheat plants to introduce the genes PV1 and Ms1.

For the insertion of PV1, a construct can be made with the wheat PV1 genomic sequence driven by its own promoter and terminator. This DNA sequence is changed minimally (e.g., 1 bp)—enough sequence variation to disrupt the possibility of a future guide RNA targeted at endogenous PV1 from editing this sequence once it is introgressed into the wheat genome and enough to be able to PCR-select for it but not for endogenous PV1. This will include changing the DNA sequence from GATGCACTTTGTGTGTTTGATGG (SEQ ID NO: 260) which is the nuclease target in the native PV1 sequence to GATGCACTTTGTGTATTTGATGG (SEQ ID NO: 261) which will not change the amino acid sequence of the protein but not allow the guide to target the introgressed PV1. This different/adapted PV1 is titled PV1′.1 hereafter. PV1′.1 DNA along with a binary vector containing a wheat optimized Cas9 driven by the maize ubiquitin promoter and guide RNA driven by a TaU6 PolIII promoter targeting PV1 can be introduced into wheat embryos either by biolistics or agrobacterium mediated transformation.

Plants can then be screened for insertion of the gene using a PCR based method where the PCR product is amplified for each homoeologue anchored to the possible insertion and sequenced to verify insertion. Plants can be selected which have the PV1′.1 insertion or the insertion of Ms1 (as follows).

For the insertion of Ms1, again an intermediate construct can be made with a Ms1-encoding sequence (e.g., genomic Ms1 or cDNA) driven by 1.5 kb of its own promoter and 1 kb of it terminator followed by BA1 driven by a) the high molecular weight glutenin promoter and 1 kb of its native terminator or b) 1.5 kb of its own promoter and 1 kb of its own terminator. This DNA sequence is changed minimally (e.g., 1 bp)—enough to disrupt the possibility of a future guide RNA targeted at Ms1 from editing the Ms1 sequence once it is introgressed into the wheat genome and enough to be able to PCR-select for it but not for endogenous Ms1. This can include changing the DNA sequence from GCGGGCTGCTGCTGGTGGCGGGGG (SEQ ID NO: 219) which is the nuclease target in the native Ms1 sequence to GCGGGCTGCTGCTGGTGGCTGGAG (SEQ ID NO: 220) which will not change the amino acid sequence of the protein but not allow the guide to target the introgressed Ms1. Alternatively, this can include changing the DNA sequence from GGCTCGCAGCACTGCGCCGTCGG (SEQ ID NO: 262) which is the nuclease target in the native Ms1 sequence to GGCTCGCAGCACTGGGCCGTCGG (SEQ ID NO: 263) which will not change the amino acid sequence of the protein but not allow the guide to target the introgressed Ms1. This different/adapted Ms1 is titled Ms1′.1 hereafter. This DNA along with a binary vector containing a wheat optimized Cas9 driven by the maize ubiquitin promoter and guide RNA driven by a TaU6 PolIII promoter targeting Ms1′.1 can be introduced into wheat embryos either by biolistics or agrobacterium mediated transformation.

Plants can then be screened for insertion of the DNA sequence using a PCR based method where the PCR product is amplified for each homoeologue anchored to the possible insertion and sequenced to verify insertion of Ms1′.1. Plants can be selected which had the Ms1′.1:BA1 insertion on the same homoeologue as the PV1 insertion above. Plants with an insertion of either PV1′.1 or Ms1′.1 can then be crossed to combine the inserted sequences in the same plant.

Immature embryos or subsequent plant tissue from this cross would then have their endogenous Ms1, and PV1 genes knocked out in all native loci except Ms1 on the chromosomes containing the above constructs, this is the basis of the maintainer line. As only the chromosomes with the above knockins have a functional Ms1 expressing MS1 protein, the other two homoeologous alleles (the B genome homoeologues as WT Ms1 is only expressed from the two B genome copies) will be knocked out.

In another embodiment for the production of the maintainer line the native Ms1 and PV1 homoeologues can be knocked out first and the resulting sterile plant could be rescued by either a WT plant containing fully fertile pollen or a WT plant containing one of the knocked in DNA sequences. This cross could then be further crossed to combine both the knocked in DNA sequences and track the mutated PV1 and Ms1 alleles to select for a plant in which the native PV1 and Ms1 sequences are knocked out and a single copy of the inserted Ms1′.1:BA1 and PV1′.1 sequences are in the wheat genome.

A similar approach can be taken utilizing Mfw2 in place of Ms1 as the MF gene. For example, for the insertion of Mfw2, again an intermediate construct can be made with a Mfw2-encoding sequence (e.g., genomic Mfw2 or cDNA) driven by 1.5 kb of its own promoter and 1 kb of it terminator followed by BA1 driven by a) the high molecular weight glutenin promoter and 1 kb of its native terminator or b) 1.5 kb of its own promoter and 1 kb of its own terminator. This DNA sequence is changed minimally (2 bp)—enough to disrupt the possibility of a future guide RNA targeted at Mfw2 from editing the Mfw2 sequence once it is introgressed into the wheat genome and enough to be able to PCR-select for it but not for endogenous Mfw2. This can include changing the DNA sequence from GGATGGCCAATGCGAGATGATGG (SEQ ID NO: 168) which targets the native Mfw2 sequence to GGATGGCCAATGCGAGACGACGG (SEQ ID NO: 169) which will not change the amino acid sequence of the protein but not allow the guide to target the introgressed Mfw2. This different/adapted Mfw2 is titled Mfw2′.1 hereafter and the complete sequence of Mfw2′.1 is provided in SEQ ID NO: 173. SEQ ID NO: 173 provides a construct for Mfw2 genomic introgression, the construct comprising Mfw2′.1 with the endogenous Mfw2 promoter, and followed by BA1 with wheat HMWG promoter, HMWG::TaBA1. The altered guide RNA target sequence (SEQ ID NO: 169) is found at nucleotides 7,257-7,279 of SEQ ID NO: 173 and the HMWG promoter is found at nucleotides 20,748-21,165 of SEQ ID NO: 173. This DNA along with a binary vector containing a wheat optimized Cas9 driven by the maize ubiquitin promoter and guide RNA driven by a TaU6 PolIII promoter targeting Mfw2′.1 can be introduced into wheat embryos either by biolistics or agrobacterium mediated transformation.

### Example 9

The maintainer lines described herein can also be created by means of a process as follows. To create the maintainer line a cassette comprising the genomic sequence of four genes will be introduced at random into the wheat genome of an elite breeding line suitable to become a parent of an F1. The cassette comprises a MF′ gene, a PV′ gene, a seed color gene (or a set of seed color genes), and a selection gene. An exemplary PV3′ is provided in Example 10.

This will result, in at least one plant, in strong expression of the introduced genes and complementation of the native sequences of Mfw2 and PV3. The introduced genomic DNA sequences of the MF gene (e.g., Mfw2) and the PV gene (e.g., PV3) will be modified slightly with subtle DNA-sequence SNP's to allow for next-stage targeting of the native sequences via CRISPR/Cas9 but with no change to the amino acid sequences of the encoded genes (these subtly different/adapted genomic sequences (which can be naturally-occurring) are titled Mfw2′ and PV3′ hereafter and similar designed/engineered sequences are described in detail elsewhere herein).

A diagram of the relevant cassette sequence for the initial (insertion stage) transformation is shown in FIG. 31. Such cassettes can be T-DNA sequences. The cassette itself comprises, in 5′ to 3′ or 3′ to 5′ order: i) the full genomic sequence of Mfw2′ and a seed color marker gene (or at least one functional ectopic allele of each member of a set of seed color genes), for example BA2 (with Mfw2′ and BA2 in either order relative to each other); this will be followed by ii) a selection gene, for example nptII, finally followed by iii) the full genomic sequence of PV3′. The cassette can be incorporated into the genome in either orientation with respect to any reference point in the genome. To ensure that both pollen fertility genes are at the same locus in the genome and that they segregate independently after the second transformation, four further sequences can also be included in the modification of the original cassette. These sequences include two 34 bp Cre-lox recognition sequences flanking the start of the Mfw2′ sequence and following the nptII selection gene; the other two can be the 34 bp flippase recognition sequences flanking the start of nptII and following PV3′ in order, at the next stage, to recombine out the selection gene and PV3′ [1-4]. It is contemplated that the location of the Cre-Lox and flippase sequences can be reversed. These sequences will be located before the start of selection marker and after the PV3′ genomic sequence. All these are shown in FIGS. 31B-D. An exemplary cassette utilizing PV1′ as the PV gene is provided as SEQ ID NO: 221. An exemplary cassette utilizing PV3′ as the PV gene is provided as SEQ ID NO: 232.

An exemplary embodiment of PV3 and PV3′ sequences include the common PV3 sequence of sequence from CCTTCTCCTCCACCGCGGGGCTG (SEQ ID NO: 264) which is the nuclease target in the native PV3 sequence to CCTTCTCATCCACCGCGGGGCTG (SEQ ID NO: 265) which will not change the amino acid sequence of the protein but not allow the guide to target the introgressed PV3′. This is naturally-occurring rare SNP, found at less than 0.01% frequency. In this embodiment, an exemplary guide is GTGGCCCAGCCCCGCGGTGGAGG (SEQ ID NO: 266).

Plants are selected based on a single copy insertion in the genome and plants which have good expression of BA2 at flowering/seed development to prove that the insertion has taken place at an effective locus for these genes to be truly expressed when they need to be and maintain fertility of the plant. Such plants are then, at a second stage, retransformed with either Cre-lox or with flippase to drop out either Mfw2′:BA2 (and nptII) to leave just the PV3′ in that/those plants or drop out PV3′ (and nptII) and leave just Mfw2′:BA2 in the other plant(s).

Using PCR-based selection (eg with appropriate KASP primers) one or more plants are selected which have just the Mfw2′:BA2 remaining and one or more plants are selected which have just the PV3′ remaining. These are crossed to produce F1 embryos which have the allelic pair Mfw2′:BA2/PV3′ at the new locus. (If there are no plants with only one or the other inserted allele, hemizygotes can be used but clearly more crosses will need to be made to ensure the correct F1 heterozygous embryos are available for the next stage.)

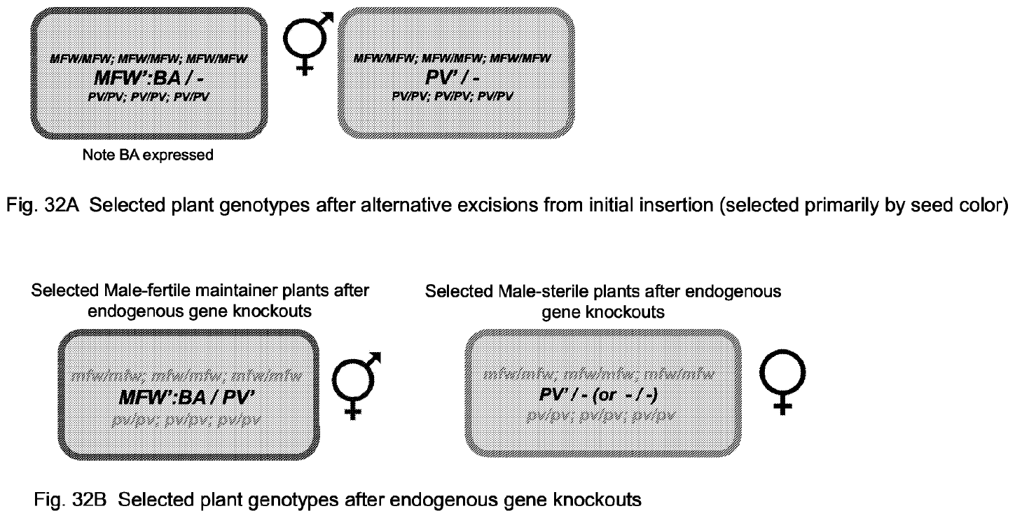

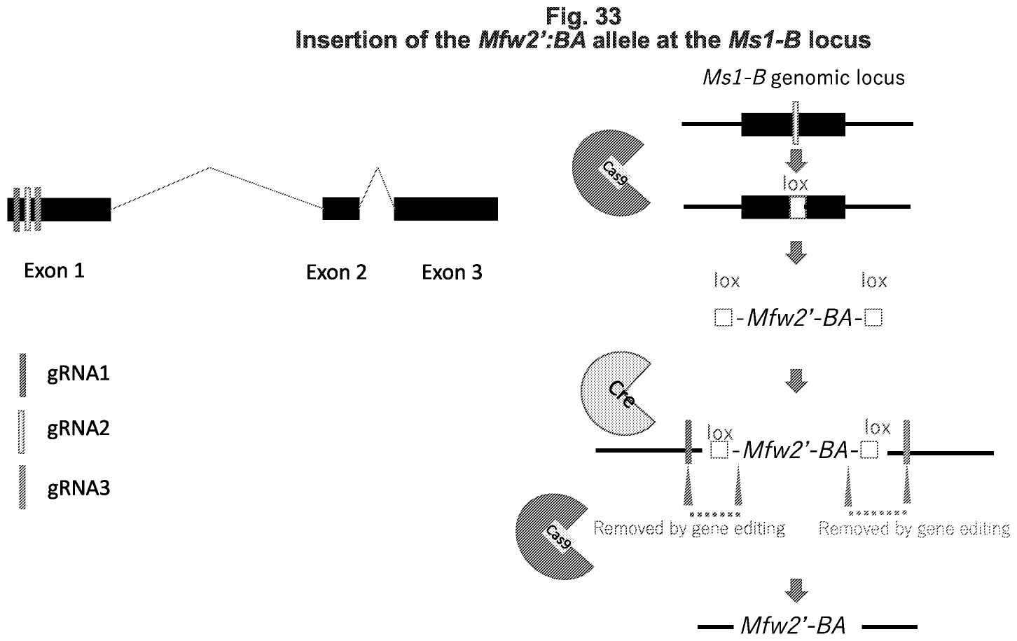

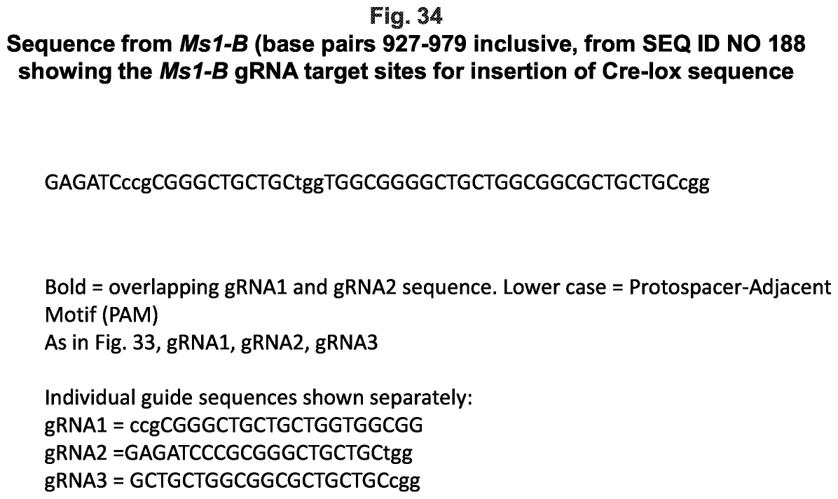

These embryos are then transformed in the following final/third stage with a CRISPR/Cas9 and appropriate sgRNAs to make knock outs of the native sequences of all six copies of endogenous Mfw2 and of PV3. This creates the final maintainer line with knockouts of the three native homeologues of Mfw2 and PV3—with necessary expression of these genes being effected by the single copies of Mfw2′ and PV3′ at the new locus in the genome. (If all six homoeologues of each gene are not knocked out in one plant, plants with hemizygous knockouts can be selected and, in their progeny, plants/segregates with all knockouts can be selected). See FIG. 32A.

The corresponding male-sterile line can also be created during this step (FIG. 32B). Specifically, when parents were hemizygous, F2 cross progeny are ˜25% of the embryos targeted for knock-out of the endogenous Mfw2 and PV3 will be heterozygous Mfw′:BA/PV′; plants and constitute the maintainer plants. At the same time, ˜50% of the embryos/plants targeted are homozygous null for the insert or hemizygous PV′/−. These plants, having no inserted Mfw′ and with successful knockouts of all endogenous Mfw and PV genes are male-sterile.

Alternatively, where all three genomes' pairs (e.g., all three homoeologue pairs) of each gene are not knocked out in one plant and there are one or more remaining unmutated Mfw and PV alleles (and thus the plant is male-fertile), such plant(s) with hemizygous knockouts can be selected and, in their progeny, plants/segregates with all knockouts can be selected. The progeny with no inserted genes are the male-sterile and those with inserts are the maintainer.

Another possibility for the third stage or as part of the original T-DNA is that the Cas9 can be fused to a reverse transcriptase to allow for prime editing of a desired location in Mfw2 and PV3, thereby creating a premature stop codon. In this instance PCR based KASP primers can be designed to differentiate between the prime edited locations and the inserted Mfw2′ and PV3′ in future generations. Such an approach makes it possible that ‘conversion’ to a maintainer and its isogenic male-sterile can be achieved in two generations.

Accordingly, one or more plants from the above—with inserted Mfw2′:BA2 and PV3′ as an allelic pair and complete knockouts/prime edits of endogenous genes—become a new maintainer-line. Its progeny will be 50% dark-seed-color maintainer and 50% WT-seed-color male-sterile as described elsewhere in this application. As already mentioned above, one or more progeny plants with only one remaining inserted PV3 gene or only one Mfw2:BA1 gene pairs and with all six homoeologous alleles of both Mfw2 and PV3 knocked out/prime edited, these become the relevant matched pair of a male-sterile and a maintainer to pollinate and maintain it.

In summary, in exemplary embodiments, the method comprises:

Step 1. The selected wild-type elite wheat line is transformed with one random-site knockin of a cassette (FIGS. 31C-31D) containing all three genes: Mfw′ linked to a color marker such as BA1 or BA2 and PV′ (e.g., ˜60 kb in total as in FIG. 31D) along with two pairs of ‘cut sequences’ to produce a number of T₀ plants comprising the cassette.

Step 2. A plant with the best expression level of BA1 or BA2 (indicating a good insertional-site) is selected for excision-transformation. Some embryos/seeds from this parent plant are retransformed to excise Mfv′:BA2:nptII and some to excise nptII:PV′ resulting in Step 2 To genotypes as in FIG. 32A. That is, Mfw′:BA2:nptII will be cut out of one offspring and nptII:PV′ cut out of another to leave these two genotypes (FIG. 32A) with ‘allelic’ inserts at homologous loci. A hemizygote or null-insert plant is also selected for Step 3.

Step 3. The two genotypes resulting from Step 2 are crossed. Embryos/plants on the resultant F1 plants (so F2 embryos) are then subjected to CRISPR-Cas knockout of all endogenous sequences of Mfv and PV—see FIG. 32B. A null-insert/Insert hemizygote plant is also selected for knockouts (to become the male-sterile).

It is contemplated that two or three of the above three stages can be combined into one or two larger-number experiment(s).

### REFERENCES

1. Mlynarova L, Conner A J, Nap J P. Directed microspore-specific recombination of transgenic alleles to prevent pollen-mediated transmission of transgenes. Plant Biotechnol J. 2006; 4:445-52.
2. Luo K, Duan H, Zhao D, Zheng X, Deng W, Chen Y, et al. “GM-gene-deletor”: fused loxP-FRT recognition sequences dramatically improve the efficiency of FLP or CRE recombinase on transgene excision from pollen and seed of tobacco plants. Plant Biotechnol J. Plant Biotechnol J; 2007; 5:263-374.
3. Khattri A, Nandy S, Srivastava V. Heat-inducible Cre-lox system for marker excision in transgenic rice. J Biosci. J Biosci; 2011; 36:37-42.
4. Djukanovic V, Lenderts B, Bidney D, Lyznik L A. A Cre::FLP fusion protein recombines FRT or loxP sites in transgenic maize plants. Plant Biotechnol J. 2008; 6:770-81.

### Example 10

The maintainer lines described herein can also be created by means of a process as follows. To create the maintainer line a cassette comprising the genomic sequence of four genes will be introduced at random into the wheat genome of an elite breeding line suitable to become a parent of an F1. The cassette comprises a MF′ gene, a PV′ gene, a seed color gene (or a set of seed color genes), and a selection gene.

This will result, in at least one plant, in strong expression of the introduced genes and complementation of the native sequences of Ms1 and PV3. The introduced genomic DNA sequences of the MF gene (e.g., Ms1) and the PV gene (e.g., PV3) will be modified slightly with subtle DNA-sequence SNP's to allow for next-stage targeting of the native sequences via CRISPR/Cas9 but with no change to the amino acid sequences of the encoded genes (these subtly different genomic sequences (which can be naturally-occurring) are titled Ms1′ and PV3′ hereafter and similar selected/engineered sequences are described in detail elsewhere herein). (As in Example 7, an alternative strategy would be to find, e.g., from an exome sequence database (see, e.g., He, F. et al. Nat. Genet. 2019 515, 51, 896-904; which is incorporated by reference herein in its entirety) a rare natural/endogenous variant whose sequence would then become MF′ or PV′.)

A diagram of the relevant cassette sequence for the initial (insertion stage) transformation is shown in FIGS. 31A-31B. Such cassettes can be T-DNA sequences. The cassette itself comprises, in 5′ to 3′ or 3′ to 5′ order: i) the full genomic sequence of Ms1′ and a seed color marker gene (or at least one functional ectopic allele of each member of a set of seed color genes), for example BA1 (with Ms1′ and BA1 in either order relative to each other); this will be followed by ii) a selection gene, for example nptII, finally followed by iii) the full genomic sequence of PV3′. The cassette can be incorporated into the genome in either orientation with respect to any reference point in the genome. To ensure that both pollen fertility genes are at the same locus in the genome and that they segregate independently after the second transformation, four further sequences can also be included in the modification of the original cassette. These sequences include two 34 bp Cre-lox recognition sequences flanking the start of the Ms1′ sequence and following the nptII selection gene; the other two can be the 34 bp flippase recognition sequences flanking the start of nptII and following PV3′ in order, at the next stage, to recombine out the selection gene and PV3′ [1-4]. It is contemplated that the location of the Cre-Lox and flippase sequences can be reversed. These sequences will be located before the start of selection marker and after the PV3′ genomic sequence. All these are shown in FIGS. 31C-D. An exemplary cassette utilizing PV1′ as the PV gene is provided as SEQ ID NO: 221. An exemplary cassette utilizing PV3′ as the PV gene is provided as SEQ ID NO: 232.

Plants are selected based on a single copy insertion in the genome and plants which have good expression of BA1 at flowering/seed development to prove that the insertion has taken place at an effective locus for these genes to be truly expressed when they need to be and maintain fertility of the plant. Such plants are then, at a second stage, retransformed with either Cre-lox or with flippase to drop out either Ms1′:BA1 (and nptII) to leave just the PV3′ in that/those plants or drop out PV3′ (and nptII) and leave just Ms1′:BA1 in the other plant(s).

Using PCR-based selection (eg with appropriate KASP primers) one or more plants are selected which have just the Ms1′:BA1 remaining and one or more plants are selected which have just the PV3′ remaining. These are crossed to produce F1 embryos which have the allelic pair Ms1′:BA1/PV3′ at the new locus. (If there are no plants with only one or the other inserted allele, hemizygotes can be used but clearly more crosses will need to be made to ensure the correct F1 heterozygous embryos or plants are available for the next stage.)

These embryos are then transformed in the following final/third stage with a CRISPR/Cas9 and appropriate sgRNAs to make knock outs of the native sequences of all eight copies of endogenous Ms1 and of PV3: two Ms1 and six PV3. This creates the final maintainer line with knockouts of the native homeologues of Ms1 and PV3—with necessary expression of these genes being effected by the single copies of Ms1′ and PV3′ at the new locus in the genome. (If both homoeologues of endogenous Ms1 and all six homoeologues of PV3 are not knocked out in one plant, plants with hemizygous knockouts can be selected and, in their progeny, plants/segregates with all knockouts can be selected). See FIG. 32A.

The corresponding male-sterile line can also be created during this step (FIG. 32B). Specifically, when parents were hemizygous, F2 cross progeny are ˜25% of the embryos targeted for knock-out of the endogenous Ms1 and PV3 will be heterozygous Mfw′:BA/PV′; plants and constitute the maintainer plants. At the same time, ˜50% of the embryos targeted are homozygous null for the insert or hemizygous PV′/−. These plants, having no inserted Mfw′ and with successful knockouts of all endogenous Mfv and PV genes are male-sterile.

Alternatively, where all three genomes' relevant pairs (e.g., all three homoeologue pairs of PV3 and both of the single-genome MF (e.g, Ms1)) of each gene are not knocked out in one plant and there are one or more remaining unmutated Mfw and PV alleles (and thus the plant is male-fertile), such plant(s) with hemizygous knockouts can be selected and, in their progeny, plants/segregates with all knockouts can be selected. The progeny with no inserted genes are the male-sterile and those with inserts are the maintainer.

Another possibility for the third stage or as part of the original T-DNA is that the Cas9 can be fused to a reverse transcriptase to allow for prime editing of a desired location in Ms1 and PV3, thereby creating a premature stop codon. In this instance PCR based KASP primers can be designed to differentiate between the prime edited locations and the inserted Ms1′ and PV3′ in future generations. Such an approach makes it possible that ‘conversion’ to a maintainer and its isogenic male-sterile can be achieved in two generations.

Accordingly, one or more plants from the above—with inserted Ms1′:BA1 and PV3′ as an allelic pair and complete knockouts/prime edits of endogenous genes—become a new maintainer-line. Its progeny will be 50% dark-seed-color maintainer and 50% WT-seed-color male-sterile as described elsewhere in this application. As already mentioned above, one or more progeny plants with only one remaining inserted PV3 gene or only one Ms1:BA1 gene pairs and, with both homoeologous alleles of Ms1 and all six of PV3 knocked out/prime edited; these become the relevant matched pair of a male-sterile and a maintainer to pollinate and maintain it.

In summary, in exemplary embodiments, the method comprises:

Step 1. The selected wild-type elite wheat line is transformed with one random-site knockin of a cassette (FIGS. 31C-31D) containing all three genes: Mfw′ linked to a color marker such as BA1 and PV′ (e.g., ˜60 kb in total) along with two pairs of ‘cut sequences’ to produce a number of T₀ plants comprising the cassette.

Step 2. A plant with the best expression level of BA1 (indicating a good insertional-site) is selected for excision-transformation. Some embryos/seeds from this parent plant are retransformed to excise Mfv′:BA:nptII and some to excise nptII:PV′ resulting in Step 2 T₀ genotypes as in FIG. 32A. That is, Mfw′:BA:nptII will be cut out of one offspring and nptII:PV′ cut out of another to leave these two genotypes (FIG. 32A) with ‘allelic’ inserts at homologous loci. A hemizygote or null-insert plant is also selected for Step 3.

Step 3. The two genotypes resulting from Step 2 are crossed. Embryos/plants on the resultant F1 plants (so F2 embryos) are then subjected to CRISPR-Cas knockout of all endogenous sequences of Mfv and PV— see FIG. 32B. A null-insert/Insert hemizygote plant is also selected for knockouts (to become the male-sterile).

It is contemplated that two or three of the above three stages can be combined into one or two larger-number experiment(s).

An exemplary PV3′ comprises a change in the DNA sequence from CCTTCTCCTCCACCGCGGGGCTG (SEQ ID NO: 255) which is the nuclease target in the native PV3 sequence to CCTTATCCTCCACCGCGGGGCTG (SEQ ID NO: 256) which will not change the amino acid sequence of the protein but not allow the guide to target the introgressed PV3. This different/adapted PV3 is titled PV3′0.1 hereafter. The CDS sequence of PV3 is provided as SEQ ID NO: 257 and the CDS of PV3′0.1 is provided as SEQ ID NO: 258. The altered guide RNA target sequence (SEQ ID NO: 256) is found at nucleotides 1,463-1,485 of SEQ ID NO: 258. An examplary guide sequence for use with PV3′0.1 is therefore SEQ ID NO: 255. A maintainer example with Ms1 HMWG:BA1 nptII and PV3/RUPO is provided in SEQ ID NO: 259.

### REFERENCES

1. Mlynárová L, Conner A J, Nap J P. Directed microspore-specific recombination of transgenic alleles to prevent pollen-mediated transmission of transgenes. Plant Biotechnol J. 2006; 4:445-52.
2. Luo K, Duan H, Zhao D, Zheng X, Deng W, Chen Y, et al. “GM-gene-deletor”: fused loxP-FRT recognition sequences dramatically improve the efficiency of FLP or CRE recombinase on transgene excision from pollen and seed of tobacco plants. Plant Biotechnol J. Plant Biotechnol J; 2007; 5:263-374.
3. Khattri A, Nandy S, Srivastava V. Heat-inducible Cre-lox system for marker excision in transgenic rice. J Biosci. J Biosci; 2011; 36:37-42.
4. Djukanovic V, Lenderts B, Bidney D, Lyznik L A. A Cre::FLP fusion protein recombines FRT or loxP sites in transgenic maize plants. Plant Biotechnol J. 2008; 6:770-81.

### Example 11

Targeted Insertion of Cre-Lox Sequence at a PV Site and Subsequent Insertion of MFW′:BA (Eg Mfw2′:BAI or Ms1′:BA1) at that Site.

A targeted insertion of a recombination type sequence, ie Cre-lox (with alternatives being sequences for recombination via flippase or I-SceI), can be inserted at a target location (the gRNA target site) using a Cas9-fusion to VirD to allow anchoring to the target site (Ali et al., 2020). Using this gRNA target site we can incorporate the 68 base pairs necessary for recombination at one or all PV sites, e.g. SEQ ID NO: 232 for PV3.

The 68 base pair Cre-lox sequence with an added right border binding sequence and homologous flanking sequence can be introduced as a phosphorothioate-modified template using modified primers. The template and Cas9 fusion will be introduced into wheat via biolistic integration for maximum copy number integration. By way of non-limiting example, it is contemplated that employing a transient-expression technique such as in-planta particle bombardment (iPB) (Liu Y et al, 2021) with a high efficiency across genotypes and which does not involve an introgressed transgene would be particularly advantageous. Plants can then be screened for the inserted 68 bp sequence and grown on to be retransformed with a T-DNA containing the necessary recombinase sequence followed by Mfw′ (eg Ms1′ or Mfw2′) genomic sequence followed by a sequence encoding BA1. For example, a genomic BA1 sequence or BA1 cds or cDNA. (The individual genes' sequences may incorporated as more than one copy in order to enhance their expression levels). In some embodiments, both genes are driven by their endogenous promoters with appropriate sequences flanking these two wheat genes for recombination at the PV target site. Plants will then be screened for integration of the Mfw′:BA1 sequences at the PV insertion sites using site-specific primers. Those with the insertion at only one copy of the desired PV homoeologue can be selected for next stages. Once this integration is confirmed sites up and downstream of, and within, the PV insertion site as well as within a Mfv known to be editableed by CRISPR will again be used specifically be to targeted as well as other known PV guide targeting sites the specifically to drop out removal of the Cre-lox, flippase or I-SceI sequences used for integration. If employing a technique requiring introduction of T-DNA, all remaining T-DNAs will then be segregated away to create the final edited line in heterozygous (Mfw′:BA/PV) form which can be used as the maintainer and parent of the male-sterile. This integration step allows for the diploid-expressed Mfw′:BA sequence of the maintainer sequences to always be on the sister chromatid to the later-needed, haploid-expressed PV sequence. By setting up how these two genes segregate—as alternative ‘alleles’—we can ensure that the Mfw′:BA maintainer sequence always segregates against that of the PV gene. Furthermore, employing the PV gene in this way means the remaining endogenous alleles of PV3 can be knocked out with guides which are specific to the two genomes whose genes need to be mutated: guide 1 targeting two of the PV3 homeologues using the guide sequence GTATTGAAGAAGTTTTATCAGGG (SEQ ID NO: 253) and guide 2 targeting the endogenous Ms1-B using the guide sequence GGCTCGCAGCACTGCGCCGTCGG (SEQ ID NO: 267), or guide 4 TATATCCTCGGACGGAGAGATGG (SEQ ID NO: 254) [PAMs in bold]). This leaves just the one PV allele which is heterozygously-paired with Mfw′:BA as the only PV allele expressed. This ensures that the 50% of haploid pollen grains with the Mfw′:BA allele cannot also have the PV allele and so cannot germinate and fertilise any egg cells.)

### REFERENCES

Ali, Z., Shami, A., Sedeek, K., Kamel, R., Alhabsi, A., Tehseen, M., et al. (2020) Fusion of the Cas9 endonuclease and the VirD2 relaxase facilitates homology-directed repair for precise genome engineering in rice. Commun. Biol. 2020 31, 3, 1-13. Yuelin Liu, Weifeng Luo, Qianyan Linghu, Fumitaka Abe, Hiroshi Hisano,
Kazuhiro Sato, Yoko Kamiya, Kanako Kawaura, Kazumitsu Onishi, Masaki Endo, Seiichi Toki, Haruyasu Hamada, Yozo Nagira, Naoaki Taoka and Ryozo Imai (2012). In planta Genome Editing in Commercial Wheat Varieties. Front. Plant Sci. 12:648841.

1. A male-fertile maintainer plant for a male-sterile polyploid plant comprising:
a first genome comprising:
on a first chromosome of a pair of homologous chromosomes, at a single target locus, at least one functional ectopic allele of a MF gene and at least one functional ectopic allele of a seed color gene;
on a second chromosome of the pair of homologous chromosomes, at the target locus corresponding to the target locus of the first chromosome of the pair of homologous chromosomes, at least one functional ectopic allele of a PV gene;
loss-of-function alleles of the endogenous MF genes at the native MF gene loci and loss-of-function alleles of the endogenous PV genes at the native PV gene loci.

2.-7. (canceled)

8. A male-fertile maintainer plant for a male-sterile polyploid plant comprising:
a first genome comprising:
on a first chromosome of a pair of homologous chromosomes, at least one functional allele of a MF gene at the MF gene locus and at least one allele of a seed color gene;
on a second chromosome of the pair of homologous chromosomes, a loss-of-function allele of the MF gene at the MF gene locus and at least one ectopic functional allele of a PV gene;
and loss-of-function alleles of the PV gene at the native PV gene loci; and
at least one further genome, each of the further genomes comprising loss-of-function alleles of the MF gene at the native MF gene loci and loss-of-function alleles of the PV gene at the native PV gene loci.

9. The plant of claim 8, wherein the at least one functional allele of a MF gene is the endogenous wild-type functional allele of the MF gene or an ectopic copy of the MF gene.

10. (canceled)

11. The plant of claim 10, wherein the at least one functional allele of a MF gene and the at least one allele of a seed color gene are part of single construct.

12. The plant of claim 8, wherein an ectopic allele or ectopic copy of a gene is a nuclease-null or CRISPR-null allele.

13. The plant of claim 8, wherein the MF gene;
displays the same type of activity and shares at least 95% sequence identity with one or more of the genes of Table 1; Mfw2; or Ms1; or is selected from Table 1; Mfw2; and Ms1.

14.-18. (canceled)

19. The plant of claim 8, wherein the PV gene:
displays the same type of activity and shares at least 95% sequence identity with one or more of the genes of Table 2, PV3, PV1, or PV2; or
is selected from Table 2, PV3, PV1, and PV2.

20.-30. (canceled)

31. The plant of claim 8, wherein the at least one allele of a seed color gene is at least one of:
a seed coat gene;
a seed endosperm gene; and
exogenous.

32. The plant of claim 8, wherein the at least one allele of a seed color gene is blue aleurone (BA).

33. (canceled)

34. The plant of claim 8, wherein at least one of:
the at least one allele of a seed color gene and the at least one ectopic functional allele of a PV gene
is located within 10 cM of the MF gene loci.

35.-37. (canceled)

38. The plant of claim 8, wherein the only exogenous sequence in the genomes is the at least one allele of a seed color gene; or wherein the only ectopic sequence in the genomes is the at least one ectopic functional allele of a PV gene.

39. (canceled)

40. The plant of claim 8, wherein the plant is tetraploid and the second genome comprises loss-of-function alleles of the MF gene at the native MF gene loci and loss-of-function alleles of the PV gene at the native PV gene loci; or wherein the plant is hexaploid and the second and third genomes both comprise loss-of-function alleles of the MF gene at the native MF gene loci and loss-of-function alleles of the PV gene at the native PV gene loci.

41. (canceled)

42. The plant of claim 8, wherein a loss-of-function allele is at least one of:
a) an allele comprising an engineered knock-out modification;
b) an allele comprising an engineered excision of at least part of a coding sequence or regulatory sequence; or
c) engineered using a site-specific guided nuclease.

43. (canceled)

44. (canceled)

45. The plant of claim 42, wherein the site-specific guided nuclease is a form of CRISPR-Cas.

46. The plant of claim 8, wherein the plant is wheat, triticale, canola/oilseed rape, indian mustard, barley, rice, oat, or rye.

47. (canceled)

48. The plant of claim 40, wherein the at least one allele of a seed color gene comprises a sequence from T. aestivum, T durum, T. monococcum or another Triticum aestivum-crossable species.

49. The plant of claim 46, wherein the plant is hexaploid wheat or tetraploid wheat, Triticum aestivum, or Triticum durum.

50. A method of preparing a male-fertile maintainer plant for a male-sterile polyploid plant, the method comprising engineering a plant to comprise:
in a first genome:
on a first chromosome of a pair of homologous chromosomes, at a single target locus, at least one functional ectopic allele of a MF gene and at least one functional ectopic allele of a seed color gene;
on a second chromosome of the pair of homologous chromosomes, at the target locus corresponding to the target locus of the first chromosome of the pair of homologous chromosomes, at least one functional ectopic allele of a PV gene; and
loss-of-function alleles of the endogenous MF genes at the native MF gene loci and loss-of-function alleles of the endogenous PV genes at the native PV gene loci.

51. A method of preparing a male-fertile maintainer plant for a male-sterile polyploid plant, the method comprising, simultaneously or sequentially:
inserting, on a first chromosome of a pair of homologous chromosomes in a first genome, at a single target locus, a construct comprising at least one functional ectopic allele of a MF gene and at least one functional ectopic allele of a seed color gene, optionally wherein the inserting comprises nuclease cleavage of the target locus and recombination or end-joining of the construct;
inserting, on a second chromosome of the pair of homologous chromosomes in the first genome, at the target locus corresponding to the target locus of the first chromosome of the pair of homologous chromosomes, a construct comprising at least one functional ectopic allele of a PV gene, optionally wherein the inserting comprises nuclease cleavage of the target locus and/or recombination or end-joining of the construct; and
mutating the the endogenous MF genes at the native MF gene loci and the endogenous PV genes at the native PV gene loci to create loss-of-function alleles, optionally wherein the loss-of-function alleles are caused by contacting the genome with a site-specific guided nuclease and one or more guide RNA sequences or multi-guide constructs.

52. A method of preparing a male-fertile maintainer plant for a male-sterile polyploid plant, the method comprising:
i. inserting, on a first chromosome of a pair of homologous chromosomes in a first genome, at a single target locus, a cassette comprising in 5′ to 3′ or 3′ to 5′ order:
a first recognition site for a first recombinase;
at least one functional ectopic nuclease null allele of a MF gene and at least one functional ectopic allele of a seed color gene in either relative order;
a first recognition site for a second recombinase;
a selection gene;
a second recognition site for the first recombinase;
at least one functional ectopic nuclease-null allele of a PV gene;
a second recognition site for the second recombinase;
thereby providing a full-cassette insertion plant;
ii. contacting a first progeny of the full-cassette insertion plant, or a cell thereof, with the first recombinase,
thereby excising:
one recognition site for the first recombinase, the at least one functional ectopic nuclease null allele of a MF gene and at least one functional ectopic allele of a seed color gene, the first recognition site for the second recombinase, and the selection gene from the genome of the first progeny and
thereby providing an excised first progeny comprising:
one recognition site for the first recombinase, the at least one functional ectopic nuclease-null allele of a PV gene, and the second recognition site for the second recombinase portions of the construct;
iii. contacting a second progeny of the full-cassette insertion plant, or a cell thereof, with the second recombinase,
thereby excising:
one recognition site for the second recombinase, the selection gene, the second recognition site for the first recombinase and at least one functional ectopic nuclease-null allele of a PV gene, and
thereby providing an excised second progeny comprising:
one recognition site for the second recombinase, the first recognition site for the first recombinase, and the at least one functional ectopic nuclease null allele of a MF gene and at least one functional ectopic allele of a seed color gene portions of the construct;
iv. crossing the excised first progeny provided in step ii) and the excised second progeny provided in step iii), thereby providing a third progeny comprising, in a first genome,
on a first chromosome of a pair of homologous chromosomes, at a single target locus, the at least one functional ectopic nuclease-null allele of a MF gene and the at least one functional ectopic allele of a seed color gene;
on a second chromosome of the pair of homologous chromosomes, at the target locus corresponding to the target locus of the first chromosome of the pair of homologous chromosomes, the at least one functional ectopic nuclease-null allele of a PV gene; and
v. mutating the endogenous MF genes at the native MF gene loci and the endogenous PV genes at the native PV gene loci to create loss-of-function alleles, optionally wherein the loss-of-function alleles are caused by contacting the genome with a site-specific guided nuclease and one or more guide RNA sequences or multi-guide constructs, thereby providing the male-fertile maintainer plant.

53.-59. (canceled)

60. A method of preparing a male-fertile maintainer plant for a male-sterile polyploid plant, the method comprising:
i. contacting a cell comprising a PV locus in a first chromosome and a second chromosome of a pair of homologous chromosomes in a first genome, with:
1. a site-specific guided nuclease;
2. one or more guide RNA sequences or multi-guide constructs specific to one or more sequences at the PV locus; and
3. an targeting insertion cassette comprising in 5′ to 3′ or 3′ to 5′ order: a first recognition site for a first recombinase and a second recognition site for the first recombinase;
thereby providing a targeting insertion plant;
ii. contacting the targeting insertion plant, or first progeny of the targeting insertion plant, or a cell thereof with the first recombinase and a cassette comprising in 5′ to 3′ or 3′ to 5′ order:
1) a first recombination site for the first recombinase;
2) at least one functional ectopic nuclease null allele of a MF gene and at least one functional ectopic allele of a seed color gene in either relative order; and
3) a second recombination site for the first recombinase;
thereby providing a cassette insertion plant;
iii. selecting a cassette insertion plant comprising a cassette insertion at one allele of the PV locus, or crossing a cassette insertion plant comprising a cassette insertion at both alleles of the PV locus with a plant with a functional PV allele at the PV locus,
thereby providing a cassette insertion plant with a cassette insertion at one PV allele in the first genome and a functional PV allele at the second PV allele in the first genome,
iv. contacting the cassette insertion plant selected in iii), or a first progeny or cell thereof, with:
1. a site-specific guided nuclease (e.g., CRISPR);
2. one or more guide RNA sequences or multi-guide constructs flanking the insertion sites, thereby excising the inserted recombination sites;
3. one or more guide RNA sequences or multi-guide constructs specific to the functional endogenous MF genes and/or flanking the functional endogenous MF genes, thereby mutating the functional endogenous MF genes at the functional native MF gene loci to create loss-of-function alleles;
thereby providing the male-fertile maintainer plant.

61.-112. (canceled)

113. A method of providing a male sterile plant seed, the method comprising selecting, from seed produced by selfing a plant of claim 8, seed not displaying a phenotype provided by the seed endosperm gene.

114. A method of providing male sterile plant seed, the method comprising selfing a plant of claim 8, whereby the resulting seed not displaying a phenotype provided by the seed endosperm gene is the male sterile plant seed.

115. A method of providing a F1 hybrid seed for crop production, the method comprising collecting the seed produced by a male-sterile plant pollinated by a male-fertile plant, wherein the male-sterile plant is
a) a plant grown from male sterile plant seed obtained by the method of claim 60; and/or
b) comprises:
i) loss-of-function alleles of an endogenous MF gene at each of the native MF gene loci;
ii) loss-of-function alleles of an endogenous PV gene at each of the native PV gene loci; and
iii) two ectopic alleles of the PV gene at a target locus.

116. A method of providing a F1 hybrid seed for crop production, the method comprising crossing a male-sterile plant with a male-fertile plant, wherein the male-sterile plant is
a) a plant grown from male sterile plant seed obtained by the method of claim 113; and/or
b) comprises:
i) loss-of-function alleles of an endogenous MF gene at each of the native MF gene loci;
ii) loss-of-function alleles of an endogenous PV gene at each of the native PV gene loci; and
iii) two ectopic alleles of the PV gene at a target locus.

117. A method of providing a F1 hybrid seed for crop production, the method comprising planting a male-sterile plant within pollination range of a male-fertile plant, wherein the male-sterile plant is
a) a plant grown from male sterile plant seed obtained by the method of claim 113; and/or
b) comprises:
i) loss-of-function alleles of an endogenous MF gene at each of the native MF gene loci;
ii) loss-of-function alleles of an endogenous PV gene at each of the native PV gene loci; and
iii) two ectopic alleles of the PV gene at a target locus; and
whereby the male-fertile plant pollinates the male-sterile plant and F1 hybrid seed is produced.

118. (canceled)

119. (canceled)

120. A method of producing a plant crop, the method comprising:
a. planting and/or harvesting a plant or portion thereof, wherein the plant:
i. is plant grown from F1 hybrid seed obtained by the method of claim 115; and/or
ii. comprises:
1) in each genome of the plant, at a native MF gene locus, one functional endogenous allele of the endogenous MF gene and one loss-of-function allele of the endogenous MF gene;
2) in each genome of the plant, at a native PV gene locus, one functional endogenous allele of the endogenous PV gene and one loss-of-function allele of the endogenous PV gene;
3) one ectopic allele of the PV gene at a target locus.
